(19) United States
(12) Patent Application Publication
(10) Pub. No.: US 2010/0130471 A1
(43) Pub. Date: May 27, 2010

(54) Renin Inhibitors

(76) Inventors: John J. Baldwin, Gwynedd Valley, PA (US); Salvacion Cacatian, Blue Bell, PA (US); David Claremon, Maple Glen, PA (US); Lawrence W. Dillard, Yardley, PA (US); Patrick T. Flaherty, Pittsburgh, PA (US); Bahman Ghavimi-Alagha, Wilmington, DE (US); Damiamo Ghirlanda, Legnago (IT); Xiaoping Hou, Pennington, NJ (US); Alexey V. Ishebenko, Somerville, MA (US); Lara S. Kallander, King of Prussia, PA (US); Beth A. Knapp-Reed, King of Prussia, PA (US); Brian Lawhorn, King of Prussia, PA (US); Qing Lu, King of Prussia, PA (US); Gerard McGeehan, Garnet Valley, PA (US); Simon Semus, Collegeville, PA (US); Robert D. Simpson, Wilmington, DE (US); Suresh B. Singh, Kendall Park, NJ (US); Lamont R. Terrell, King of Prussia, PA (US); Colin Tice, Ambler, PA (US); Tritin Tran, King of Prussia, PA (US); Zhenrong Xu, Horsham, PA (US); Jing Yuan, Lansdale, PA (US); Jing Zhang, Waltham, MA (US); Wei Zhao, Eagleville, PA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/450,619

(22) PCT Filed: Apr. 4, 2008
(86) PCT No.: PCT/US08/59399

§ 371 Date: Feb. 2, 2010

Related U.S. Application Data
(60) Provisional application No. 60/910,224, filed on Apr. 5, 2007.

(51) Int. Cl. A61K 31/445; A61K 31/554; A61K 31/55; A61K 31/551; A61K 31/549; A61K 31/5377; A61K 31/5375; A61K 31/4545; C07D 417/12; C07D 265/30; C07D 401/06; C07D 401/10; C07D 211/34; A61P 25/00; A61P 9/12; A61P 9/10

(52) U.S. Cl. 514/211.07; 514/212.07; 514/221; 514/223.5; 514/235.5; 514/236.2; 514/237.5; 514/316; 514/318; 514/330; 544/133; 544/165; 546/189; 546/194; 546/226

(57) ABSTRACT

Described are compounds which bind to aspartic proteases to inhibit their activity. They are useful in the treatment or amelioration of diseases associated with aspartic protease activity. Also described are methods of use of the compounds described herein in ameliorating or treating aspartic protease related disorders in a subject in need thereof.

### BACKGROUND OF THE INVENTION

Aspartic proteases, including renin, β-secretase (BACE), Candida albicans secreted aspartyl proteases, HIV protease, HTLV protease and plasmepsins I and II, are implicated in a number of disease states. In hypertension elevated levels of angiotensin I, the product of renin catalyzed cleavage of angioteninogen are present. Elevated levels of βamyloid, the product of BACE activity on amyloid precursor protein, are widely believed to be responsible for the amyloid plaques present in the brains of Alzheimer's disease patients. Secreted aspartyl proteases play a role in the virulence of the pathogen Candida albicans. The viruses HIV and HTLV depend on their respective aspartic proteases for viral maturation. Plasmodium falciparum uses plasmepsins I and II to degrade hemoglobin.

In the renin-angiotensin-aldosterone system (RAAS) the biologically active peptide angiotensin II (Ang II) is generated by a two-step mechanism. The highly specific aspartic protease renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAAS represents a major advance in the treatment of cardiovascular diseases (Zaman, M. A. et al Nature Reviews Drug Discovery 2002, 1, 621-636). ACE inhibitors and AT₁ blockers have been accepted as treatments of hypertension (Waeber B. et al., “The renin-angiotensin system: role in experimental and human hypertension”, in Berkenhager W. H., Reid J. L. (eds): Hypertension, Amsterdam, Elsevier Science Publishing Co, 1996, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N Engl. J: Med, 1992, 327, 669).

Interest in the development of renin inhibitors stems from the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be bypassed by chymase, a serine protease (Husain A., J. Hypertens., 1993, 11, 1155). In patients, inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). Chymase is not inhibited by ACE inhibitors. Therefore, the formation of Ang II is still possible in patients treated with ACE inhibitors. Blockade of the ATI receptor (e.g., by losartan) on the other hand overexposes other AT-receptor subtypes to Ang II, whose concentration is dramatically increased by the blockade of ATI receptors. In summary, renin inhibitors are not only expected to be superior to ACE inhibitors and AT₁ blockers with regard to safety, but more importantly also with regard to their efficacy in blocking the RAAS.

Only limited clinical experience (Azizi M. et al., J. Hypertens., 1994, 12, 419; Neutel J. M. et al., Am. Heart, 1991, 122, 1094) has been generated with renin inhibitors because their peptidomimetic character imparts insufficient oral activity (Kleinert H. D., Cardiovasc. Drugs, 1995, 9, 645). The clinical development of several compounds has been stopped because of this problem together with the high cost of goods. It appears as though only one compound has entered clinical trials (Rahuel J. et al., Chem. Biol., 2000, 7, 493; Mealy N. E., Drugs of the Future, 2001, 26, 1139). Thus, metabolically stable, orally bioavailable and sufficiently soluble renin inhibitors that can be prepared on a large scale are not available. Recently, the first non-peptide renin inhibitors were described which show high in vitro activity (Oefner C. et al., Chem. Biol., 1999, 6, 127; Patent Application WO 97/09311; Maerki H. P. et al., II Farmaco, 2001, 56, 21). The present invention relates to the unexpected identification of renin inhibitors of a non-peptidic nature and of low molecular weight. Orally active renin inhibitors which are active in indications beyond blood pressure regulation where the tissular renin-chymase system may be activated leading to pathophysiologically altered local functions such as renal, cardiac and vascular remodeling, atherosclerosis, and restenosis, are described.

All documents cited herein are incorporated by reference.

### SUMMARY OF THE INVENTION

Compounds have now been found which are orally active and bind to aspartic proteases to inhibit their activity. They are useful in the treatment or amelioration of diseases associated with aspartic protease activity.

It will be appreciated by those skilled in the art, that the compounds of this invention contain 1, 2 or more chiral centers and may exist in different enantiomeric and/or diastereomeric forms. The following compounds are recited without reference to the relative or absolute configuration of any of the chiral centers present therein, but such recitation is intended to encompass each enantiomeric and/or diastereomeric form of these compounds and all mixtures thereof, such as enantiomerically and/or diastereomerically enriched mixtures and racemic mixtures. The following are compounds of the invention:

| Cpd. No. | Name |
|---|---|
| I-1 | methyl 4-(1-(3-aminocyclopentanecarbonyl)piperidin-3-yl)-4-hydroxy- |
| | 4-(2-(pyridin-4-yl)phenyl)butylcarbamate |
| I-2 | 2-((4-(3-aminocyclopentanecarbonyl)morpholin-2-yl)(6-fluoro-3′- |
| | methylbiphenyl-2-yl)methoxy)-N-ethylacetamide |
| I-3 | N-(4-(1-(3-aminocyclopentanecarbonyl)piperidin-3-yl)-4-hydroxy-4- |
| | (2-(o-tolyloxy)phenyl)butyl)acetamide |
| I-4 | (3-aminocyclopentyl)(3-(1-(2-(2,6-dimethylphenoxy)phenyl)-1- |
| | hydroxy-5-methoxypentyl)piperidin-1-yl)methanone |
| I-5 | methyl 4-(1-(3-amino-4-hydroxycyclopentanecarbonyl)piperidin-3-yl)- |
| | 4-hydroxy-4-(2-(pyridin-3-yl)phenyl)butylcarbamate |
| I-6 | (3-amino-4-hydroxycyclopentyl)(2-(1-(6-chloro-3′-ethylbiphenyl-2-yl)- |
| | 1-hydroxypent-4-enyl)morpholino)methanone |
| I-7 | methyl 2-((4-(3-aminocyclopentanecarbonyl)morpholin-2-yl)(6-fluoro- |
| | 3′-methylbiphenyl-2-yl)methoxy)ethylcarbamate |
| I-8 | 2-((4-(3-amino-4-hydroxycyclopentanecarbonyl)morpholin-2-yl)(6- |
| | fluoro-3′-methylbiphenyl-2-yl)methoxy)-N-ethylacetamide |
| I-9 | (3-aminocyclopentyl)(2-(1-(4′,6-difluoro-3′-methylbiphenyl-2-yl)-1- |
| | hydroxy-5-methoxypentyl)morpholino)methanone |
| I-10 | (3-amino-4-hydroxycyclopentyl)(2-(1-(3-chloro-2-(pyridin-3- |
| | yl)phenyl)-1-hydroxy-5-methoxypentyl)morpholino)methanone |
| I-11 | (3-amino-4-hydroxycyclopentyl)(2-(1-(3-chloro-2-(3-methyl-1,2,4- |
| | oxadiazol-5-yl)phenyl)-1-hydroxy-5-methoxypentyl)morpholino)methanone |
| I-12 | methyl 4-(1-(3-aminocyclopentanecarbonyl)piperidin-3-yl)-4-hydroxy- |
| | 4-(2-(o-tolyloxy)phenyl)butylcarbamate |
| I-13 | N-(4-(1-(3-amino-4-hydroxycyclopentanecarbonyl)piperidin-3-yl)-4- |
| | hydroxy-4-(2-(o-tolyloxy)phenyl)butyl)acetamide |
| I-14 | (3-amino-4-hydroxycyclopentyl)(3-(1-(2-(2,6- |
| | dimethylphenoxy)phenyl)-1-hydroxy-5-methoxypentyl)piperidin-1- |
| | yl)methanone |
| I-15 | (3-aminocyclopentyl)(3-(1-(6-fluoro-3′-methoxy-5′-methylbiphenyl-2- |
| | yl)-1-hydroxy-5-methoxypentyl)piperidin-1-yl)methanone |
| I-16 | (3-aminocyclopentyl)(3-(1-(6-chloro-3′-ethylbiphenyl-2-yl)-1-hydroxy- |
| | 5-methoxypentyl)piperidin-1-yl)methanone |
| I-17 | (3-aminocyclopentyl)(3-(1-(3-chloro-2-(2-methylbenzyl)phenyl)-1- |
| | hydroxy-5-methoxypentyl)piperidin-1-yl)methanone |
| I-18 | (3-aminocyclopentyl)(3-(1-(3-chloro-2-(3-methylbenzyl)phenyl)-1- |
| | hydroxy-5-methoxypentyl)piperidin-1-yl)methanone |
| I-19 | (3-aminocyclopentyl)(2-(1-(6-fluoro-3′-methoxy-5′-methylbiphenyl-2- |
| | yl)-1-hydroxy-5-methoxypentyl)morpholino)methanone |
| I-20 | (3-aminocyclopentyl)(2-(1-(6-chloro-3′-ethylbiphenyl-2-yl)-1-hydroxy- |
| | 5-methoxypentyl)morpholino)methanone |
| I-21 | (3-aminocyclopentyl)(3-(1-(3-chloro-2-(o-tolyloxy)phenyl)-1-hydroxy- |
| | 5-methoxypentyl)piperidin-1-yl)methanone |
| I-22 | (3-aminocyclopentyl)(3-(1-(2-(2-chloro-6-methylphenoxy)phenyl)-1- |
| | hydroxy-5-methoxypentyl)piperidin-1-yl)methanone |
| I-23 | (3-amino-4-hydroxycyclopentyl)(2-(1-(4′,6-difluoro-3′-methylbiphenyl- |
| | 2-yl)-1-hydroxy-5-methoxypentyl)morpholino)methanone |
| I-24 | methyl 4-(1-(3-amino-4-hydroxycyclopentanecarbonyl)piperidin-3-yl)- |
| | 4-hydroxy-4-(2-(o-tolyloxy)phenyl)butylcarbamate |
| I-25 | methyl 4-(1-(2-aminocyclopentanecarbonyl)piperidin-3-yl)-4-(3′-ethyl- |
| | 6-fluorobiphenyl-2-yl)-4-hydroxybutylcarbamate |
| I-26 | methyl 4-(1-(3-aminocyclopentanecarbonyl)piperidin-3-yl)-4-(3′-ethyl- |
| | 6-fluorobiphenyl-2-yl)-4-hydroxybutylcarbamate |
| I-27 | (4-aminocyclohexyl)(3-(1-(6-chloro-3′-ethylbiphenyl-2-yl)-1-hydroxy- |
| | 5-methoxypentyl)piperidin-1-yl)methanone |
| I-29 | (3-amino-4-hydroxycyclopentyl)(3-(1-(6-fluoro-3′-methoxy-5′- |
| | methylbiphenyl-2-yl)-1-hydroxy-5-methoxypentyl)piperidin-1- |
| | yl)methanone |
| I-28 | 1-(1-{[6-(aminomethyl)-3-pyridinyl]carbonyl}-3-piperidinyl)-1-(6- |
| | chloro-3′-ethyl-2-biphenylyl)-5-(methyloxy)-1-pentanol |
| I-30 | (3-amino-4-hydroxycyclopentyl)(3-(1-(6-chloro-3′-ethylbiphenyl-2-yl)- |
| | 1-hydroxy-5-methoxypentyl)piperidin-1-yl)methanone |
| I-31 | (3-aminocyclopentyl)(3-(1-(3-chloro-2-(2-ethylphenoxy)phenyl)-1- |
| | hydroxy-5-methoxypentyl)piperidin-1-yl)methanone |
| I-32 | (3-aminocyclopentyl)(3-(1-(3-chloro-2-(3-ethylphenoxy)phenyl)-1- |
| | hydroxy-5-methoxypentyl)piperidin-1-yl)methanone |
| I-33 | (3-amino-4-hydroxycyclopentyl)(3-(1-(3-chloro-2-(2- |
| | methylbenzyl)phenyl)-1-hydroxy-5-methoxypentyl)piperidin-1- |
| | yl)methanone |
| I-34 | (3-amino-4-hydroxycyclopentyl)(3-(1-(3-chloro-2-(3- |
| | methylbenzyl)phenyl)-1-hydroxy-5-methoxypentyl)piperidin-1- |
| | yl)methanone |
| I-35 | (3-amino-4-hydroxycyclopentyl)(2-(1-(6-fluoro-3′-methoxy-5′- |
| | methylbiphenyl-2-yl)-1-hydroxy-5-methoxypentyl)morpholino)methanone |
| I-36 | (3-amino-4-hydroxycyclopentyl)(3-(1-(3-chloro-2-(o-tolyloxy)phenyl)- |
| | 1-hydroxy-5-methoxypentyl)piperidin-1-yl)methanone |
| I-37 | (3-amino-4-hydroxycyclopentyl)(2-(1-(6-chloro-3′-ethylbiphenyl-2-yl)- |
| | 1-hydroxy-5-methoxypentyl)morpholino)methanone |
| I-38 | (3-amino-4-hydroxycyclopentyl)(2-(1-(6-chloro-2′-fluoro-5′- |
| | methylbiphenyl-2-yl)-1-hydroxy-5-methoxypentyl)morpholino)methanone |
| I-39 | (3-aminocyclopentyl)(3-(1-(3-chloro-2-(quinolin-3-yl)phenyl)-1- |
| | hydroxy-5-methoxypentyl)piperidin-1-yl)methanone |
| I-40 | (3-aminocyclopentyl)(2-(1-(3-chloro-2-(naphthalen-2-yl)phenyl)-1- |
| | hydroxy-5-methoxypentyl)morpholino)methanone |
| I-41 | (3-aminocyclopentyl)(2-(1-(3-chloro-2-(quinolin-3-yl)phenyl)-1- |
| | hydroxy-5-methoxypentyl)morpholino)methanone |
| I-42 | methyl 4-hydroxy-4-(1-{[3-(methylamino)cyclopentyl]carbonyl}-3- |
| | piperidinyl)-4-{2-[(2-methylphenyl)oxy]phenyl}butyl)carbamate |
| I-43 | methyl 4-(6-fluoro-3′-methoxybiphenyl-2-yl)-4-hydroxy-4-(1-(3- |
| | (methylamino)cyclopentanecarbonyl)piperidin-3-yl)butylcarbamate |
| I-44 | N-(4-(1-(3-amino-4-hydroxycyclopentanecarbonyl)piperidin-3-yl)-4- |
| | (6-chloro-3′-ethylbiphenyl-2-yl)-4-hydroxybutyl)acetamide |
| I-45 | methyl 4-(1-(3-aminocyclopentanecarbonyl)piperidin-3-yl)-4-(6- |
| | chloro-3′-ethylbiphenyl-2-yl)-4-hydroxybutylcarbamate |
| I-46 | methyl 4-(4-(3-aminocyclopentanecarbonyl)morpholin-2-yl)-4-(6- |
| | chloro-3′-ethylbiphenyl-2-yl)-4-hydroxybutylcarbamate |
| I-47 | (3-amino-4-hydroxycyclopentyl)(3-(1-(3-chloro-2-(3- |
| | ethylphenoxy)phenyl)-1-hydroxy-5-methoxypentyl)piperidin-1- |
| | yl)methanone |
| I-48 | (3-amino-4-hydroxycyclopentyl)(2-(1-(6-fluoro-3′,5′- |
| | dimethoxybiphenyl-2-yl)-1-hydroxy-5- |
| | methoxypentyl)morpholino)methanone |
| I-49 | (3-amino-4-hydroxycyclopentyl)(2-(1-(6-chloro-3′- |
| | (methoxymethyl)biphenyl-2-yl)-1-hydroxy-5- |
| | methoxypentyl)morpholino)methanone |
| I-50 | (4-(aminomethyl)cyclohexyl)(2-(1-(6-chloro-2′-fluoro-5′- |
| | methylbiphenyl-2-yl)-1-hydroxy-5-methoxypentyl)morpholino)methanone |
| I-51 | (3-amino-4-hydroxycyclopentyl)(3-(1-(3-chloro-2-(quinolin-3- |
| | yl)phenyl)-1-hydroxy-5-methoxypentyl)piperidin-1-yl)methanone |
| I-52 | (3-amino-4-hydroxycyclopentyl)(2-(1-(3-chloro-2-(naphthalen-2- |
| | yl)phenyl)-1-hydroxy-5-methoxypentyl)morpholino)methanone |
| I-53 | (3-amino-4-hydroxycyclopentyl)(2-(1-(3-chloro-2-(quinolin-3- |
| | yl)phenyl)-1-hydroxy-5-methoxypentyl)morpholino)methanone |
| I-54 | (3-amino-4-hydroxycyclopentyl)(2-(1-(3-chloro-2-(isoquinolin-4- |
| | yl)phenyl)-1-hydroxy-5-methoxypentyl)morpholino)methanone |
| I-55 | methyl 4-(6-chloro-3′-ethylbiphenyl-2-yl)-4-hydroxy-4-(1-(3- |
| | (methylamino)cyclopentanecarbonyl)piperidin-3-yl)butylcarbamate |
| I-56 | methyl 4-(1-(3-aminocyclopentanecarbonyl)piperidin-3-yl)-4-(6- |
| | chloro-3′-isopropylbiphenyl-2-yl)-4-hydroxybutylcarbamate |
| I-57 | methyl 4-(1-(3-amino-4-hydroxycyclopentanecarbonyl)piperidin-3-yl)- |
| | 4-(6-chloro-3′-ethylbiphenyl-2-yl)-4-hydroxybutylcarbamate |
| I-58 | methyl 4-(6-chloro-3′-methoxybiphenyl-2-yl)-4-hydroxy-4-(1-(3- |
| | (methylamino)cyclopentanecarbonyl)piperidin-3-yl)butylcarbamate |
| I-59 | (3-amino-4-hydroxycyclopentyl)(2-(1-(6-chloro-3′,5′- |
| | dimethoxybiphenyl-2-yl)-1-hydroxy-5- |
| | methoxypentyl)morpholino)methanone |
| I-60 | methyl 4-(1-(4-(aminomethyl)cyclohexanecarbonyl)piperidin-3-yl)-4- |
| | (6-chloro-3′-ethylbiphenyl-2-yl)-4-hydroxybutylcarbamate |
| I-61 | methyl 4-(1-(3-amino-4-hydroxycyclopentanecarbonyl)piperidin-3-yl)- |
| | 4-(6-chloro-3′-isopropylbiphenyl-2-yl)-4-hydroxybutylcarbamate |
| I-62 | (3-aminocyclopentyl)(3-(1-(3′-ethoxy-6-fluoro-5′- |
| | (trifluoromethyl)biphenyl-2-yl)-1-hydroxy-5-methoxypentyl)piperidin-1- |
| | yl)methanone |
| I-63 | methyl 4-(1-(3-amino-4-hydroxycyclopentanecarbonyl)piperidin-3-yl)- |
| | 4-(3-chloro-2-(quinolin-3-yl)phenyl)-4-hydroxybutylcarbamate |
| I-64 | (3-aminocyclopentyl)(2-(1-(3′-ethoxy-6-fluoro-5′- |
| | (trifluoromethyl)biphenyl-2-yl)-1-hydroxy-5- |
| | methoxypentyl)morpholino)methanone |
| I-65 | (3-amino-4-hydroxycyclopentyl)(3-(1-(3′-ethoxy-6-fluoro-5′- |
| | (trifluoromethyl)biphenyl-2-yl)-1-hydroxy-5-methoxypentyl)piperidin-1- |
| | yl)methanone |
| I-66 | (3-amino-4-hydroxycyclopentyl)(2-(1-(3′-ethoxy-6-fluoro-5′- |
| | (trifluoromethyl)biphenyl-2-yl)-1-hydroxy-5- |
| | methoxypentyl)morpholino)methanone |
| I-67 | (3-aminocyclopentyl)(2-(1-(6-fluoro-3′,5′-dimethoxybiphenyl-2-yl)-1- |
| | hydroxy-5-methoxypentyl)morpholino)methanone |
| I-68 | (3-aminocyclopentyl)(2-(1-(3-chloro-2-(isoquinolin-4-yl)phenyl)-1- |
| | hydroxy-5-methoxypentyl)morpholino)methanone |
| I-69 | methyl (4-(1-{[3-aminocyclopentyl]carbonyl}-3-piperidinyl)-4-{3- |
| | chloro-2-[(2-methylphenyl)oxy]phenyl}-4-hydroxybutyl)carbamate |
| I-70 | methyl (4-(1-{[3-aminocyclopentyl]carbonyl}-3-piperidinyl)-4-{3- |
| | chloro-2-[(2-ethylphenyl)oxy]phenyl}-4-hydroxybutyl)carbamate |
| I-71 | methyl (4-(1-{[3-amino-4-hydroxycyclopentyl]carbonyl}-3- |
| | piperidinyl)-4-{3-chloro-2-[(2-methylphenyl)oxy]phenyl}-4- |
| | hydroxybutyl)carbamate |
| I-72 | methyl (4-(1-{[3-amino-4-hydroxycyclopentyl]carbonyl}-3- |
| | piperidinyl)-4-{3-chloro-2-[(2-ethylphenyl)oxy]phenyl}-4- |
| | hydroxybutyl)carbamate |
| I-73 | methyl [4-(1-{[3-amino-4-hydroxycyclopentyl]carbonyl}-3- |
| | piperidinyl)-4-(3′-ethyl-6-fluoro-2-biphenylyl)-4-hydroxybutyl]carbamate |
| I-74 | methyl {4-(6-chloro-3′-ethyl-2-biphenylyl)-4-hydroxy-4-[1-({trans-4- |
| | [(methylamino)methyl]cyclohexyl}carbonyl)-3- |
| | piperidinyl]butyl}carbamate |
| I-75 | methyl [4-(1-{[2-aminocyclopentyl]carbonyl}-3-piperidinyl)-4-(3′- |
| | ethyl-6-fluoro-2-biphenylyl)-4-hydroxybutyl]carbamate |
| I-76 | 1-(4-{[6-(aminomethyl)-3-pyridinyl]carbonyl}-2-morpholinyl)-1-(6- |
| | chloro-3′-ethyl-2-biphenylyl)-5-(methyloxy)-1-pentanol |
| I-77 | methyl [4-(1-{[6-(aminomethyl)-3-pyridinyl]carbonyl}-3-piperidinyl)- |
| | 4-(6-chloro-3′-ethyl-2-biphenylyl)-4-hydroxybutyl]carbamate |
| I-78 | 1-{1-[(cis-4-aminocyclohexyl)acetyl]-3-piperidinyl}-1-(6-chloro-3′- |
| | ethyl-2-biphenylyl)-5-(methyloxy)-1-pentanol |
| I-79 | 1-{1-[(trans-4-aminocyclohexyl)acetyl]-3-piperidinyl}-1-(6-chloro-3′- |
| | ethyl-2-biphenylyl)-5-(methyloxy)-1-pentanol |
| I-80 | 1-(1-{[-2-aminocyclopentyl]carbonyl}-3-piperidinyl)-1-(6-chloro-3′- |
| | ethyl-2-biphenylyl)-5-(methyloxy)-1-pentanol |
| I-81 | 1-(6-chloro-3′-ethyl-2-biphenylyl)-1-(4-{[3- |
| | (methylamino)cyclopentyl]carbonyl}-2-morpholinyl)-5-(methyloxy)-1- |
| | pentanol |
| I-82 | 1-(6-chloro-3′-ethyl-2-biphenylyl)-1-(1-{[3- |
| | (methylamino)cyclopentyl]carbonyl}-3-piperidinyl)-5-(methyloxy)-1- |
| | pentanol |
| I-83 | 4-({2-[1-(6-chloro-3′-ethyl-2-biphenylyl)-1-hydroxy-5- |
| | (methyloxy)pentyl]-4-morpholinyl}carbonyl)-2-[(2- |
| | hydroxyethyl)amino]cyclopentanol |
| I-84 | methyl {4-(6-chloro-3′-ethyl-2-biphenylyl)-4-hydroxy-4-{1-({3- |
| | hydroxy-4-[(2-hydroxyethyl)amino]cyclopentyl}carbonyl)-3- |
| | piperidinyl]butyl}carbamate |
| I-85 | 1-(6-chloro-3′-ethyl-2-biphenylyl)-1-[1-({trans-4- |
| | [(methylamino)methyl]cyclohexyl}carbonyl)-3-piperidinyl]-5-(methyloxy)- |
| | 1-pentanol |
| I-86 | 4-({2-[1-(6-chloro-3′-ethyl-2-biphenylyl)-1-hydroxy-5- |
| | (methyloxy)pentyl]-4-morpholinyl}carbonyl)-2-[(1,3-thiazol-2- |
| | ylmethyl)amino]cyclopentanol |
| I-87 | methyl [4-(1-{[3-aminocyclohexyl]carbonyl}-3-piperidinyl)-4-(6- |
| | chloro-3′-ethyl-2-biphenylyl)-4-hydroxybutyl]carbamate |
| I-88 | N-{4-(6-chloro-3′-methyl-2-biphenylyl)-4-hydroxy-4-[1-({trans-4- |
| | [(methylamino)methyl]cyclohexyl}carbonyl)-3-piperidinyl]butyl}acetamide |
| I-89 | N-[4-(6-chloro-3′-methyl-2-biphenylyl)-4-hydroxy-4-(1-{[3- |
| | (methylamino)cyclopentyl]carbonyl}-3-piperidinyl)butyl]acetamide |
| I-90 | 1-(6-chloro-3′-ethyl-2-biphenylyl)-1-{4-[(3-{[(2,5-dimethyl-1,3- |
| | oxazol-4-yl)methyl]amino}cyclopentyl)carbonyl]-2-morpholinyl}-5- |
| | (methyloxy)-1-pentanol |
| I-91 | N-[4-(1-{[trans-4-(aminomethyl)cyclohexyl]carbonyl}-3-piperidinyl)- |
| | 4-(6-chloro-3′-methyl-2-biphenylyl)-4-hydroxybutyl]acetamide |
| I-92 | methyl {4-(4,6-difluoro-3′-methyl-2-biphenylyl)-4-hydroxy-4-[1- |
| | ({trans-4-[(methylamino)methyl]cyclohexyl}carbonyl)-3- |
| | piperidinyl]butyl}carbamate |
| I-93 | methyl [4-hydroxy-4-(1-{[3-(methylamino)cyclopentyl]carbonyl}-3- |
| | piperidinyl)-4-(2′,4,6-trifluoro-5′-methyl-2-biphenylyl)butyl]carbamate |
| I-94 | methyl [4-hydroxy-4-[1-({trans-4- |
| | [(methylamino)methyl]cyclohexyl}carbonyl)-3-piperidinyl]-4-(2′,4,6- |
| | trifluoro-5′-methyl-2-biphenylyl)butyl]carbamate |
| I-95 | methyl [4-(6-chloro-3′-methyl-2-biphenylyl)-4-hydroxy-4-(1-{[3- |
| | (methylamino)cyclopentyl]carbonyl}-3-piperidinyl)butyl]carbamate |
| I-96 | 1-(6-chloro-3′-ethyl-2-biphenylyl)-1-[4-({trans-4- |
| | [(methylamino)methyl]cyclohexyl}carbonyl)-2-morpholinyl]-5- |
| | (methyloxy)-1-pentanol |
| I-97 | methyl [4-(4-{[3-amino-4-hydroxycyclopentyl]carbonyl}-2- |
| | morpholinyl)-4-(3′-ethyl-6-fluoro-2-biphenylyl)-4-hydroxybutyl]carbamate |
| I-98 | methyl {4-(3′-ethyl-6-fluoro-2-biphenylyl)-4-hydroxy-4-[4-({trans-4- |
| | [(methylamino)methyl]cyclohexyl}carbonyl)-2- |
| | morpholinyl]butyl}carbamate |
| I-99 | 2-amino-4-({2-[1-[2-chloro-3-(3-ethylphenyl)-4-pyridinyl]-1-hydroxy- |
| | 5-(methyloxy)pentyl]-4-morpholinyl}carbonyl)cyclopentanol |
| I-100 | 1-(4-{[3-aminocyclopentyl]carbonyl}-2-morpholinyl)-1-[2-chloro-3-(3- |
| | ethylphenyl)-4-pyridinyl]-5-(methyloxy)-1-pentanol |
| I-101 | 2-amino-4-({2-[1-[2-(1-benzothien-3-yl)-3-chlorophenyl]-1-hydroxy-5- |
| | (methyloxy)pentyl]-4-morpholinyl}carbonyl)cyclopentanol |
| I-102 | 1-(4-{[3-aminocyclopentyl]carbonyl}-2-morpholinyl)-1-[2-(1- |
| | benzothien-3-yl)-3-chlorophenyl]-5-(methyloxy)-1-pentanol |
| I-103 | 1-[4-chloro-3-(3-ethylphenyl)-2-pyridinyl]-1-(4-{[3- |
| | (methylamino)cyclopentyl]carbonyl}-2-morpholinyl)-5-(methyloxy)-1- |
| | pentanol |
| I-104 | methyl {4-[2-chloro-3-(3-ethylphenyl)-4-pyridinyl]-4-hydroxy-4-[1- |
| | ({trans-4-[(methylamino)methyl]cyclohexyl}carbonyl)-3- |
| | piperidinyl]butyl}carbamate |
| I-105 | methyl [4-[2-chloro-3-(3-ethylphenyl)-4-pyridinyl]-4-hydroxy-4-(1- |
| | {[3-(methylamino)cyclopentyl]carbonyl}-3-piperidinyl)butyl]carbamate |
| I-106 | methyl {4-{3-chloro-2-[4-(1-methylethyl)-2-quinazolinyl]phenyl}-4- |
| | hydroxy-4-[1-({trans-4-[(methylamino)methyl]cyclohexyl}carbonyl)-3- |
| | piperidinyl]butyl}carbamate |
| I-107 | methyl [4-{3-chloro-2-[4-(1-methylethyl)-2-quinazolinyl]phenyl}-4- |
| | hydroxy-4-(1-{[3-(methylamino)cyclopentyl]carbonyl}-3- |
| | piperidinyl)butyl]carbamate |
| I-108 | 1-(1-{[(1R,3S)-3-aminocyclopentyl]carbonyl}-3-piperidinyl)-1-{3- |
| | chloro-2-[(3-methylphenyl)oxy]phenyl}-5-(methyloxy)-1-pentanol |
| I-109 | 2-amino-4-({3-[1-{3-chloro-2-[(3-methylphenyl)oxy]phenyl}-1- |
| | hydroxy-5-(methyloxy)pentyl]-1-piperidinyl}carbonyl)cyclopentanol |
| I-110 | methyl (4-(1-{[3-aminocyclopentyl]carbonyl}-3-piperidinyl)-4-{3- |
| | chloro-2-[(3-methylphenyl)oxy]phenyl}-4-hydroxybutyl)carbamate |
| I-111 | methyl (4-(1-{[3-amino-4-hydroxycyclopentyl]carbonyl}-3- |
| | piperidinyl)-4-{3-chloro-2-[(3-methylphenyl)oxy]phenyl}-4- |
| | hydroxybutyl)carbamate |
| I-112 | methyl (4-(1-{[3-aminocyclopentyl]carbonyl}-3-piperidinyl)-4-{2- |
| | [(2,6-dimethylphenyl)oxy]phenyl}-4-hydroxybutyl)carbamate |
| I-113 | methyl (4-(1-{[3-amino-4-hydroxycyclopentyl]carbonyl}-3- |
| | piperidinyl)-4-{2-[(2,6-dimethylphenyl)oxy]phenyl}-4- |
| | hydroxybutyl)carbamate |
| I-114 | methyl {4-{3-chloro-2-[8-(1-methylethyl)-2-quinolinyl]phenyl}-4- |
| | hydroxy-4-[1-({trans-4-[(methylamino)methyl]cyclohexyl}carbonyl)-3- |
| | piperidinyl]butyl}carbamate |
| I-115 | methyl {4-[3-chloro-2-(8-methyl-2-quinolinyl)phenyl]-4-hydroxy-4-[1- |
| | ({trans-4-[(methylamino)methyl]cyclohexyl}carbonyl)-3- |
| | piperidinyl]butyl}carbamate |
| I-116 | methyl [4-[3-fluoro-2-(3-quinolinyl)phenyl]-4-hydroxy-4-(1-{[3- |
| | (methylamino)cyclopentyl]carbonyl}-3-piperidinyl)butyl]carbamate |
| I-117 | methyl [4-{2-chloro-3-[3-(1-methylethyl)phenyl]-4-pyridinyl}-4- |
| | hydroxy-4-(1-{[3-(methylamino)cyclopentyl]carbonyl}-3- |
| | piperidinyl)butyl]carbamate |
| I-118 | methyl [4-[3-chloro-2-(5-methyl-2-furanyl)phenyl]-4-hydroxy-4-(1- |
| | {[3-(methylamino)cyclopentyl]carbonyl}-3-piperidinyl)butyl]carbamate |
| I-119 | methyl {4-1-{[3-amino-4-hydroxycyclopentyl]carbonyl}-3- |
| | piperidinyl)-4-[3-chloro-2-(5-methyl-2-furanyl)phenyl]-4- |
| | hydroxybutyl}carbamate |
| I-120 | 1-{2-chloro-3-[3-(1-methylethyl)phenyl]-4-pyridinyl}-1-[4-({trans-4- |
| | [(methylamino)methyl]cyclohexyl}carbonyl)-2-morpholinyl]-5- |
| | (methyloxy)-1-pentanol |
| I-121 | 1-{2-chloro-3-[3-(1-methylethyl)phenyl]-4-pyridinyl}-1-(4-{[3- |
| | (methylamino)cyclopentyl]carbonyl}-2-morpholinyl)-5-(methyloxy)-1- |
| | pentanol |
| I-122 | 2-amino-4-({2-[1-{2-chloro-3-[3-(1-methylethyl)phenyl]-4-pyridinyl}- |
| | 1-hydroxy-5-(methyloxy)pentyl]-4-morpholinyl}carbonyl)cyclopentanol |
| I-123 | methyl {4-[5-chloro-4-(3-ethylphenyl)-3-pyridinyl]-4-hydroxy-4-[1- |
| | ({trans-4-[(methylamino)methyl]cyclohexyl}carbonyl)-3- |
| | piperidinyl]butyl}carbamate |
| I-124 | methyl [4-[5-chloro-4-(3-ethylphenyl)-3-pyridinyl]-4-hydroxy-4-(1- |
| | {[3-(methylamino)cyclopentyl]carbonyl}-3-piperidinyl)butyl]carbamate |
| I-125 | methyl {4-(5-chloro-4-[3-(1-methylethyl)phenyl]-3-pyridinyl}-4- |
| | hydroxy-4-[1-({trans-4-[(methylamino)methyl]cyclohexyl}carbonyl)-3- |
| | piperidinyl]butyl}carbamate |
| I-126 | methyl [4-{5-chloro-4-[3-(1-methylethyl)phenyl]-3-pyridinyl}-4- |
| | hydroxy-4-(1-{[3-(methylamino)cyclopentyl]carbonyl}-3- |
| | piperidinyl)butyl]carbamate |
| I-127 | methyl {4-[6-fluoro-3′-(1-methylethyl)-2-biphenylyl]-4-hydroxy-4-{1- |
| | ({trans-4-[(methylamino)methyl]cyclohexyl}carbonyl)-3- |
| | piperidinyl]butyl}carbamate |
| I-128 | methyl [4-(6-chloro-3′-fluoro-5′-methyl-2-biphenylyl)-4-hydroxy-4-(1- |
| | {[3-(methylamino)cyclopentyl]carbonyl}-3-piperidinyl)butyl]carbamate |
| I-129 | methyl [4-(3′,6-difluoro-5′-methyl-2-biphenylyl)-4-hydroxy-4-(1-{[3- |
| | (methylamino)cyclopentyl]carbonyl}-3-piperidinyl)butyl]carbamate |
| I-130 | 2-amino-4-({3-[1-{3-chloro-2-[(2-ethylphenyl)oxy]phenyl}-1-hydroxy- |
| | 5-(methyloxy)pentyl]-1-piperidinyl}carbonyl)cyclopentanol |
| I-131 | methyl {4-(3′-ethyl-6-fluoro-2-biphenylyl)-4-hydroxy-4-[1-([trans-4- |
| | [(methylamino)methyl]cyclohexyl}carbonyl)-3- |
| | piperidinyl]butyl}carbamate |
| I-132 | 2-amino-4-({3-[1-{2-[(2-chloro-6-methylphenyl)oxy]phenyl}-1- |
| | hydroxy-5-(methyloxy)pentyl]-1-piperidinyl}carbonyl)cyclopentanol |
| I-133 | 2-amino-4-({3-[1-{3-fluoro-2-[(3-methylphenyl)oxy]phenyl}-1- |
| | hydroxy-5-(methyloxy)pentyl]-1-piperidinyl}carbonyl)cyclopentanol |
| I-134 | 1-(1-{[3-aminocyclopentyl]carbonyl}-3-piperidinyl)-1-{3-fluoro-2-[(3- |
| | methylphenyl)oxy]phenyl}-5-(methyloxy)-1-pentanol |
| I-135 | methyl (4-(1-{[3-amino-4-hydroxycyclopentyl]carbonyl}-3- |
| | piperidinyl)-4-{3-chloro-2-[(3-ethylphenyl)oxy]phenyl}-4- |
| | hydroxybutyl)carbamate |
| I-136 | methyl (4-(1-{[3-aminocyclopentyl]carbonyl}-3-piperidinyl)-4-{3- |
| | chloro-2-[(3-ethylphenyl)oxy]phenyl}-4-hydroxybutyl)carbamate |
| I-137 | methyl (4-(1-{[3-amino-4-hydroxycyclopentyl]carbonyl}-3- |
| | piperidinyl)-4-{3-fluoro-2-[(3-methylphenyl)oxy]phenyl}-4- |
| | hydroxybutyl)carbamate |
| I-138 | methyl (4-(1-{[3-aminocyclopentyl]carbonyl}-3-piperidinyl)-4-{3- |
| | fluoro-2-[(3-methylphenyl)oxy]phenyl}-4-hydroxybutyl)carbamate |
| I-139 | methyl (4-(1-{[3-amino-4-hydroxycyclopentyl]carbonyl}-3- |
| | piperidinyl)-4-{3-chloro-2-[(3-methylphenyl)methyl]phenyl}-4- |
| | hydroxybutyl)carbamate |
| I-140 | methyl [4-{3-chloro-2-[(3-methylphenyl)methyl]phenyl}-4-hydroxy-4- |
| | (1-{[3-(methylamino)cyclopentyl]carbonyl}-3-piperidinyl)butyl]carbamate |
| I-141 | methyl (4-(1-{[3-aminocyclopentyl]carbonyl}-3-piperidinyl)-4-{3- |
| | chloro-2-[(3-methylphenyl)methyl]phenyl}-4-hydroxybutyl)carbamate |
| I-142 | methyl (2-{[(4-{[3-amino-4-hydroxycyclopentyl]carbonyl}-2- |
| | morpholinyl)(6-chloro-3′-ethyl-2-biphenylyl)methyl]oxy}ethyl)carbamate |
| I-143 | methyl (2-{[(1-{[3-amino-4-hydroxycyclopentyl]carbonyl}-3- |
| | piperidinyl)(6-chloro-3′-ethyl-2-biphenylyl)methyl]oxy}ethyl)carbamate |
| I-144 | methyl [4-(1-{[4-(aminomethyl)-5-methyl-2-furanyl]carbonyl}-3- |
| | piperidinyl)-4-(6-chloro-3′-ethyl-2-biphenylyl)-4-hydroxybutyl]carbamate |
| I-145 | methyl {4-(6-chloro-3′-ethyl-2-biphenylyl)-4-hydroxy-4-[1-({6- |
| | [(methylamino)methyl]-3-pyridinyl}carbonyl)-3- |
| | piperidinyl]butyl}carbamate |
| I-146 | methyl [4-{1-[(2-amino-4-oxo-1,4-dihydro-5-pyrimidinyl)carbonyl]-3- |
| | piperidinyl}-4-(6-chloro-3′-methyl-2-biphenylyl)-4- |
| | hydroxybutyl]carbamate |
| I-147 | methyl [4-(6-chloro-3′-methyl-2-biphenylyl)-4-hydroxy-4-(1-{[2- |
| | (methylamino)-5-pyrimidinyl]carbonyl}-3-piperidinyl)butyl]carbamate |
| I-148 | methyl [4-(1-{[5-(aminomethyl)-3-isoxazolyl]carbonyl}-3-piperidinyl)- |
| | 4-(6-chloro-3′-methyl-2-biphenylyl)-4-hydroxybutyl]carbamate |
| I-149 | methyl {4-(6-chloro-3′-ethyl-2-biphenylyl)-4-hydroxy-4-[1-({5- |
| | [(methylamino)methyl]-2-thienyl}carbonyl)-3-piperidinyl]butyl}carbamate |
| I-150 | methyl [4-{1-[(6-amino-3-pyridinyl)carbonyl]-3-piperidinyl}-4-(3′,6- |
| | difluoro-5′-methyl-2-biphenylyl)-4-hydroxybutyl]carbamate |
| I-151 | methyl [2-({(6-chloro-3′-ethyl-2-biphenylyl)[1-({trans-4- |
| | [(methylamino)methyl]cyclohexyl}carbonyl)-3- |
| | piperidinyl]methyl}oxy)ethyl]carbamate |
| I-152 | methyl [2-({(6-chloro-3′-ethyl-2-biphenylyl)[-4-({trans-4- |
| | [(methylamino)methyl]cyclohexyl}carbonyl)-2- |
| | morpholinyl]methyl}oxy)ethyl]carbamate |
| I-153 | N-[4-(1-{[6-(aminomethyl)-3-pyridinyl]carbonyl}-3-piperidinyl)-4-(6- |
| | chloro-3′-methyl-2-biphenylyl)-4-hydroxybutyl]acetamide |
| I-154 | methyl [4-(1-{[4-(aminomethyl)-1-piperidinyl]carbonyl}-3- |
| | piperidinyl)-4-(3′-ethyl-6-fluoro-2-biphenylyl)-4-hydroxybutyl]carbamate |
| I-155 | methyl {4-(3′-ethyl-6-fluoro-2-biphenylyl)-4-hydroxy-4-[1-((4- |
| | [(methylamino)methyl]-1-piperidinyl}carbonyl)-3- |
| | piperidinyl]butyl}carbamate |
| I-156 | methyl [4-(1-{[4-(aminomethyl)-1-piperidinyl]carbonyl}-3- |
| | piperidinyl)-4-(6-chloro-3′-ethyl-2-biphenylyl)-4-hydroxybutyl]carbamate |
| I-157 | methyl [4-(1-{[4-(aminomethyl)-4-phenyl-1-piperidinyl]carbonyl}-3- |
| | piperidinyl)-4-(6-chloro-3′-ethyl-2-biphenylyl)-4-hydroxybutyl]carbamate |
| I-158 | N-[4-(1-{[4-(aminomethyl)-1-piperidinyl]carbonyl}-3-piperidinyl)-4- |
| | (2′,6-difluoro-5′-methyl-2-biphenylyl)-4-hydroxybutyl]-2- |
| | hydroxyacetamide |
| I-159 | methyl {4-(6-chloro-3′-ethyl-2-biphenylyl)-4-hydroxy-4-[1-({4- |
| | [(methylamino)methyl]-1-piperidinyl}carbonyl)-3- |
| | piperidinyl]butyl}carbamate |
| I-160 | methyl {4-(6-chloro-3′-ethyl-2-biphenylyl)-4-hydroxy-4-[1-({4-[(2- |
| | propen-1-ylamino)methyl]-1-piperidinyl}carbonyl)-3- |
| | piperidinyl]butyl}carbamate |

or a diastereomer, enantiomer or salt thereof.

In another embodiment the present invention is directed to pharmaceutical compositions comprising a compound described herein or enantiomers, diastereomers, or salts thereof and a pharmaceutically acceptable carrier or excipient.

In another embodiment the present invention is directed to a method of inhibiting an aspartic protease in a subject in need thereof comprising administering to the subject a therapeutically effective amount of a compound described herein or an enantiomer, diastereomer, or salt thereof.

In another embodiment the present invention is directed to method for treating or ameliorating an aspartic protease mediated disorder in a subject in need thereof comprising administering to said subject a therapeutically effective amount of a compound described herein or an enantiomer, diastereomer, or salt thereof.

In another embodiment the present invention is directed to a method for treating or ameliorating a renin mediated disorder in a subject in need thereof comprising administering to the subject an effective amount of a compound described herein or an enantiomer, diastereomer, or salt thereof.

In another embodiment the present invention is directed to a method for the treatment of hypertension in a subject in need thereof comprising administering to the subject a compound described herein in combination therapy with one or more additional agents said additional agent selected from the group consisting of α-blockers, β-blockers, calcium channel blockers, diuretics, angiotensin converting enzyme (ACE) inhibitors, dual ACE and neutral endopeptidase (NEP) inhibitors, angiotensin-receptor blockers (ARBs), aldosterone synthase inhibitors, aldosterone-receptor antagonists, and endothelin receptor antagonists.

### DETAILED DESCRIPTION OF THE INVENTION

A description of embodiments of the compounds of the invention follows. It will be appreciated by those skilled in the art that each enantiomer and diastereomer of the compounds of this invention will likely demonstrate a different level of effectiveness of inhibiting the action of aspartic proteases, particularly renin. It will be further appreciated that for the most active compounds, all or most of the enantiomers and/or diastereomers may demonstrate some level of activity, but that for compounds with lower activity, certain enantiomers and/or diastereomers may demonstrate such low levels of activity as to be considered inactive. It is understood that the following represent the preferred relative and absolute configuration of the compounds of the invention. It will be appreciated that each of the different enantiomeric and/or diastereomeric forms of the compounds of this invention, including the stereoisomeric forms depicted below, may be separately obtained using conventional procedures (e.g. stereospecific synthesis or resolution via chiral chromatography, crystallization, etc.).

| Cpd. | | |
|---|---|---|
| No. | Structure | Name |
| I-1a | 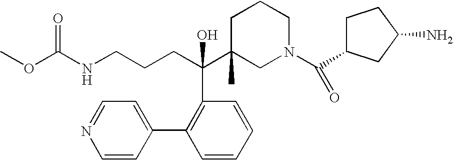 | methyl (S)-4-((R)-1-((1R,3S)-3- aminocyclopentanecarbonyl)piperidin- 3-yl)-4-hydroxy-4-(2-(pyridin-4- yl)phenyl)butylcarbamate |
| I-2a | 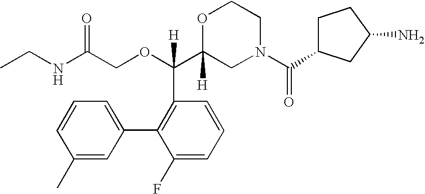 | 2-((S)-((R)-4-((1R,3S)-3- aminocyclopentanecarbonyl)morpholin- 2-yl)(6-fluoro-3′-methylbiphenyl-2- yl)methoxy)-N-ethylacetamide |
| I-3a | 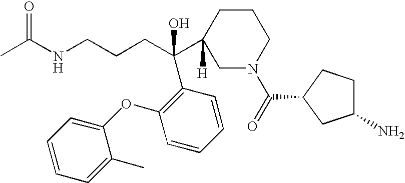 | N-((S)-4-((R)-1-((1R,3S)-3- aminocyclopentanecarbonyl)piperidin- 3-yl)-4-hydroxy-4-(2-(o- tolyloxy)phenyl)butyl)acetamide |
| I-4a | 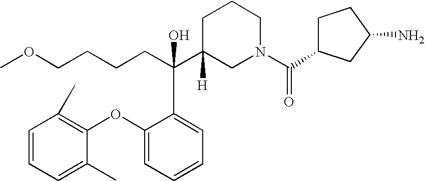 | ((1R,3S)-3-aminocyclopentyl) ((R)- 3-((S)-1-(2-(2,6-dimethyl phenoxy)phenyl)-1-hydroxy-5- methoxypentyl)piperidin-1- yl)methanone |
| I-5a | 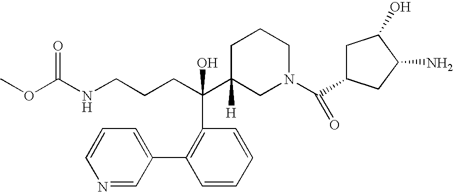 | methyl (S)-4-((R)-1-((1S,3R,4S)-3- amino-4-hydroxycyclopentane carbonyl) piperidin-3-yl)-4-hydroxy-4- (2-(pyridin-3-yl)phenyl)butylcarbamate |
| I-6a | 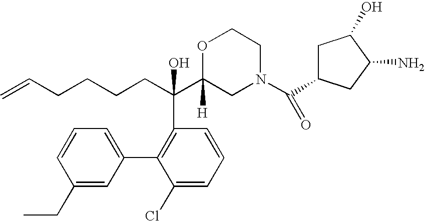 | ((1S,3R,4S)-3-amino-4- hydroxycyclopentyl)((R)-2-((R)-1-(6- chloro-3′-ethylbiphenyl-2-yl)-1- hydroxypent-4-enyl)morpholino) methanone |
| I-7a | 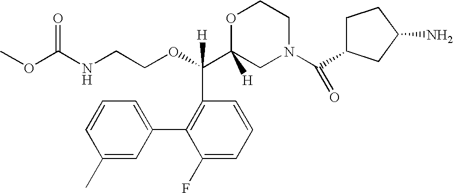 | methyl 2-((S)-((R)-4-((1R,3S)-3- aminocyclopentanecarbonyl) morpholin-2-yl)(6-fluoro-3′- methylbiphenyl-2-yl)methoxy) ethylcarbamate |
| I-8a | 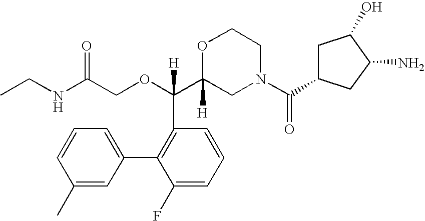 | 2-((S)-((R)-4-((1S,3R,4S)-3-amino- 4-hydroxycyclopentane carbonyl)morpholin-2-yl)(6-fluoro-3′- methylbiphenyl-2-yl)methoxy)-N- ethylacetamide |
| I-9a | 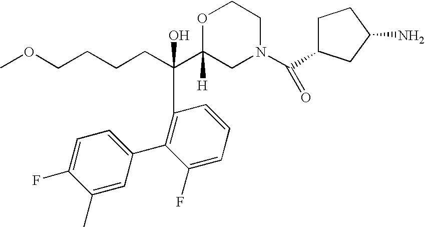 | ((1R,3S)-3-aminocyclopentyl) ((R)- 2-((R)-1-(4′,6-difluoro-3′- methylbiphenyl-2-yl)-1-hydroxy-5- methoxypentyl)morpholino) methanone |
| I-10a | 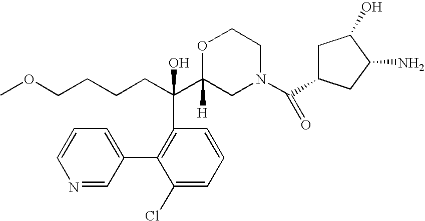 | ((1S,3R,4S)-3-amino-4- hydroxycyclopentyl)((R)-2-((R)-1-(3- chloro-2-(pyridin-3-yl)phenyl)-1- hydroxy-5-methoxypentyl) morpholino)methanone |
| I-11a | 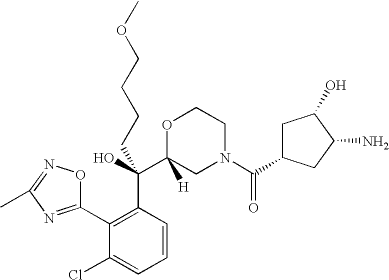 | ((1S,3R,4S)-3-amino-4- hydroxycyclopentyl)((R)-2-((R)-1-(3- chloro-2-(3-methyl-1,2,4-oxadiazol-5- yl)phenyl)-1-hydroxy-5- methoxypentyl)morpholino) methanone |
| I-12a | 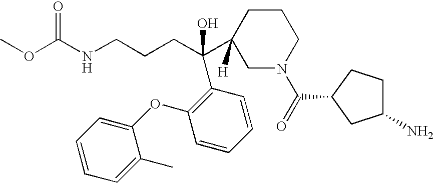 | methyl (S)-4-((R)-1-((1R,3S)-3- aminocyclopentanecarbonyl)piperidin- 3-yl)-4-hydroxy-4-(2-(o- tolyloxy)phenyl)butylcarbamate |
| I-13a | 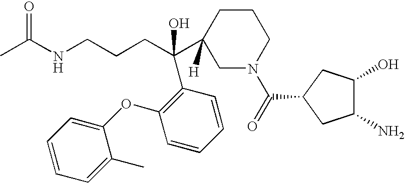 | N-((S)-4-((R)-1-((1S,3R,4S)-3- amino-4-hydroxycyclopentane carbonyl)piperidin-3-yl)-4-hydroxy-4- (2-(o-tolyloxy)phenyl)butyl) acetamide |
| I-14a | 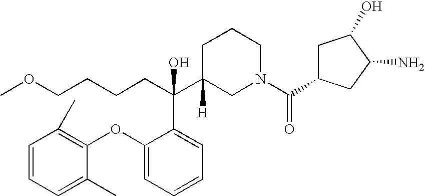 | ((1S,3R,4S)-3-amino-4- hydroxycyclopentyl)((R)-3-((S)-1-(2- (2,6-dimethylphenoxy)phenyl)-1- hydroxy-5-methoxypentyl) piperidin-1- yl)methanone |
| I-15a | 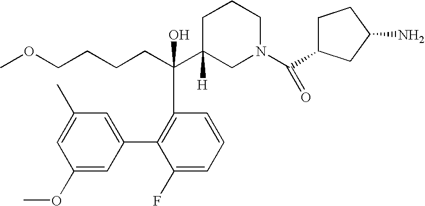 | ((1R,3S)-3-aminocyclopentyl)((R)- 3-((S)-1-(6-fluoro-3′-methoxy-5′- methylbiphenyl-2-yl)-1-hydroxy-5- methoxypentyl)piperidin-1- yl)methanone |
| I-16a | 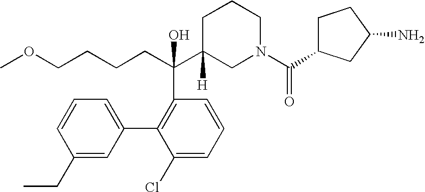 | ((1R,3S)-3-aminocyclopentyl)((R)- 3-((S)-1-(6-chloro-3′-ethylbiphenyl-2- yl)-1-hydroxy-5-methoxypentyl) piperidin-1-yl)methanone |
| I-17a | 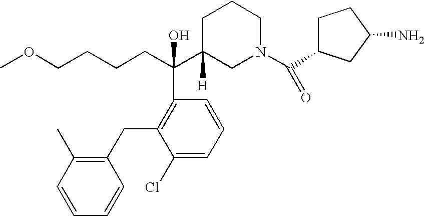 | ((1R,3S)-3-aminocyclopentyl)((R)- 3-((S)-1-(3-chloro-2-(2- methylbenzyl)phenyl)-1-hydroxy-5- methoxypentyl) piperidin-1- yl)methanone |
| I-18a | 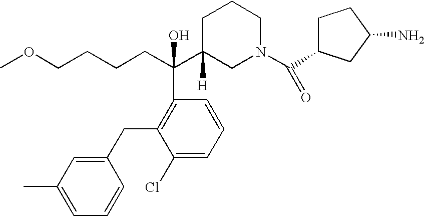 | ((1R,3S)-3-aminocyclopentyl)((R)- 3-((S)-1-(3-chloro-2-(3- methylbenzyl)phenyl)-1-hydroxy-5- methoxypentyl) piperidin-1- yl)methanone |
| I-19a | 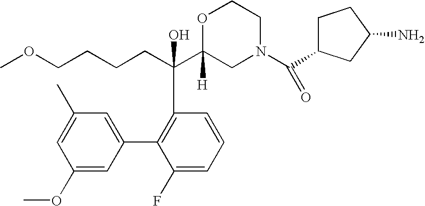 | ((1R,3S)-3-aminocyclopentyl)((R)- 2-((R)-1-(6-fluoro-3′-methoxy-5′- methylbiphenyl-2-yl)-1-hydroxy-5- methoxypentyl)morpholino)methanone |
| I-20a | 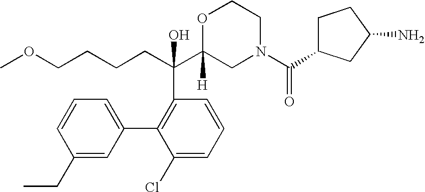 | ((1R,3S)-3-aminocyclopentyl)((R)- 2-((R)-1-(6-chloro-3′-ethylbiphenyl-2- yl)-1-hydroxy-5-methoxypentyl) morpholino) methanone |
| I-21a | 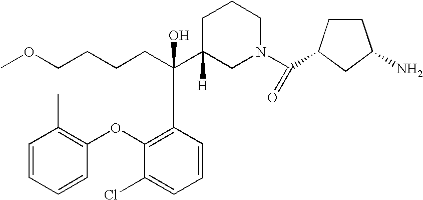 | ((1R,3S)-3-aminocyclopentyl)((R)- 3-((S)-1-(3-chloro-2-(o- tolyloxy)phenyl)-1-hydroxy-5- methoxypentyl) piperidin-1- yl)methanone |
| I-22a | 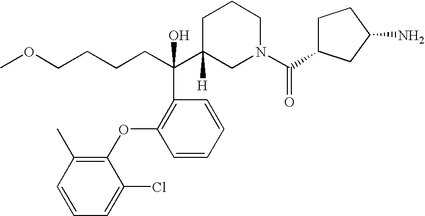 | ((1R,3S)-3-aminocyclopentyl)((R)- 3-((S)-1-(2-(2-chloro-6-methylphenoxy) phenyl)-1-hydroxy-5- methoxypentyl)piperidin-1- yl)methanone |
| I-23a | 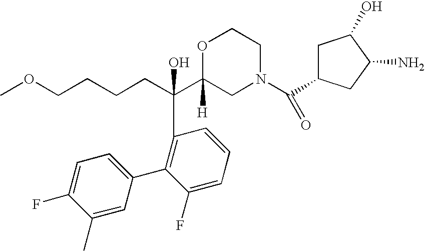 | ((1S,3R,4S)-3-amino-4- hydroxycyclopentyl)((R)-2-((R)-1-(4′,6- difluoro-3′-methylbiphenyl-2-yl)-1- hydroxy-5-methoxypentyl) morpholino) methanone |
| I-24a | 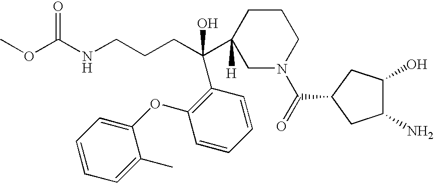 | methyl (S)-4-((R)-1-((1S,3R,4S)-3- amino-4-hydroxycyclopentanecarbonyl) piperidin-3-yl)-4-hydroxy-4-(2-(o- tolyloxy)phenyl)butylcarbamate |
| I-25a | 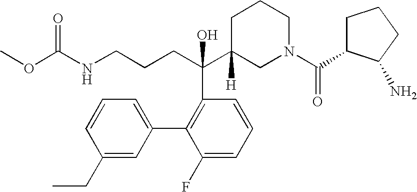 | methyl (S)-4-((R)-1-((1R,2S)-2- aminocyclopentanecarbonyl)piperidin- 3-yl)-4-(3′-ethyl-6-fluorobiphenyl-2- yl)-4-hydroxybutylcarbamate |
| I-26a | 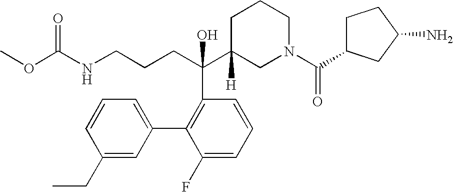 | methyl (S)-4-((R)-1-((1R,3S)-3- aminocyclopentanecarbonyl)piperidin- 3-yl)-4-(3′-ethyl-6-fluorobiphenyl-2- yl)-4-hydroxybutylcarbamate |
| I-27a | 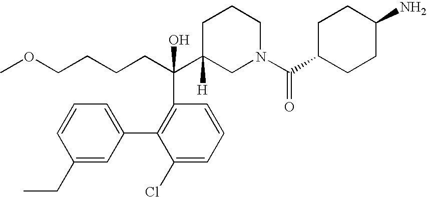 | (trans-4-aminocyclohexyl)((R)-3- ((S)-1-(6-chloro-3′-ethylbiphenyl-2-yl)- 1-hydroxy-5-methoxypentyl)piperidin- 1-yl)methanone |
| I-28a | 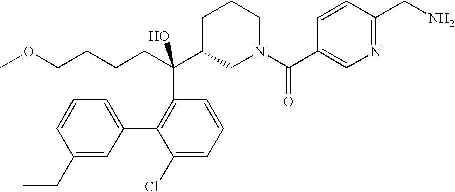 | (1S)-1-((3R)-1-{[6-(aminomethyl)- 3-pyridinyl]carbonyl}-3-piperidinyl)-1- (6-chloro-3′-ethyl-2-biphenylyl)-5- (methyloxy)-1-pentanol |
| I-29a | 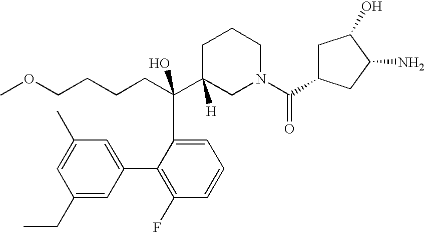 | ((1S,3R,4S)-3-amino-4- hydroxycyclopentyl)((R)-3-((S)-1-(6- fluoro-3′-methoxy-5′-methylbiphenyl-2- yl)-1-hydroxy-5- methoxypentyl)piperidin-1-yl) methanone |
| I-30a | 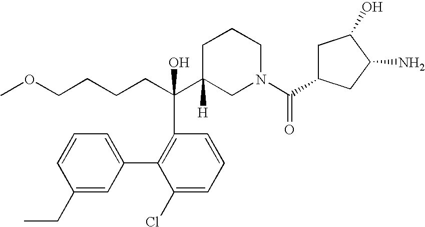 | ((1S,3R,4S)-3-amino-4- hydroxycyclopentyl)((R)-3-((S)-1-(6- chloro-3′-ethylbiphenyl-2-yl)-1- hydroxy-5-methoxypentyl) piperidin-1- yl)methanone |
| I-31a | 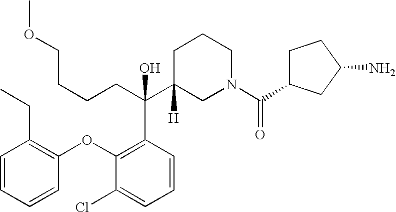 | ((1R,3S)-3-aminocyclopentyl)((R)- 3-((S)-1-(3-chloro-2-(2- ethylphenoxy)phenyl)-1-hydroxy-5- methoxypentyl) piperidin-1- yl)methanone |
| I-32a | 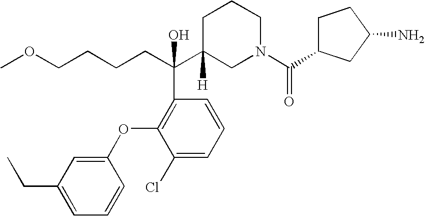 | ((1R,3S)-3-aminocyclopentyl)((R)- 3-((S)-1-(3-chloro-2-(3- ethylphenoxy)phenyl)-1-hydroxy-5- methoxypentyl) piperidin-1- yl)methanone |
| I-33a | 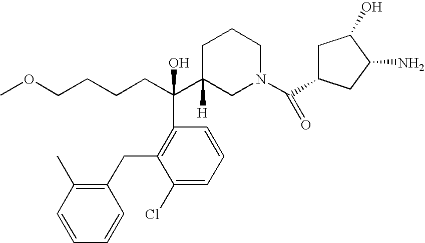 | ((1S,3R,4S)-3-amino-4- hydroxycyclopentyl)((R)-3-((S)-1-(3- chloro-2-(2-methylbenzyl) phenyl)-1- hydroxy-5-methoxypentyl)piperidin-1- yl)methanone |
| I-34a | 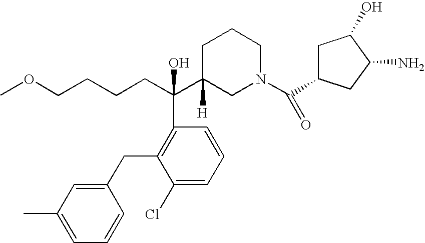 | ((1S,3R,4S)-3-amino-4- hydroxycyclopentyl)((R)-3-((S)-1-(3- chloro-2-(3-methylbenzyl) phenyl)-1- hydroxy-5-methoxypentyl)piperidin-1- yl)methanone |
| I-35a | 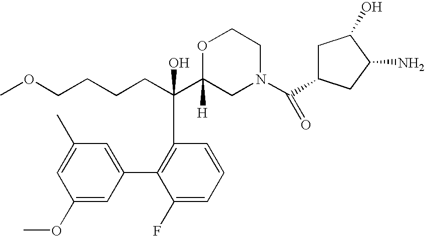 | ((1S,3R,4S)-3-amino-4- hydroxycyclopentyl)((R)-2-((R)-1-(6- fluoro-3′-methoxy-5′-methylbiphenyl-2- yl)-1-hydroxy-5- methoxypentyl)morpholino)methanone |
| I-36a | 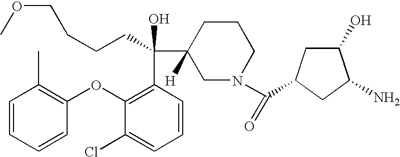 | ((1S,3R,4S)-3-amino-4- hydroxycyclopentyl)((R)-3-((S)-1-(3- chloro-2-(o-tolyloxy)phenyl)-1- hydroxy-5-methoxypentyl) piperidin-1- yl)methanone |
| I-37a | 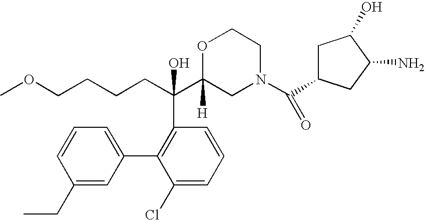 | ((1S,3R,4S)-3-amino-4- hydroxycyclopentyl)((R)-2-((R)-1-(6- chloro-3′-ethylbiphenyl-2-yl)-1- hydroxy-5-methoxypentyl) morpholino)methanone |
| I-38a | 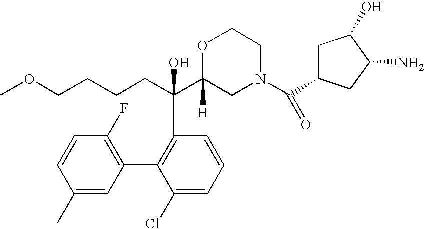 | ((1S,3R,4S)-3-amino-4- hydroxycyclopentyl)((2R)-2-((1R)-1-(6- chloro-2′-fluoro-5′-methylbiphenyl-2- yl)-1-hydroxy-5- methoxypentyl)morpholino) methanone |
| I-39a | 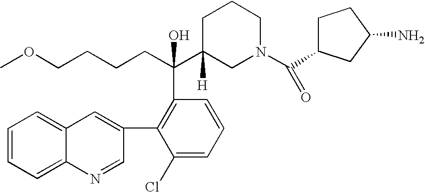 | ((1R,3S)-3-aminocyclopentyl) ((R)- 3-((S)-1-(3-chloro-2-(quinolin-3- yl)phenyl)-1-hydroxy-5- methoxypentyl) piperidin-1- yl)methanone |
| I-40a | 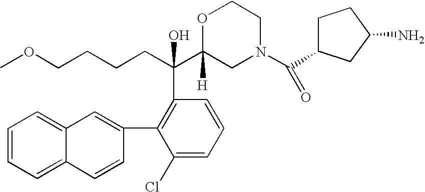 | ((1R,3S)-3-aminocyclopentyl) ((R)- 2-((R)-1-(3-chloro-2-(naphthalen-2- yl)phenyl)-1-hydroxy-5- methoxypentyl) morpholino) methanone |
| I-41a | 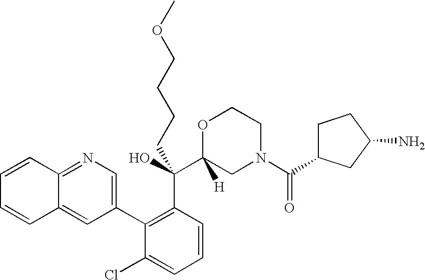 | ((1R,3S)-3-aminocyclopentyl) ((R)- 2-((R)-1-(3-chloro-2-(quinolin-3- yl)phenyl)-1-hydroxy-5- methoxypentyl) morpholino) methanone |
| I-42a | 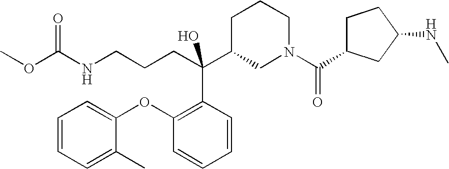 | methyl ((4S)-4-hydroxy-4-((3R)-1- {((1R,3S)-3-(methylamino) cyclopentyl]carbonyl}-3-piperidinyl)-4- {2-[(2-methylphenyl) oxy]phenyl}butyl)carbamate |
| I-43a | 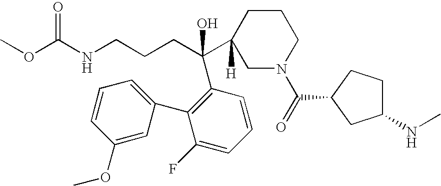 | methyl (S)-4-(6-fluoro-3′- methoxybiphenyl-2-yl)-4-hydroxy-4- ((R)-1-((1R,3S)-3-(methylamino) cyclopentanecarbonyl)piperidin-3- yl)butylcarbamate |
| I-44a | 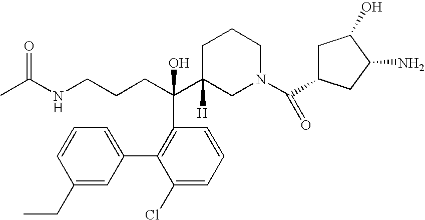 | N-((S)-4-((R)-1-((1S,3R,4S)-3- amino-4-hydroxycyclopentane carbonyl)piperidin-3-yl)-4-(6-chloro-3′- ethylbiphenyl-2-yl)-4- hydroxybutyl)acetamide |
| I-45a | 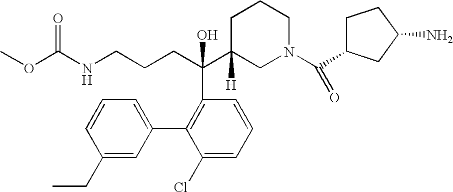 | methyl (S)-4-((R)-1-((1R,3S)-3- aminocyclopentanecarbonyl)piperidin- 3-yl)-4-(6-chloro-3′-ethylbiphenyl-2- yl)-4-hydroxybutylcarbamate |
| I-46a | 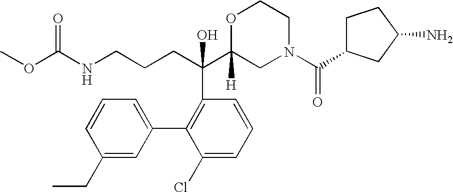 | methyl (R)-4-((R)-4-((1R,3S)-3- aminocyclopentanecarbonyl)morpholin- 2-yl)-4-(6-chloro-3′-ethylbiphenyl-2- yl)-4-hydroxybutylcarbamate |
| I-47a | 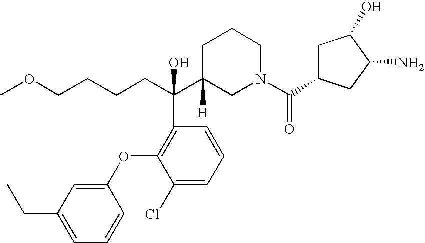 | ((1S,3R,4S)-3-amino-4- hydroxycyclopentyl)((R)-3-((S)-1-(3- chloro-2-(3-ethylphenoxy) phenyl)-1- hydroxy-5-methoxypentyl)piperidin-1- yl)methanone |
| I-48a | 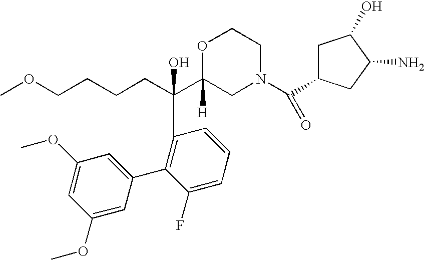 | ((1S,3R,4S)-3-amino-4- hydroxycyclopentyl)((R)-2-((R)-1-(6- fluoro-3′,5′-dimethoxybiphenyl-2-yl)-1- hydroxy-5-methoxypentyl) morpholino) methanone |
| I-49a | 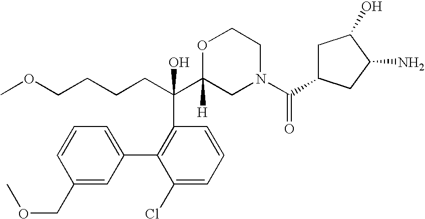 | ((1S,3R,4S)-3-amino-4- hydroxycyclopentyl)((R)-2-((R)-1-(6- chloro-3′-(methoxymethyl) biphenyl-2- yl)-1-hydroxy-5- methoxypentyl)morpholino) methanone |
| I-50a | 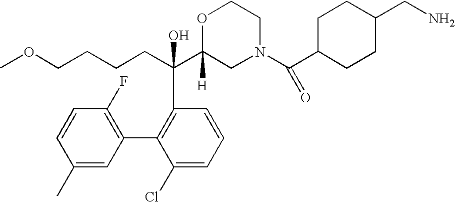 | (4-(aminomethyl)cyclohexyl) ((2R)-2-((1R)-1-(6-chloro-2′-fluoro-5′- methylbiphenyl-2-yl)-1-hydroxy-5- methoxypentyl) morpholino)methanone |
| I-51a | 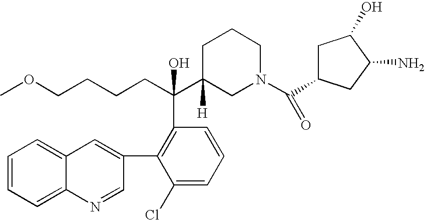 | ((1S,3R,4S)-3-amino-4- hydroxycyclopentyl)((R)-3-((S)-1-(3- chloro-2-(quinolin-3-yl)phenyl)-1- hydroxy-5-methoxypentyl) piperidin-1- yl)methanone |
| I-52a | 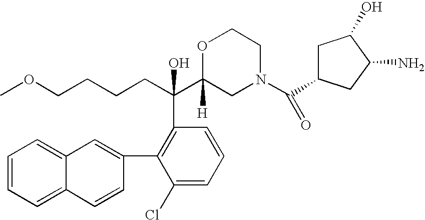 | ((1S,3R,4S)-3-amino-4- hydroxycyclopentyl)((R)-2-((R)-1-(3- chloro-2-(naphthalen-2-yl)phenyl)-1- hydroxy-5-methoxypentyl)morpholino) methanone |
| I-53a | 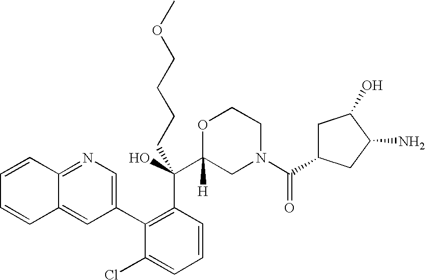 | ((1S,3R,4S)-3-amino-4- hydroxycyclopentyl)((R)-2-((R)-1-(3- chloro-2-(quinolin-3-yl)phenyl)-1- hydroxy-5-methoxypentyl)morpholino) methanone |
| I-54a | 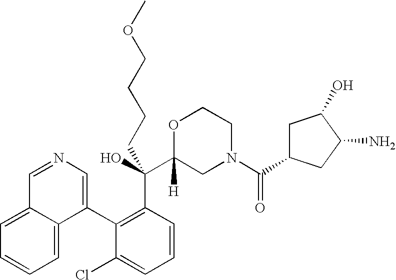 | ((1S,3R,4S)-3-amino-4- hydroxycyclopentyl)((2R)-2-((1R)-1-(3- chloro-2-(isoquinolin-4-yl)phenyl)-1- hydroxy-5-methoxypentyl)morpholino) methanone |
| I-55a | 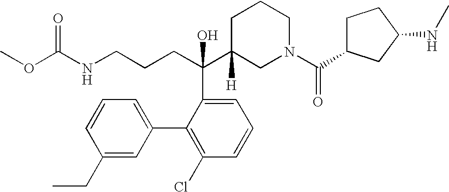 | methyl (S)-4-(6-chloro-3′- ethylbiphenyl-2-yl)-4-hydroxy-4-((R)- 1-((1R,3S)-3- (methylamino)cyclopentanecarbonyl) piperidin-3-yl)butylcarbamate |
| I-56a | 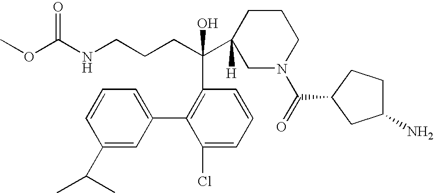 | methyl (S)-4-((R)-1-((1R,3S)-3- aminocyclopentanecarbonyl)piperidin- 3-yl)-4-(6-chloro-3′-isopropylbiphenyl- 2-yl)-4-hydroxybutylcarbamate |
| I-57a | 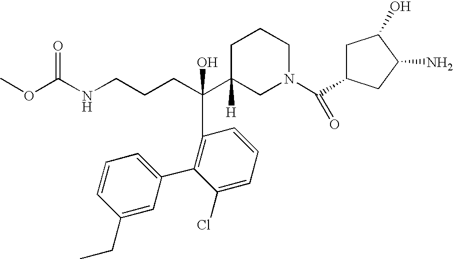 | methyl (S)-4-((R)-1-((1S,3R,4S)-3- amino-4-hydroxycyclopentanecarbonyl) piperidin-3-yl)-4-(6-chloro-3′- ethylbiphenyl-2-yl)-4- hydroxybutylcarbamate |
| I-58a | 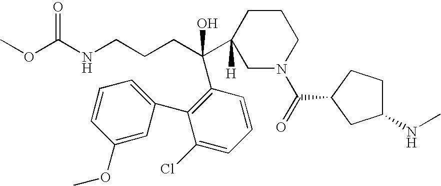 | methyl (S)-4-(6-chloro-3′- methoxybiphenyl-2-yl)-4-hydroxy-4- ((R)-1-((1R,3S)-3-(methylamino) cyclopentanecarbonyl)piperidin-3- yl)butylcarbamate |
| I-59a | 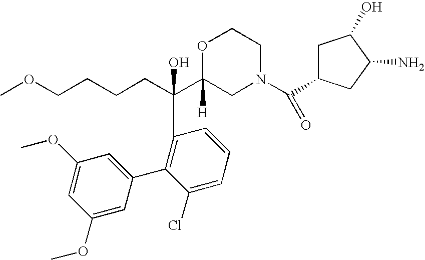 | ((1S,3R,4S)-3-amino-4- hydroxycyclopentyl)((R)-2-((R)-1-(6- chloro-3′,5′-dimethoxybiphenyl-2-yl)-1- hydroxy-5-methoxypentyl) morpholino) methanone |
| I-60a | 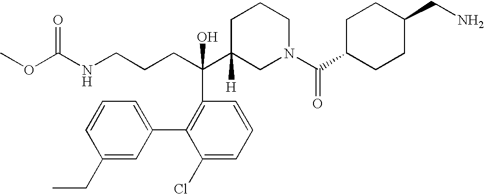 | methyl (S)-4-((R)-1-(trans-4- (aminomethyl)cyclohexanecarbonyl) piperidin-3-yl)-4-(6-chloro-3′- ethylbiphenyl-2-yl)-4- hydroxybutylcarbamate |
| I-61a | 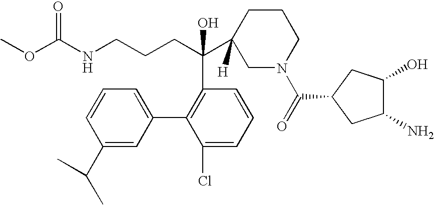 | methyl (S)-4-((R)-1-((1S,3R,4S)-3- amino-4-hydroxycyclopentanecarbonyl) piperidin-3-yl)-4-(6-chloro-3′- isopropylbiphenyl-2-yl)-4- hydroxybutylcarbamate |
| I-62a | 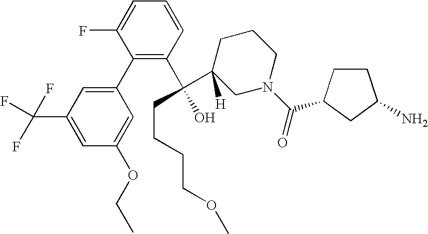 | ((1R,3S)-3-aminocyclopentyl)((R)- 3-((S)-1-(3′-ethoxy-6-fluoro-5′- (trifluoromethyl)biphenyl-2-yl)-1- hydroxy-5-methoxypentyl)piperidin-1- yl)methanone |
| I-63a | 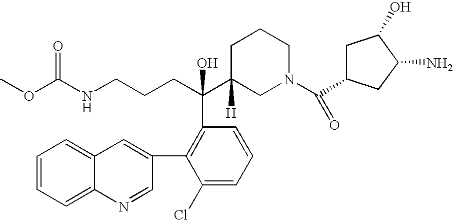 | methyl (S)-4-((R)-1-((1S,3R,4S)-3- amino-4-hydroxycyclopentanecarbonyl) piperidin-3-yl)-4-(3-chloro-2-(quinolin- 3-yl)phenyl)-4-hydroxybutylcarbamate |
| I-64a | 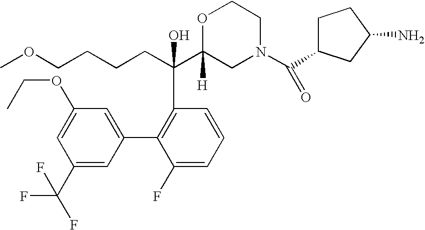 | ((1R,3S)-3-aminocyclopentyl)((R)- 2-((R)-1-(3′-ethoxy-6-fluoro-5′- (trifluoromethyl)biphenyl-2-yl)-1- hydroxy-5-methoxypentyl)morpholino) methanone |
| I-65a | 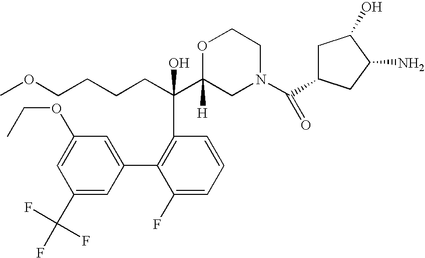 | ((1S,3R,4S)-3-amino-4- hydroxycyclopentyl)((R)-3-((S)-1-(3′- ethoxy-6-fluoro-5′- (trifluoromethyl)biphenyl-2-yl)-1- hydroxy-5-methoxypentyl)piperidin-1- yl)methanone |
| I-66a | 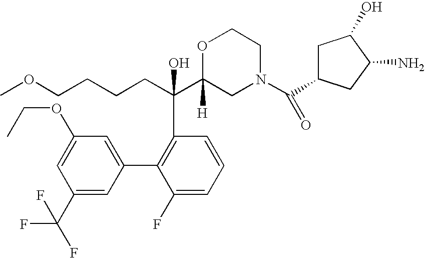 | ((1S,3R,4S)-3-amino-4- hydroxycyclopentyl)((R)-2-((R)-1-(3′- ethoxy-6-fluoro-5′- (trifluoromethyl)biphenyl-2-yl)-1- hydroxy-5-methoxypentyl)morpholino) methanone |
| I-67a | 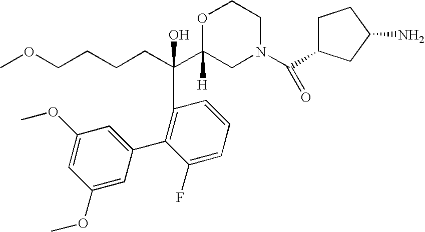 | ((1R,3S)-3-aminocyclopentyl)((R)- 2-((R)-1-(6-fluoro-3′,5′- dimethoxybiphenyl-2-yl)-1-hydroxy-5- methoxypentyl)morpholino) methanone |
| I-68a | 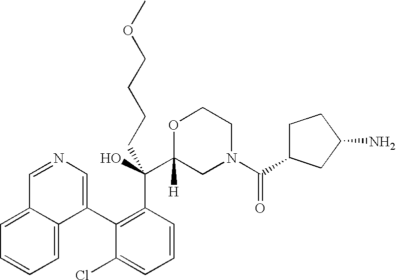 | ((1R,3S)-3- aminocyclopentyl)((2R)-2-((1R)-1-(3- chloro-2-(isoquinolin-4-yl)phenyl)-1- hydroxy-5-methoxypentyl) morpholino)methanone |
| I-69a | 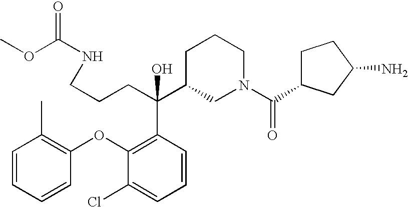 | methyl (4-((3R)-1-{[(1R,3S)-3- aminocyclopentyl]carbonyl}-3- piperidinyl)-4-{3-chloro-2-[(2- methylphenyl)oxy]phenyl}-4- hydroxybutyl)carbamate |
| I-70a | 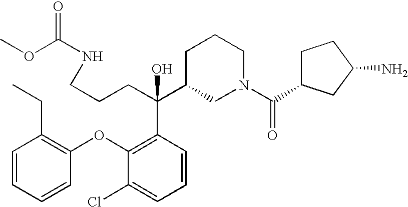 | methyl (4-((3R)-1-{[(1R,3S)-3- aminocyclopentyl]carbonyl}-3- piperidinyl)-4-{3-chloro-2-[(2- ethylphenyl)oxy]phenyl}-4- hydroxybutyl)carbamate |
| I-71a | 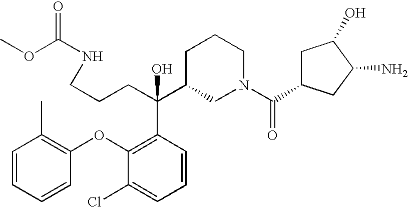 | methyl (4-((3R)-1-{[(1S,3R,4S)-3- amino-4- hydroxycyclopentyl]carbonyl}-3- piperidinyl)-4-{3-chloro-2-[(2- methylphenyl)oxy]phenyl}-4- hydroxybutyl)carbamate |
| I-72a | 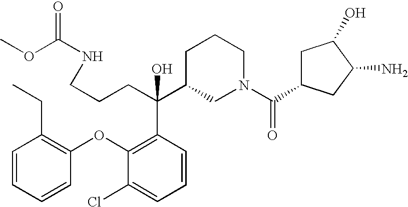 | methyl (4-((3R)-1-{[(1S,3R,4S)-3- amino-4- hydroxycyclopentyl]carbonyl}-3- piperidinyl)-4-{3-chloro-2-[(2- ethylphenyl)oxy]phenyl}-4- hydroxybutyl)carbamate |
| I-73a | 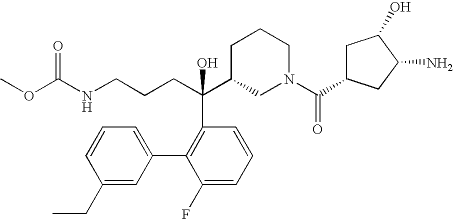 | methyl [4-((3R)-1-{[(1S,3R,4S)-3- amino-4- hydroxycyclopentyl]carbonyl}-3- piperidinyl)-4-(3′-ethyl-6-fluoro-2- biphenylyl)-4-hydroxybutyl]carbamate |
| I-74a | 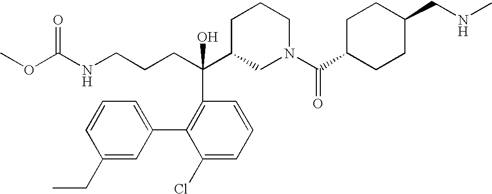 | methyl {4-(6-chloro-3′-ethyl-2- biphenylyl)-4-hydroxy-4-[(3R)-1- ({trans-4-[(methylamino)methyl] cyclohexyl}carbonyl)-3- piperidinyl]butyl}carbamate |
| I-75a | 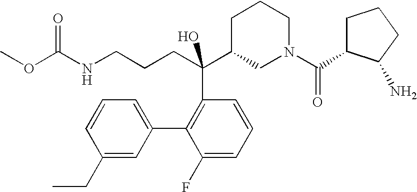 | methyl [(4S)-4-((3R)-1-{[(1R,2S)- 2-aminocyclopentyl]carbonyl}-3- piperidinyl)-4-(3′-ethyl-6-fluoro-2- biphenylyl)-4-hydroxybutyl]carbamate |
| I-76a | 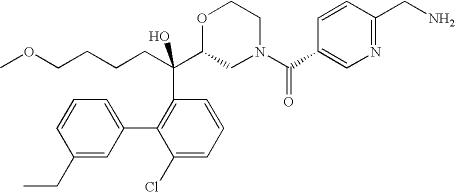 | (1R)-1-((2R)-4-{(6-(aminomethyl)- 3-pyridinyl]carbonyl}-2-morpholinyl)- 1-(6-chloro-3′-ethyl-2-biphenylyl)-5- (methyloxy)-1-pentanol |
| I-77a | 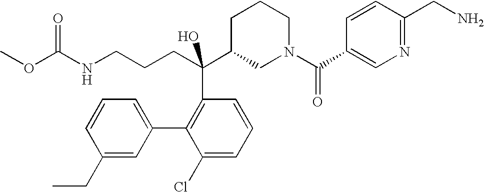 | methyl [(4S)-4-((3R)-1-{(6- (aminomethyl)-3-pyridinyl]carbonyl}- 3-piperidinyl)-4-(6-chloro-3′-ethyl-2- biphenylyl)-4-hydroxybutyl]carbamate |
| I-78a | 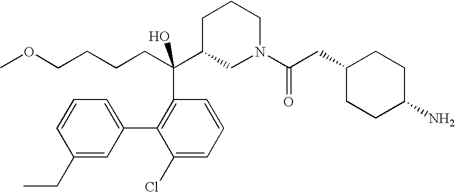 | (1S)-1-{(3R)-1-[(cis-4- aminocyclohexyl)acetyl]-3- piperidinyl}-1-(6-chloro-3′-ethyl-2- biphenylyl)-5-(methyloxy)-1-pentanol |
| I-79a | 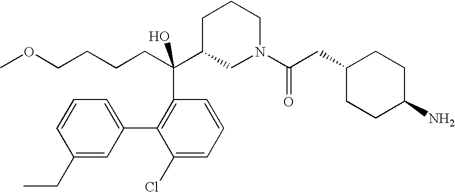 | (1S)-1-{(3R)-1-[(trans-4- aminocyclohexyl)acetyl]-3- piperidinyl}-1-(6-chloro-3′-ethyl-2- biphenylyl)-5-(methyloxy)-1-pentanol |
| I-80a | 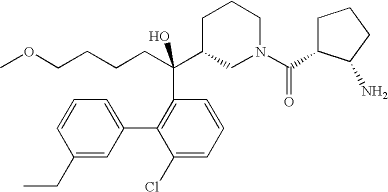 | (1S)-1-((3R)-1-{((1R,2S)-2- aminocyclopentyl]carbonyl}-3- piperidinyl)-1-(6-chloro-3′-ethyl-2- biphenylyl)-5-(methyloxy)-1-pentanol |
| I-81a | 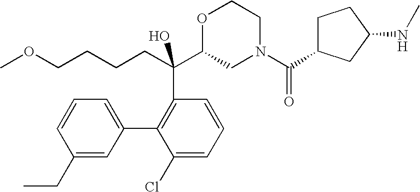 | (1R)-1-(6-chloro-3′-ethyl-2- biphenylyl)-1-((2R)-4-{[(1R,3S)-3- (methylamino)cyclopentyl]carbonyl}-2- morpholinyl)-5-(methyloxy)-1-pentanol |
| I-82a | 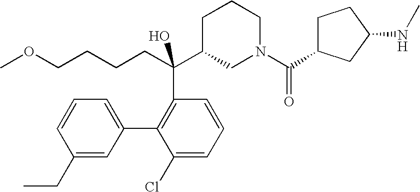 | (1S)-1-(6-chloro-3′-ethyl-2- biphenylyl)-1-((3R)-1-{((1R,3S)-3- (methylamino)cyclopentyl]carbonyl}-3- piperidinyl)-5-(methyloxy)-1-pentanol |
| I-83a | 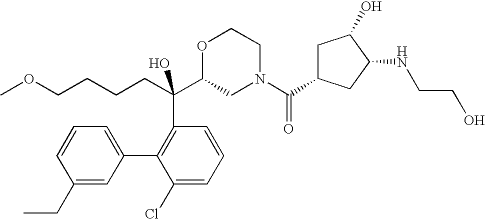 | (1S,2R,4S)-4-({(2R)-2-[(1R)-1-(6- chloro-3′-ethyl-2-biphenylyl)-1- hydroxy-5-(methyloxy)pentyl]-4- morpholinyl}carbonyl)-2-[(2- hydroxyethyl)amino]cyclopentanol |
| I-84a | 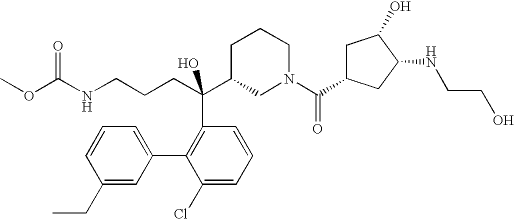 | methyl {(4S)-4-(6-chloro-3′-ethyl- 2-biphenylyl)-4-hydroxy-4-[(3R)-1- ({(1S,3S,4R)-3-hydroxy-4-[(2- hydroxyethyl)amino]cyclopentyl} carbonyl)-3-piperidinyl]butyl}carbamate |
| I-85a | 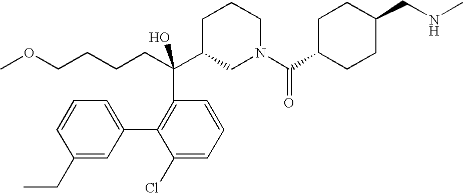 | (1S)-1-(6-chloro-3′-ethyl-2- biphenylyl)-1- [(3R)-1-({trans-4- [(methylamino)methyl]cyclohexyl} carbonyl)-3-piperidinyl]-5-(methyloxy)-1- pentanol |
| I-86a | 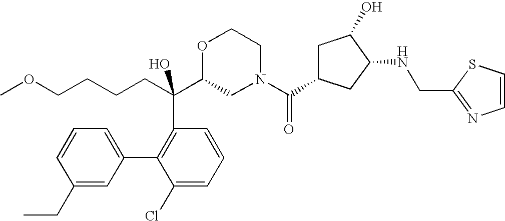 | (1S,2R,4S)-4-({(2R)-2-[(1R)-1-(6- chloro-3′-ethyl-2-biphenylyl)-1- hydroxy-5-(methyloxy)pentyl]-4- morpholinyl}carbonyl)-2-[(1,3-thiazol- 2-ylmethyl)amino]cyclopentanol |
| I-87a | 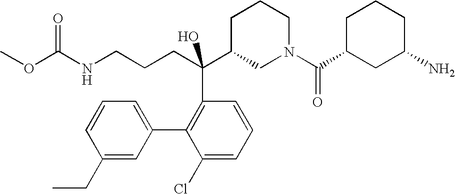 | methyl [(4S)-4-((3R)-1-{[(1R,3S)- 3-aminocyclohexyl]carbonyl}-3- piperidinyl)-4-(6-chloro-3′-ethyl-2- biphenylyl)-4-hydroxybutyl]carbamate |
| I-88a | 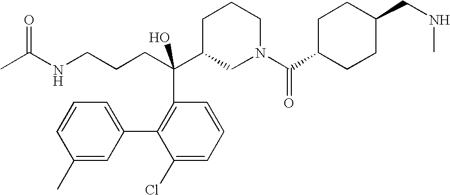 | N-{(4S)-4-(6-chloro-3′-methyl-2- biphenylyl)-4-hydroxy-4-[(3R)-1- ({trans-4- [(methylamino)methyl]cyclohexyl} carbonyl)-3-piperidinyl]butyl}acetamide |
| I-89a | 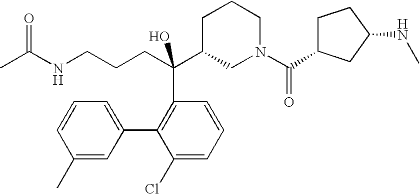 | N-[(4S)-4-(6-chloro-3′-methyl-2- biphenylyl)-4-hydroxy-4-((3R)-1- {[(1R,3S)-3- (methylamino)cyclopentyl]carbonyl}-3- piperidinyl)butyl]acetamide |
| I-90a | 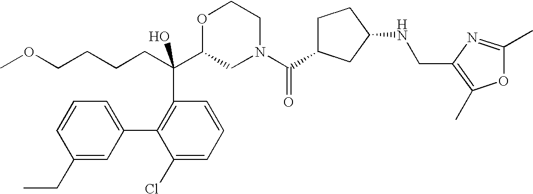 | (1R)-1-(6-chloro-3′-ethyl-2- biphenylyl)-1-{(2R)-4-[((1R,3S)-3- {((2,5-dimethyl-1,3-oxazol-4- yl)methyl]amino}cyclopentyl)carbonyl]- 2-morpholinyl}-5-(methyloxy)-1- pentanol |
| I-91a | 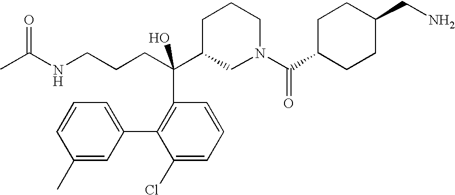 | N-[(4S)-4-((3R)-1-{[trans-4- (aminomethyl)cyclohexyl]carbonyl}-3- piperidinyl)-4-(6-chloro-3′-methyl-2- biphenylyl)-4-hydroxybutyl]acetamide |
| I-92a | 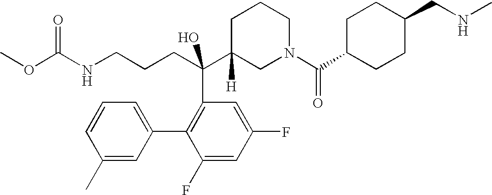 | methyl {(4S)-4-(4,6-difluoro-3′- methyl-2-biphenylyl)-4-hydroxy-4- [(3R)-1-({trans-4- [(methylamino)methyl]cyclohexyl} carbonyl)-3-piperidinyl]butyl}carbamate |
| I-93a | 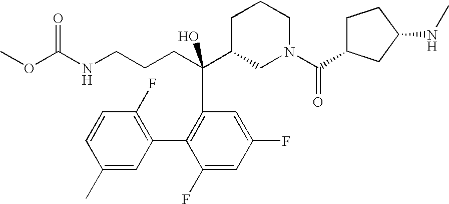 | methyl [(4S)-4-hydroxy-4-((3R)-1- {[(1R,3S)-3-(methylamino) cyclopentyl]carbonyl}-3-piperidinyl)-4- (2′,4,6-trifluoro-5′-methyl-2- biphenylyl)butyl] carbamate |
| I-94a | 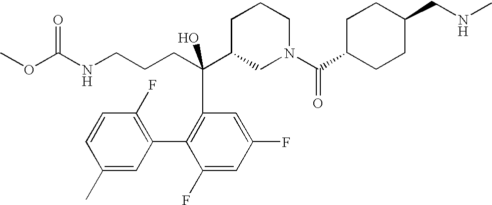 | methyl [(4S)-4-hydroxy-4-[(3R)-1- ({trans-4-[(methylamino) methyl]cyclohexyl}carbonyl)-3- piperidinyl]-4-(2′,4,6-trifluoro-5- methyl-2-biphenylyl)butyl] carbamate |
| I-95a | 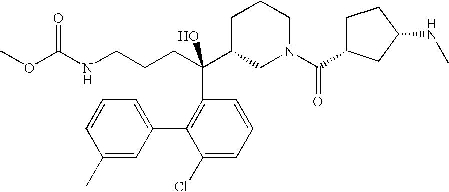 | methyl [(4S)-4-(6-chloro-3′- methyl-2-biphenylyl)-4-hydroxy-4- ((3R)-1-{[(1R,3S)-3-(methylamino) cyclopentyl]carbonyl}-3- piperidinyl)butyl]carbamate |
| I-96a | 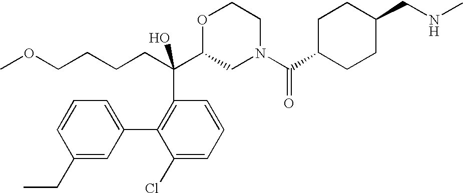 | (1R)-1-(6-chloro-3′-ethyl-2- biphenylyl)-1-[(2R)-4-({trans-4- [(methylamino)methyl]cyclohexyl} carbonyl)-2-morpholinyl]-5-(methyloxy)-1- pentanol |
| I-97a | 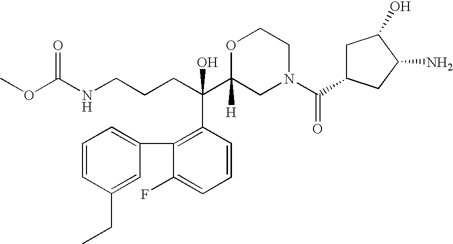 | methyl [(4R)-4-((2R)-4- {[(1S,3R,4S)-3-amino-4- hydroxycyclopentyl]carbonyl}-2- morpholinyl)-4-(3′-ethyl-6-fluoro-2- biphenylyl)-4-hydroxybutyl] carbamate |
| I-98a | 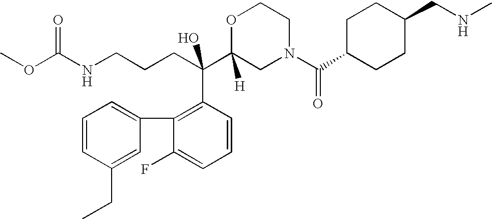 | methyl {(4R)-4-(3′-ethyl-6-fluoro- 2-biphenylyl)-4-hydroxy-4-[(2R)-4- ({trans-4- [(methylamino)methyl]cyclohexyl} carbonyl)-2-morpholinyl]butyl} carbamate |
| I-99a | 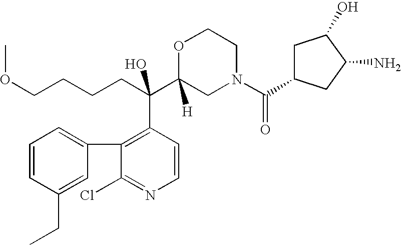 | (1S,2R,4S)-2-amino-4-({(2R)-2- [(1R)-1-[2-chloro-3-(3-ethylphenyl)-4- pyridinyl]-1-hydroxy-5- (methyloxy)pentyl]-4- morpholinyl}carbonyl) cyclopentanol |
| I-100a | 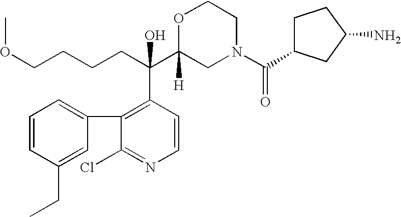 | (1R)-1-((2R)-4-{[(1R,3S)-3- aminocyclopentyl] carbonyl}-2- morpholinyl)-1-[2-chloro-3-(3- ethylphenyl)-4-pyridinyl]-5- (methyloxy)-1-pentanol |
| I-101a | 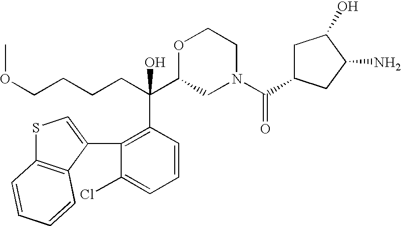 | (1S,2R,4S)-2-amino-4-({(2R)-2- [(1R)-1-[2-(1-benzothien-3-yl)-3- chlorophenyl]-1-hydroxy-5- (methyloxy)pentyl]-4-morpholinyl} carbonyl)cyclopentanol |
| I-102a | 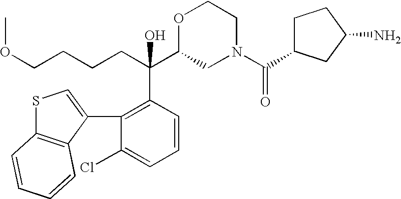 | (1R)-1-((2R)-4-{[(1R,3S)-3- aminocyclopentyl]carbonyl}-2- morpholinyl)-1-[2-(1-benzothien-3-yl)- 3-chlorophenyl]-5-(methyloxy)-1- pentanol |
| I-103a | 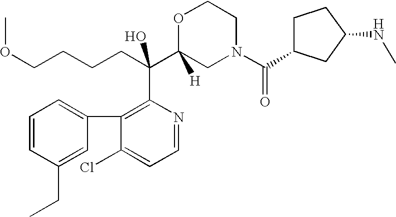 | (1R)-1-[4-chloro-3-(3- ethylphenyl)-2-pyridinyl]-1-((2R)-4- {[(1R,3S)-3-(methylamino) cyclopentyl]carbonyl}-2-morpholinyl)- 5-(methyloxy)-1-pentanol |
| I-104a | 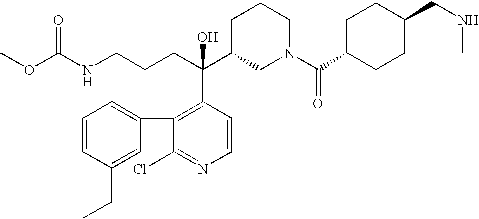 | methyl {(4S)-4-[2-chloro-3-(3- ethylphenyl)-4-pyridinyl]-4-hydroxy-4- [(3R)-1-({trans-4- [(methylamino)methyl]cyclohexyl carbonyl)-3-piperidinyl]butyl} carbamate |
| I-105a | 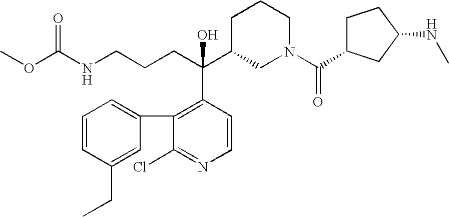 | methyl [(4S)-4-[2-chloro-3-(3- ethylphenyl)-4-pyridinyl]-4-hydroxy-4- ((3R)-1-{[(1R,3S)-3- (methylamino)cyclopentyl] carbonyl}- 3-piperidinyl)butyl] carbamate |
| I-106a | 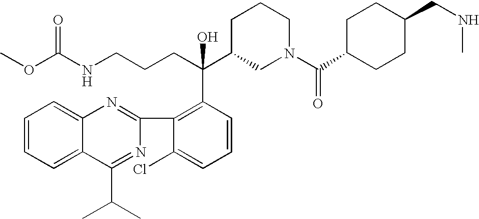 | methyl {(4S)-4-{3-chloro-2-[4-(1- methylethyl)-2-quinazolinyl]phenyl}-4- hydroxy-4-[(3R)-1-({trans-4- [(methylamino)methyl]cyclohexyl} carbonyl)-3-piperidinyl]butyl}carbamate |
| I-107a | 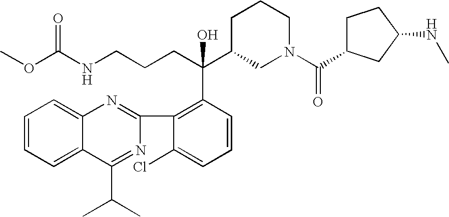 | methyl [(4S)-4-{3-chloro-2-[4-(1- methylethyl)-2-quinazolinyl] phenyl}- 4-hydroxy-4-((3R)-1-{[(1R,3S)-3- (methylamino) cyclopentyl]carbonyl}- 3-piperidinyl)butyl]carbamate |
| I-108a | 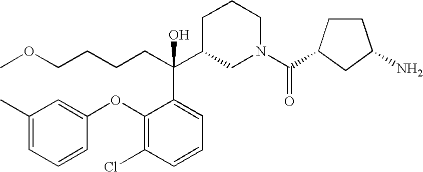 | (1S)-1-((3R)-1-{[(1R,3S)-3- aminocyclopentyl]carbonyl}-3- piperidinyl)-1-{3-chloro-2-[(3- methylphenyl)oxy]phenyl}-5- (methyloxy)-1-pentanol |
| I-109a | 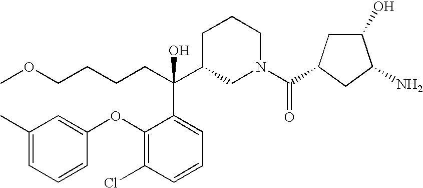 | (1S,2R,4S)-2-amino-4-({(3R)-3- [(1S)-1-{3-chloro-2-[(3- methylphenyl)oxy]phenyl}-1-hydroxy- 5-(methyloxy)pentyl]-1- piperidinyl}carbonyl)cyclopentanol |
| I-110a | 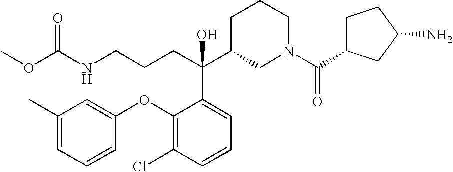 | methyl ((4S)-4-((3R)-1-{[(1R,3S)- 3-aminocyclopentyl] carbonyl}-3- piperidinyl)-4-{3-chloro-2-[(3- methylphenyl)oxy] phenyl}-4- hydroxybutyl)carbamate |
| I-111a | 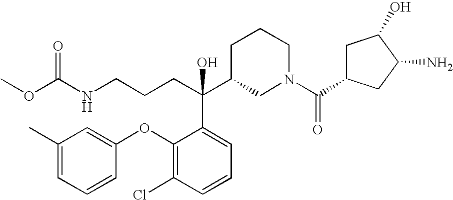 | methyl ((4S)-4-((3R)-1- {[(1S,3R,4S)-3-amino-4- hydroxycyclopentyl]carbonyl}-3- piperidinyl)-4-{3-chloro-2-[(3- methylphenyl)oxy]phenyl}-4- hydroxybutyl)carbamate |
| I-112a | 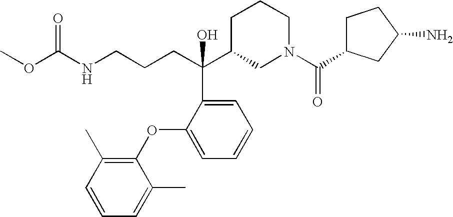 | methyl ((4S)-4-((3R)-1-{[(1R,3S)- 3-aminocyclopentyl] carbonyl}-3- piperidinyl)-4-{2-[(2,6- dimethylphenyl)oxy]phenyl}-4- hydroxybutyl)carbamate |
| I-113a | 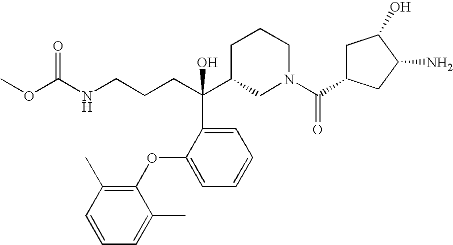 | methyl ((4S)-4-((3R)-1- {[(1S,3R,4S)-3-amino-4- hydroxycyclopentyl]carbonyl}-3- piperidinyl)-4-{2-[(2,6- dimethylphenyl)oxy]phenyl}-4- hydroxybutyl)carbamate |
| I-114a | 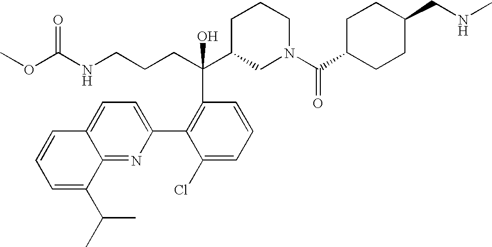 | methyl {(4S)-4-{3-chloro-2-[8-(1- methylethyl)-2-quinolinyl] phenyl}-4- hydroxy-4-[(3R)-1-({trans-4- [(methylamino)methyl] cyclohexyl}carbonyl)-3- piperidinyl]butyl}carbamate |
| I-115a | 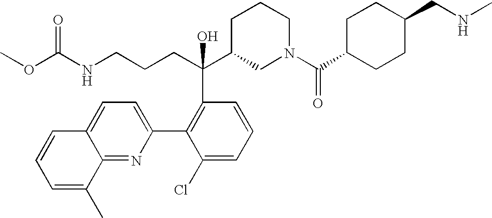 | methyl {(4S)-4-[3-chloro-2-(8- methyl-2-quinolinyl)phenyl]-4- hydroxy-4-[(3R)-1-({trans-4- [(methylamino)methyl]cyclohexyl} carbonyl)-3-piperidinyl]butyl} carbamate |
| I-116a | 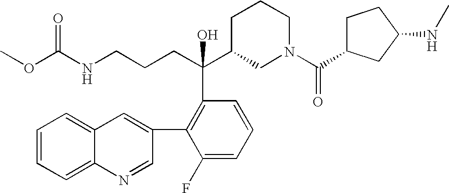 | methyl [(4S)-4-[3-fluoro-2-(3- quinolinyl)phenyl]-4-hydroxy-4-((3R)- 1-{[(1R,3S)-3-(methylamino) cyclopentyl]carbonyl}-3- piperidinyl)butyl]carbamate |
| I-117a | 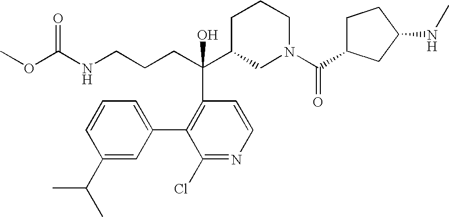 | methyl [(4S)-4-{2-chloro-3-[3-(1- methylethyl)phenyl]-4-pyridinyl}-4- hydroxy-4-((3R)-1-{[(1R,3S)-3- (methylamino) cyclopentyl]carbonyl}- 3-piperidinyl)butyl]carbamate |
| I-118a | 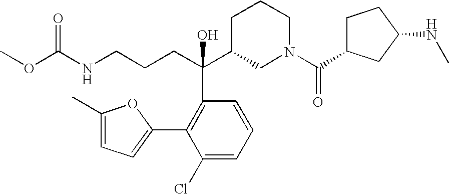 | methyl [(4S)-4-[3-chloro-2-(5- methyl-2-furanyl)phenyl]-4-hydroxy-4- ((3R)-1-{[(1R,3S)-3- (methylamino)cyclopentyl] carbonyl}- 3-piperidinyl)butyl] carbamate |
| I-119a | 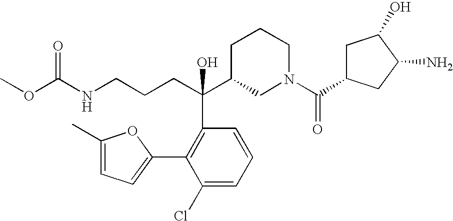 | methyl {(4S)-4-((3R)-1- {[(1S,3R,4S)-3-amino-4- hydroxycyclopentyl]carbonyl}-3- piperidinyl)-4- [3-chloro-2-(5-methyl-2- furanyl)phenyl]-4- hydroxybutyl}carbamate |
| I-120a | 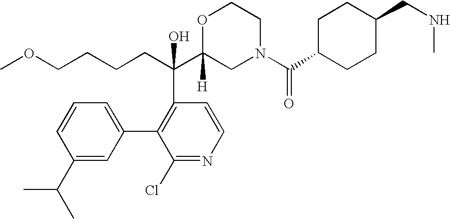 | (1R)-1-{2-chloro-3-[3-(1- methylethyl)phenyl]-4-pyridinyl}-1- [(2R)-4-({trans-4- [(methylamino)methyl]cyclohexyl} carbonyl)-2-morpholinyl]-5- (methyloxy)-1-pentanol |
| I-121a | 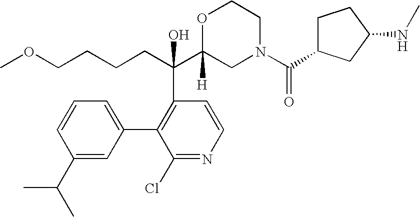 | (1R)-1-{2-chloro-3-[3-(1- methylethyl)phenyl]-4-pyridinyl}-1- ((2R)-4-{[(1R,3S)-3- (methylamino)cyclopentyl] carbonyl}- 2-morpholinyl)-5-(methyloxy)-1- pentanol |
| I-122a | 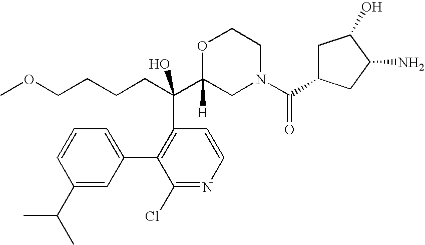 | (1S,2R,4S)-2-amino-4-({(2R)-2- [(1R)-1-{2-chloro-3-[3-(1- methylethyl)phenyl]-4-pyridinyl}-1- hydroxy-5-(methyloxy)pentyl]-4- morpholinyl}carbonyl) cyclopentanol |
| I-123a | 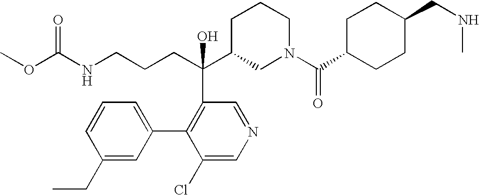 | methyl {(4S)-4-[5-chloro-4-(3- ethylphenyl)-3-pyridinyl]-4-hydroxy-4- [(3R)-1-({trans-4- [(methylamino)methyl]cyclohexyl} carbonyl)-3-piperidinyl]butyl} carbamate |
| I-124a | 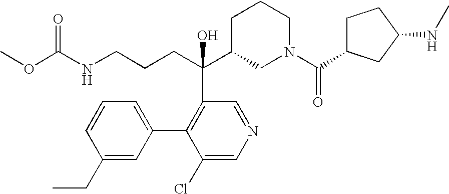 | methyl [(4S)-4-[5-chloro-4-(3- ethylphenyl)-3-pyridinyl]-4-hydroxy-4- ((3R)-1-{[(1R,3S)-3- (methylamino)cyclopentyl] carbonyl}- 3-piperidinyl)butyl] carbamate |
| I-125a | 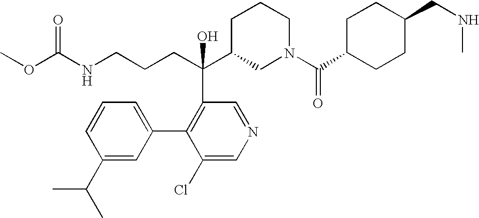 | methyl {(4S)-4-{5-chloro-4-[3-(1- methylethyl)phenyl]-3-pyridinyl}-4- hydroxy-4-[(3R)-1-({trans-4- [(methylamino) methyl]cyclohexyl}carbonyl)-3- piperidinyl]butyl}carbamate |
| I-126a | 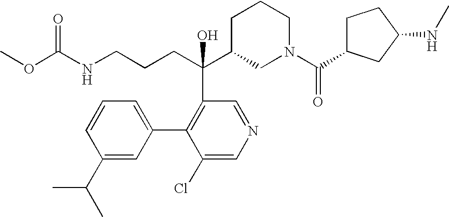 | methyl [(4S)-4-{5-chloro-4-[3-(1- methylethyl)phenyl]-3-pyridinyl}-4- hydroxy-4-((3R)-1-{[(1R,3S)-3- (methylamino)cyclopentyl]carbonyl}- 3-piperidinyl)butyl]carbamate |
| I-127a | 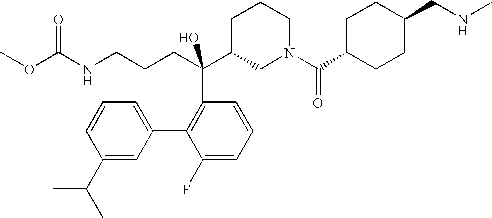 | methyl {(4S)-4-[6-fluoro-3′-(l- methylethyl)-2-biphenylyl]-4-hydroxy- 4-[(3R)-1-({trans-4- [(methylamino)methyl]cyclohexyl} carbonyl)-3-piperidinyl]butyl} carbamate |
| I-128a | 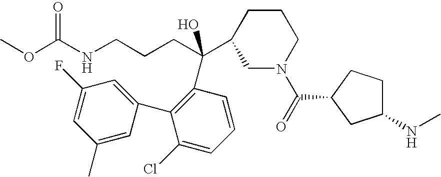 | methyl [(4S)-4-(6-chloro-3′-fluoro- 5′-methyl-2-biphenylyl)-4-hydroxy-4- ((3R)-1-{[(1R,3S)-3- (methylamino)cyclopentyl] carbonyl}- 3-piperidinyl)butyl] carbamate |
| I-129a | 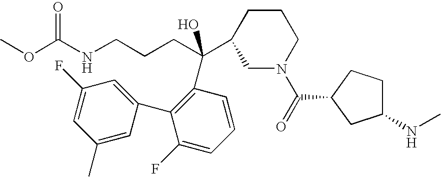 | methyl [(4S)-4-(3′,6-difluoro-5′- methyl-2-biphenylyl)-4-hydroxy-4- ((3R)-1-{[(1R,3S)-3- (methylamino)cyclopentyl] carbonyl}- 3-piperidinyl)butyl] carbamate |
| I-130a | 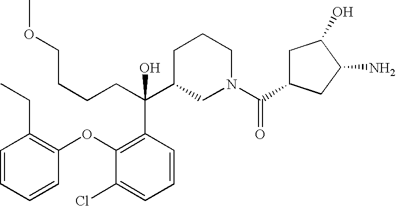 | (1S,2R,4S)-2-amino-4-({(3R)-3- [(1S)-1-{3-chloro-2-[(2- ethylphenyl)oxy]phenyl}-1-hydroxy-5- (methyloxy)pentyl]-1- piperidinyl}carbonyl)cyclopentanol |
| I-131a | 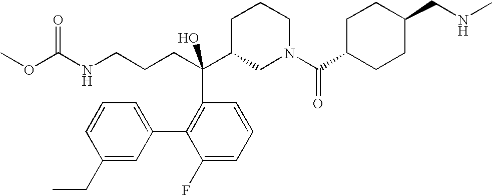 | methyl {(4S)-4-(3′-ethyl-6-fluoro- 2-biphenylyl)-4-hydroxy-4-[(3R)-1- ({trans-4-[(methylamino) methyl]cyclohexyl}carbonyl)-3- piperidinyl]butyl}carbamate |
| I-132a | 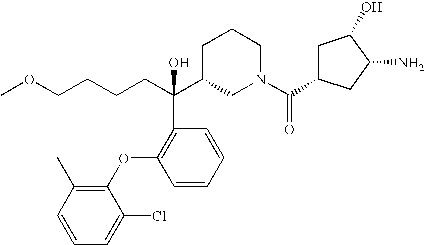 | (1S,2R,4S)-2-amino-4-({(3R)-3- [(1S)-1-{2-[(2-chloro-6- methylphenyl)oxy]phenyl}-1-hydroxy 5-(methyloxy)pentyl]-1- piperidinyl}carbonyl)cyclopentanol |
| I-133a | 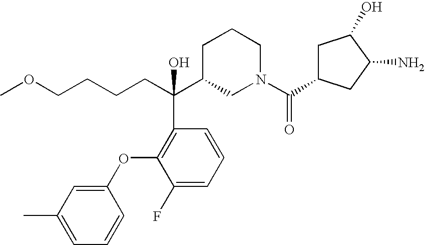 | (1S,2R,4S)-2-amino-4-({(3R)-3- [(1S)-1-{3-fluoro-2-[(3- methylphenyl)oxy]phenyl}-1-hydroxy 5-(methyloxy)pentyl]-1- piperidinyl}carbonyl)cyclopentanol |
| I-134a | 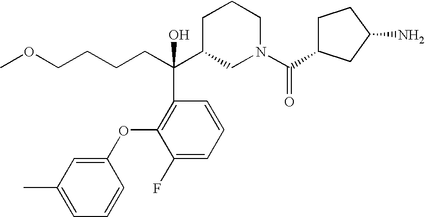 | (1S)-1-((3R)-1-{[(1R,3S)-3- aminocyclopentyl]carbonyl}-3- piperidinyl)-1-{3-fluoro-2-[(3- methylphenyl)oxy]phenyl}-5- (methyloxy)-1-pentanol |
| I-135a | 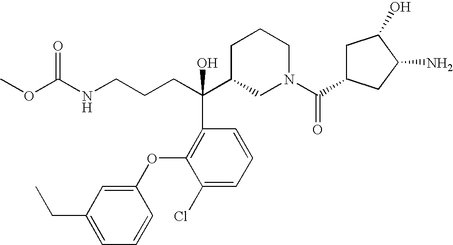 | methyl ((4S)-4-((3R)-1- {[(1S,3R,4S)-3-amino-4- hydroxycyclopentyl]carbonyl}-3- piperidinyl)-4-{3-chloro-2-[(3- ethylphenyl)oxy]phenyl}-4- hydroxybutyl)carbamate |
| I-136a | 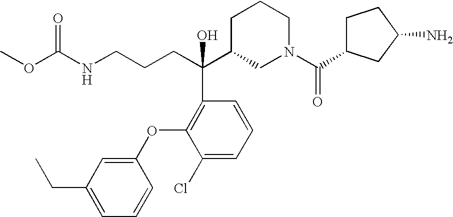 | methyl ((4S)-4-((3R)-1-{[(1R,3S)- 3-aminocyclopentyl]carbonyl}-3- piperidinyl)-4-{3-chloro-2-[(3- ethylphenyl)oxy] phenyl}-4- hydroxybutyl)carbamate |
| I-137a | 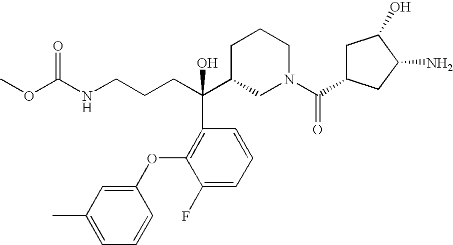 | methyl ((4S)-4-((3R)-1- {[(1S,3R,4S)-3-amino-4- hydroxycyclopentyl] carbonyl}-3- piperidinyl)-4-{3-fluoro-2-[(3- methylphenyl)oxy]phenyl}-4- hydroxybutyl)carbamate |
| I-138a | 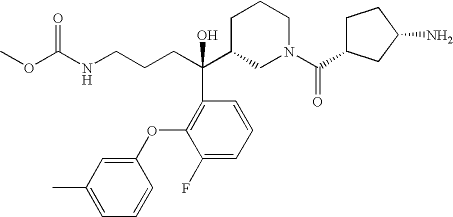 | methyl ((4S)-4-((3R)-1-{[(1R,3S)- 3-aminocyclopentyl] carbonyl}-3- piperidinyl)-4-{3-fluoro-2-[(3- methylphenyl)oxy] phenyl}-4- hydroxybutyl)carbamate |
| I-139a | 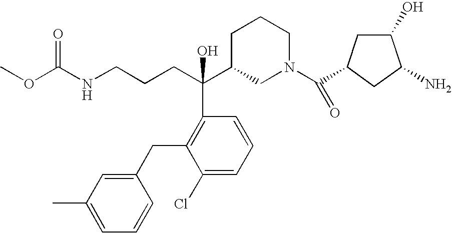 | methyl ((4S)-4-((3R)-1- {[(1S,3R,4S)-3-amino-4- hydroxycyclopentyl]carbonyl}-3- piperidinyl)-4-{3-chloro-2-[(3- methylphenyl)methyl]phenyl}-4- hydroxybutyl)carbamate |
| I-140a | 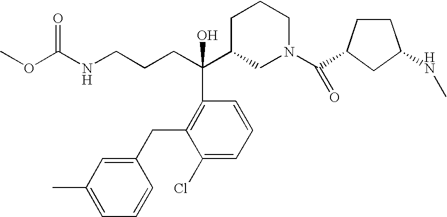 | methyl [(4S)-4-{3-chloro-2-[(3- methylphenyl)methyl]phenyl}-4- hydroxy-4-((3R)-1-{[(1R,3S)-3- (methylamino)cyclopentyl] carbonyl}- 3-piperidinyl)butyl] carbamate |
| I-141a | 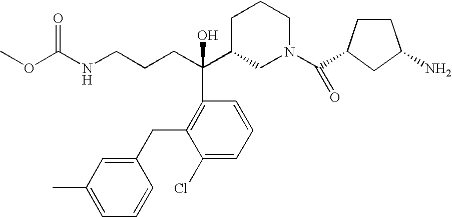 | methyl ((4S)-4-((3R)-1-{[(1R,3S)- 3-aminocyclopentyl] carbonyl}-3- piperidinyl)-4-{3-chloro-2-[(3- methylphenyl)methyl] phenyl}-4- hydroxybutyl)carbamate |
| I-142a | 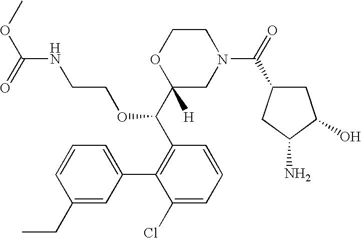 | methyl (2-{[(S)-((2R)-4- {[(1S,3R,4S)-3-amino-4- hydroxycyclopentyl]carbonyl}-2- morpholinyl)(6-chloro-3′-ethyl-2- biphenylyl)methyl]oxy}ethyl) carbamate |
| I-143a | 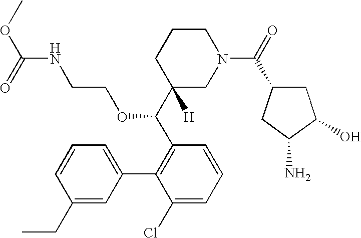 | methyl (2-{[(R)-((3R)-1- {[(1S,3R,4S)-3-amino-4- hydroxycyclopentyl]carbonyl}-3- piperidinyl)(6-chloro-3′-ethyl-2- biphenylyl)methyl]oxy}ethyl)carbamate |
| I-144a | 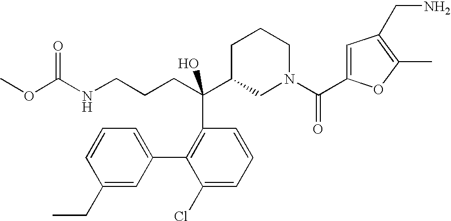 | methyl [(4S)-4-((3R)-1-{[4- (aminomethyl)-5-methyl-2- furanyl]carbonyl}-3-piperidinyl)-4-(6- chloro-3′-ethyl-2-biphenylyl)-4- hydroxybutyl]carbamate |
| I-145a | 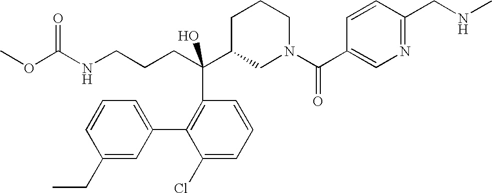 | methyl {(4S)-4-(6-chloro-3′-ethyl- 2-biphenylyl)-4-hydroxy-4-[(3R)-1-({6- [(methylamino) methyl]-3- pyridinyl}carbonyl)-3- piperidinyl]butyl}carbamate |
| I-146a | 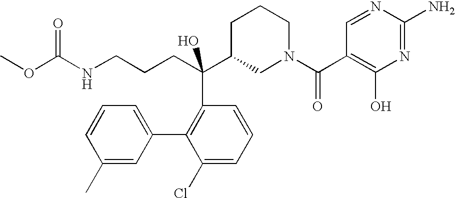 | methyl [(4S)-4-{(3R)-1-[(2-amino- 4-oxo-1,4-dihydro-5- pyrimidinyl)carbonyl]-3-piperidinyl}-4- (6-chloro-3′-methyl-2-biphenylyl)-4- hydroxybutyl] carbamate |
| I-147a | 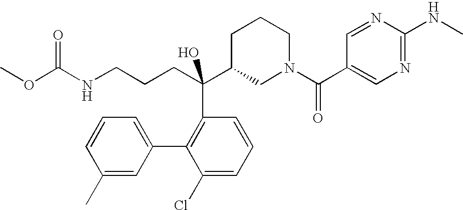 | methyl [(4S)-4-(6-chloro-3′- methyl-2-biphenylyl)-4-hydroxy-4- ((3R)-1-{[2-(methylamino)-5- pyrimidinyl]carbonyl}-3- piperidinyl)butyl]carbamate |
| I-148a | 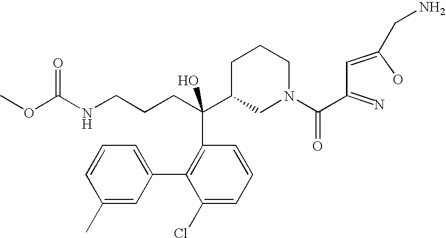 | methyl [(4S)-4-((3R)-1-{[5- (aminomethyl)-3-isoxazolyl] carbonyl}- 3-piperidinyl)-4-(6-chloro-3′-methyl-2- biphenylyl)-4-hydroxybutyl] carbamate |
| I-149a | 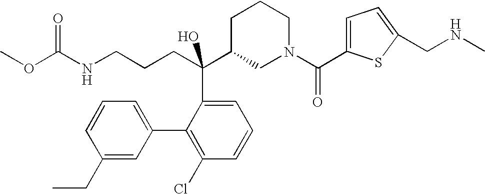 | methyl {(4S)-4-(6-chloro-3′-ethyl- 2-biphenylyl)-4-hydroxy-4-[(3R)-1-({5- [(methylamino) methyl]-2- thienyl}carbonyl)-3- piperidinyl]butyl}carbamate |
| I-150a | 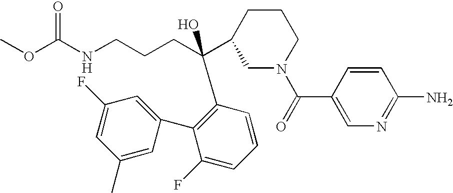 | methyl [(4S)-4-{(3R)-1-[(6-amino- 3-pyridinyl)carbonyl]-3-piperidinyl}-4- (3′,6-difluoro-5′-methyl-2-biphenylyl)- 4-hydroxybutyl]carbamate |
| I-151a | 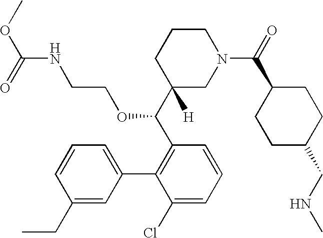 | methyl [2-({(R)-(6-chloro-3′-ethyl- 2-biphenylyl)[(3R)-1-({trans-4- [(methylamino)methyl] cyclohexyl}carbonyl)-3-piperidinyl] methyl}oxy)ethyl]carbamate |
| I-152a | 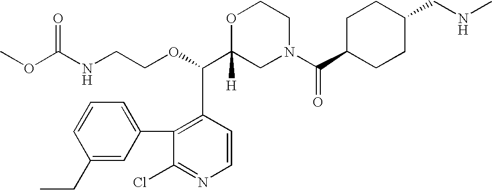 | methyl [2-({(S)-(6-chloro-3′-ethyl- 2-biphenylyl)[(2R)-4-({trans-4- [(methylamino)methyl] cyclohexyl}carbonyl)-2- morpholinyl]methyl}oxy)ethyl] carbamate |
| I-153a | 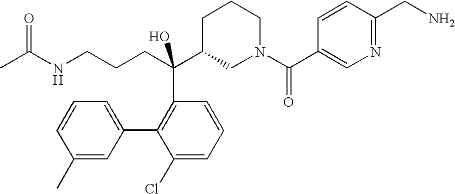 | N-[(4S)-4-((3R)-1-{[6- (aminomethyl)-3-pyridinyl] carbonyl}- 3-piperidinyl)-4-(6-chloro-3′-methyl-2- biphenylyl)-4-hydroxybutyl]acetamide |
| I-154a | 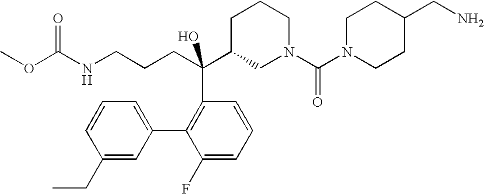 | methyl [(4S)-4-((3R)-1-{[4- (aminomethyl)-1-piperidinyl] carbonyl}-3-piperidinyl)-4-(3′-ethyl-6- fluoro-2-biphenylyl)-4-hydroxybutyl] carbamate |
| I-155a | 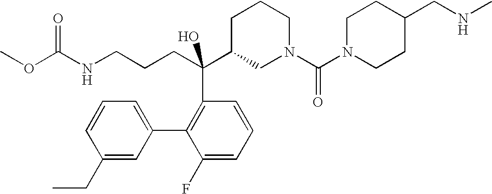 | methyl {(4S)-4-(3′-ethyl-6-fluoro- 2-biphenylyl)-4-hydroxy-4-[(3R)-1-({4- [(methylamino) methyl]-1- piperidinyl}carbonyl)-3- piperidinyl]butyl}carbamate |
| I-156a | 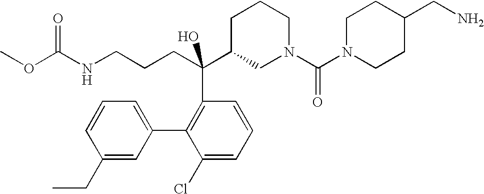 | methyl [(4S)-4-((3R)-1-{[4- (aminomethyl)-1-piperidinyl] carbonyl}-3-piperidinyl)-4-(6-chloro-3′- ethyl-2-biphenylyl)-4- hydroxybutyl]carbamate |
| I-157a | 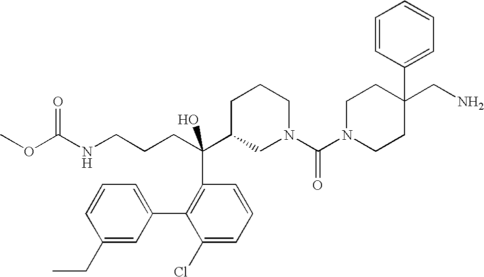 | methyl [(4S)-4-((3R)-1-{[4- (aminomethyl)-4-phenyl-1- piperidinyl]carbonyl}-3-piperidinyl)-4- (6-chloro-3′-ethyl-2-biphenylyl)-4- hydroxybutyl]carbamate |
| I-158a | 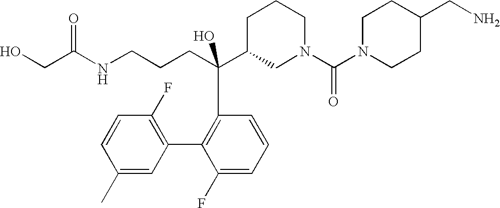 | N-[(4S)-4-((3R)-1-{[4- (aminomethyl)-1-piperidinyl] carbonyl}-3-piperidinyl)-4-(2′,6- difluoro-5′-methyl-2-biphenylyl)-4- hydroxybutyl]-2-hydroxyacetamide |
| I-159a | 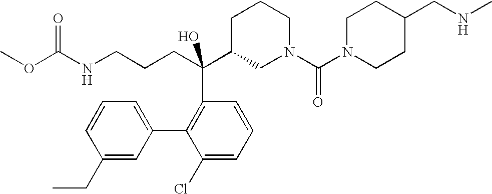 | methyl {(4S)-4-(6-chloro-3′-ethyl- 2-biphenylyl)-4-hydroxy-4-[(3R)-1-({4- [(methylamino) methyl]-1- piperidinyl}carbonyl)-3- piperidinyl]butyl}carbamate |
| I-160a | 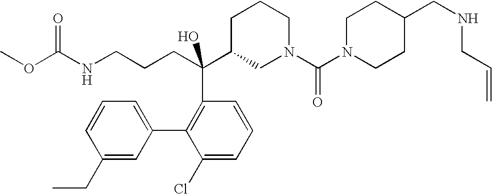 | methyl {(4S)-4-(6-chloro-3′-ethyl- 2-biphenylyl)-4-hydroxy-4-[(3R)-1-({4- [(2-propen-1-ylamino)methyl]-1- piperidinyl} carbonyl)-3- piperidinyl]butyl} carbamate |

or the salts thereof.

The following Compound Nos. represent the preferred compounds of this invention: I-3a, I-6a, I-7a, I-12a, I-15a, I-28a, I-29a, I-32a, I-33a, I-35a, I-38a, I-39a, I-40a, I-48a, I-49a, I-51a, I-53a, I-54a, I-59a, I-76a, I-77a, I-78a, I-79a, I-83a, I-85a, I-87a, I-88a, I-92a, I-95a, I-96a, I-99a, I-101a, I-102a, I-104a, I-105a, I-106a, I-107a, I-108a, I-113a, I-116a, I-117a, I-122a, I-132a, 134a, I-148a, I-150a, and I-153a, or the salts thereof. The following Compound Nos. represent the more preferred compounds of this invention: I-13a, I-20a, I-21a, I-24a, I-26a, I-30a, I-31a, I-36a, I-37a, I-42a, I-43a, I-44a, I-45a, I-46a, I-47a, I-52a, I-55a, I-56a, I-57a, I-58a, I-60a, I-61a, I-63a, I-69a, I-70a, I-72a, I-73a, I-74a, I-81a, I-82a, I-84a, I-89a, I-91a, I-93a, I-94a, I-97a, I-98a, I-109a, I-110a, I-111a, I-114a, I-115a, I-123a, I-124a, I-125a, I-126a, I-127a, I-128a, I-129a, I-130a, I-131a, I-133a, I-135a, I-136a, I-137a, I-138a, I-142a, I-143a, I-145a, I-149a, I-154a, I-155a, I-156a, I-157a, I-158a, I-159a, and I-160a, or the salts thereof.

The compounds of the invention (Compound #1-160) exhibit 50% renin inhibition (as determined using the method of Example 17) at concentrations of from approximately 5000 nM to approximately 0.01 nM. Preferred compounds of the invention exhibit 50% inhibition at concentrations of from approximately 50 nM to approximately 0.01 nM. More preferred compounds of the invention exhibit 50% inhibition at concentrations of from approximately 5 nM to approximately 0.01 nM.

### Enantiomers, Diastereomers, and Salts

Certain compounds of this invention may exist in various stereoisomeric or tautomeric forms. The invention encompasses all such forms, including active compounds in the form of essentially pure enantiomers, racemic mixtures, and tautomers, including forms those not depicted structurally.

The compounds of the invention may be present in the form of pharmaceutically acceptable salts. For use in medicines, the salts of the compounds of the invention refer to non-toxic “pharmaceutically acceptable salts.” Pharmaceutically acceptable salt forms include pharmaceutically acceptable acidic/anionic or basic/cationic salts.

Pharmaceutically acceptable acidic/anionic salts include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, and triethiodide salts.

The compounds of the invention include pharmaceutically acceptable anionic salt forms, wherein the anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, and triethiodide salts.

The anionic salt form of a compound of the invention includes the acetate, bromide, camsylate, chloride, edisylate, fumarate, hydrobromide, hydrochloride, iodide, isethionate, lactate, mesylate, maleate, napsylate, salicylate, sulfate, and tosylate salts.

When a disclosed compound or its pharmaceutically acceptable salt is named or depicted by structure, it is to be understood that solvates or hydrates of the compound or its pharmaceutically acceptable salts are also included. “Solvates” refer to crystalline forms wherein solvent molecules are incorporated into the crystal lattice during crystallization. Solvate may include water or non-aqueous solvents such as ethanol, isopropanol, DMSO, acetic acid, ethanolamine, and EtOAc. Solvates, wherein water is the solvent molecule incorporated into the crystal lattice, are typically referred to as “hydrates”. Hydrates include stoichiometric hydrates as well as compositions containing variable amounts of water.

When a disclosed compound or its pharmaceutically acceptable salt is named or depicted by structure, it is to be understood that the compound, including solvates thereof, may exist in crystalline forms, non-crystalline forms or a mixture thereof.

The compound or its pharmaceutically acceptable salts or solvates may also exhibit polymorphism (i.e. the capacity to occur in different crystalline forms). These different crystalline forms are typically known as “polymorphs.” It is to be understood that when named or depicted by structure, the disclosed compound and its pharmaceutically acceptable salts, solvates or hydrates also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in solidifying the compound. For example, changes in temperature, pressure, or solvent may result in different polymorphs. In addition, one polymorph may spontaneously convert to another polymorph under certain conditions.

It may be necessary and/or desirable during synthesis to protect sensitive or reactive groups on any of the molecules concerned. Representative conventional protecting groups are described in T. W. Greene and P. G. M. Wuts “Protective Groups in Organic Synthesis” John Wiley & Sons, Inc., New York 1999. Protecting groups may be added and removed using methods well known in the art.

The invention also includes various isomers and mixtures thereof. “Isomer” refers to compounds that have the same composition and molecular weight but differ in physical and/or chemical properties. The structural difference may be in constitution (geometric isomers) or in the ability to rotate the plane of polarized light (stereoisomers).

Certain of the disclosed aspartic protease inhibitors may exist in various stereoisomeric forms. Stereoisomers are compounds which differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. The symbol “*” in a structural formula represents the presence of a chiral carbon center. “R” and “S” represent the configuration of substituents around one or more chiral carbon atoms. Thus, “R*” and “S*” denote the relative configurations of substituents around one or more chiral carbon atoms. When a chiral center is not defined as R or S, a mixture of both configurations is present.

“Racemate” or “racemic mixture” means a compound of equimolar quantities of two enantiomers, wherein such mixtures exhibit no optical activity; i.e., they do not rotate the plane of polarized light.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration.

Atoms (other than H) attached to a carbocyclic ring may be in a cis or trans configuration. In the “cis” configuration, the substituents are on the same side in relationship to the plane of the ring; in the “trans” configuration, the substituents are on opposite sides in relationship to the plane of the ring. A mixture of “cis” and “trans” species is designated “cis/trans”.

The point at which a group or moiety is attached to the remainder of the compound or another group or moiety can be indicated by [symbol] which represents “ [symbol] ”, [symbol] , or

[symbol]

“R,” “S,” “S*,” “R*,” “E,” “Z,” “cis,” and “trans,” indicate configurations relative to the core molecule.

The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99% or 99.9% by weight pure relative to the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

When a disclosed compound is named or depicted by structure without indicating the stereochemistry, and the inhibitor has at least one chiral center, it is to be understood that the name or structure encompasses one enantiomer of inhibitor free from the corresponding optical isomer, a racemic mixture of the inhibitor and mixtures enriched in one enantiomer relative to its corresponding optical isomer.

When a disclosed aspartic protease inhibitor is named or depicted by structure without indicating the stereochemistry and has at least two chiral centers, it is to be understood that the name or structure encompasses a diastereomer free of other diastereomers, a pair of diastereomers free from other diastereomeric pairs, mixtures of diastereomers, mixtures of diastereomeric pairs, mixtures of diastereomers in which one diastereomer is enriched relative to the other diastereomer(s) and mixtures of diastereomeric pairs in which one diastereomeric pair is enriched relative to the other diastereomeric pair(s).

The compounds of the invention are useful for ameliorating or treating disorders or diseases in which decreasing the levels of aspartic protease products is effective in treating the disease state or in treating infections in which the infectious agent depends upon the activity of an aspartic protease. In hypertension elevated levels of angiotensin I, the product of renin catalyzed cleavage of angiotensinogen are present. Thus, the compounds of the invention can be used in the treatment of hypertension, heart failure such as (acute and chronic) congestive heart failure; left ventricular dysfunction; cardiac hypertrophy; cardiac fibrosis; cardiomyopathy (e.g., diabetic cardiac myopathy and post-infarction cardiac myopathy); supraventricular and ventricular arrhythmias; arial fibrillation; atrial flutter; detrimental vascular remodeling; myocardial infarction and its sequelae; atherosclerosis; angina (whether unstable or stable); renal failure conditions, such as diabetic nephropathy; glomerulonephritis; renal fibrosis; scleroderma; glomerular sclerosis; microvascular complications, for example, diabetic retinopathy; renal vascular hypertension; vasculopathy; neuropathy; complications resulting from diabetes, including nephropathy, vasculopathy, retinopathy and neuropathy, diseases of the coronary vessels, proteinuria, albumenuria, post-surgical hypertension, metabolic syndrome, obesity, restenosis following angioplasty, eye diseases and associated abnormalities including raised intra-ocular pressure, glaucoma, retinopathy, abnormal vascular growth and remodeling, angiogenesis-related disorders, such as neovascular age related macular degeneration; hyperaldosteronism, anxiety states, and cognitive disorders (Fisher N. D.; Hollenberg N. K. Expert Opin. Investig. Drugs. 2001, 10, 417-26).

Elevated levels of βamyloid, the product of the activity of the well-characterized aspartic protease β-secretase (BACE) activity on amyloid precursor protein, are widely believed to be responsible for the development and progression of amyloid plaques in the brains of Alzheimer's disease patients. The secreted aspartic proteases of Candida albicans are associated with its pathogenic virulence (Naglik, J. R.; Challacombe, S. J.; Hube, B. Microbiology and Molecular Biology Reviews 2003, 67, 400-428). The viruses HIV and HTLV depend on their respective aspartic proteases for viral maturation. Plasmodium falciparum uses plasmepsins I and II to degrade hemoglobin.

A pharmaceutical composition of the invention may, alternatively or in addition to a compound of this invention, comprise a pharmaceutically acceptable salt of a compound of this invention or a prodrug or pharmaceutically active metabolite of such a compound or salt and one or more pharmaceutically acceptable carriers therefore.

The compositions of the invention are aspartic protease inhibitors. Said compositions contain compounds having a mean inhibition constant (IC₅₀) against aspartic proteases of between about 5,000 nM to about 0.01 nM; preferably between about 50 nM to about 0.01 nM; and more preferably between about 5 nM to about 0.01 nM.

The compositions of the invention reduce blood pressure. Said compositions include compounds having an IC₅₀ for renin of between about 5,000 nM to about 0.01 nM; preferably between about 50 nM to about 0.01 nM; and more preferably between about 5 nM to about 0.01 nM.

The invention includes a therapeutic method for treating or ameliorating an aspartic protease mediated disorder in a subject in need thereof comprising administering to a subject in need thereof an effective amount of a compound of this invention, or the enantiomers, diastereomers, or salts thereof or composition thereof.

Administration methods include administering an effective amount (i.e., a therapeutically effective amount) of a compound or composition of the invention at different times during the course of therapy or concurrently in a combination form. The methods of the invention include all known therapeutic treatment regimens.

“Prodrug” means a pharmaceutically acceptable form of an effective derivative of a compound (or a salt thereof) of the invention, wherein the prodrug may be: 1) a relatively active precursor which converts in vivo to a compound of the invention; 2) a relatively inactive precursor which converts in vivo to a compound of the invention; or 3) a relatively less active component of the compound that contributes to therapeutic activity after becoming available in vivo (i.e., as a metabolite). See “Design of Prodrugs”, ed. H. Bundgaard, Elsevier, 1985.

“Metabolite” means a pharmaceutically acceptable form of a metabolic derivative of a compound (or a salt thereof) of the invention, wherein the derivative is an active compound that contributes to therapeutic activity after becoming available in vivo.

“Effective amount” means that amount of active compound agent that elicits the desired biological response in a subject. Such response includes alleviation of the symptoms of the disease or disorder being treated. The effective amount of a compound of the invention in such a therapeutic method is from about 10 mg/kg/day to about 0.01 mg/kg/day, preferably from about 0.5 mg/kg/day to 5 mg/kg/day.

The invention includes the use of a compound of the invention for the preparation of a composition for treating or ameliorating an aspartic protease mediated chronic disorder or disease or infection in a subject in need thereof, wherein the composition comprises a mixture one or more compounds of the invention and an optional pharmaceutically acceptable carrier.

“Pharmaceutically acceptable carrier” means compounds and compositions that are of sufficient purity and quality for use in the formulation of a composition of the invention and that, when appropriately administered to an animal or human, do not produce an adverse reaction.

“Aspartic protease mediated disorder or disease” includes disorders or diseases associated with the elevated expression or overexpression of aspartic proteases and conditions that accompany such diseases.

An embodiment of the invention includes administering a renin inhibiting compound of this invention or composition thereof in a combination therapy (U.S. Pat. No. 5,821,232, U.S. Pat. No. 6,716,875, U.S. Pat. No. 5,663,188, Fossa, A. A.; DePasquale, M. J.; Ringer, L. J.; Winslow, R. L. “Synergistic effect on reduction in blood pressure with coadministration of a renin inhibitor or an angiotensin-converting enzyme inhibitor with an angiotensin II receptor antagonist” Drug Development Research 1994, 33(4), 422-8) with one or more additional agents for the treatment of hypertension including α-blockers, β-blockers, calcium channel blockers, diuretics, natriuretics, saluretics, centrally acting antiphypertensives, angiotensin converting enzyme (ACE) inhibitors, dual ACE and neutral endopeptidase (NEP) inhibitors, angiotensin-receptor blockers (ARBs), aldosterone synthase inhibitor, aldosterone-receptor antagonists, or endothelin receptor antagonist. α-Blockers include doxazosin, prazosin, tamsulosin, and terazosin. β-Blockers for combination therapy are selected from atenolol, bisoprol, metoprolol, acetutolol, esmolol, celiprolol, taliprolol, acebutolol, oxprenolol, pindolol, propanolol, bupranolol, penbutolol, mepindolol, carteolol, nadolol, carvedilol, and their pharmaceutically acceptable salts. Calcium channel blockers include dihydropyridines (DHPs) and non-DHPs. The preferred DHPs are selected from the group consisting of amlodipine, felodipine, ryosidine, isradipine, lacidipine, nicardipine, nifedipine, nigulpidine, niludipine, nimodiphine, nisoldipine, nitrendipine, and nivaldipine and their pharmaceutically acceptable salts. Non-DHPs are selected from flunarizine, prenylamine, diltiazem, fendiline, gallopamil, mibefradil, anipamil, tiapamil, and verampimil and their pharmaceutically acceptable salts. A diuretic is, for example, a thiazide derivative selected from amiloride, chlorothiazide, hydrochlorothiazide, methylchlorothiazide, and chlorothalidon. ACE inhibitors include alacepril, benazepril, benazaprilat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril. Preferred ACE inhibitors are benazepril, enalpril, lisinopril, and ramipril. Dual ACE/NEP inhibitors are, for example, omapatrilat, fasidotril, and fasidotrilat. Preferred ARBs include candesartan, eprosartan, irbesartan, losartan, olmesartan, tasosartan, telmisartan, and valsartan. Preferred aldosterone synthase inhibitors are anastrozole, fadrozole, and exemestane. Preferred aldosterone-receptor antagonists are spironolactone and eplerenone. A preferred endothelin antagonist is, for example, bosentan, enrasentan, atrasentan, darusentan, sitaxentan, and tezosentan and their pharmaceutically acceptable salts.

An embodiment of the invention includes administering an HIV protease inhibiting compound of this invention or composition thereof in a combination therapy with one or more additional agents for the treatment of AIDS including reverse transcriptase inhibitors, non-nucleoside reverse transcriptase inhibitors, other HIV protease inhibitors, HIV integrase inhibitors, attachment and fusion inhibitors, antisense drugs and immune stimulators. Preferred reverse transcriptase inhibitors are zidovudine, didanosine, zalcitabine, stavudine, lamivudine, abacavir, tenofovir, and emtricitabine. Preferred non-nucleoside reverse transcriptase inhibitors are nevirapine, delaviridine, and efavirenz. Preferred HIV protease inhibitors are saquinavir, ritonavir, indinavir, nelfinavir, amprenavir, lopinavir, atazanavir, and fosamprenavir. Preferred HIV integrase inhibitors are L-870,810 and S-1360. A preferred attachment and fusion inhibitor is enfuvirtide.

An embodiment of the invention includes administering β-secretase inhibiting compound of this invention or composition thereof in a combination therapy with one or more additional agents for the treatment of Alzheimer's disease including tacrine, donepezil, rivastigmine, galantamine, and memantine.

An embodiment of the invention includes administering a plasmepsin inhibiting compound of this invention or composition thereof in a combination therapy with one or more additional agents for the treatment of malaria including artemisinin, chloroquine, halofantrine, hydroxychloroquine, mefloquine, primaquine, pyrimethamine, quinine, sulfadoxine

Combination therapy includes co-administration of the compound of the invention and said other agent, sequential administration of the compound and the other agent, administration of a composition containing the compound and the other agent, or simultaneous administration of separate compositions containing of the compound and the other agent.

The invention further includes the process for making the composition comprising mixing one or more of the present compounds and an optional pharmaceutically acceptable carrier; and includes those compositions resulting from such a process, which process includes conventional pharmaceutical techniques.

The compositions of the invention include ocular, oral, nasal, transdermal, topical with or without occlusion, intravenous (both bolus and infusion), and injection (intraperitoneally, subcutaneously, intramuscularly, intratumorally, or parenterally). The composition may be in a dosage unit such as a tablet, pill, capsule, powder, granule, liposome, ion exchange resin, sterile ocular solution, or ocular delivery device (such as a contact lens and the like facilitating immediate release, timed release, or sustained release), parenteral solution or suspension, metered aerosol or liquid spray, drop, ampoule, auto-injector device, or suppository; for administration ocularly, orally, intranasally, sublingually, parenterally, or rectally, or by inhalation or insufflation.

Compositions of the invention suitable for oral administration include solid forms such as pills, tablets, caplets, capsules (each including immediate release, timed release, and sustained release formulations), granules and powders; and, liquid forms such as solutions, syrups, elixirs, emulsions, and suspensions. Forms useful for ocular administration include sterile solutions or ocular delivery devices. Forms useful for parenteral administration include sterile solutions, emulsions, and suspensions.

The compositions of the invention may be administered in a form suitable for once-weekly or once-monthly administration. For example, an insoluble salt of the active compound may be adapted to provide a depot preparation for intramuscular injection (e.g., a decanoate salt) or to provide a solution for ophthalmic administration.

The dosage form containing the composition of the invention contains a therapeutically effective amount of the active ingredient necessary to provide a therapeutic effect. The composition may contain from about 5,000 mg to about 0.5 mg (preferably, from about 1,000 mg to about 0.5 mg) of a compound of the invention or salt form thereof and may be constituted into any form suitable for the selected mode of administration. The composition may be administered about 1 to about 5 times per day. Daily administration or post-periodic dosing may be employed.

For oral administration, the composition is preferably in the form of a tablet or capsule containing, e.g., 500 to 0.5 milligrams of the active compound. Dosages will vary depending on factors associated with the particular patient being treated (e.g., age, weight, diet, and time of administration), the severity of the condition being treated, the compound being employed, the mode of administration, and the strength of the preparation.

The oral composition is preferably formulated as a homogeneous composition, wherein the active ingredient is dispersed evenly throughout the mixture, which may be readily subdivided into dosage units containing equal amounts of a compound of the invention. Preferably, the compositions are prepared by mixing a compound of the invention (or pharmaceutically acceptable salt thereof) with one or more optionally present pharmaceutical carriers (such as a starch, sugar, diluent, granulating agent, lubricant, glidant, binding agent, and disintegrating agent), one or more optionally present inert pharmaceutical excipients (such as water, glycols, oils, alcohols, flavoring agents, preservatives, coloring agents, and syrup), one or more optionally present conventional tableting ingredients (such as corn starch, lactose, sucrose, sorbitol, talc, stearic acid, magnesium stearate, dicalcium phosphate, and any of a variety of gums), and an optional diluent (such as water).

Binder agents include starch, gelatin, natural sugars (e.g., glucose and beta-lactose), corn sweeteners and natural and synthetic gums (e.g., acacia and tragacanth). Disintegrating agents include starch, methyl cellulose, agar, and bentonite.

Tablets and capsules represent an advantageous oral dosage unit form. Tablets may be sugarcoated or filmcoated using standard techniques. Tablets may also be coated or otherwise compounded to provide a prolonged, control-release therapeutic effect. The dosage form may comprise an inner dosage and an outer dosage component, wherein the outer component is in the form of an envelope over the inner component. The two components may further be separated by a layer which resists disintegration in the stomach (such as an enteric layer) and permits the inner component to pass intact into the duodenum or a layer which delays or sustains release. A variety of enteric and non-enteric layer or coating materials (such as polymeric acids, shellacs, acetyl alcohol, and cellulose acetate or combinations thereof) may be used.

Compounds of the invention may also be administered via a slow release composition; wherein the composition includes a compound of the invention and a biodegradable slow release carrier (e.g., a polymeric carrier) or a pharmaceutically acceptable non-biodegradable slow release carrier (e.g., an ion exchange carrier).

Biodegradable and non-biodegradable slow release carriers are well known in the art. Biodegradable carriers are used to form particles or matrices which retain an active agent(s) and which slowly degrade/dissolve in a suitable environment (e.g., aqueous, acidic, basic and the like) to release the agent. Such particles degrade/dissolve in body fluids to release the active compound(s) therein. The particles are preferably nanoparticles (e.g., in the range of about 1 to 500 nm in diameter, preferably about 50-200 nm in diameter, and most preferably about 100 nm in diameter). In a process for preparing a slow release composition, a slow release carrier and a compound of the invention are first dissolved or dispersed in an organic solvent. The resulting mixture is added into an aqueous solution containing an optional surface-active agent(s) to produce an emulsion. The organic solvent is then evaporated from the emulsion to provide a colloidal suspension of particles containing the slow release carrier and the compound of the invention.

The compound of this invention may be incorporated for administration orally or by injection in a liquid form such as aqueous solutions, suitably flavored syrups, aqueous or oil suspensions, flavored emulsions with edible oils such as cottonseed oil, sesame oil, coconut oil or peanut oil and the like, or in elixirs or similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone, and gelatin. The liquid forms in suitably flavored suspending or dispersing agents may also include synthetic and natural gums. For parenteral administration, sterile suspensions and solutions are desired. Isotonic preparations, which generally contain suitable preservatives, are employed when intravenous administration is desired.

The compounds may be administered parenterally via injection. A parenteral formulation may consist of the active ingredient dissolved in or mixed with an appropriate inert liquid carrier. Acceptable liquid carriers usually comprise aqueous solvents and other optional ingredients for aiding solubility or preservation. Such aqueous solvents include sterile water, Ringer's solution, or an isotonic aqueous saline solution. Other optional ingredients include vegetable oils (such as peanut oil, cottonseed oil, and sesame oil), and organic solvents (such as solketal, glycerol, and formyl). A sterile, non-volatile oil may be employed as a solvent or suspending agent. The parenteral formulation is prepared by dissolving or suspending the active ingredient in the liquid carrier whereby the final dosage unit contains from 0.005 to 10% by weight of the active ingredient. Other additives include preservatives, isotonizers, solubilizers, stabilizers, and pain-soothing agents. Injectable suspensions may also be prepared, in which case appropriate liquid carriers, suspending agents and the like may be employed.

Compounds of the invention may be administered intranasally using a suitable intranasal vehicle.

Compounds of the invention may also be administered topically using a suitable topical transdermal vehicle or a transdermal patch.

For ocular administration, the composition is preferably in the form of an ophthalmic composition. The ophthalmic compositions are preferably formulated as eye-drop formulations and filled in appropriate containers to facilitate administration to the eye, for example a dropper fitted with a suitable pipette. Preferably, the compositions are sterile and aqueous based, using purified water. In addition to the compound of the invention, an ophthalmic composition may contain one or more of: a) a surfactant such as a polyoxyethylene fatty acid ester; b) a thickening agents such as cellulose, cellulose derivatives, carboxyvinyl polymers, polyvinyl polymers, and polyvinylpyrrolidones, typically at a concentration n the range of about 0.05 to about 5.0% (wt/vol); c) (as an alternative to or in addition to storing the composition in a container containing nitrogen and optionally including a free oxygen absorber such as Fe), an anti-oxidant such as butylated hydroxyanisol, ascorbic acid, sodium thiosulfate, or butylated hydroxytoluene at a concentration of about 0.00005 to about 0.1% (wt/vol); d) ethanol at a concentration of about 0.01 to 0.5% (wt/vol); and e) other excipients such as an isotonic agent, buffer, preservative, and/or pH-controlling agent. The pH of the ophthalmic composition is desirably within the range of 4 to 8.

The compounds of this invention may be generically defined by the following Formula I:

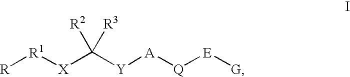

or an enantiomer, diastereomer or salt thereof, wherein R is:

a) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)alkoxy, (C₃-C₈)alkenyloxy, (C₃-C₈)alkynyloxy, (C₃-C₇)cycloalkoxy, (C₅-C₇)cyclo-alkenyloxy, (C₃-C₇)cycloalkoxy(C₁-C₃)alkyl, (C₃-C₇)cycloalkyl(C₁-C₃)alkoxy, (C₅-C₇)cycloalkenyl(C₁-C₃)alkoxy, (C₁-C₈)alkylthio, (C₃-C₈)alkenylthio, (C₃-C₈)alkynylthio, (C₃-C₇)cycloalkylthio(C₁-C₃)alkyl, (C₃-C₇)cycloalkyl(C₁-C₃)alkylthio, (C₅-C₇)cycloalkenyl(C₁-C₃)alkylthio, (C₁-C₈)alkylamino, di(C₁-C₈)alkylamino, azepano, azetidino, piperidino, pyrrolidino, (C₃-C₇)cycloalkylamino, ((C₃-C₇)cycloalkyl(C₁-C₃)alkyl)amino or tri(C₁-C₄)alkylsilyl, each optionally substituted with up to four substituents independently selected from the group consisting of fluorine, hydroxy, (C₁-C₆)alkyl, halo(C₁-C₆)alkyl, (C₃-C₆)cycloalkyl, (C₁-C₆)alkoxy, (C₁-C₆)cycloalkoxy and oxo;

b) aryl, heteroaryl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, aryl(C₂-C₃))alkenyl, aryl(C₂-C₃)alkynyl, heteroaryl(C₂-C₃))alkenyl, or heteroaryl(C₂-C₃))alkynyl, each optionally substituted with up to three substituents independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylhio, (C₄-C₇)cycloalkylalkylthio, halo(C₁-C₆)alkylthio, halo(C₃-C₆)cycloalkylhio, halo(C₄-C₇)cycloalkylalkylthio, (C₁-C₆)alkanesulflnyl, (C₃-C₆)cycloalkanesulfinyl, (C₄-C₇)cycloalkylalkanesulflnyl, halo(C₁-C₆)alkane-sulflnyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkyl, halo(C₁-C₆)alkoxy(C₁-C₆)alkyl, (C₁-C₆)alkoxy(C₁-C₆)alkoxy, halo(C₁-C₆)alkoxy(C₁-C₆)alkoxy, (C₁-C₆)alkoxycarbonyl, H₂NCO, H₂NSO₂, (C₁-C₆)alkylaminocarbonyl, and di(C₁-C₆)alkylaminocarbonyl, (C₁-C₆)alkylaminosulfonyl, and di(C₁-C₆)alkylaminosulfonyl; or

c) a divalent radical selected from —(CH₂)₃—, —(CH₂)₄—, —(CH₂)₅— or —(CH₂)₆—, which is attached to R¹ to form a fused or spiro-fused ring system, and is optionally substituted with up to four substituents independently selected from the group consisting of fluorine, hydroxy, (C₁-C₆)alkyl, halo(C₁-C₆)alkyl, (C₁-C₆)alkoxy and oxo;

R¹ is phenyl, monocyclic heteroaryl, bicyclic heteroaryl, benzo-1,3-dioxole, benzo-1,3-dioxine, 2,3-dihydrobenzo-1,4-dioxine or (C₃-C₇)cycloalkyl, each optionally substituted with up to four substituents independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylhio, (C₄-C₇)cycloalkylalkylthio, halo(C₁-C₆)alkylthio, halo(C₃-C₆)cycloalkylhio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulflnyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NSO₂, H₂NCO, (C₁-C₆)alkylaminosulfonyl, di(C₁-C₆)alkylaminosulfonyl, (C₁-C₆)alkylaminocarbonyl and di(C₁-C₆)alkylaminocarbonyl;

X and Y are each independently CH₂ or a single bond;

R² is a) —H; or b) (C₁-C₁₂)alkyl, (C₂-C₁₂)alkenyl, (C₂-C₁₂)alkynyl, (C₁-C₁₂)alkoxy, (C₁-C₁₂)alkylthio, (C₁-C₁₂)alkylamino, oxo(C₁-C₁₂)alkyl, oxo(C₂-C₁₂)alkenyl, oxo(C₂-C₁₂)alkynyl, oxo(C₁-C₁₂)alkoxy, oxo(C₁-C₁₂)alkylthio, oxo(C₁-C₁₂)alkylamino, (C₁-C₆)alkoxy(C₁-C₆)alkyl, (C₁-C₆)alkylthio(C₁-C₆)alkyl, (C₁-C₆)alkylamino(C₁-C₆)alkyl, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio (C₁-C₆)alkoxy, (C₁-C₆)alkylthio(C₁-C₆)alkylamino, (C₁-C₆)alkylthio(C₁-C₆)alkylthio, (C₁-C₆)alkylamino (C₁-C₆)alkoxy, (C₁-C₆)alkylamino (C₁-C₆)alkylthio, (C₁-C₆)alkylamino(C₁-C₆)alkylamino, (C₁-C₄)alkoxy(C₁-C₄)alkoxy(C₁-C₄)alkyl, amino carbonylamino (C₁-C₁₂)alkyl, amino carbonylamino (C₁-C₁₂)alkoxy, amino carbonylamino (C₁-C₁₂)alkylthio, amino carbonylamino (C₁-C₁₂)alkylamino, (C₁-C₆)-alkanoylamino(C₁-C₆)alkyl, (C₁-C₆)alkanoylamino(C₁-C₆)alkoxy, (C₁-C₆)alkanoylamino(C₁-C₆)alkylthio, (C₁-C₆)alkanoylamino (C₁-C₆)alkylamino, (C₁-C₆)alkoxycarbonyl(C₁-C₆)alkyl, (C₁-C₆)alkoxycarbonyl(C₁-C₆)alkoxy, (C₁-C₆)alkoxycarbonyl(C₁-C₆)alkylthio, (C₁-C₆)alkoxy-carbonyl(C₁-C₆)alkylamino, (C₁-C₆)acyloxy(C₁-C₆)alkyl, (C₁-C₆)acyloxy(C₁-C₆)alkoxy, (C₁-C₆)acyloxy(C₁-C₆)alkylthio, (C₁-C₆)acyloxy(C₁-C₆)alkylamino, amino sulfonylamino (C₁-C₁₂)alkyl, amino sulfonylamino (C₁-C₁₂)alkoxy, amino sulfonylamino (C₁-C₁₂)alkylthio, amino sulfonyl-amino (C₁-C₁₂)alkylamino, (C₁-C₆)alkanesulfonylamino(C₁-C₆)alkyl, (C₁-C₆)alkanesulfonyl-amino(C₁-C₆)alkoxy, (C₁-C₆)alkane sulfonylamino (C₁-C₆)alkylthio, (C₁-C₆)alkanesulfonyl-amino (C₁-C₆)alkylamino, formylamino(C₁-C₆)alkyl, formylamino(C₁-C₆)alkoxy, formylamino(C₁-C₆)alkylthio, formylamino(C₁-C₆)alkylamino, (C₁-C₆)alkoxycarbonylamino (C₁-C₆)alkyl, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino (C₁-C₆)alkylamino, (C₁-C₆)alkylaminocarbonyl-amino(C₁-C₆)alkyl, (C₁-C₆)alkylaminocarbonylamino(C₁-C₆)alkoxy, (C₁-C₆)alkylamino carbonyl-amino (C₁-C₆)alkylthio, (C₁-C₆)alkylaminocarbonylamino(C₁-C₆)alkylamino, amino carbonyl(C₁-C₆)alkyl, amino carbonyl(C₁-C₆)alkoxy, amino carbonyl(C₁-C₆)alkylthio, amino carbonyl(C₁-C₆)alkylamino, (C₁-C₆)alkylamino carbonyl(C₁-C₆)alkyl, (C₁-C₆)alkylaminocarbonyl(C₁-C₆)alkoxy, (C₁-C₆)alkylamino carbonyl(C₁-C₆)alkylthio, (C₁-C₆)alkylaminocarbonyl(C₁-C₆)alkylamino, amino carboxy(C₁-C₆)alkyl, aminocarboxy(C₁-C₆)alkoxy, aminocarboxy(C₁-C₆)alkylthio, aminocarboxy(C₁-C₆)alkylamino, (C₁-C₆)alkylamino carboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, each optionally substituted by: 1) 1 to 5 halogen atoms; and 2) 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy, where the divalent sulfur atoms in R² are independently optionally oxidized to sulfoxide or sulfone and wherein the carbonyl groups are optionally independently changed to a thiocarbonyl groups;

R³ is —H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkanoylamino, (C₁-C₆)alkoxycarbonylamino, (C₁-C₆)alkylamino-carbonylamino, di(C₁-C₆)alkylaminocarbonylamino, (C₁-C₆)alkanesulfonylamino, (C₁-C₆)alkylaminosulfonylamino, di(C₁-C₆)alkylaminosulfonyl-amino, phenylamino or heteroarylamino in which each phenylamino or heteroarylamino group is optionally substituted with 1 to 5 groups independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylhio, (C₄-C₇)cycloalkylalkylthio, halo(C₁-C₆)alkylthio, halo(C₃-C₆)cycloalkylhio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulflnyl, halo(C₃-C₆)cycloalkanesulfinyl, halo(C₄-C₇)-cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)-cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, amino-carbonyl, (C₁-C₆)alkylaminocarbonyl, and di(C₁-C₆)alkylaminocarbonyl, provided that i) R² and R³ are not both hydrogen; and ii) when R³ is hydroxyl, halogen, or optionally substituted phenylamino or heteroarylamino, R² is not (C₁-C₁₂)alkoxy, (C₁-C₁₂)alkylthio, (C₁-C₁₂)alkylamino, oxo(C₁-C₁₂)alkoxy, oxo(C₁-C₁₂)alkylthio, oxo(C₁-C₁₂)alkylamino, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)-alkylthio (C₁-C₆)alkylthio, (C₁-C₆)alkylamino (C₁-C₆)alkoxy, (C₁-C₆)alkylamino(C₁-C₆)alkylthio, (C₁-C₆)alkylamino(C₁-C₆)alkylamino, amino carbonylamino (C₁-C₁₂)alkoxy, amino carbonyl-amino (C₁-C₁₂)alkylthio, amino carbonylamino (C₁-C₁₂)alkylamino, (C₁-C₆)alkanoylamino(C₁-C₆)alkoxy, (C₁-C₆)alkanoylamino (C₁-C₆)alkylthio, (C₁-C₆)alkanoylamino (C₁-C₆)alkylamino, (C₁-C₆)alkoxycarbonyl(C₁-C₆)alkoxy, (C₁-C₆)alkoxycarbonyl(C₁-C₆)alkylthio, (C₁-C₆)alkoxycarbonyl-(C₁-C₆)alkylamino, (C₁-C₆)acyloxy(C₁-C₆)alkoxy, (C₁-C₆) acyloxy(C₁-C₆)alkylthio, (C₁-C₆)-acyloxy(C₁-C₆)alkylamino, aminosulfonylamino(C₁-C₁₂)alkoxy, aminosulfonylamino(C₁-C₁₂)alkylthio, amino sulfonylamino (C₁-C₁₂)alkylamino, (C₁-C₆)alkanesulfonylamino(C₁-C₆)alkoxy, (C₁-C₆)alkanesulfonylamino(C₁-C₆)alkylthio, (C₁-C₆)alkanesulfonylamino(C₁-C₆)alkylamino, formylamino(C₁-C₆)alkoxy, formylamino(C₁-C₆)alkylthio, formylamino(C₁-C₆)alkylamino, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino (C₁-C₆)alkylamino, (C₁-C₆)alkylamino carbonyl-amino (C₁-C₆)alkoxy, (C₁-C₆)alkylaminocarbonylamino(C₁-C₆)alkylthio, (C₁-C₆)alkylamino-carbonylamino(C₁-C₆)alkylamino, aminocarbonyl(C₁-C₆)alkoxy, aminocarbonyl(C₁-C₆)alkylthio, amino carbonyl(C₁-C₆)alkylamino, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkylamino, aminocarboxy(C₁-C₆)alkoxy, aminocarboxy(C₁-C₆)alkylthio, amino carboxy(C₁-C₆)alkylamino, (C₁-C₆)alkylaminocarboxy(C₁-C₆)alkoxy, (C₁-C₆)alkylamino carboxy(C₁-C₆)alkylthio, (C₁-C₆)alkylamino carboxy(C₁-C₆)alkylamino, (C₁-C₁₂)alkoxycarbonylamino, (C₁-C₁₂)alkylamino-carbonylamino, or (C₁-C₁₂)alkanoylamino, each optionally substituted by: 1) 1 to 5 halogen atoms; and 2) 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, or halo(C₃-C₆)cycloalkoxy; where the divalent sulfur atoms in R³ are independently optionally oxidized to sulfoxide or sulfone and wherein the carbonyl groups in R³ are optionally independently changed to thiocarbonyl groups;

A is a saturated or unsaturated 4-, 5-, 6-, or 7-membered ring which is optionally bridged by (CH₂)ₘ via bonds to two members of said ring, wherein said ring is composed of carbon atoms and 0-2 hetero atoms selected from the group consisting of 0, 1, or 2 nitrogen atoms, 0 or 1 oxygen atoms, and 0 or 1 sulfur atoms, said ring being optionally substituted with up to four independently selected halogen atoms, (C₁-C₆)alkyl groups, halo(C₁-C₆)alkyl groups or oxo groups such that when there is substitution with one oxo group on a carbon atom it forms a carbonyl group and when there is substitution of one or two oxo groups on sulfur it forms sulfoxide or sulfone groups, respectively, where m is 1 to 3;

Q and Y are attached to carbon or nitrogen atoms in ring A in a 1,2 or 1,3, or 1,4 relationship;

Q is a divalent radical selected from

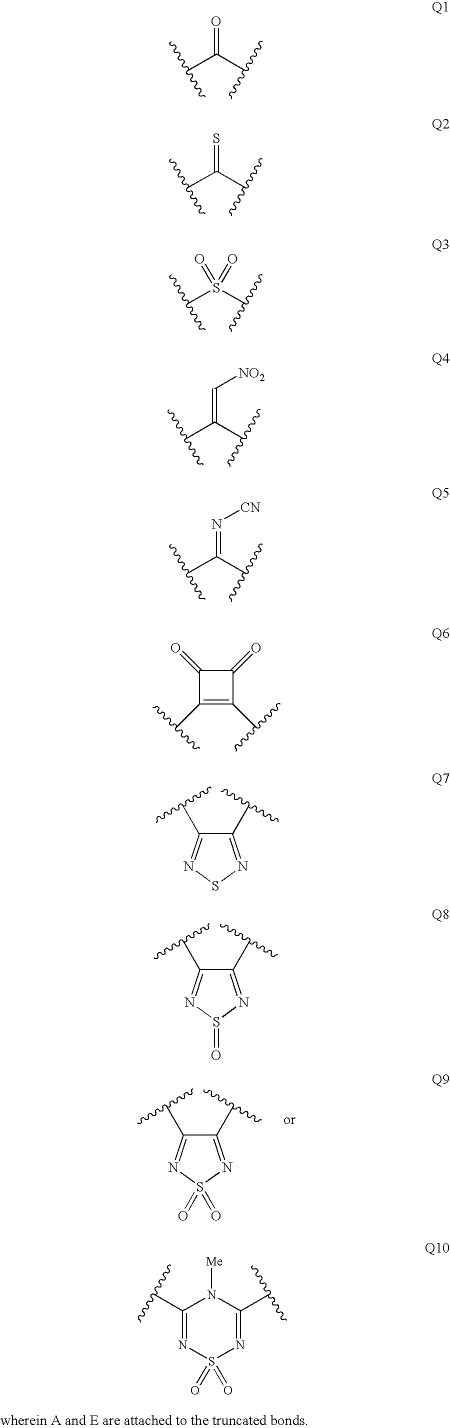

E is a saturated or unsaturated 3-, 4-, 5-, 6-, or 7-membered ring which is optionally bridged by (CH₂) via bonds to two members of said ring, wherein said ring is composed of carbon atoms, and 0-3 hetero atoms selected from 0, 1, 2, or 3 nitrogen atoms, 0 or 1 oxygen atoms, and 0 or 1 sulfur atoms, said ring being optionally substituted with up to four groups independently selected from halogen, hydroxy, (C₁-C₆)alkyl, halo(C₁-C₆)alkyl, hydroxy(C₁-C₆)alkyl, aryl, and oxo groups such that when there is substitution with one oxo group on a carbon atom it forms a carbonyl group and when there is substitution of one or two oxo groups on sulfur it forms sulfoxide or sulfone groups, respectively, where n is 1 to 3; and

G is hydroxy, hydroxy(C₁-C₆)alkyl, amino, (C₁-C₆)alkylamino, hydroxy(C₁-C₆)alkylamino, amino (C₁-C₆)alkyl, (C₁-C₆)alkylamino(C₁-C₆)alkyl, (C₂-C₆)alkenylamino(C₁-C₆)alkyl, heteroaryl(C₁-C₆)alkyl, C(═NH)NH₂, C(═NH)NHR⁴, NHC(═NH)NH₂, or NHC(═NH)NHR⁴, where R⁴ is (C₁-C₃)alkyl, and wherein the heteroaryl moiety of the heteroaryl(C₁-C₆)alkyl group is optionally substituted with 1 or two substituents independently selected from the group consisting of (C₁-C₄)alkyl, hydroxy(C₁-C₄)alkyl, amino(C₁-C₄)alkyl, or (C₁-C₄)alkylamino(C₁-C₄)alkyl;

wherein Ring A in Formula I may be depicted as follows:

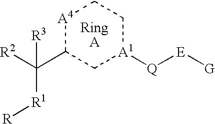

wherein Ring A is a benzene ring (A¹ and A⁴ are CH and the bonds in ring A are aromatic bonds); or Ring A is a piperidine ring (A¹ is N, A⁴ is CH₂ and the bonds in ring A are single bonds); or Ring A is a morpholine ring (A¹ is N, A⁴ is O and the bonds in ring A are single bonds).

In the discussion below the substituent variables R, R¹, R², R³, X, Y, A, Q, E, and G are as defined above:

When any variable (e.g., aryl, alkyl, R′, R², etc.) occurs more than once in a compound, its definition on each occurrence is independent of any other occurrence.

“Alkyl” means a saturated aliphatic branched or straight-chain mono- or divalent hydrocarbon radical having the specified number of carbon atoms. Thus, “(C₁-C₈)alkyl” means a radical having from 1-8 carbon atoms in a linear or branched arrangement. “(C₁-C₆)alkyl” includes methyl, ethyl, propyl, butyl, pentyl, and hexyl.

“Cycloalkyl” means a saturated aliphatic cyclic hydrocarbon radical having the specified number of carbon atoms. Thus, (C₃-C₇)cycloalkyl means a radical having from 3-8 carbon atoms arranged in a ring. (C₃-C₇)cycloalkyl includes cyclopropyl, cyclobutyl, cyclopentyl, cyclohexyl, and cycloheptyl.

Haloalkyl and halocycloalkyl include mono, poly, and perhaloalkyl groups where the halogens are independently selected from fluorine, chlorine, and bromine.

“Heteroaryl” means a monovalent heteroaromatic monocyclic and polycylic ring radical. Heteroaryl rings are 5- and 6-membered aromatic heterocyclic rings containing 1 to 4 heteroatoms independently selected from N, O, and S, and include furan, thiophene, pyrrole, imidazole, pyrazole, oxazole, isoxazole, thiazole, isothiazole, 1,2,3-triazole, 1,2,4-triazole, 1,3,4-oxadiazole, 1,2,5-thiadiazole, 1,2,5-thiadiazole 1-oxide, 1,2,5-thiadiazole 1,1-dioxide, 1,3,4-thiadiazole, pyridine, pyridine-N-oxide, pyrazine, pyrimidine, pyridazine, 1,2,4-triazine, 1,3,5-triazine, and tetrazole. Bicyclic heteroaryl rings are bicyclo[4.4.0] and bicyclo[4,3.0] fused ring systems containing 1 to 4 heteroatoms independently selected from N, O, and S, and include indolizine, indole, isoindole, benzo[b]furan, benzo[b]thiophene, indazole, benzimidazole, benzthiazole, purine, 4H-quinolizine, quinoline, isoquinoline, cinnoline, phthalazine, quinazoline, quinoxaline, 1,8-naphthyridine, and pteridine.

“Alkoxy” means an alkyl radical attached through an oxygen linking atom. “(C₁-C₄)-alkoxy” includes methoxy, ethoxy, propoxy, and butoxy.

“Aromatic” means an unsaturated cycloalkyl ring system.

“Aryl” means an aromatic monocyclic, or polycyclic ring system. Aryl systems include phenyl, naphthalenyl, fluorenyl, indenyl, azulenyl, and anthracenyl.

“Hetero” refers to the replacement of at least one carbon atom member in a ring system with at least one heteroatom selected from N, S, and O. A hetero ring may have 1, 2, 3, or 4 carbon atom members replaced by a heteroatom.

“Unsaturated ring” means a ring containing one or more double bonds and include cyclopentene, cyclohexene, cyclopheptene, cyclohexadiene, benzene, pyrroline, pyrazole, 4,5-dihydro-1H-imidazole, imidazole, 1,2,3,4-tetrahydropyridine, 1,2,3,6-tetrahydropyridine, pyridine and pyrimidine.

In cases where the synthetic intermediates and final products of this invention described below contain potentially reactive functional groups, for example amino, hydroxyl, thiol and carboxylic acid groups, that may interfere with the desired reaction, it may be advantageous to employ protected forms of the intermediate. Methods for the selection, introduction and subsequent removal of protecting groups are well known to those skilled in the art. (T. W. Greene and P. G. M. Wuts “Protective Groups in Organic Synthesis” John Wiley & Sons, Inc., New York 1999). In the discussion below all intermediates are assumed to be protected when necessary and protection/deprotection are generally not described.

In the first process of the invention, a compound of Formula I, in which a nitrogen atom that is part of A is attached to Q, is prepared by reaction of an amine of Formula II and an intermediate of Formula III:

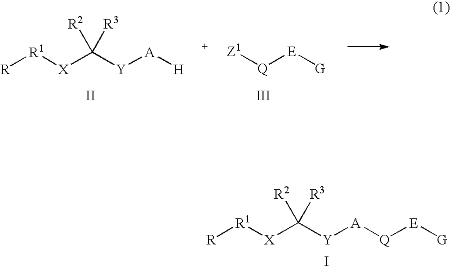

wherein Z¹ in III is a leaving group such as halide, alkanesulfonate, haloalkanesulfonate, carboxylate, arylsulfonate, aryloxy, heteroaryloxy, azole, azolium salt, alkoxy, alkylthio, or arylthio.

Intermediates of formula II wherein H is attached to a nitrogen atom that is part of A are prepared from intermediates of Formula IV:

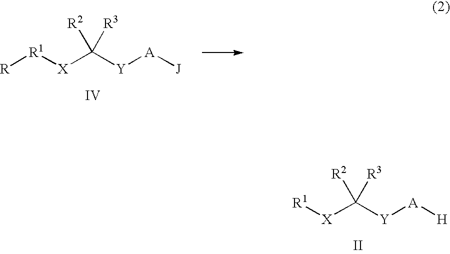

wherein J is an amine protecting group, including carbamate, amide, and sulfonamide protecting groups known in the art (T. W. Greene and P. G. M. Wuts “Protective Groups in Organic Synthesis” John Wiley & Sons, Inc., New York 1999).

Intermediates of Formula IV wherein R³═OH are prepared from ketone intermediates of formula V by addition of an organometallic reagent of formula VI, where M is for example Li, MgCl, MgBr, or MgI, to the carbonyl group of V:

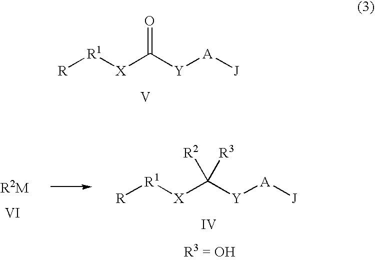

Intermediates of Formula IV wherein R³═H and R² is a group attached by an ether linkage are prepared from alcohol intermediates of formula VII by reaction with an alkylating agent under basic conditions or by reaction with an alcohol of formula R²OH under acidic conditions.

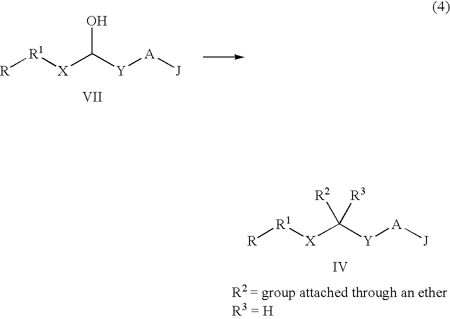

Alcohol intermediates of formula VII are prepared by reduction of ketone intermediates of formula V:

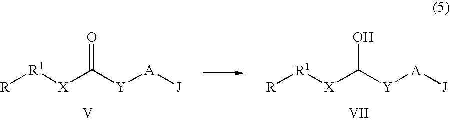

or by addition of an organometallic reagent of formula VIII, wherein M is, for example Li, MgCl, MgBr, or MgI, to an aldehyde of Formula IX:

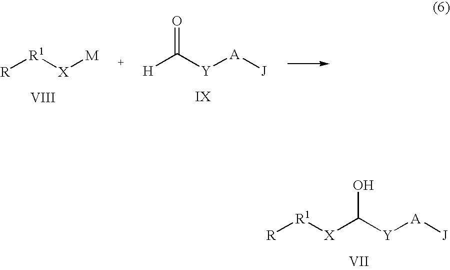

Ketone intermediates of formula V are prepared by the addition of an organometallic reagent of formula VIII, wherein M is Li, MgCl, MgBr, MgI, to a carboxylic acid derivative of formula X wherein Z² is an alkoxy, dialkylamino group, or an N-alkoxy-N-alkylamino group:

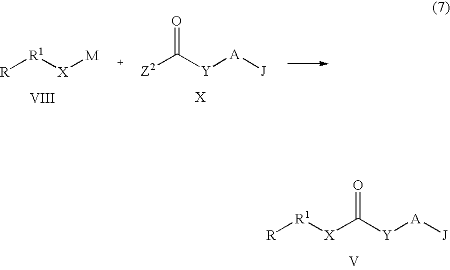

Intermediates of Formula III, wherein Q is Q1 attached to a carbon atom of E and Z¹ is alkanesulfonate, haloalkanesulfonate, carboxylate, arylsulfonate, or represents an active ester are prepared by activation of carboxylic acids of Formula XV:

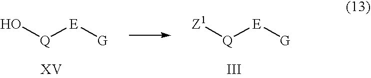

Reagents used to effect carboxylic activation are well known in the literature and include thionyl chloride and oxalyl chloride used to prepare acid chlorides, alkanesulfonyl chlorides used to prepare mixed anhydrides, alkyl chloroformates used to prepare mixed anhydrides, and carbodiimides used to prepare active esters. Intermediates of formula III are often prepared and used in situ without isolation.

In another process of the invention, a compound of Formula I, in which a nitrogen atom that is part of E is attached to Q, is prepared by reaction of an intermediate of Formula XVIII and an amine of Formula XVI:

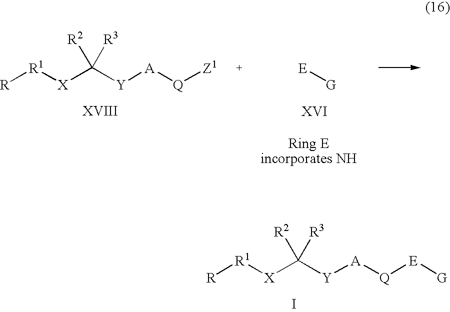

wherein Z¹ is as defined above.

Intermediates of Formula XVIII wherein Q is attached to a nitrogen atom of ring A and Q is Q1, Q4, Q5, Q6, Q8, Q9, or Q10 are prepared from amine intermediates of Formula II and intermediates of Formula XVII wherein Z¹ is halide, alkanesulfonate, haloalkanesulfonate, carboxylate, arylsulfonate, aryloxy, heteroaryloxy, azole, azolium salt, alkoxy, alkylthio, or arylthio:

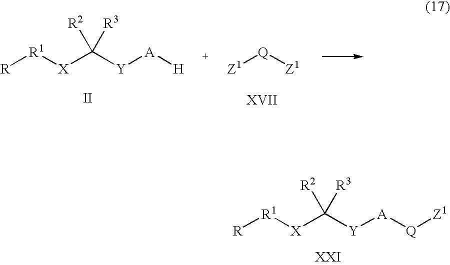

In another process of the invention, a compound of Formula I, in which R is an alkoxy, cycloalkoxy, cycloalkylalkoxy or arylalkoxy group, is prepared by reaction of an alkylating agent of Formula XXII, in which Z³ is chloride, bromide, iodide, methanesulfonate, arenesulfonate or trifluoromethanesulfonate and R^{c} is an alkyl, cycloalkyl, cycloalkylalkyl or arylalkyl, with a hydroxy compound of Formula XXIII:

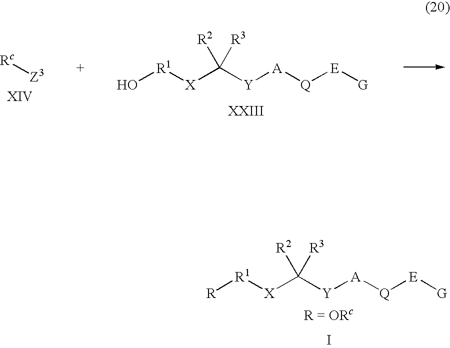

Intermediates of Formula XXIII are prepared by routes analogous to those shown for compounds of Formula I in equations 1 and 16.

In the first process of the invention, a compound of Formula Ia, in which A¹ is a nitrogen atom is prepared by reaction of an amine of Formula IIa and an intermediate of Formula IIIa:

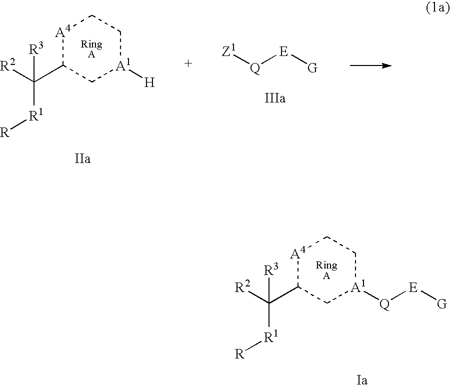

wherein Z¹ in III is a leaving group such as halide, alkanesulfonate, haloalkanesulfonate, carboxylate, arylsulfonate, aryloxy, heteroaryloxy, azole, azolium salt, alkoxy, alkylthio, or arylthio.

Intermediates of formula IIa in which A¹ is a nitrogen atom are prepared from intermediates of Formula IVa:

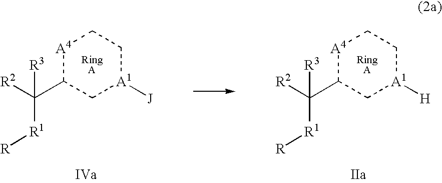

wherein J is an amine protecting group, including carbamate, amide and sulfonamide protecting groups known in the art (T. W. Greene and P. G. M. Wuts “Protective Groups in Organic Synthesis” John Wiley & Sons, Inc., New York 1999).

Intermediates of Formula IVa wherein R³═OH are prepared from ketone intermediates of formula Va by addition of an organometallic reagent of formula VIa, where M is for example Li, MgCl, MgBr, or MgI, to the carbonyl group of Va:

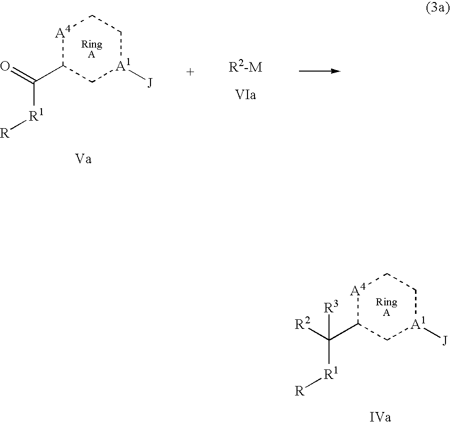

Intermediates of Formula IVa wherein R³═H and R² is a group attached by an ether linkage are prepared from alcohol intermediates of formula VIIa by reaction with an alkylating agent under basic conditions or by reaction with an alcohol under acidic conditions.

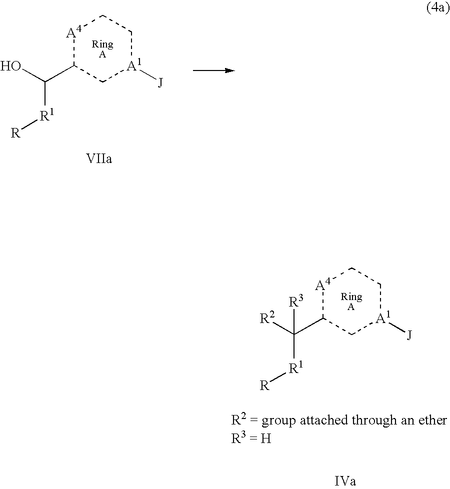

Alcohol intermediates of formula VIIa are prepared by reduction of ketone intermediates of formula Va using reagents known in the art (Handbook of Reagents for Organic Synthesis: Oxidizing and Reducing Reagents Ed. S. D. Burke and R. L. Danheiser, John Wiley & Sons, New York, 1999):

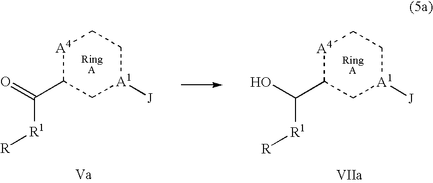

or by addition of an organometallic reagent of formula VIIIa, wherein M is, for example Li, MgCl, MgBr, or MgI, to an aldehyde of Formula IXa:

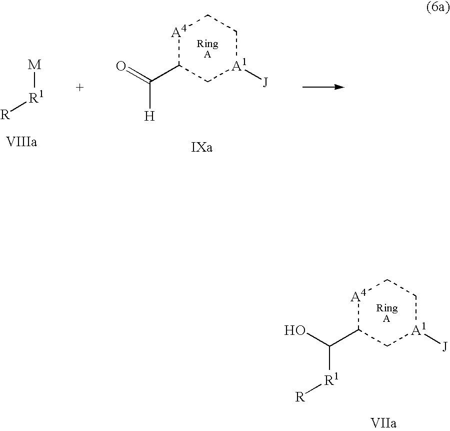

Ketone intermediates of formula Va are prepared by the addition of an organometallic reagent of formula VIIIa, wherein M is Li, MgCl, MgBr, MgI, to a carboxylic acid derivative of formula Xa wherein Z² is an alkoxy, dialkylamino group, or an N-alkoxy-N-alkylamino group:

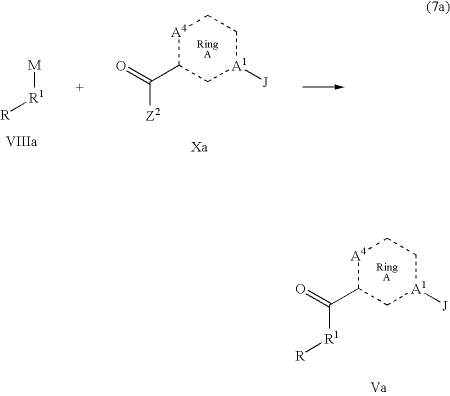

Intermediates of formula Va are also prepared by oxidation of alcohol intermediates of formula VIIa using reagents known in the art (Handbook of Reagents for Organic Synthesis: Oxidizing and Reducing Reagents Ed. S. D. Burke and R. L. Danheiser, John Wiley & Sons, New York, 1999):

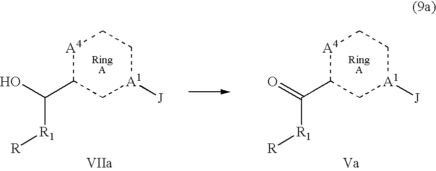

Intermediates of Formula IVa, wherein the R is group attached to R¹ through an ether linkage, are also prepared by alkylation of intermediates of formula XIIIa, in which Z³ is a hydroxyl group with alkylating agents of formula XIVa, wherein X is a halogen, alkanesulfonate, haloalkanesulfonate, or arenesulfonate leaving group:

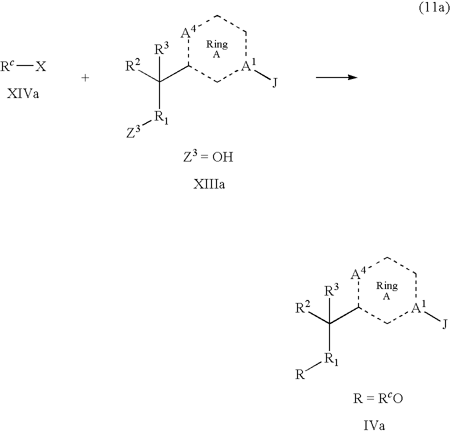

The intermediates of Formula XIIIa used in equation 11a are available by processes analogous to those described for IVa (equations 3a and 4a).

Intermediates of Formula Ma, wherein Q is Q1 attached to a carbon atom of E and Z¹ is alkanesulfonate, haloalkanesulfonate, carboxylate, arylsulfonate, or represents an active ester are prepared by activation of carboxylic acids of Formula XVa:

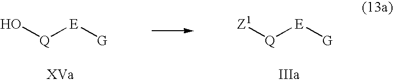

Reagents used to effect carboxylic activation are well known in the literature and include thionyl chloride and oxalyl chloride used to prepare acid chlorides, alkanesulfonyl chlorides used to prepare mixed anhydrides, alkyl chloroformates used to prepare mixed anhydrides, and carbodiimides used to prepare active esters. Intermediates of formula IIIa are often prepared and used in situ without isolation.

In another process of the invention, a compound of Formula Ia, in which a nitrogen atom that is part of E is attached to Q, is prepared by reaction of an intermediate of Formula XVIIIa and an amine of Formula XVIa:

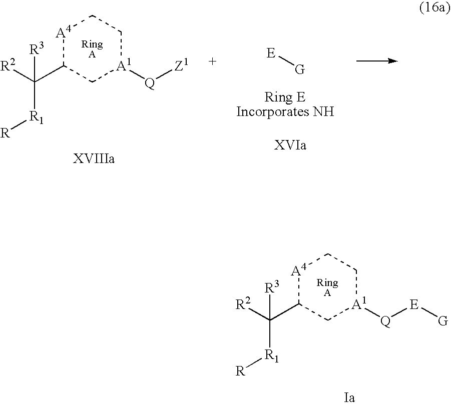

wherein Z¹ is as defined above.

Intermediates of Formula XVIIIa wherein Q is attached to a nitrogen atom of ring A and Q is Q1, Q4, Q5, Q6, Q8, Q9, or Q10 are prepared from amine intermediates of Formula IIa and intermediates of Formula XVIIa wherein Z¹ is halide, alkanesulfonate, haloalkanesulfonate, carboxylate, arylsulfonate, aryloxy, heteroaryloxy, azole, azolium salt, alkoxy, alkylthio, or arylthio:

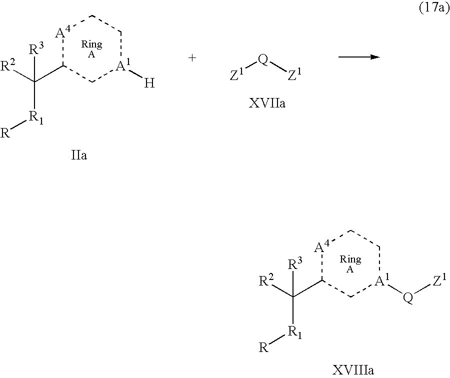

In another process of the invention, a compound of Formula Ia, in which R is an alkoxy, cycloalkoxy, cycloalkylalkoxy or arylalkoxy group, is prepared by reaction of an alkylating agent of Formula XIVa, in which Z³ is chloride, bromide, iodide, methanesulfonate, arenesulfonate or trifluoromethanesulfonate and Rc is an alkyl, cycloalkyl, cycloalkylalkyl or arylalkyl group, with a hydroxy compound of Formula XXIIa:

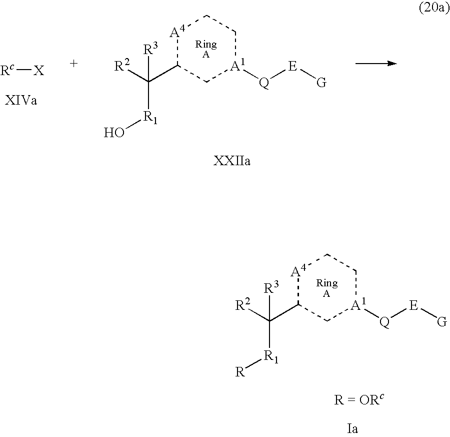

Intermediates of Formula XXIIa are prepared by routes analogous to those shown for compounds of Formula Ia in reaction schemes 1a and 16a.

The invention is further defined by reference to the examples, which are intended to be illustrative and not limiting.

Representative compounds of the invention can be synthesized in accordance with the general synthetic schemes described above and are illustrated in the examples that follow. The methods for preparing the various starting materials used in the schemes and examples are well within the knowledge of persons skilled in the art. During the course of preparing aryl 3-piperidinyl ketones, as described in the following protocols (e.g. Preparations 5-7 and 11), racemization of the stereocenter adjacent to the carbonyl group can occur and was specifically observed during the preparation of (R)-tent-butyl 3-(6-chloro-3′-ethylbiphenylcarbonyl)piperidine-1-carboxylate. In this case, the racemic product was detected when the reaction mixture was allowed to stir at room temperature for prolonged times (e.g. overnight) but was not observed when the ketone forming reaction was quenched at −78° C. (by addition of aqueous ammonium chloride). When racemization does occur, the resulting stereoisomers may be resolved using conventional methods well known to those skilled in the art. Accordingly, it will be appreciated by those skilled in the art, that in the following Experimental section, any identification of a specific stereoisomer (e.g., assignment of configuration of a chiral center) in a final or intermediate product compound name or structure is to be understood to represent the intended relative or absolute configuration of that chiral center, but not necessarily the only stereoisomer obtained.

The following abbreviations have the indicated meanings:

| Abbreviation | Meaning |
|---|---|
| Aq | aqueous |
| Boc | tert-butoxy carbonyl or t-butoxy carbonyl |
| (Boc)₂O | di-tert-butyl dicarbonate |
| Brine | saturated aqueous NaCl |
| CH₂Cl₂ | methylene chloride |
| CH₃CN or MeCN | acetonitrile |
| Cpd | compound |
| D | day |
| DBU | 1,8-diazabicyclo[5.4.0]undec-7-ene |
| DIEA | N,N-diisopropylethylamine |
| DMAP | 4-(dimethylamino)pyridine |
| DMF | N,N-dimethylformamide |
| DMPU | 1,3-dimethyl-3,4,5,6-tetrahydro-2(1H)-pyrimidinone |
| EDC•HCl | 1-[3-(dimethylamino)propyl]-3-ethylcarbodiimide |
| | hydrochloride |
| Equiv | equivalents |
| Et | ethyl |
| Et₂O | ethyl ether |
| EtOAc | ethyl acetate |
| Fmoc | 1-[[(9H-fluoren-9-ylmethoxy)carbonyl]oxy]- |
| Fmoc-OSu | 1-[[(9H-fluoren-9-ylmethoxy)carbonyl]oxy]-2,5- |
| | pyrrolidinedione |
| h, hr | hour |
| HOBt | 1-hydroxybenzotriazole |
| HATU | 2-(7-Aza-1H-benzotriazole-1-yl)-1,1,3,3- |
| | tetramethyluronium |
| | hexafluorophosphate |
| HBTU | 2-(1H-Benzotriazole-1-yl)-1,1,3,3-tetramethyluronium |
| | hexafluorophosphate |
| KHMDS | potassium hexamethyldisilazane |
| LAH or LiAlH₄ | lithium aluminum hydride |
| LC-MS | liquid chromatography-mass spectroscopy |
| LHMDS | lithium hexamethyldisilazane |
| Me | methyl |
| MeCN | aceronitrile |
| MeOH | methanol |
| MsCl | methanesulfonyl chloride |
| min | minute |
| MS | mass spectrum |
| NaH | sodium hydride |
| NaHCO₃ | sodium bicarbonate |
| NaN₃ | sodium azide |
| NaOH | sodium hydroxide |
| Na₂SO₄ | sodium sulfate |
| NMP | N-methylpyrrolidinone |
| P₄-t-Bu | 1-tert-butyl-4,4,4-tris(dimethylamino)- |
| | 2,2-bis[tris(dimethylamino)- |
| | phosphoranylidenamino]-2/\⁵,4/\⁵- |
| | catenadi(phosphazene) |
| Pd₂(dba)₃ | tris(dibenzylideneacetone)dipalladium(0) |
| Ph | phenyl |
| rt | room temperature |
| satd | saturated |
| SOCl₂ | thionyl chloride |
| TBAF | tetrabutylammonium fluoride |
| TEA | triethylamine or Et₃N |
| TEAF | tetraethylammonium fluoride |
| TEMPO | 2,2,6,6-tetramethyl-1-piperidinyloxy, free radical |
| Teoc | 1-[2-(trimethylsilyl)ethoxycarbonyloxy]- |
| Teoc-OSu | 1-[2-(trimethylsilyl)ethoxycarbonyloxy]pyrrolidin- |
| | 2,5-dione |
| TFA | trifluoroacetic acid |
| THF | tetrahydrofuran |
| TMSCl | chlorotrimethylsilane or trimethylsilyl chloride |
| t_{R} | retention time |

### LC-MS Methods

Method 1 [Instrument 1]: Analytical LC-MS was conducted on an Agilent 1100 Series LC/MSD SL or VL using electrospray positive [ES+ve to give MH⁺] equipped with a Sunfire C₁₈ 5.0 μm column (3.050 mm×50 3.0 mm, i.d.), eluting with 0.05% TFA in water (solvent A) and 0.05% TFA in acetonitrile (solvent B), using the following elution gradient 10%±99% (solvent B) over 3.0 min and holding at 99% for 1.0 min at a flow rate of 1.0 ml/min.

Method 2 [Instrument 2]: Analytical LC-MS was conducted on an PE Sciex API 150 single quadrupole mass spectrometer using electrospray positive [ES+ve to give MH+] equipped with a Aquasil C18 5 μm column (1 mm×40 mm), eluting with 0.02% TFA in water (solvent A) and 0.018% TFA in acetonitrile (solvent B), using the following elution gradient 4.5%-90% (solvent B) over 3.2 min and holding at 90% for 0.4 min at a flow rate of 0.3 ml/min.

Method 3: Analytical LC-MS was conducted on an Agilent 1200 Series LC/MSD VL using electrospray positive [ES+ve to give MH⁺] equipped with a YMC C₁₈ 5.0 μm column (2.0 mm×50, 2.0 mm, i.d.), eluting with 0.0375% TFA in water (solvent A) and 0.01875% TFA in acetonitrile (solvent B), using the following elution gradient 10%-80% (solvent B) over 2.0 min and holding at 80% for 0.5 min at a flow rate of 1.0 ml/min.

### Chiral HPLC Method

Column: Chiralpak AD-H, 0.46 cm×25 cm

### Solvent A: 0.025% Diethylamine in Hexane

### Solvent B: Isopropanol

Flow rate: 1 mL/min.
40 min. run

### Gradient:

| Time (min) | A(%) | B(%) |
|---|---|---|
| 0 | 95 | 5 |
| 40 | 90 | 10 |

The following procedures describe preparation of intermediates used in the synthesis of compounds of this invention.

### Preparation 1

### Weinreb Amide

### (R)-tert-butyl 3-(N-methoxy-N-methylcarbamoyl)piperidine-1-carboxylate

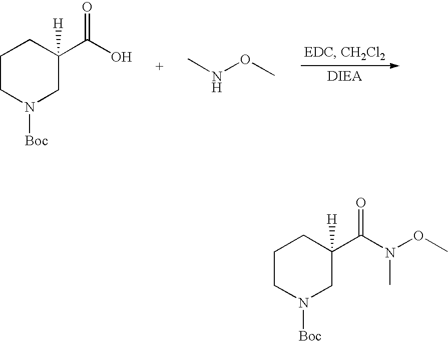

(R)-1-(tent-butoxy carbonyl)piperidine-3-carboxylic acid (25 g, 0.11 mol, 1.0 equiv), N,O-dimethylhydroxylamine hydrochloride, (10.5 g, 0.14 mol, 1.25 equiv), EDC.HCl (26.3 g, 0.14 mol, 1.25 equiv) and DIEA (48 mL, 0.28 mol, 2.5 equiv) were dissolved in CH₂Cl₂ (400 mL) and stirred overnight at rt. The reaction mixture was diluted with EtOAc, washed with 5% aq HCl (2×150 mL), satd aq NaHCO₃ (150 mL), brine (100 mL), and dried over Na₂SO₄. Concentration afforded (R)-tent-butyl 3-(N-methoxy-N-methylcarbamoyl)-piperidine-1-carboxylate (24.42 g, 82%) as a clear oil.

### Preparation 2

### Halodiphenyl Ethers from Halophenols and Benzeneboronic Acids

### 1-(3-Fluorophenoxy)-2-bromobenzene

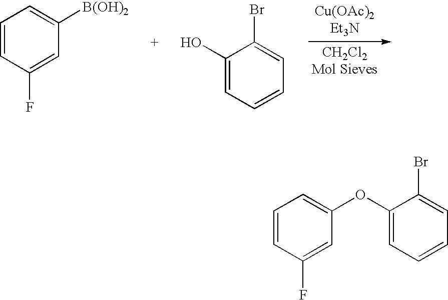

To a stirred solution of 3-fluorophenylboronic acid (2.10 g, 15 mmol), 2-bromophenol (1.77 g, 10 mmol) and Cu(OAc)₂ (0.93 g, 5 mmol) in anhydrous CH₂Cl₂ (25 mL) was added activated 4 Å molecular sieves (˜0.1 g), followed by anhydrous Et₃N (3.5 mL, 25 mmol). The resulting dark green solution was stirred at rt for 48 h. The mixture was evaporated under reduced pressure and the residue was washed several times with Et₂O (˜150 mL). The Et₂O solution was washed with satd aq NH₄Cl, and 1 N aq HCl. The organic layer was evaporated and the crude product was purified by flash column chromatography to give 1-(3-fluorophenoxy)-2-bromobenzene (1.28 g, 48%) as clear oil.

The following halodiphenyl ethers were prepared following the procedure described above.

| Halodiphenyl ether | Phenol | Boronic Acid |
|---|---|---|
| 1-bromo-3-chloro-2-[(3- | 2-bromo-6- | 3-ethylphenylboronic acid |
| ethylphenyl)oxy]benzene | chlorophenol | |
| 1-bromo-3-chloro-2-[(2- | 2-bromo-6- | 2-methylphenylboronic |
| methylphenyl)oxy]benzene | chlorophenol | acid |
| 1-bromo-3-chloro-2-[(2- | 2-bromo-6- | 2-ethylphenylboronic acid |
| ethylphenyl)oxy]benzene | chlorophenol | |
| 1-bromo-2-[(3- | 2-bromo-6- | (3-methylphenyl)boronic |
| methylphenyl)oxy]-3- | fluorophenol | acid |
| fluorobenzene | | |
| 1-bromo-3-chloro-2-[(3- | 2-bromo-6- | (3-methylphenyl)boronic |
| methylphenyl)oxy]benzene | chlorophenol | acid |

### Preparation 3

### Halodiphenyl Ethers From Phenoxyanilines

### 1-(O-tolyloxy)-2-iodobenzene

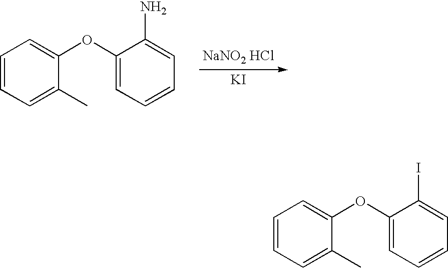

To a solution of 2-(o-tolyloxy)aniline (40 g, 0.2 mol) in 1N aq HCl (400 mL, 0.4 mol, 2 equiv) cooled to 0° C. was added dropwise a solution of NaNO₂ (18 g, 0.26 mol, 1.3 equiv) in water (520 ml). The mixture was stirred for 1 h at 0° C. and a solution of KI (83 g, 0.5 mol, 2.5 equiv) in water (500 mL) was added dropwise with vigorous stirring. After 0.5 h the mixture was warmed to 90-100° C. for 1 h, cooled to rt and washed with satd NaHSO₃ until the aqueous layer become clear. The mixture was extracted with EtOAc (3×200 mL) and the combined organic layers were washed with aq Na₂S₂O₄ and dried over Na₂SO₄. After evaporation of the solvent, the solution was passed through a short silica gel column to afford 1-(o-tolyloxy)-2-iodobenzene (40.0 g, 65%).

### Preparation 4

### Halodiphenyl Ethers from Phenols and Fluoronitrobenzenes

### 1-(2-Iodophenoxy)-2-chlorobenzene

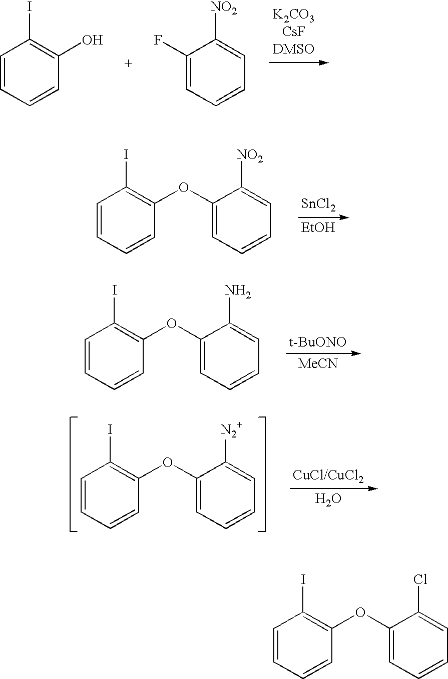

Step 1. 1-(2-Iodophenoxy)-2-nitrobenzene: To a solution of 2-iodophenol (11. 82 g, 52.7 mmol) and 1-fluoro-2-nitrobenzene (5.0 g, 35.1 mmol) in DMSO (50 mL was added K₂CO₃ (14.5 g, 105.3 mmol), followed by CsF (8.0 g, 52.7 mmol). The resulting suspension was stirred at 50° C. until no starting material remained (˜5 h), cooled to rt and partitioned between water (50 mL) and CH₂Cl₂ (50 mL). The water layer was separated and extracted with CH₂Cl₂ (2×10 mL). The combined organic layers were washed with 1 aq N NaOH (10 mL) and brine, and dried over Na₂SO₄. Solvent was removed under vacuum to give 1-(2-iodophenoxy)-2-nitrobenzene (11.2 g, 93%) as an oil, which was used for next step without purification.

Step 2. 2-(2-Iodophenoxy)benzenamine: A solution of 1-(2-iodophenoxy)-2-nitrobenzene (9.60 g, 28.1 mmol) and SnCl.2H₂O (13.0 g, 56.0 mmol) in ethanol (25 mL) and water (5 mL) was refluxed until no starting material remained (˜1 h). The ethanol was removed in vacuo and the aq layer was basified to pH>10 and extracted with CH₂Cl₂ (4×10 mL). The combined organic layers were dried over Na₂SO₄, and the solvent was removed to give a crude 2-(2-Iodophenoxy)benzenamine (8.57 g, 98%), which was used for the next step without purification.

Step 3. 1-(2-Iodophenoxy)-2-chlorobenzene: A solution of crude 2-(2-iodophenoxy)benzenamine (8.57 g, 27.6 mmol) in MeCN (60 mL) was cooled to 0° C. and treated with HBF₄ (54 wt % in Et₂O, 4.93 mL, 35.9 mmol). The reaction mixture was stirred at 0° C. for 5 min and of t-BuONO (4.10 g, 35.9 mmol) was added dropwise. The resulting mixture was stirred at 0° C. for 10 min, cooled to −20° C., and added to a solution of CuCl (41 g, 414.1 mmol) and CuCl₂ (70 g, 414.1 mmol) in water (500 mL) at 0° C. The mixture was stirred vigorously at 25° C. for 2 h, and partitioned between EtOAc and water. The water layer was extracted with EtOAc (3×10 mL) and the combined organic layers were washed with brine, dried over Na₂SO₄ and concentrated under vacuum. Flash column chromatography gave 1-(2-iodophenoxy)-2-chlorobenzene (5.35 g, 58%).

The following halodiphenyl ethers were prepared following the procedures described above using the starting materials and reagents indicated:

| | | Halide |
|---|---|---|
| Halopdiphenyl ether | Phenol in Step 1 | in Step 3 |
| 2-[(2-bromophenyl)oxy]-1,3- | 2,6-dimethylphenol | CuBr/CuBr₂ |
| dimethylbenzene | | |
| 2-[(2-bromophenyl)oxy]-1-chloro-3- | 2-chloro-6- | CuBr/CuBr₂ |
| methylbenzene | methylphenol | |
| 1-bromo-3-chloro-2-[(2- | 2-methylphenol | CuBr/CuBr₂ |
| methylphenyl)oxy]benzene | | |

### Preparation 5

### Piperidines from Weinreb Amides and Halodiphenylethers

### 1-(2-(3-Fluorophenoxy)phenyl)-5-methoxy-1-((R)-piperidin-3-yl)pentan-1-ol

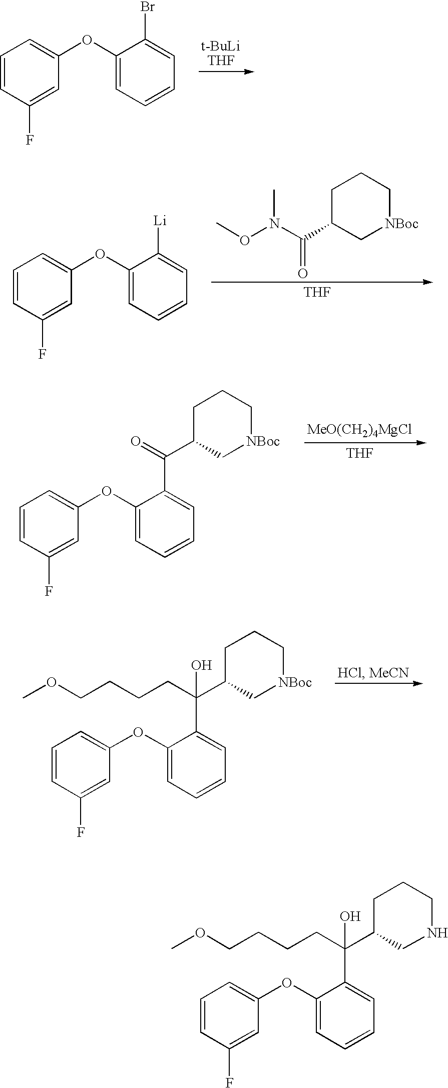

Step 1. 2-(3-Fluorophenoxy)phenyllithium: To a stirred solution of 1-(3-fluorophenoxy)-2-bromobenzene (1.27 g, 4.75 mmol) in THF (10 mL) at −70° C. was added 1.7 M t-BuLi in pentane (5.6 mL, 9.50 mmol) dropwise to keep the temperature below −70° C. The resulting solution was stirred at −70° C. for 30 min, and used for the next step directly.

Step 2. (3R)-1-(tent-butoxycarbonyl)-3-((3-fluorophenoxy)benzoyl)piperidine: To a solution of (R)-tent-butyl 3-(N-methoxy-N-methylcarbamoyl)piperidine-1-carboxylate (0.65 g, 2.37 mmol) in THF (4 mL) at −20° C. was added dropwise the solution of 2-(3-fluorophenoxy)phenyllithium prepared in Step 2 above. After the addition was complete, the resulting solution was allowed to warm to rt slowly, and left at rt for 1 h. The reaction was quenched with 1N HCl (˜6 mL), and extracted with Et₂O (4×10 mL). The combined organic layers were washed with satd aq NaHCO₃ and brine, and dried over Na₂SO₄. Removal of the solvent left the crude ketone (1.49 g, quantitative), which was used for next step without further purification.

Step 3. (3R)-tert-butyl 3-(1-(2-(3-fluorophenoxy)phenyl)-1-hydroxy-5-methoxypentyl)piperidine-1-carboxylate: To a solution of (3R)-1-(tert-butoxycarbonyl)-3-((3-fluorophenoxy)benzoyl)piperidine (0.95 g, 2.37 mmol) in THF (3 mL) at −20° C. was added 1.45 M 4-methoxybutyl magnesium chloride in THF (3.3 mL, 4.76 mmol) dropwise. The resulting solution was warmed to rt slowly, and the completion of reaction was confirmed by LC-MS (˜20 min). The reaction was quenched with satd aq NH₄Cl (4 mL) and extracted with Et₂O (4×5 mL). The combined organic layers were washed with water and brine, and the solvent was removed in vacuo to give a crude product which was purified by flash column chromatography to afford (3R)-tert-butyl 3-(1-(2-(3-fluorophenoxy)phenyl)-1-hydroxy-5-methoxypentyl)piperidine-1-carboxylate (0.50 g, 43%).

Step 4. 1-(2-(3-Fluorophenoxy)phenyl)-5-methoxy-1-((R)-piperidin-3-yl)pentan-1-ol: To a solution of (3R)-tert-butyl 3-(1-(2-(3-fluorophenoxy)phenyl)-1-hydroxy-5-methoxypentyl)piperidine-1-carboxylate (0.50 g, 1.03 mmol) in MeCN (60 mL) was added 2 N aq HCl (60 mL) slowly at rt. The resulting solution was stirred at rt overnight, then basified to pH=10 with 10 N aq NaOH. The mixture was evaporated under reduced pressure to remove MeCN. The aq layer was extracted with CH₂Cl₂ (4×10 mL), and the combined organic layers were washed with brine and dried over Na₂SO₄. The solvent was removed under vacuum to give 1-(2-(3-fluorophenoxy)phenyl)-5-methoxy-1-((R)-piperidin-3-yl)pentan-1-ol (0.40 g, quantitative) as a free amine.

The following piperidines prepared using the above procedures using the halodiphenyl ethers listed below in Step 1.

| Piperidine | Halodiphenyl ether |
|---|---|
| 1-(2-(2,6-dimethylphenoxy)phenyl)-5- | 2-(2-bromophenoxy)-1,3- |
| methoxy-1-((R)-piperidin-3-yl)pentan-1-ol | dimethylbenzene |
| 1-(3-chloro-2-(o-tolyloxy)phenyl)-5-methoxy- | 1-bromo-3-chloro-2-(o- |
| 1-((R)-piperidin-3-yl)pentan-1-ol | tolyloxy)benzene |
| 1-(2-(2-chloro-6-methylphenoxy)phenyl)-5- | 2-(2-bromophenoxy)-1- |
| methoxy-1-((R)-piperidin-3-yl)pentan-1-ol | chloro-3-methylbenzene |
| 1-(3-chloro-2-(2-ethylphenoxy)phenyl)-5- | 1-bromo-3-chloro-2-(2- |
| methoxy-1-((R)-piperidin-3-yl)pentan-1-ol | ethylphenoxy)benzene |
| 1-(3-chloro-2-(3-ethylphenoxy)phenyl)-5- | 1-bromo-3-chloro-2-(3- |
| methoxy-1-((R)-piperidin-3-yl)pentan-1-ol | ethylphenoxy)benzene |
| 1-{3-chloro-2-[(3-methylphenyl)oxy]phenyl}- | 1-bromo-3-chloro-2-[(3- |
| 5-(methyloxy)-1-[(3R)-3-piperidinyl]-1- | methyl- |
| pentanol | phenyl)oxy]benzene |
| 1-{3-fluoro-2-[(3-methylphenyl)oxy]phenyl}- | 1-bromo-3-fluoro-2-[(3- |
| 5-(methyloxy)-1-[(3R)-3-piperidinyl]-1- | methyl- |
| pentanol | phenyl)oxy]benzene |

### Preparation 6

### Boc Protected Piperidines from Weinreb Amides and Iododiphenyl Ethers

### 5-(methyloxy)-1-{2-[(2-methylphenyl)oxy]phenyl}-1-[(3R)-3-piperidinyl]-1-pentanol

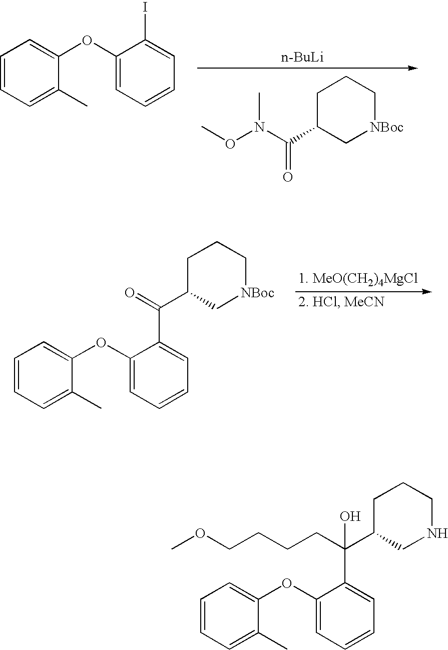

Step 1. (2-(O-tolyloxy)phenyl)((R)-1-(tert-butoxycarbonyl)piperidin-3-yl)methanone: To a solution of 1-(o-tolyloxy)-2-iodobenzene (40 g, 0.13 mol) in anhydrous THF (500 mL) cooled to −78° C. was added dropwise 1.6 M n-BuLi in hexanes (52 mL, 0.13 mol). After stirring for 1 h at −78° C., a solution of (R)-tent-butyl 3-(N-methoxy-N-methylcarbamoyl)-piperidine-1-carboxylate (35 g, 0.13 mol) in anhydrous THF (500 mL) was added dropwise. The mixture was allowed to warm to rt and stirred overnight. Saturated aq NH₄Cl (500 mL) was added and the mixture was extracted with EtOAc (3×150 mL). The combined organic layers were dried over Na₂SO₄. Solvent removal and flash column chromatography afforded (2-(o-tolyloxy)phenyl)((R)-1-(tert-butoxycarbonyl)piperidin-3-yl)methanone (23 g, 45%).

Step 2. (3R)-tert-butyl 3-(1-(2-(o-tolyloxy)phenyl)-1-hydroxy-5-methoxypentyl)piperidine-1-carboxylate: A 500-mL, three-necked flask was charged with magnesium turnings (12 g, 0.5 mol) and a small crystal of iodine. The flask was evacuated and refilled with N₂. A solution of 1-chloro-4-methoxybutane (50 g, 0.4 mol) in THF (200 mL) was added dropwise to the mixture. The reaction mixture was stirred at reflux for 2 h and most of magnesium was consumed. The solution of Grignard reagent was cooled to rt.

A 1000 mL, three-necked flask was charged with the (2-(o-tolyloxy)phenyl)((R)-1-(tert-butoxycarbonyl)piperidin-3-yl)methanone (20 g, 0.05 mol) and THF (250 mL). The flask was evacuated and refilled with N₂, the mixture was cooled with a dry ice-acetone bath and the Grignard reagent was added dropwise. The mixture was allowed to warm slowly to rt and stirred overnight. After quenching with satd aq NH₄Cl (500 mL), the mixture was extracted with EtOAc (3×150 mL) and the combined organic layers were dried over Na₂SO₄. The solvent was removed and the crude product was purified by flash column chromatography to afford (3R)-tert-butyl 3-(1-(2-(o-tolyloxy)phenyl)-1-hydroxy-5-methoxypentyl)piperidine-1-carboxylate (20 g, 83%).

Step 3. 5-(methyloxy)-1-{2-[(2-methylphenyl)oxy]phenyl}-1-[(3R)-3-piperidinyl]-1-pentanol: The Boc protecting group was removed using the protocol described in Preparation 5 Step 4.

### Preparation 6b

### Alternate Piperidines from Weinreb Amides and Halodiphenylethers

### methyl {4-{3-fluoro-2-[3-methylphenyl)oxy]phenyl}-4-hydroxy-4-[(3R)-3-piperidinyl]butyl}carbamate

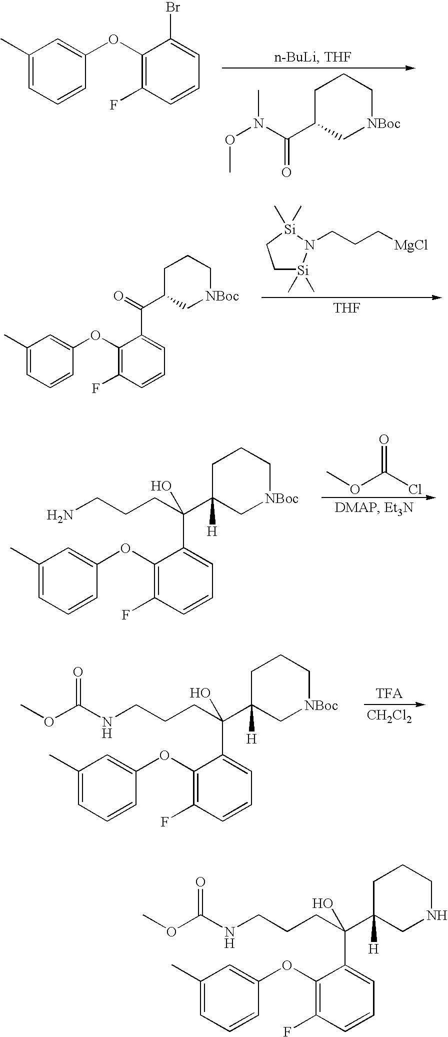

### Step 1. 1,1-dimethylethyl (3R)-3-({3-fluoro-2-[(3-methylphenyl)oxy]phenyl}carbonyl)-1-piperidinecarboxylate

To a solution of 1-bromo-3-fluoro-2-[(3-methylphenyl)oxy]benzene (3.27 g, 11.7 mmol) in THF at −78° C., was added n-BuLi (2.5 M, 5.5 mL, 13.8 mmol). The resulting solution was stirred at −78° C. for 1 h. A solution of 1,1-dimethylethyl (3R)-3-{[methyl(methyloxy)amino]carbonyl}-1-piperidinecarboxylate (2.89 g, 10.6 mmol) in THF was then added dropwise and the resulting mixture warmed to room temperature for 2 h before it was quenched with saturated NH₄Cl. The organic layer was separated and aqueous layer extracted with ethyl acetate. Combined organic layers are washed with brine, concentrated in vacuo to give crude 1,1-dimethylethyl (3R)-3-({3-fluoro-2-[(3-methylphenyl)oxy]phenyl}carbonyl)-1-piperidinecarboxylate (5.1 g) which was used in the next reaction without further purification.

### Step 2. 1,1-dimethylethyl (3R)-3-(4-amino-1-{3-fluoro-2-[(3-methylphenyl)oxy]phenyl}-1-hydroxybutyl)-1-piperidinecarboxylate

To a solution of 1,1-dimethylethyl (3R)-3-({3-fluoro-2-[(3-methylphenyl)oxy]phenyl}carbonyl)-1-piperidinecarboxylate (5 g, 12.1 mmol) in THF at −78° C. was added dropwise a solution of (3-(2,2,5,5-tetramethyl-1,2,5-azadisilolidin-1-yl)propyl)magnesium chloride (1.45 M, 10.5 mL, 36.5 mmol). After the addition is complete, the resulting solution is allowed to warm to rt slowly, and left at rt for 1 h. The reaction was quenched with saturated NH₄Cl and extracted with ethyl acetate. The combined organic layers were washed with brine, dried over Na₂SO₄, filtered and concentrated in vacuo to give crude 1,1-dimethylethyl (3R)-3-(4-amino-1-{3-fluoro-2-[(3-methylphenyl)oxy]phenyl}-1-hydroxybutyl)-1-piperidinecarboxylate (6.6 g) which was used in the next step without further purification. MS (E/Z): 473.1 (M+H⁺)

### Step 3. 1,1-dimethylethyl (3R)-3-(1-{3-fluoro-2-[(3-methylphenyl)oxy]phenyl}-1-hydroxy-4-{[(methyloxy)carbonyl]amino}butyl)-1-piperidinecarboxylate

To a solution of 1,1-dimethylethyl (3R)-3-(4-amino-1-{3-fluoro-2-[(3-methylphenyl)oxy]phenyl}-1-hydroxybutyl)-1-piperidinecarboxylate (5.93 g, 13.2 mmol) and DMAP (0.81 g, 0.6 mmol) in CH₂Cl₂ was added Et₃N (4.0 g, 39.6 mmol). The resulting mixture was cooled to 5° C. and methyl chloroformate (6.2 g, 66 mmol) added and the mixture maintained at 5° C. for 2 h. The reaction was quenched with water and extracted with CH₂Cl₂. The combined organic layers are washed with 10% citric acid and brine, dried over Na₂SO₄, filtered and concentrated in vacuo to give a crude product which is purified by flash column chromatography to afford 1,1-dimethylethyl (3R)-3-(1-{3-fluoro-2-[(3-methylphenyl)oxy]phenyl}-1-hydroxy-4-{[(methyloxy)carbonyl]amino}butyl)-1-piperidinecarboxylate (3.2 g, 48%). MS (E/Z): 531.1 (M+H⁺)

### Step 4. Methyl {4-{3-fluoro-2-[(3-methylphenyl)oxy]phenyl}-4-hydroxy-4-[(3R)-3-piperidinyl]butyl}carbamate

To a solution of 1,1-dimethylethyl (3R)-3-(1-{3-fluoro-2-[(3-methylphenyl)oxy]phenyl}-1-hydroxy-4-{[(methyloxy)carbonyl]amino}butyl)-1-piperidinecarboxylate (3.19 g, 6.0 mmol) in CH₂Cl₂ (31.9 mL) was added TFA (31.9 mL) slowly at rt. The resulting mixture was stirred at rt for 15 min then neutralized to pH=7 with aqueous NaHCO₃ and extracted with CH₂Cl₂. The combined extracts were washed with brine, dried over Na₂SO₄, filtered and concentrated in vacuo to give methyl {4-{3-fluoro-2-[(3-methylphenyl)oxy]phenyl}-4-hydroxy-4-[(3R)-3-piperidinyl]butyl}carbamate (2.9 g) which was used in the next step without further purification. MS (E/Z): 431.1 (M+H⁺)

The following piperidines prepared using the above procedures using the halodiphenyl ethers listed below in Step 1.

| Piperidine | Halodiphenyl ether |
|---|---|
| methyl {4-{3-chloro-2-[(3- | 1-bromo-3-chloro-2-[(3- |
| methylphenyl)oxy]phenyl}-4-hydroxy-4- | methylphenyl)oxy]benzene |
| [(3R)-3-piperidinyl]butyl}carbamate | |
| methyl {4-{2-[(2,6- | 2-[(2-bromophenyl)oxy]-1,3- |
| dimethylphenyl)oxy]phenyl}-4-hydroxy-4- | dimethylbenzene |
| [(3R)-3-piperidinyl]butyl}carbamate | |
| methyl 4-(3-chloro-2-(3- | 1-bromo-3-fluoro-2-(m- |
| ethylphenoxy)phenyl)-4-hydroxy-4-((R)- | tolyloxy)benzene |
| piperidin-3-yl)butylcarbamate | |

### Preparation 7

### Piperidines from Weinreb Amides and Bromobiaryls

### 1-(2′-chloro-2-biphenylyl)-5-(methyloxy)-1-[(3R)-3-piperidinyl]-1-pentanol

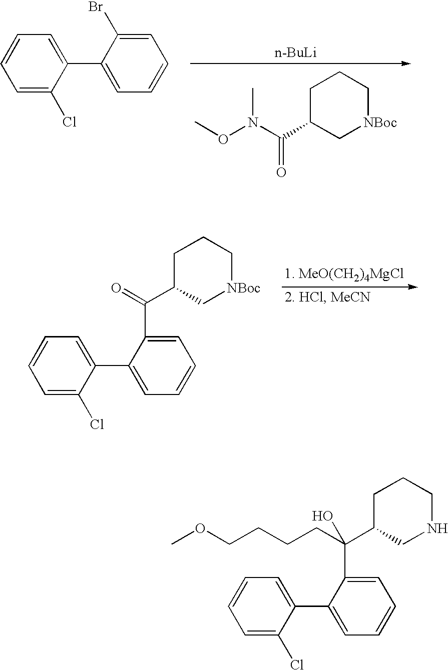

Step 1. (3R)-1-(tent-butoxycarbonyl)-3-((2-(2-chlorophenyl))benzoyl)piperidine: To a solution of 2′-bromo-2-chloro-biphenyl (5.34 g, 20 mmol) in anhydrous THF (50 mL) cooled to −78° C. was added dropwise a solution of 1.6 M n-BuLi in hexane (12.5 mL, 20 mmol). The reaction mixture was stirred at −78° C. for 1 h and a solution of (R)-tent-butyl 3-(N-methoxy-N-methylcarbamoyl)-piperidine-1-carboxylate (5.44 g, 20 mmol) in anhydrous THF (50 mL) was added. The mixture was allowed to warm to rt and stirred overnight. The mixture was quenched with satd aq NH₄Cl (100 mL) and extracted with EtOAc (3×75 mL). The combined organic layers were dried over Na₂SO₄ and concentrated to give the crude product, which was purified by flash column chromatography to afford (3R)-1-(tert-butoxycarbonyl)-3-42-(2-chlorophenyl))benzoyl)piperidine (4.43 g, 55%).

Step 2. 1,1-dimethylethyl (3R)-3-[1-(2′-chloro-2-biphenylyl)-1-hydroxy-5-(methyloxy)pentyl]-1-piperidinecarboxylate: A 250 mL three-necked flask was charged with magnesium turning (2.88 g, 0.12 mol) and a small crystal of iodine. The flask was evacuated and refilled with N₂. A solution of 1-chloro-4-methoxybutane (15 g, 0.12 mol) in THF (60 ml) was added dropwise to the above mixture. After heating under reflux for 2 h most of magnesium had been consumed and the Grignard solution was cooled to rt. A 250 mL three-necked flask was charged with (3R)-1-(tert-butoxycarbonyl)-3-((2-(2-chlorophenyl))benzoyl)piperidine (4.43 g, 11 mmol) and THF (50 mL), evacuated and refilled with N₂. The mixture was cooled in a dry ice-acetone bath and the Grignard reagent was added dropwise. The mixture was allowed to warm slowly to rt and stirred overnight. The mixture was quenched with satd aq NH₄Cl (100 mL) and extracted with EtOAc. The combined organic layers were dried over Na₂SO₄ and concentrated to give the crude product which was purified by flash column chromatography to afford pure 1,1-dimethylethyl (3R)-3-[1-(2′-chloro-2-biphenylyl)-1-hydroxy-5-(methyloxy)pentyl]-1-piperidinecarboxylate (2.5 g, 47%).

Step 3. 1-(2′-chloro-2-biphenylyl)-5-(methyloxy)-1-[(3R)-3-piperidinyl]-1-pentanol: The Boc protecting group was removed using the protocol described in Preparation 5 Step 4.

The following piperidines were prepared using procedures analogous to those described above substituting the bromobiphenyls indicated in Step 1:

| Piperidine | Bromobiphenyl |
|---|---|
| 1-(6-fluoro-3′-methoxy-5′-methylbiphenyl- | 2-bromo-6-fluoro-3′-methoxy- |
| 2-yl)-5-methoxy-1-((R)-piperidin-3- | 5′-methylbiphenyl |
| yl)pentan-1-ol | |
| 1-(6-chloro-3′-ethylbiphenyl-2-yl)-5- | 2-bromo-6-chloro-3′- |
| methoxy-1-((R)-piperidin-3-yl)pentan-1-ol | ethylbiphenyl |
| 1-(3-chloro-2-(2-methylbenzyl)phenyl)-5- | 1-bromo-3-chloro-2-(2- |
| methoxy-1-((R)-piperidin-3-yl)pentan-1-ol | methylbenzyl)benzene |
| 1-(3-chloro-2-(3-methylbenzyl)phenyl)-5- | 1-bromo-3-chloro-2-(3- |
| methoxy-1-((R)-piperidin-3-yl)pentan-1-ol | methylbenzyl)benzene |
| 1-(3-chloro-2-(quinolin-3-yl)phenyl)-5- | 3-(2-bromo-6- |
| methoxy-1-((R)-piperidin-3-yl)pentan-1-ol | chlorophenyl)quinoline |
| 1-(3′-ethoxy-6-fluoro-5′- | 2-bromo-3′-ethoxy-6-fluoro- |
| (trifluoromethyl)biphenyl-2-yl)-5- | 5′-(trifluoromethyl)biphenyl |
| methoxy-1-((R)-piperidin-3-yl)pentan-1-ol | |

### Preparation 8

### Ester Hydrolysis

### (1S,3S,4R)-3-hydroxy-4-(tert-butoxycarbonylamino)cyclopentane-1-carboxylic acid

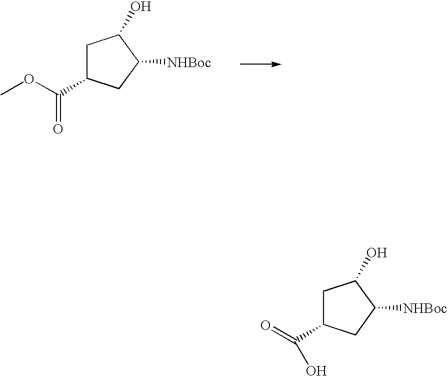

To a solution of tert-butyl (1R,2S,4S)-4-(methoxycarbonyl)-2-hydroxycyclopentyl-carbamate (115 mg, 0.444 mmol) in THF (1 mL) and ethanol (1 mL), was added 1M aq NaOH solution (1 mL). The mixture was stirred for 1 h. The solvent was evaporated and the filtrate was redissolved in water. The solution was neutralized with 1M aq HCl and extracted with EtOAc. The organic layer was washed with brine and dried over sodium sulfate. The solvent was removed by evaporation and to afford tert-butyl (1S,3R,4S)-3-(tert-butoxycarbonylamino)-4-hydroxycyclopentanecarboxylic acid (94 mg, 87%).

(1S,3R,4R)-3-(tert-butoxycarbonylamino)-4-hydroxycyclopentanecarboxylic acid was prepared from (1R,2R,4S)—N—BOC-1-amino-2-hydroxycyclopentane-4-carboxylic acid methyl ester using the above procedure.

### Preparation 9

### Biaryl Syntheses

### a) 6-Bromo-2-fluoro-3′-methylbiphenyl

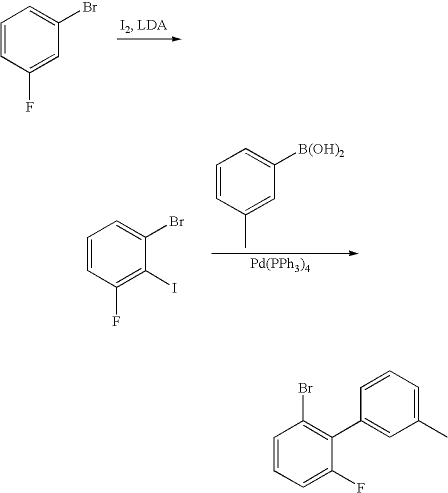

Step 1. 1-Bromo-3-fluoro-2-iodobenzene: To a solution of diisopropylamine (76 mL, 0.4 mol) in dry THF (664 mL) and n-hexane (220 mL) was added 2.5 M n-BuLi (160 mL. 0.4 mol) dropwise at −78° C. during a period of 1 h. The mixture was stirred for 1 h at −78° C. Then a solution of 1-bromo-3-fluoro-benzene (69 g, 0.4 mol) in dry THF (300 mL) at −78° C. was added to the above mixture dropwise. After stirring for an additional 1 h at −78° C., the mixture was added a solution of iodine (101 g, 0.4 mol) in dry THF (400 mL) dropwise at −78° C. The temperature was raised from −78° C. to rt during 2 h. After stirring for 18 h at rt, the mixture was concentrated in vacuo to give crude product (120 g) which was distilled under reduced pressure to afford 1-bromo-3-fluoro-2-iodobenzene (110 g). ¹H NMR (400 MHz, DMSO): 7.24-7.19 (t, 1H), 7.38-7.32 (m, 1H), 7.55-7.53 (d, 1H).

Step 2. 6-Bromo-2-fluoro-3′-methylbiphenyl: Pd(Ph₃P)₄ in a 500-mL round-bottom flask under N₂ atmosphere was treated sequentially with a solution of 1-bromo-3-fluoro-2-iodo-benzene (30 g, 0.1 mol) in toluene (250 mL), a solution of 2N aq Na₂CO₃ (200 mL) and 3-methyl phenylboronic acid in ethanol (62 mL). This mixture was heated at reflux under N₂ for 12 h, then cooled to rt. The mixture was partitioned between water and EtOAc. The combined organic layers were washed with brine, dried over MgSO₄, evaporated and purified by column chromatography to give 6-bromo-2-fluoro-3′-methyl-biphenyl (12 g). ¹H NMR (400 MHz, CD₃OD): 7.03 (m, 2H), 7.48-7.04 (m, 4H), 7.50 (d, 1H).

### b) 6-Bromo-2-chloro-3′-methyl-biphenyl

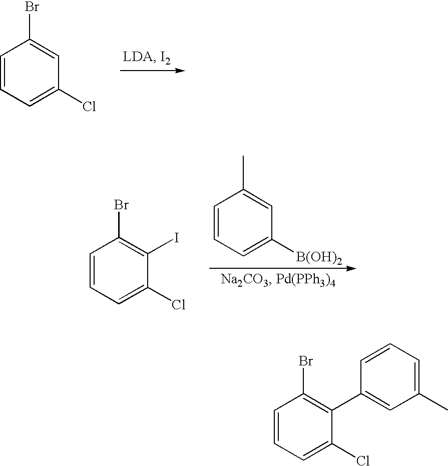

Step 1. 1-bromo-3-chloro-2-iodobenzene: To a solution of diisopropylamine (76 mL, 0.4 mol) in anhydrous THF (664 mL) and n-hexane (220 mL) was added 2.5 M n-BuLi (160 mL, 0.4 mol) dropwise at −78° C. over 1 h. The mixture was stirred for 1 h at −78° C. and a solution of 1-bromo-3-chlorobenzene (76 g, 0.4 mol) in anhydrous THF (300 mL) was added dropwise at −78° C. After stirring for an additional 1 h at the same temperature, a solution of iodine (101 g, 0.4 mol) in anhydrous THF (400 mL) was added dropwise at −78° C. The temperature was raised from −78° C. to rt during 2 h. After stirring for 18 h at rt, the mixture was concentrated in vacuo to give the crude product (120 g) which was distilled under reduced pressure to give 1-bromo-3-fluoro-2-iodobenzene (115 g, 91%). ¹H NMR (400 MHz, CDCl₃): 7.12-7.18 (t, 1H), 7.35-7.41 (dd, 1H), 7.49-7.54 (dd, 1H); MS (E/Z): 317 (M+H⁺)

Step 2. 6-bromo-2-chloro-3′-methyl-biphenyl: A 500-mL round-bottom flask under N₂ atmosphere was charged sequentially with Pd(Ph₃P)₄, 1-bromo-3-fluoro-2-iodobenzene (10 g, 0.032 mol) in toluene (80 mL), 2N aqueous sodium carbonate (55 mL) and 3-methylphenylboronic acid (5.16 g, 0.032 mol) dissolved in ethanol (40 mL). This mixture was heated at reflux under N₂ for 12 h and cooled to rt. The mixture was partitioned between water and EtOAc. The combined organic layers were washed with brine, dried over MgSO₄, and concentrated. The residue was purified by column chromatography to give 6-bromo-2-chloro-3′-methyl-biphenyl (6 g, 67%). ¹H NMR (400 MHz, CD₃OD): 6.90-7.00 (t, 2H), 7.14-7.24 (m, 2H), 7.26-7.33 (t, 1H), 7.44-7.50 (d, 1H), 7.58-7.62 (d, 1H); MS (E/Z): 281 (M+H⁺)

The following biaryls were prepared from aryl halides and the boronic acids indicated using the procedures described in Preparations 9a Step 2 and 9b Step 2:

| Biaryl | Aryl halide | Boronic acid |
|---|---|---|
| 2-bromo-6-chloro-3′- | 1-bromo-3-chloro- | 3-ethylphenylboronic acid |
| ethylbiphenyl | 2-iodobenzene | |
| 2-bromo-3′-ethyl-6- | 1-bromo-3-fluoro- | 3-ethylphenylboronic acid |
| fluorobiphenyl | 2-iodobenzene | |
| 2-bromo-6-chloro-3′- | 1-bromo-3-chloro- | 3-isopropylphenylboronic acid |
| isopropylbiphenyl | 2-iodobenzene | |
| 2-bromo-4′,6-difluoro-3′- | 1-bromo-3-fluoro- | (4-fluoro-3-methylphenyl)boronic |
| methylbiphenyl | 2-iodobenzene | acid |
| 2-bromo-6-fluoro-4′-fluoro- | 1-bromo-3-fluoro- | (4-fluoro-3-methylphenyl)boronic |
| 3′-methylbiphenyl | 2-iodobenzene | acid |
| 2-bromo-6-chloro-3′,5′-bis | 1-bromo-3-chloro- | [3,5-bis (methoxy) phenyl]boronic |
| (methoxy) biphenyl | 2-iodobenzene | acid |
| 2-bromo-6-fluoro-3′,5′-bis | 1-bromo-3-fluoro- | [3,5-bis (methoxy) phenyl]boronic |
| (methoxy) biphenyl | 2-iodobenzene | acid |
| 2-bromo-6-chloro-3′- | 1-bromo-3-chloro- | [3-(methoxy)phenyl]boronic acid |
| (methoxy)biphenyl | 2-iodobenzene | |
| 2-bromo-6-fluoro-3′- | 1-bromo-3-fluoro- | [3-(methoxy)phenyl]boronic acid |
| (methoxy)biphenyl | 2-iodobenzene | |
| 2-bromo-6-fluoro-3′-methyl- | 1-bromo-3-fluoro- | [3-methyl-5-(methoxy)phenyl]boronic |
| 5′-(methoxy)biphenyl | 2-iodobenzene | acid |
| 2-bromo-3′-(ethyloxy)-6- | 1-bromo-3-fluoro- | [3-(ethyloxy)-5-(trifluoromethyl)phenyl]boronic |
| fluoro-5′- | 2-iodobenzene | acid |
| (trifluoromethyl)biphenyl | | |
| 3-(2-bromo-6- | 1-bromo-3-chloro- | 3-quinolinylboronic acid |
| chlorophenyl)quinoline | 2-iodobenzene | |
| 2-(2-bromo-6- | 1-bromo-3-chloro- | 2-naphthalenylboronic acid |
| chlorophenyl)naphthalene | 2-iodobenzene | |
| 3-(2-bromophenyl)pyridine | 1-bromo-2- | 3-pyridinylboronic acid |
| | iodobenzene | |
| 3-(2-bromo-6- | 1-bromo-3-chloro- | 3-pyridinylboronic acid |
| chlorophenyl)pyridine | 2-iodobenzene | |
| 4-(2-bromophenyl)pyridine | 1-bromo-2- | 4-pyridinylboronic acid |
| | iodobenzene | |
| 4-(2-bromo-6- | 1-bromo-3-chloro- | 4-isoquinolinylboronic acid |
| chlorophenyl)isoquinoline | 2-iodobenzene | |
| 2-bromo-6-fluoro-2′-fluoro- | 1-bromo-3-fluoro- | (2-fluoro-5-methylphenyl)boronic |
| 5′-methylbiphenyl | 2-iodobenzene | acid |
| 2-bromo-6-chloro-3′- | 1-bromo-3-chloro- | {3-[(methoxy)methyl]phenyl}boronic |
| [(methoxy)methyl]biphenyl | 2-iodobenzene | acid |
| 2-bromo-5-fluoro-3′- | 1-bromo-4-fluoro- | 3-methylphenylboronic acid |
| methylbiphenyl | 2-iodobenzene | |
| 2-bromo-2′,4,6-trifluoro-5′- | 1-bromo-3,5- | (2-fluoro-5-methylphenyl)boronic |
| methylbiphenyl | difluoro-2- | acid |
| | iodobenzene | |
| 2-bromo-4,6-difluoro-3′- | 1-bromo-3,5- | 3-methylphenylboronic acid |
| methylbiphenyl | difluoro-2- | |
| | iodobenzene | |
| 2-bromo-3′,6-difluoro-5′- | 1-bromo-3-fluoro- | 3-fluoro-5-methylphenylboronic |
| methylbiphenyl | 2-iodobenzene | acid |
| 2-bromo-6-chloro-3′-fluoro- | 1-bromo-3-chloro- | 3-fluoro-5-methylphenylboronic |
| 5′-methylbiphenyl | 2-iodobenzene | acid |
| 3-(2-bromo-6- | 1-bromo-3-fluoro- | 3-quinolinylboronic acid |
| fluorophenyl)quinoline | 2-iodobenzene | |
| 2-(2-bromo-6-fluorophenyl)- | 1-bromo-3-chloro- | 5-methylfuran-2-ylboronic acid |
| 5-methylfuran | 2-iodobenzene | |
| 2-bromo-6-fluoro-3′- | 1-bromo-3-chloro- | 3-isopropylphenylboronic acid |
| isopropylbiphenyl | 2-iodobenzene | |
| 3-(2-bromo-6- | 1-bromo-3-chloro- | 1-benzothien-2-ylboronic acid |
| chlorophenyl)-1- | 2-iodobenzene | |
| benzothiophene | | |
| 4-bromo-2-chloro-3-(3- | 4-bromo-2-chloro- | (3-ethylphenyl)boronic acid |
| ethylphenyl)pyridine | 3-iodopyridine | |
| 2-bromo-4-chloro-3-(3- | 2-bromo-4-chloro- | (3-ethylphenyl)boronic acid |
| ethylphenyl)pyridine | 3-iodopyridine | |
| 4-bromo-2-chloro-3-[3-(1- | 4-bromo-2-chloro- | [3-(1-methylethyl)phenyl]boronic |
| methylethyl)phenyl]pyridine | 3-iodopyridine | acid |

### c) 2-(2-bromo-6-chlorophenyl)-8-(1-methylethyl)quinoline

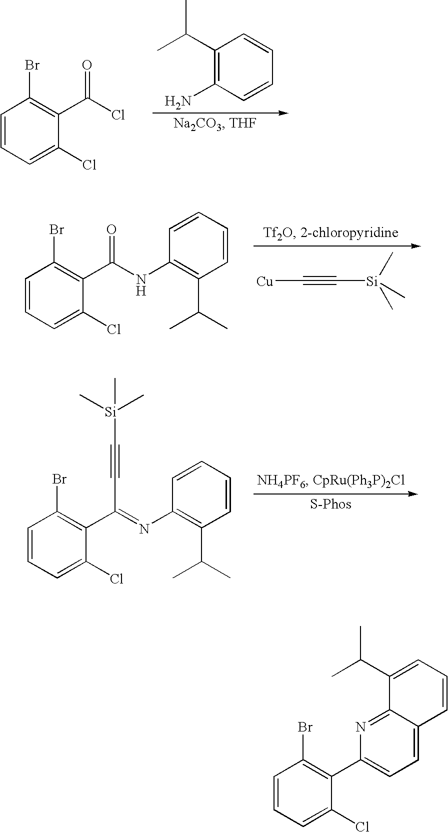

### Step 1. 2-bromo-6-chloro-N-[2-(1-methylethyl)phenyl]benzamide

To a solution of 2-bromo-6-chlorobenzoyl chloride (39.3 g, 149.1 mmol) and Na₂CO₃ (31.4 g, 318.3 mmol) in THF (232 mL) and water (23 mL) was added [2-(1-methylethyl)phenyl]amine (22.2 g, 164.1 mmol). The resulting mixture was stirred at room temperature for 1 h. The mixture was extracted with ethyl acetate and the pH adjusted to 2. The organic layers were then washed with Na₂CO₃, brine, dried over Na₂SO₄, filtered and concentrated to give 2-bromo-6-chloro-N-[2-(1-methylethyl)phenyl]benzamide (35.5 g) which was used in the subsequent reaction without further purification. MS (E/Z): 353.9 (M+H⁺)

### Step 2. N-[(1)-1-(2-bromo-6-chlorophenyl)-3-(trimethylsilyl)-2-propyn-1-ylidene]-2-(1-methylethyl)aniline

To a stirred solution of 2-bromo-6-chloro-N-[2-(1-methylethyl)phenyl]benzamide (50 g, 137.8 mmol) and 2-chloropyridine (50.5 mL, 554.9 mmol) in CH₂Cl₂ (300 mL) under argon at −78° C. was added Tf₂O (28.2 mL, 167.6 mmol). After 5 min, the reaction mixture was warmed to 0° C. and maintained at that temperature for 20 min before recooling to −78° C. [(Trimethylsilyl)ethynyl]copper (730 mL, 383.4 mmol) was then added via cannula as a solution in THF. The resulting mixture was maintained at −78° C. for 5 min before warming to 0° C. After 10 min the crude mixture was filtered through Celite and the filtrate concentrated in vacuo. The crude material was purified via column chromatography to give N-[(1)-1-(2-bromo-6-chlorophenyl)-3-(trimethylsilyl)-2-propyn-1-ylidene]-2-(1-methylethyl)aniline (9.58 g, 16%). MS (E/Z): 433.1 (M+H⁺)

### Step 3. 2-(2-bromo-6-chlorophenyl)-8-(1-methylethyl)quinoline

To a mixture of Ammonium hexafluorophosphate purum (3.7 g, 22.2 mmol) and CpRu(Ph₃P)₂Cl (1.64 g, 2.3 mmol) in toluene (110 mL) was added N-[(1)-1-(2-bromo-6-chlorophenyl)-3-(trimethylsilyl)-2-propyn-1-ylidene]-2-(1-methylethyl)aniline (9.1 g, 21.0 mmol). The resulting mixture was then heated to 115° C. for 19 h. The reaction was then cooled to rt, diluted with CH₂Cl₂ and the solvent removed in vacuo. The residue was then purified via column chromatography to afford 2-(2-bromo-6-chlorophenyl)-8-(1-methylethyl)quinoline (3.7 g, 49%). MS (E/Z): 360.0 (M+H⁺)

The following biaryl was prepared from the aniline indicated using the procedures described in Preparations 9c Steps 1-3:

| Biaryl | Aniline |
|---|---|
| 2-(2-bromo-6-chlorophenyl)-8- | 2-methyl aniline |
| methylquinoline | |

### d) 2-(2-bromo-6-chlorophenyl)-4-(1-methylethyl)quinazoline

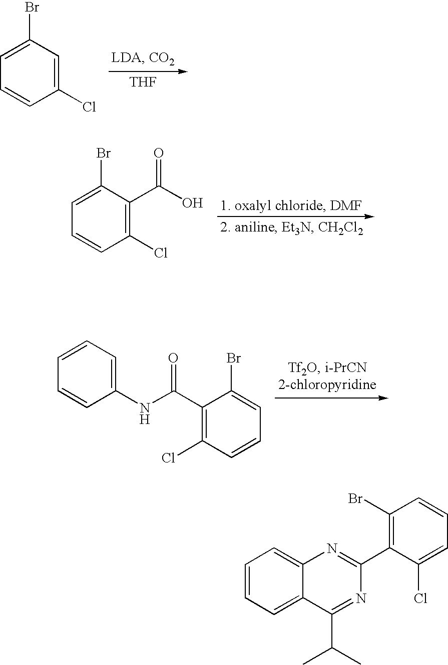

### Step 1. 2-bromo-6-chlorobenzoic acid

To a stirred solution of n-BuLi (90.0 mmol, 36 ml of a 2.5 M solution in hexanes) in 160 mL of dry THF at −78° C., was added dropwise diisopropylamine (12.4 ml, 90 mmol) in 20 mL of dry THF. The resulting solution was stirred for 0.5 h at −78° C. A solution of 1-bromo-3-chlorobenzene (14.3 g, 75.0 mmol) in 20 ml of dry THF was added and the resulting mixture was stirred for an additional hour −78° C. Then dry ice (CO₂) was added in small portions (large gas evolution) and after 20 min the solution was quenched with 100 mL of 2N HCl. The mixture was extracted with ethyl acetate (1000 ml) and the crude 2-bromo-6-chlorobenzoic acid (white solid) was triturated with Et₂O and used in the next step without other purification. MS (E/Z): 234.9 (M+H⁺)

### Step 2. 2-bromo-6-chloro-N-phenylbenzamide

To a stirred solution of 2-bromo-6-chlorobenzoic acid (3.15 g, 13.4 mmol) in 20.0 mL of dry methylene chloride, were added DMF (catalytic amount) and oxalyl chloride (1.45 mL, 16.1 mmol) dropwise. The resulting solution was stirred for 2 h at room temperature. The solvent was removed in vacuo and the crude dissolved in 20.0 mL of dry DCM. Triethylamine (3.7 mL, 26.8 mmol) and aniline (1.78 mL, 18.7 mmol) were added and the resulting mixture was stirred over night at room temperature. HPLC/MS showed that the reaction was completed at this time. The reaction mixture was quenched with 0.6N HCl and extracted with methylene chloride. The organic layer was then dried, filtered and concentrated to afford 2-bromo-6-chloro-N-phenylbenzamide, which was used in the next step without further purification. MS (E/Z): 309.9 (M+H⁺)

### Step 3. 2-(2-bromo-6-chlorophenyl)-4-(1-methylethyl)quinazoline

To a stirred solution of 2-bromo-6-chloro-N-phenylbenzamide (930 mg, 3.0 mmol) and 2-chloropyridine (406 μl, 3.6 mmol) in 10 ml of dry methylene chloride was added at −78° C. followed by Tf₂O (1015 μl, 3.6 mmol). The solution was stirred at −78° C. and then was warmed to 0° C. and i-PrCN (354 μl, 3.6 mmol) added. The resulting solution was stirred overnight 70° C. in a microwave vial. The HPLC/MS showed product as well as starting material. The reaction mixture was quenched with 0.6N HCl and extracted with methylene chloride. The organic layer was then dried, filtered, and concentrated to afford the crude material. Column chromatography then gave 2-(2-bromo-6-chlorophenyl)-4-(1-methylethyl)quinazoline (0.418 g, 40%). MS (E/Z): 361.0 (M+H⁺)

### e) 2-bromo-4-chloro-3-(3-ethylphenyl)pyridine

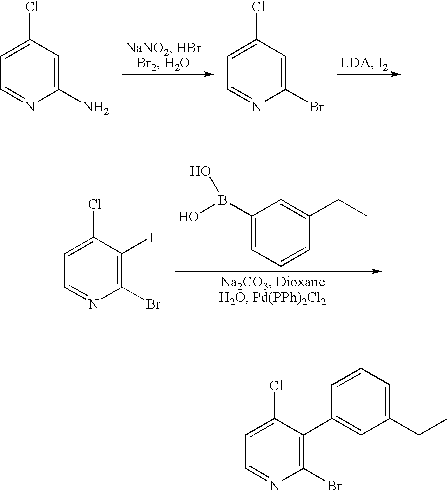

### Step 1. 2-bromo-4-chloropyridine

To an aqueous solution of 48% strength hydrobromic acid (82 mL) at 0° C. was added 4-chloro-2-pyridinamine (8.9 g, 69.2 mmol) followed by addition of bromine (33.4 g, 209 mmol) over 10 min. The resulting mixture was cooled to −10° C. and a solution of NaNO₂ (10.65 g, 154 mmol) in H₂O (20 mL) was poured in over a period of 30 min. The mixture was warmed at room temperature and stirred overnight. The mixture was recooled to 0° C. and NaOH (35%) added until the pH >10. The mixture was then extracted with ethyl acetate. The organic layer was then dried, filtered, and concentrated in vacuo. The product was purified via column chromatography (0-20% ethyl acetate/hexane) to afford 2-bromo-4-chloropyridine (12.1 g, 92%).

### Step 2. 2-bromo-4-chloro-3-iodopyridine

To a stirred solution of diisopropylamine (8.24 mL, 60.0 mmol) in THF (100 mL) at −78° C. was added n-BuLi (24.0 mL, 60.0 mmol) and the solution was stirred at this temperature for 30 min. Then, a solution of 2-bromo-4-chloropyridine (12.1 g, 60.0 mmol) dissolved in THF (100 mL) was added dropwise. The resulting mixture was stirred for 1 h at −78° C. Then I₂ (21.0 g, 66.0 mmol) was added in three portions. The solution was warmed to room temperature and stirred overnight. The mixture was diluted with ethyl acetate and washed with water. The organic layer was then dried, filtered, and concentrated in vacuo. The crude material was purified via column chromatography to give 2-bromo-4-chloro-3-iodopyridine (7.3 g, 38%). MS (E/Z): 317.8 (M+H⁺).

### Step 3. 2-bromo-4-chloro-3-(3-ethylphenyl)pyridine

To a solution of 2-bromo-4-chloro-3-iodopyridine (3.17 g, 10 mmol) in dioxane (20 mL) and water (10 mL) was added (3-ethylphenyl)boronic acid (1.9 g, 13.0 mmol) followed by Pd(Ph)₂Cl₂ (0.350 g, 0.5 mmol). The resulting mixture was then heated at 80° C. overnight. In the morning, the reaction mixture was diluted with ethyl acetate, washed with water then brine, dried, filtered and concentrated in vacuo. The crude material was then purified via column chromatography to afford 2-bromo-4-chloro-3-(3-ethylphenyl)pyridine (1.5 g, 50%).

### f.) 3-Bromo-5-chloro-4-[3-(1-methylethyl)phenyl]pyridine

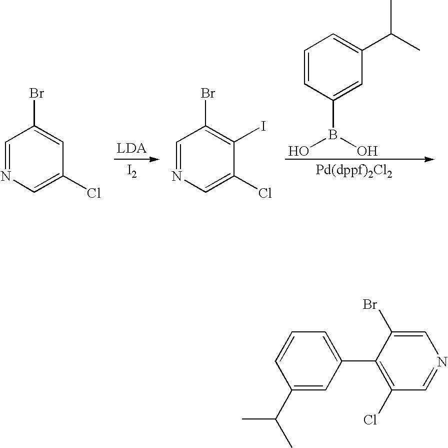

### Step 1: 3-bromo-5-chloro-4-iodopyridine

To a −78° C. solution of diisopropylamine (3.7 mL, 26 mmol) in anhydrous THF (50 mL) was added n-BuLi (10.4 mL, 2.5 M hexanes, 26 mmol). After stirring for 30 minutes, a solution of 3-bromo-5-chloropyridine (5.0 g, 26 mmol) in THF (10 mL) was added dropwise. After stirring for an additional 1 hour at −78° C., a solution of iodine (7.9 g, 31.2 mmol) in THF (25 mL) was added. The reaction was slowly allowed to warm to room temperature and continued to stir overnight. The reaction was quenched with water (25 ml) and a saturated sodium thiosulfate solution (25 mL). The phases were separated. The organic layer was washed with Na₂S₂O₃ solution (25 mL). The aqueous phase was back extracted with EtOAc (3×25 mL). The combined organic extracts were washed with brine, dried over MgSO₄, filtered and concentrated to give 7 g of a brown solid. The crude product was triturated with Et₂O twice and isolated 3-bromo-5-chloro-4-iodopyridine (3.3 g, 40% yield) as a brown solid (powder).

### Step 2: 3-bromo-5-chloro-4-(3-isopropylphenyl)pyridine

3-Bromo-5-chloro-4-iodopyridine (1.6 g, 5.0 mmol), (3-isopropylphenyl)boronic acid (0.99 g, 6.0 mmol), Na₂CO₃ (1.1 g, 10 mmol) and Pd(PPh₃)₂Cl₂ (0.50 g, 0.50 mmol) were added to a flask with 1,4-dioxane/water (2:1) (15 mL). The reaction mixture was immersed into a preheated oil bath (85° C.) and stirred overnight (18 hours). The reaction mixture was allowed to cool to room temperature and then diluted with EtOAc and water. The phases were separated and the aqueous phase was extracted with EtOAc (2×). The combined organics were washed with brine, dried over MgSO₄, filtered and concentrated to give 2 g of a brown oil. The crude residue was purified by flash chromatography on silica gel and isolated 0.78 g (50% yield) of 3-bromo-5-chloro-4-(3-isopropylphenyl)pyridine as a clear oil.

The following biaryl was prepared from the indicated boronic acid using the procedures described in Preparations 9f Steps 1-2:

| Biaryl | Boronic Acid |
|---|---|
| 3-bromo-5-chloro-4-(3- | (3-ethylphenyl)boronic acid |
| ethylphenyl)pyridine | |

### Preparation 10

### Morpholine Synthesis

### (R)-1-(6-Fluoro-3′-methylbiphenyl-2-yl)-5-methoxy-1-((R)-morpholin-2-yl)pentan-1-ol

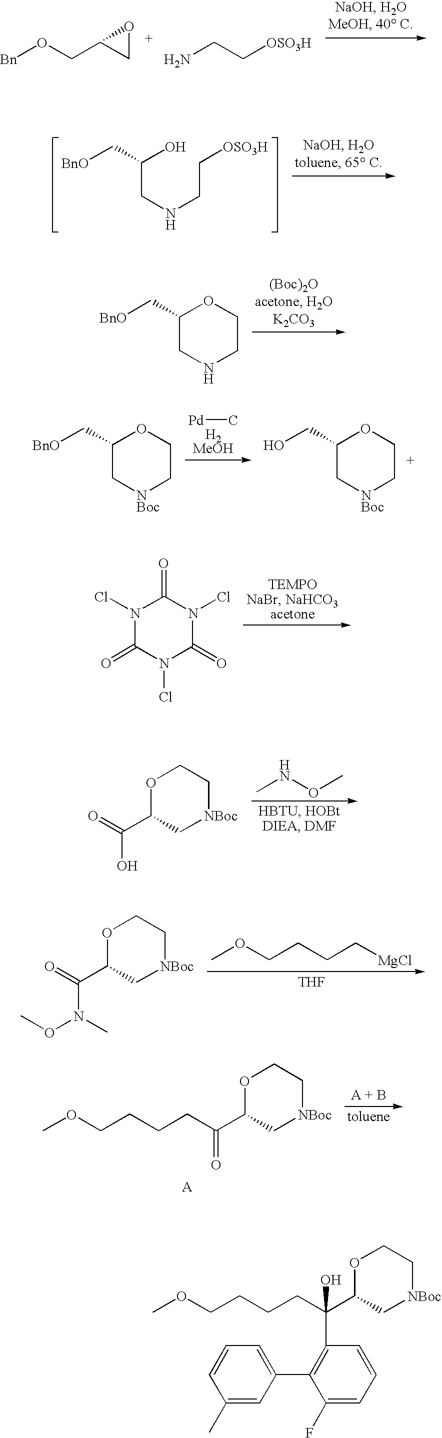

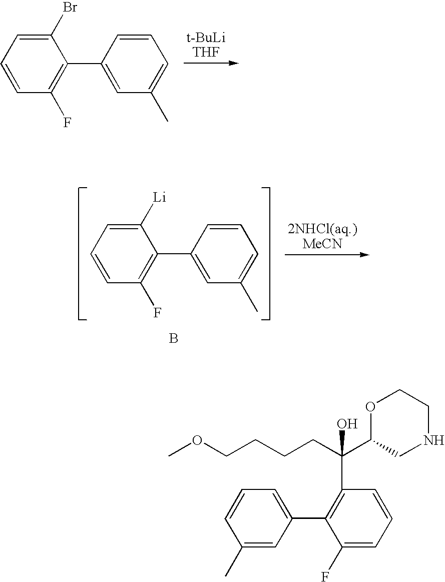

Step 1. (R)-2-(Benzyloxymethyl)morpholine: To a stirred mixture of (R)-2-(benzyloxymethyl)oxirane (10.0 g, 60.9 mmol) and NaOH (19.49 g, 487.2 mmol) in H₂O (46 mL) and MeOH (18 mL), there was added 2-aminoethyl hydrogen sulfate (36.8 g, 255.8 mmol) in portions. After addition was complete, the reaction mixture was stirred at 40° C. for 2 h. After cooling, the mixture was treated with NaOH (15.0 g, 375.0 mmol), followed by toluene (70 mL), and stirred at 65° C. overnight. The mixture was cooled, diluted with toluene (27 mL) and H₂O (92 mL). The toluene layer was separated and the aqueous layer was extracted with CH₂Cl₂ (2×50 mL). The combined organic layers were concentrated to give crude (R)-2-(benzyloxymethyl)morpholine (˜14 g), which was used without purification. MS m/z 208 (M+H⁺).

Step 2. (R)-tent-Butyl 2-(benzyloxymethyl)morpholine-4-carboxylate: To a solution of crude (R)-2-(benzyloxymethyl)morpholine (˜14 g) in acetone (100 mL) and H₂O (30 mL) at 0° C., there was added K₂CO₃ (25.2 g, 182.7 mmol), followed by (Boc)₂O (14.6 g, 67.0 mmol). The resulting solution was warmed to rt, and stirred until no starting material remained (˜30 min). Acetone was removed under vacuum, and the aqueous solution was extracted with CH₂Cl₂ (4×10 mL). The combined organic layers were washed with H₂O (10 mL) and the solvent was removed. The residue was purified by flash column chromatography to give (R)-tent-butyl 2-(benzyloxymethyl)morpholine-4-carboxylate (8.33 g, 44% over 2 steps). ¹H NMR (400 MHz, CDCl₃): 7.34 (m, 5H), 4.56 (s, 2H), 3.88 (d, 2H), 3.82 (br, 1H), 3.40 (m, 1H), 3.48 (m, 3H), 2.94 (m, 1H), 2.76 (m, 1H), 1.44 (s, 9H); MS m/z 330 (M+Na⁺).

Step 3. (R)-tent-Butyl 2-(hydroxymethyl)morpholine-4-carboxylate: To a solution of (R)-tent-butyl 2-(benzyloxymethyl)morpholine-4-carboxylate (8.33 g, 27.1 mmol) in EtOH was added Pd—C (wet, 3.6 g), and the resulting mixture was stirred at rt under a H₂ balloon overnight. After filtration, the solvent was removed under vacuum, and the residue was purified by flash column chromatography to give (R)-tert-butyl 2-(hydroxymethyl)morpholine-4-carboxylate (5.84 g, 99%) as a clear oil. ¹H NMR (400 MHz, CDCl₃): 3.88 (d, 2H), 3.82 (br, 1H), 3.64 (d, 1H), 3.56 (m, 3H), 2.94 (m, 1H), 2.76 (m, 1H), 1.90 (br, 1H), 1.44 (s, 9H); MS m/z 218 (M+H⁺).

Step 4. (R)-4-(tert-Butoxycarbonyl)morpholine-2-carboxylic acid: Satd aq NaHCO₃ (15 mL) was added to a solution of (R)-tent-butyl 2-(hydroxymethyl)-morpholine-4-carboxylate (1.09 g, 5.0 mmol) in acetone (50 mL), stirred and maintained at 0° C. Solid NaBr (0.1 g, 1 mmol) and TEMPO (0.015 g, 0.1 mmol) were added. Trichloroisocyanuric acid (2.32 g, 10.0 mmol) was then added slowly within 20 min at 0° C. After addition, the mixture was warmed to rt and stirred overnight. 2-Propanol (3 mL) was added, and the resulting solution was stirred at rt for 30 min, filtered through a pad of Celite, concentrated under vacuum, and treated with satd aq Na₂CO₃ (15 mL). The aqueous solution was washed with EtOAc (5 mL), acidified with 6 N HCl, and extracted with EtOAc (5×10 mL). The combined organic layers were dried over Na₂SO₄ and the solvent was removed to give (R)-4-(tert-butoxycarbonyl)morpholine-2-carboxylic acid (1.07 g, 92%) as a white solid. ¹H NMR (400 MHz, CDCl₃): 4.20 (br, 1H), 4.12 (d, 1H), 4.02 (d, 1H), 3.84 (m, 1H), 3.62 (m, 1H), 3.04 (m, 2H), 1.44 (s, 9H); MS m/z 232 (M+H).

Step 5. (R)-tent-Butyl 2-(methoxy(methyl)carbamoyl)morpholine-4-carboxylate: To a solution of (R)-4-(tert-butoxycarbonyl)morpholine-2-carboxylic acid (1.05 g, 4.54 mmol) in DMF (10 mL) at 0° C. C, was added DIEA (3.9 mL, 22.7 mmol), followed by HBTU (1.89 g, 4.99 mmol) and HOBt (0.67 g, 4.99 mmol). MeONHMe.HCl (0.48 g, 4.92 mmol) was added and the resulting solution was warmed to rt and stirred until no starting material remained (˜2 h). The mixture was diluted with H₂O (10 mL) and extracted with EtOAc (4×10 mL). The combined organic layers were washed with 1 N aq HCl (10 mL), 1 N aq NaOH (3×10 mL), water (2×10 mL) and brine (10 mL), and dried over Na₂SO₄. The solvent was removed under vacuum to give (R)-tent-butyl 2-(methoxy(methyl)carbamoyl)morpholine-4-carboxylate (1.40 g, quant.), which was used without further purification. ¹H NMR (400 MHz, CDCl₃): 4.36 (br, 1H), 4.08 (m, 1H), 4.00 (d, 1H), 3.84 (m, 1H), 3.76 (s, 3H), 3.58 (m, 1H), 3.20 (s, 3H), 3.04 (m, 2H), 1.44 (s, 9H); MS m/z 297 (M+Na⁺).

Step 6. (R)-tent-Butyl 2-(5-methoxypentanoyl)morpholine-4-carboxylate: To a stirred solution of (R)-tent-butyl 2-(methoxy(methyl)carbamoyl)morpholine-4-carboxylate (1.37 g, 5.0 mmol) in THF (10 mL) at −20° C., there was added 1.47 M 4-methoxybutylmagnesium chloride in THF (10.2 mL, 15.0 mmol) dropwise to keep the temperature below −20° C. After addition, the resulting solution was warmed to rt and quenched with 1 N aq HCl (10 mL). The organic layer was separated, and the aqueous layer was extracted with ether (3×5 mL). Combined organic layers were washed with satd aq NaHCO₃ (10 mL) and brine (5 mL) and dried over Na₂SO₄. Removal of the solvent under vacuum gave (R)-tent-butyl 2-(5-methoxypentanoyl)morpholine-4-carboxylate (1.41 g, 93%), which was used without purification. MS m/s 324 (M+Na⁺).

Step 7. (R)-tent-Butyl 2-((R)-1-(6-fluoro-3′-methylbiphenyl-2-yl)-1-hydroxy-5-methoxypentyl)-morpholine-4-carboxylate: To a solution of 2-bromo-6-fluoro-3′-methylbiphenyl (1.90 g, 7.17 mmol) in ether (8 mL) at −78° C., there was added t-BuLi in pentane (1.70 M, 8.43 mL, 14.33 mmol) dropwise to keep the temperature below −70° C. The resulting solution was stirred at −78° C.

To a solution of (R)-tert-butyl 2-(5-methoxypentanoyl)morpholine-4-carboxylate (0.68 g, 2.26 mmol) in toluene (8 mL) at −20° C. there was added the above lithium reagent dropwise to keep the solution temperature below −20° C. After addition, the resulting mixture was warmed to rt slowly, and quenched with saturated NH₄Cl (8 mL). The organic layer was separated, and aqueous layer was extracted with ether (3×5 mL). Combined organic layers were washed with water (10 mL), concentrated, and the residue was purified by flash column chromatography to give (R)-tert-butyl 2-((R)-1-(6-fluoro-3′-methylbiphenyl-2-yl)-1-hydroxy-5-methoxypentyl)-morpholine-4-carboxylate (0.48 g, 44%) as a foam. ¹H NMR (400 MHz, CDCl₃): 7.40 (m, 1H), 7.32 (m, 2H), 7.20 (d, 1H), 7.04 (m, 3H), 3.84 (m, 1H), 3.78 (m, 2H), 3.40-3.24 (ms, 7H), 2.82 (s, 3H), 1.70-1.20 (m, 5H), 1.44 (s, 9H), 0.94 (m, 1H); MS m/z 510 (M+Na⁺).

Step 8. (R)-1-(6-Fluoro-3′-methylbiphenyl-2-yl)-5-methoxy-1-((R)-morpholin-2-yl)-pentan-1-ol: To a solution of (R)-tert-butyl 2-((R)-1-(6-fluoro-3′-methylbiphenyl-2-yl)-1-hydroxy-5-methoxypentyl)morpholine-4-carboxylate (0.46 g, 0.96 mmol) in acetonitrile (50 mL) was added 2 N aq HCl (50 mL). The resulting solution was stirred at rt overnight and basified with 10 N aq NaOH to pH 10. Acetonitrile was removed under vacuum, and the aqueous residue was extracted with CH₂Cl₂ (4×5 mL). The combined organic layers were washed with brine (5 mL), dried over Na₂SO₄, and concentrated to give (R)-1-(6-fluoro-3′-methylbiphenyl-2-yl)-5-methoxy-1-((R)-morpholin-2-yl)pentan-1-ol (0.38, quant.). MS m/z 388 (M+H⁺).

The following morpholines were prepared using procedures analogous to those described above:
(R)-1-(4′,6-difluoro-3′-methylbiphenyl-2-yl)-5-methoxy-1-((R)-morpholin-2-yl)pentan-1-ol using 2-bromo-4′,6-difluoro-3′-methylbiphenyl in Step 7.
(R)-1-(3-chloro-2-(pyridin-3-yl)phenyl)-5-methoxy-1-((R)-morpholin-2-yl)pentan-1-ol using 3-(2-bromo-6-chlorophenyl)pyridine in Step 7.
(R)-1-(3-chloro-2-(3-methyl-1,2,4-oxadiazol-5-yl)phenyl)-5-methoxy-1-((R)-morpholin-2-yl)pentan-1-ol using 5-(2-bromo-6-chlorophenyl)-3-methyl-1,2,4-oxadiazole in Step 7.
(R)-1-(6-fluoro-3′-methoxy-5′-methylbiphenyl-2-yl)-5-methoxy-1-((R)-morpholin-2-yl)pentan-1-ol using 2-bromo-6-fluoro-3′-methoxy-5′-methylbiphenyl in Step 7.
(R)-1-(6-chloro-3′-ethylbiphenyl-2-yl)-5-methoxy-1-((R)-morpholin-2-yl)pentan-1-ol using 2-bromo-6-chloro-3′-ethylbiphenyl in Step 7.
(1R)-1-(6-chloro-2′-fluoro-5′-methylbiphenyl-2-yl)-5-methoxy-1-((R)-morpholin-2-yl)pentan-1-ol using 2′-bromo-6′-chloro-2-fluoro-5-methylbiphenyl in Step 7.
(R)-1-(3-chloro-2-(naphthalen-2-yl)phenyl)-5-methoxy-1-((R)-morpholin-2-yl)pentan-1-ol using 2-(2-bromo-6-chlorophenyl)naphthalene in Step 7.
(R)-1-(3-chloro-2-(quinolin-3-yl)phenyl)-5-methoxy-1-(R)-morpholin-2-yl)pentan-1-ol using 3-(2-bromo-6-chlorophenyl)quinoline in Step 7.
(R)-1-(6-fluoro-3′,5′-dimethoxybiphenyl-2-yl)-5-methoxy-1-((R)-morpholin-2-yl)pentan-1-ol using 2-bromo-6-fluoro-3′,5′-dimethoxybiphenyl in Step 7.
(R)-1-(6-chloro-3′-(methoxymethyl)biphenyl-2-yl)-5-methoxy-1-((R)-morpholin-2-yl)pentan-1-ol using 2-bromo-6-chloro-3′-(methoxymethyl)biphenyl in Step 7.
(1R)-1-(3-chloro-2-(isoquinolin-4-yl)phenyl)-5-methoxy-1-(R)-morpholin-2-yl)pentan-1-ol using 4-(2-bromo-6-chlorophenyl)isoquinoline in Step 7.
(R)-1-(6-chloro-3′,5′-dimethoxybiphenyl-2-yl)-5-methoxy-1-(R)-morpholin-2-yl)pentan-1-ol using 2-bromo-6-chloro-3′,5′-dimethoxybiphenyl in Step 7.
(R)-1-(6-fluoro-3′,5′-dimethoxybiphenyl-2-yl)-5-methoxy-1-((R)-morpholin-2-yl)pentan-1-ol using 2-bromo-6-fluoro-3′,5′-dimethoxybiphenyl in Step 7.
(R)-1-(3′-ethoxy-6-fluoro-5′-(trifluoromethyl)biphenyl-2-yl)-5-methoxy-1-((R)-morpholin-2-yl)pentan-1-ol using 2-bromo-3′-ethoxy-6-fluoro-5′-(trifluoromethyl)biphenyl in Step 7.
(1R)-1-[2-chloro-3-(3-ethylphenyl)-4-pyridinyl]-5-(methyloxy)-1-[(2R)-2-morpholinyl]-1-pentanol using 4-bromo-2-chloro-3-(3-ethylphenyl)pyridine in Step 7.
1-[2-(1-benzothien-3-yl)-3-chlorophenyl]-5-(methyloxy)-1-[(2R)-2-morpholinyl]-1-pentanol using 3-(2-bromo-6-chlorophenyl)-1-benzothiophene in Step 7.
(1R)-1-[4-chloro-3-(3-ethylphenyl)-2-pyridinyl]-5-(methyloxy)-1-[(2R)-2-morpholinyl]-1-pentanol using 2-bromo-4-chloro-3-(3-ethylphenyl)pyridine in Step 7.

### Preparation 10b

### Alternate Morpholines

### Methyl {(4R)-4-(3′-ethyl-6-fluoro-2-biphenylyl)-4-hydroxy-4-[(2R)-2-morpholinyl]butyl}carbamate

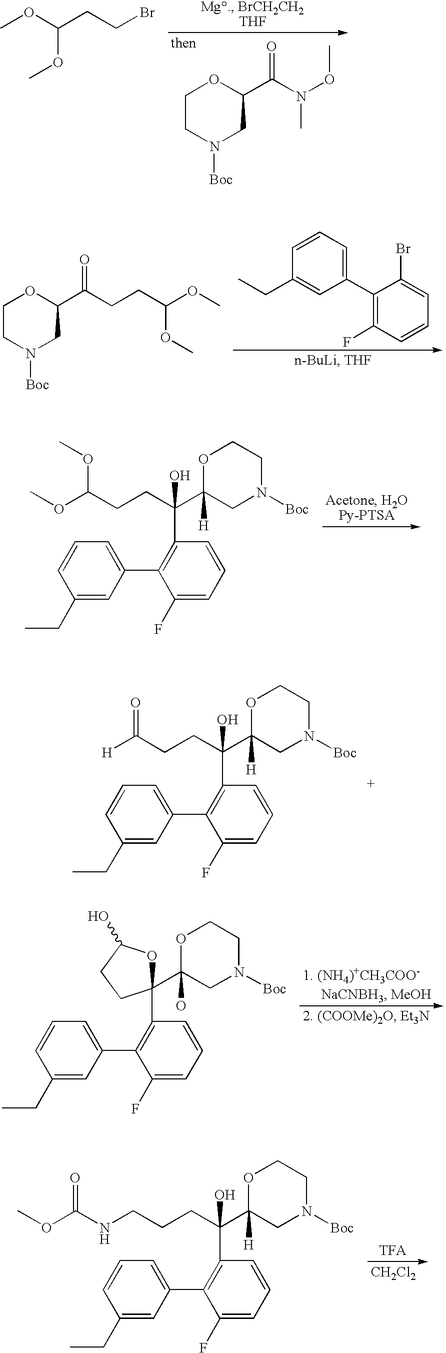

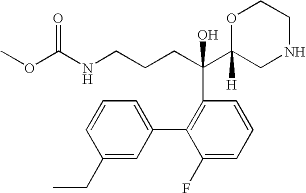

### Step 1. 1,1-dimethylethyl (2R)-2-[4,4-bis(methyloxy)butanoyl]-4-morpholinecarboxylate

To a stirred solution of Mg° (960 mg, 40.0 mmol) in 35.0 mL of dry THF, was added at reflux 0.3 mL of BrCH₂CH₂Br (0.3 mL, 3.5 mmol) and 3-bromo-1,1-bis(methyloxy)propane (6.45 g, 35.0 mmol). The resulting mixture was then heated at reflux for 1 h. Then the solution was cooled to room temperature and added to a solution of 1,1-dimethylethyl (2R)-2-{[methyl(methyloxy)amino]carbonyl}-4-morpholinecarboxylate (5.46 g, 20.0 mmol) dissolved in 20.0 mL of dry THF at −30° C. The mixture was warmed to room temperature and stirred over night. The mixture was quenched with NH₄Cl and extracted with ethyl acetate. The organic layer was then dried, filtered, and concentrated in vacuo. The crude material was then purified via column chromatography to afford 1,1-dimethylethyl (2R)-2-[4,4-bis(methyloxy)butanoyl]-4-morpholinecarboxylate (5.06 g, 81%).

### Step 2. 1,1-dimethylethyl (2R)-2-[(1R)-1-(3′-ethyl-6-fluoro-2-biphenylyl)-1-hydroxy-4,4-bis(methyloxy)butyl]-4-morpholinecarboxylate

To a stirred solution of 2-bromo-3′-ethyl-6-fluorobiphenyl (6.6 g, 23.6 mmol) in 15.0 mL of dry THF at −78° C., was added n-BuLi (10 mL, 25 mmol). The resulting mixture was stirred for 30 min at −78° C. Then, a solution of 1,1-dimethylethyl (2R)-2-[4,4-bis(methyloxy)butanoyl]-4-morpholinecarboxylate (5.0 g, 15.7 mmol) dissolved in 15.0 ml of dry THF was added. The resulting solution was warmed to room temperature over 3 h and quenched with 0.5N HCl. The mixture was then extracted with ethyl acetate. The organic layer was then dried, filtered, and concentrated in vacuo. The crude residue was purified via column chromatography to give 1,1-dimethylethyl (2R)-2-[(1R)-1-(3′-ethyl-6-fluoro-2-biphenylyl)-1-hydroxy-4,4-bis(methyloxy)butyl]-4-morpholinecarboxylate (6.7 g, 82%).

### Step 3. Mixture of 1,1-dimethylethyl (2R)-2-[(1R)-1-(3′-ethyl-6-fluoro-2-biphenylyl)-1-hydroxy-4-oxobutyl]-4-morpholinecarboxylate and 1,1-dimethylethyl (2R)-2-[(2R)-2-(3′-ethyl-6-fluoro-2-biphenylyl)-5-hydroxytetrahydro-2-furanyl]-4-morpholinecarboxylate

To a microwave vial containing 1,1-dimethylethyl (2R)-2-[(1R)-1-(3′-ethyl-6-fluoro-2-biphenylyl)-1-hydroxy-4,4-bis(methyloxy)butyl]-4-morpholinecarboxylate (180 mg, 0.35 mmol) dissolved in acetone (2 mL) and H₂O (0.8 mL), a catalytic amount of pyridinium p-toluenesulfonate (22 mg, 0.088 mmol) was added and the mixture was stirred under microwave irradiation for 30 min 100° C. The mixture was quenched with NaHCO₃ and then extracted with ethyl acetate. The organic layer was then dried, filtered, and concentrated in vacuo. The crude mixture of 1,1-dimethylethyl (2R)-2-[(1R)-1-(3′-ethyl-6-fluoro-2-biphenylyl)-1-hydroxy-4-oxobutyl]-4-morpholinecarboxylate and 1,1-dimethylethyl (2R)-2-[(2R)-2-(3′-ethyl-6-fluoro-2-biphenylyl)-5-hydroxytetrahydro-2-furanyl]-4-morpholinecarboxylate was used in the next step without other purification.

### Step 4. 1,1-dimethylethyl (2R)-2-((1R)-1-(3′-ethyl-6-fluoro-2-biphenylyl)-1-hydroxy-4-{[(methyloxy)carbonyl]amino}butyl)-4-morpholinecarboxylate

To a microwave vial containing the mixture of 1,1-dimethylethyl (2R)-2-[(1R)-1-(3′-ethyl-6-fluoro-2-biphenylyl)-1-hydroxy-4-oxobutyl]-4-morpholinecarboxylate and 1,1-dimethylethyl (2R)-2-[(2R)-2-(3′-ethyl-6-fluoro-2-biphenylyl)-5-hydroxytetrahydro-2-furanyl]-4-morpholinecarboxylate (500 mg, 1.1 mmol) dissolved in dry MeOH (8 mL), (NH₄)⁺CH₃COO⁻ (3.0 g) was added followed by NaCNBH₃ (135 mg, 2.2 mmol). The mixture was then stirred for 30 min at 100° C. under microwave irradiation. The solvent was removed in vacuo and the residue redissolved in methylene chloride and washed with NaHCO₃. The organic layer was then dried, filtered, and concentrated in vacuo. The crude material was then purified via SCX (10 g) column. The amine was then dissolved in methylene chloride (8.0 mL) and Et₃N (0.300 g, 3.0 mmol) and (COOMe)₂O (0.328 g, 2.0 mmol) added. The resulting mixture was stirred for 20 min at room temperature. The reaction was diluted with methylene chloride and washed with NaHCO₃. The organic layer was then dried, filtered, and concentrated in vacuo. The crude material was then purified by flash chromatography to afford 1,1-dimethylethyl (2R)-241R)-1-(3′-ethyl-6-fluoro-2-biphenylyl)-1-hydroxy-4-{[(methyloxy)carbonyl]amino}butyl)-4-morpholinecarboxylate (0.135 g, 23%). MS (E/Z): 431.2 (M-Boc+H⁺)

### Step 5. Methyl {(4R)-4-(3′-ethyl-6-fluoro-2-biphenylyl)-4-hydroxy-4-[(2R)-2-morpholinyl]butyl}carbamate

To a solution of 1,1-dimethylethyl (2R)-2-41R)-1-(3′-ethyl-6-fluoro-2-biphenylyl)-1-hydroxy-4-{[(methyloxy)carbonyl]amino}butyl)-4-morpholinecarboxylate (135 mg, 0.25 mmol) in methylene chloride (4 mL) at 0° C., was added TFA (1.0 mL, 25% v/v). The solution was stirred for 1.5 h at room temperature before the solvent was removed in vacuo and the crude material filtered through an SCX column (5 g) to afford methyl {(4R)-4-(3′-ethyl-6-fluoro-2-biphenylyl)-4-hydroxy-4-[(2R)-2-morpholinyl]butyl}carbamate (0.100 g, >99%).

The following morpholines were prepared using procedures analogous to those described above:

### Methyl (R)-4-(3-fluoro-2-(quinolin-3-yl)phenyl)-4-hydroxy-4-((R)-morpholin-2-yl)butylcarbamate using 3-(2-bromo-6-fluorophenyl)quinoline in Step 2

### Preparation 11

### Methyl {4-(6-chloro-3′-methyl-2-biphenylyl)-4-hydroxy-4-[(3R)-3-piperidinyl]butyl}carbamate

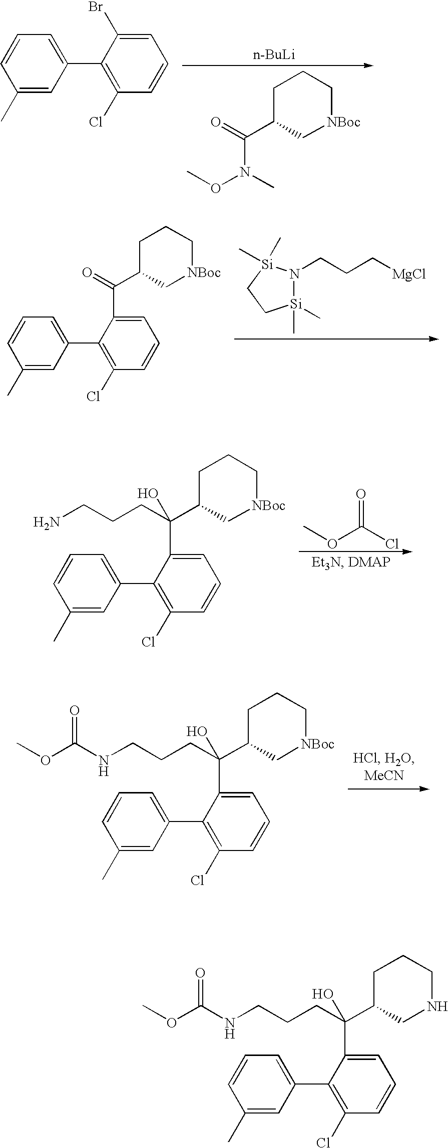

Step 1. (R)-tert-butyl 3-(6-chloro-3′-methylbiphenylcarbonyl)piperidine-1-carboxylate: To a solution of 6-bromo-2-fluoro-3′-methylbiphenyl (2 g, 7.14 mmol) in anhydrous THF (30 mL) cooled to ±78° C. was added dropwise a solution of 1.6 M of n-BuLi in hexane (4.46 mL). The reaction mixture was stirred at −78° C. for 1 h and a solution of (R)-tent-butyl 3-(methoxy(methyl)carbamoyl)piperidine-1-carboxylate (1.94 g, 7.14 mmol) in anhydrous THF (20 mL)was added. The mixture was allowed to warm to rt and stirred overnight. The mixture was quenched with satd aq NH₄Cl (40 mL) and extracted with EtOAc (40 mL). The combined organic layers were dried over Na₂SO₄ and concentrated to give crude product, which was purified by flash column chromatography to afford (R)-tent-butyl 3-(6-chloro-3′-methylbiphenylcarbonyl)piperidine-1-carboxylate (1 g, 34%). ¹H NMR (400 MHz, CD₃OD): 0.80-1.20 (m, 8H), 1.30 (s, 1H), 1.40 (s, 1H), 1.40-1.60 (m, 2H), 2.00-2.18 (s, 1H), 2.30-2.40 (s, 3H), 2.60-2.80 (m, 2H), 3.50-3.80 (m, 2H), 7.00-7.15 (s, 2H), 7.20-7.30 (d, 1H), 7.30-7.40 (t, 2H), 7.39-7.48 (t, 1H), 7.60-7.70 (d, 1H); MS (E/Z): 414 (M+H⁺)

Step 2. 1,1-dimethylethyl (3R)-3-[4-amino-1-(6-chloro-3′-methyl-2-biphenylyl)-1-hydroxybutyl]-1-piperidinecarboxylate: To a solution of (R)-tent-butyl 3-(6-chloro-3′-methylbiphenylcarbonyl)piperidine-1-carboxylate (800 mg, 1.94 mmol) in anhydrous THF (15 mL) cooled to ±78° C. was added dropwise a solution of 2 M (3-(2,2,5,5-tetramethyl-1,2,5-azadisilolidin-1-yl)propyl)magnesium chloride in THF (0.968 mL, 1.94 mmol). After addition, the reaction mixture was allowed to warm slowly to rt while stirring overnight. The mixture was quenched with satd aq NH₄Cl (15 mL) and extracted with CH₂Cl₂ (3×). The combined organic layers were dried over Na₂SO₄ and concentrated to give crude 1,1-dimethylethyl (3R)-3-[4-amino-1-(6-chloro-3′-methyl-2-biphenylyl)-1-hydroxybutyl]-1-piperidinecarboxylate (900 mg), which was used in the next step without further purification.

Step 3. 1,1-dimethylethyl (3R)-3-(1-(6-chloro-3′-methyl-2-biphenylyl)-1-hydroxy-4-{[(methyloxy)carbonyl]amino}butyl)-1-piperidinecarboxylate: To a solution of 1,1-dimethylethyl (3R)-3-[4-amino-1-(6-chloro-3′-methyl-2-biphenylyl)-1-hydroxybutyl]-1-piperidinecarboxylate (800 mg, 1.69 mmol) in anhydrous CH₂Cl₂ (15 mL) were added 4-dimethyaminopyridine (1.24 g, 10.17 mmol) and Et₃N (2.35 mL, 16.95 mmol). The mixture was cooled with an ice bath and methyl chloroformate (0.65 mL, 8.47 mmol) in CH₂Cl₂ (5 mL) was added. The reaction mixture was allowed to warm slowly to rt while stirring overnight. The solvent was removed in vacuo and the residue was purified by column chromatography to afford 1,1-dimethylethyl (3R)-3-(1-(6-chloro-3′-methyl-2-biphenylyl)-1-hydroxy-4-{[(methyloxy)carbonyl]amino}butyl)-1-piperidinecarboxylate (700 mg, 78%). ¹H NMR (400 MHz, CD₃OD): 1.00-1.70 (m, 17H), 2.30-2.50 (d, 3H), 2.50-2.70 (s, 1H), 2.90-2.31 (m, 2H), 3.50-3.52 (m, 3H), 3.80-4.20 (m, 2H), 6.0-7.15 (m, 3H), 7.15-7.40 (m, 3H), 7.50-7.70 (m, 1H); MS (E/Z): 531 (M+H⁺)

Step 4. Methyl {4-(6-chloro-3′-methyl-2-biphenylyl)-4-hydroxy-4-[(3R)-3-piperidinyl]butyl}carbamate: To a solution of 1,1-dimethylethyl (3R)-3-(1-(6-chloro-3′-methyl-2-biphenylyl)-1-hydroxy-4-{[(methyloxy)carbonyl]amino}butyl)-1-piperidinecarboxylate (600 mg, 1.13 mg) in CH₃CN (18 mL) was added 2N aq HCl (15 mL) and the reaction mixture was vigorously stirred overnight at rt. The solvents were removed in vacuo to give methyl {4-(6-chloro-3′-methyl-2-biphenylyl)-4-hydroxy-4-[(3R)-3-piperidinyl]butyl}carbamate as its hydrochloride salt (500 mg, 95.8%). ¹H NMR (400 MHz, CD₃OD): 1.00-1.20 (m, 1H), 1.30-1.80 (m, 8H), 1.80-2.00 (m, 2H), 2.40-2.50 (d, 3H), 2.75-2.90 (t, 1H), 2.90-3.05 (m, 3H), 3.05-3.12 (t, 1H), 3.20-3.30 (m, 1H), 3.30-3.40 (m, 1H), 3.60-3.70 (d, 4H), 6.90-6.98 (d, 1H), 7.00-7.12 (m, 1H), 7.25-7.50 (m, 4H), 7.75-7.85 (d, 1H); MS (E/Z): 431 (M+H⁺)

The following piperidines were prepared using procedures analogous to those described above:
Methyl 4-hydroxy-4-((R)-piperidin-3-yl)-4-(2-(pyridin-4-yl)phenyl)butylcarbamate using 4-(2-bromophenyl)pyridine in Step 1.
N-(4-hydroxy-4-((R)-piperidin-3-yl)-4-(2-(o-tolyloxy)phenyl)butyl)acetamide using 1-bromo-2-(o-tolyloxy)benzene in Step 1 and acetyl chloride in place of methyl chloroformate in Step 3.
Methyl 4-hydroxy-4-((R)-piperidin-3-yl)-4-(2-(o-tolyloxy)phenyl)butylcarbamate using 1-bromo-2-(o-tolyloxy)benzene in Step 1.
Methyl 4-(3′-ethyl-6-fluorobiphenyl-2-yl)-4-hydroxy-4-((R)-piperidin-3-yl)butylcarbamate using 2-bromo-3′-ethyl-6-fluorobiphenyl in Step 1.
Methyl 4-(6-fluoro-3′-methoxybiphenyl-2-yl)-4-hydroxy-4-((R)-piperidin-3-yl)butylcarbamate using 2-bromo-6-fluoro-3′-methoxybiphenyl in Step 1.
Methyl 4-(6-chloro-3′-isopropylbiphenyl-2-yl)-4-hydroxy-4-((R)-piperidin-3-yl)butylcarbamate using 2-bromo-6-chloro-3′-isopropylbiphenyl in Step 1.
Methyl 4-(6-chloro-3′-methoxybiphenyl-2-yl)-4-hydroxy-4-((R)-piperidin-3-yl)butylcarbamate using 2-bromo-6-chloro-3′-methoxybiphenyl in Step 1.
Methyl 4-(3-chloro-2-(quinolin-3-yl)phenyl)-4-hydroxy-4-((R)-piperidin-3-yl)butylcarbamate using 3-(2-bromo-6-chlorophenyl)quinoline in Step 1.
Methyl 4-(6-chloro-3′-ethylbiphenyl-2-yl)-4-hydroxy-4-((R)-piperidin-3-yl)butylcarbamate using 2-bromo-6-chloro-3′-ethylbiphenyl in Step 1.
N-(4-(6-chloro-3′-ethylbiphenyl-2-yl)-4-hydroxy-4-((R)-piperidin-3-yl)butyl)acetamide using 2-bromo-6-chloro-3′-ethylbiphenyl in Step 1 and acetyl chloride in place of methyl chloroformate in Step 3.
Methyl 4-(3-chloro-2-(o-tolyloxy)phenyl)-4-hydroxy-4-(R)-piperidin-3-yl)butylcarbamate using 1-bromo-3-chloro-2-(o-tolyloxy)benzene in Step 1.
Methyl 4-(3-chloro-2-(2-ethylphenoxy)phenyl)-4-hydroxy-4-(R)-piperidin-3-yl)butylcarbamate using 1-bromo-3-chloro-2-(2-ethylphenoxy)benzene in Step 1.
N-(4-(6-chloro-3′-methylbiphenyl-2-yl)-4-hydroxy-4-((R)-piperidin-3-yl)butyl)acetamide using 2-bromo-6-chloro-3′ methylbiphenyl in Step 1 and acetyl chloride in place of methyl chloroformate in Step 3.
Methyl 4-(4,6-difluoro-3′-methylbiphenyl-2-yl)-4-hydroxy-4-((R)-piperidin-3-yl)butylcarbamate using 2-bromo-4,6-difluoro-3′-methylbiphenyl in Step 1.
Methyl 4-hydroxy-4-((R)-piperidin-3-yl)-4-(2′,4,6-trifluoro-5′-methylbiphenyl-2-yl)butylcarbamate using 2-bromo-2′,4,6-trifluoro-5′-methylbiphenyl in Step 1.
Methyl 4-(6-fluoro-3′-isopropylbiphenyl-2-yl)-4-hydroxy-4-((R)-piperidin-3-yl)butylcarbamate using 2-bromo-6-fluoro-3′-isopropylbiphenyl in Step 1.
Methyl 4-(6-chloro-3′-fluoro-5′-methylbiphenyl-2-yl)-4-hydroxy-4-((R)-piperidin-3-yl)butylcarbamate using 2-bromo-6-chloro-3′-fluoro-5′-methylbiphenyl in Step 1.
Methyl 4-(3′,6-difluoro-5′-methylbiphenyl-2-yl)-4-hydroxy-4-((R)-piperidin-3-yl)butylcarbamate using 2-bromo-3′,6-difluoro-5′-methylbiphenyl in Step 1.
Methyl 4-(3-chloro-2-(3-methylbenzyl)phenyl)-4-hydroxy-4-(R)-piperidin-3-yl)butylcarbamate using 1-bromo-3-chloro-2-(3-methylbenzyl)benzene in Step 1.
Methyl {4-{3-chloro-2-[4-(1-methylethyl)-2-quinazolinyl]phenyl}-4-hydroxy-4-[(3R)-3-piperidinyl]butyl}carbamate using 2-(2-bromo-6-chlorophenyl)-4-(1-methylethyl)quinazoline in Step 1.
Methyl {4-[2-chloro-3-(3-ethylphenyl)-4-pyridinyl]-4-hydroxy-4-[(3R)-3-piperidinyl]butyl}carbamate using 4-bromo-2-chloro-3-(3-ethylphenyl)pyridine in Step 1.
Methyl {4-{3-chloro-2-[8-(1-methylethyl)-2-quinolinyl]phenyl}-4-hydroxy-4-[(3R)-3-piperidinyl]butyl}carbamate using 2-(2-bromo-6-chlorophenyl)-8-(1-methylethyl)quinoline in Step 1.
Methyl {4-[3-fluoro-2-(3-quinolinyl)phenyl]-4-hydroxy-4-[(3R)-3-piperidinyl]butyl}carbamate using 3-(2-bromo-6-fluorophenyl)quinoline in Step 1.
Methyl {4-[3-chloro-2-(5-methyl-2-furanyl)phenyl]-4-hydroxy-4-[(3R)-3-piperidinyl]butyl}carbamate using 2-(2-bromo-6-chlorophenyl)-5-methylfuran in Step 1.
Methyl {4-{2-chloro-3-[3-(1-methylethyl)phenyl]-4-pyridinyl}-4-hydroxy-4-[(3R)-3-piperidinyl]butyl}carbamate using 4-bromo-2-chloro-3-[3-(1-methylethyl)phenyl]pyridine in Step 1.
Methyl {4-[3-chloro-2-(8-methyl-2-quinolinyl)phenyl]-4-hydroxy-4-[(3R)-3-piperidinyl]butyl}carbamate using 2-(2-bromo-6-chlorophenyl)-8-methylquinoline in Step 1.

### Preparation 11b

### Methyl {4-{5-chloro-4-[3-(1-methylethyl)phenyl]-3-pyridinyl}-4-hydroxy-4-[(3R)-3-piperidinyl]butyl}carbamate

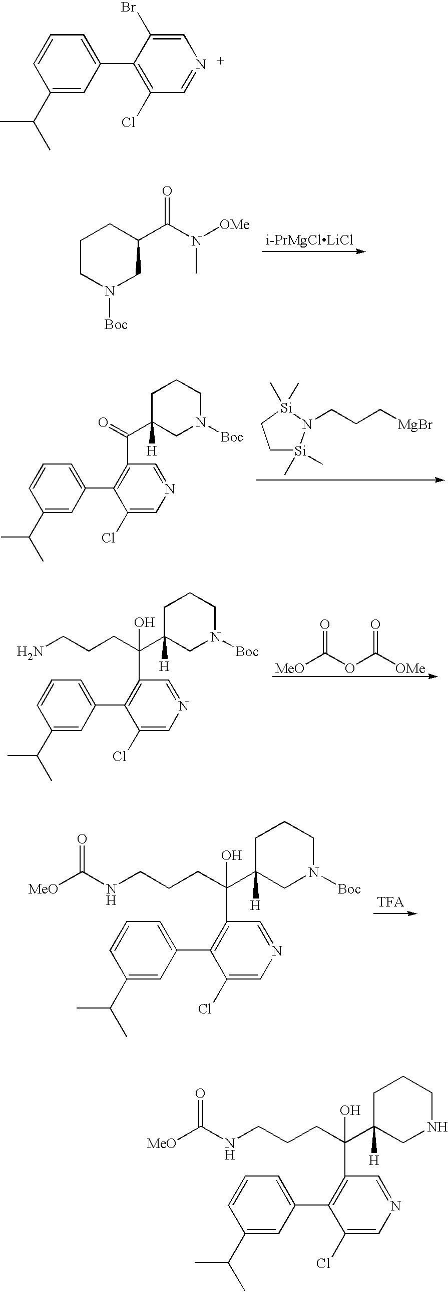

### Step 1: 1,1-dimethylethyl (3R)-3-({5-chloro-4-[3-(1-methylethyl)phenyl]-3-pyridinyl}carbonyl)-1-piperidinecarboxylate

To a cold (0° C.) solution of 3-bromo-5-chloro-4-(3-isopropylphenyl)pyridine (0.78 g, 2.51 mmol) in THF (1.5 mL) was added a i-PrMgCl.LiCl solution (2.6 mL, 1.0 M in THF, 2.6 mmol). After stirring for 45 minutes at 0° C., a solution 1,1-dimethylethyl (3R)-3-{[methyl(methyloxy)amino]carbonyl}-1-piperidinecarboxylate (0.53 g, 1.93 mmol) in THF (1.5 mL) was added. The reaction was stirred at 0° C. for 2 hours and then room temperature for 22 hours. The reaction was quenched with a sat. NH₄Cl solution (3 mL). EtOAc (10 mL) and water (2 mL) were added and then the phases separated. The organic phase was washed with brine, dried over MgSO₄, filtered and concentrated to give 1.3 g of a dark brown oil. The crude ketone was used without purification in the subsequent reaction.

### Step 2: 1,1-dimethylethyl (3R)-3-(4-amino-1-{5-chloro-4-[3-(1-methylethyl)phenyl]-3-pyridinyl}-1-hydroxybutyl)-1-piperidinecarboxylate

To a solution of 1,1-dimethylethyl (3R)-3-({5-chloro-4-[3-(1-methylethyl)phenyl]-3-pyridinyl}carbonyl)-1-piperidinecarboxylate (1.93 mmol) in THF (3 mL) at −50° C. was added a hot solution of the alkyl Grignard (9 mL, 0.72 M THF, 5.80 mmol) quickly. The reaction was slowly allowed to warm to room temperature and stirred overnight. The reaction was quenched with a sat. NH₄Cl solution (3 mL). EtOAc and water was added and then the phases were separated. The organic phase was washed with brine, dried over MgSO₄, filtered and concentrated to give 1.6 g of a dark brown oil. The crude 1,1-dimethylethyl (3R)-3-(4-amino-1-{5-chloro-4-[3-(1-methylethyl)phenyl]-3-pyridinyl}-1-hydroxybutyl)-1-piperidinecarboxylate was used without further purification in the subsequent reaction.

### Step 3. 1,1-dimethylethyl (3R)-3-(1-{5-chloro-4-[3-(1-methylethyl)phenyl]-3-pyridinyl}-1-hydroxy-4-{[(methyloxy)carbonyl]amino}butyl)-1-piperidinecarboxylate

To a cold (0° C.) solution of the 1,1-dimethylethyl (3R)-3-(4-amino-1-{5-chloro-4-[3-(1-methylethyl)phenyl]-3-pyridinyl}-1-hydroxybutyl)-1-piperidinecarboxylate (1.93 mmol) in CH₂Cl₂ were added i-Pr₂NEt (1.3 mL, 7.72 mmol) and then dimethyldicarbonate (0.62 mL, 5.79 mmol). After stirring for 1.5 hour at 0° C., the reaction was quenched with a sat. NH₄Cl solution (2 mL). The reaction was diluted with CH₂Cl₂ and phases separated. The aqueous phase was extracted with CH₂Cl₂. The combined organic extracts were washed with brine, dried over MgSO₄, filtered and concentrated to give 2 g of a dark brown oil. The crude residue was purified by flash chromatography on silica gel and isolated 350 mg (33% yield, 3 steps) of the desired 1,1-dimethylethyl (3R)-3-(1-{5-chloro-4-[3-(1-methylethyl)phenyl]-3-pyridinyl}-1-hydroxy-4-{[(methyloxy)carbonyl]amino}butyl)-1-piperidinecarboxylate as a dark yellow solid.

### Step 4. Methyl {4-{5-chloro-4-[3-(1-methylethyl)phenyl]-3-pyridinyl}-4-hydroxy-4-[(3R)-3-piperidinyl]butyl}carbamate

To a solution of 1,1-dimethylethyl (3R)-3-(1-{5-chloro-4-[3-(1-methylethyl)phenyl]-3-pyridinyl}-1-hydroxy-4-{[(methyloxy)carbonyl]amino}butyl)-1-piperidinecarboxylate (0.35 g, 0.62 mmol) in CH₂Cl₂ (5 mL) was added trifluoroacetic acid (1 mL). After stirring for 2 hours at room temperature, the reaction was concentrated to give a yellow oil. The crude residue was purified with a 5 gm Strata SCX ion exchange resin eluting with 0.6 M NH₃ in MeOH and isolated 278 mg (97% yield) of methyl {4-{5-chloro-4-[3-(1-methylethyl)phenyl]-3-pyridinyl}-4-hydroxy-4-[(3R)-3-piperidinyl]butyl}carbamate as a yellow oil.

The following piperidine was prepared using procedures analogous to those described above:

### Methyl {4-[5-chloro-4-(3-ethylphenyl)-3-pyridinyl]-4-hydroxy-4-[(3R)-3-piperidinyl]butyl}carbamate using 3-bromo-5-chloro-4-(3-ethylphenyl)pyridine in Step 1

### Preparation 11c

### N-{4-(6-chloro-3′-ethyl-2-biphenylyl)-4-hydroxy-4-[(3R)-3-piperidinyl]butyl}-2-hydroxyacetamide

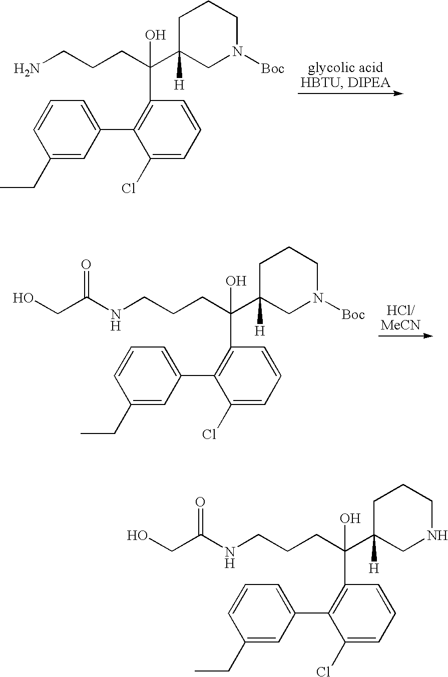

### Step 1. 1,1-dimethylethyl (3R)-3-{1-(6-chloro-3′-ethyl-2-biphenylyl)-1-hydroxy-4-[(hydroxyacetyl)amino]butyl}-1-piperidine carboxylate

A solution of 1,1-dimethylethyl (3R)-3-[4-amino-1-(6-chloro-3′-ethyl-2-biphenylyl)-1-hydroxybutyl]-1-piperidinecarboxylate (75 mg, 0.14 mmol) in 0.5 mL of DMF at 25° C. was treated with glycolic acid (13 mg, 0.17 mmol), i-Pr₂NEt (0.122 mL, 0.7 mmol), and HBTU (64 mg, 0.17 mmol). After 24 h, H₂O was added and the mixture was extracted with EtOAc. The organic extracts were washed (1N aq HCl, 1N aq NaOH, H₂O, brine), dried (Na₂SO₄), concentrated under reduced pressure, and subjected to flash chromatography to provide 1,1-dimethylethyl (3R)-3-{1-(6-chloro-3′-ethyl-2-biphenylyl)-1-hydroxy-4-[(hydroxyacetyl)amino]butyl}-1-piperidinecarboxylate as a colorless oil (39 mg, 51%). MS (m/z) 567.2 (M+Na⁺).

### Step 2. N-{4-(6-chloro-3′-ethyl-2-biphenylyl)-4-hydroxy-4-[(3R)-3-piperidinyl]butyl}-2-hydroxyacetamide

A solution of 1,1-dimethylethyl (3R)-3-{1-(6-chloro-3′-ethyl-2-biphenylyl)-1-hydroxy-4-(hydroxyacetyl)amino)butyl}-1-piperidinecarboxylate (45 mg, 0.08 mmol) in 3 mL of CH₃CN at 25° C. was treated with 3 mL of aq 2N HCl. After 24 h, the mixture was concentrated under reduced pressure to provide N-{4-(6-chloro-3′-ethyl-2-biphenylyl)-4-hydroxy-4-[(3R)-3-piperidinyl]butyl}-2-hydroxyacetamide as a white solid (41 mg, quantitative). MS (m/z) 445.2 (M+H⁺).

The following piperidines were prepared following procedures analogous to those described above using the appropriate piperidine and the indicated acid:

| Structure | Name | Acid Used in Step 1 |
|---|---|---|
| 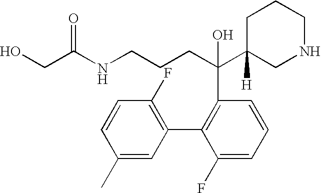 | N-(4-(2′,6-difluoro-5′- methylbiphenyl-2-yl)-4-hydroxy-4- ((3R)-piperidin-3-yl)butyl)-2- hydroxyacetamide | glycolic acid |

### Preparation 12

### N-(2-((S)-(6-fluoro-3′-methylbiphenyl-2-yl)((R)-morpholin-2-yl)methoxy)ethyl)acetamide

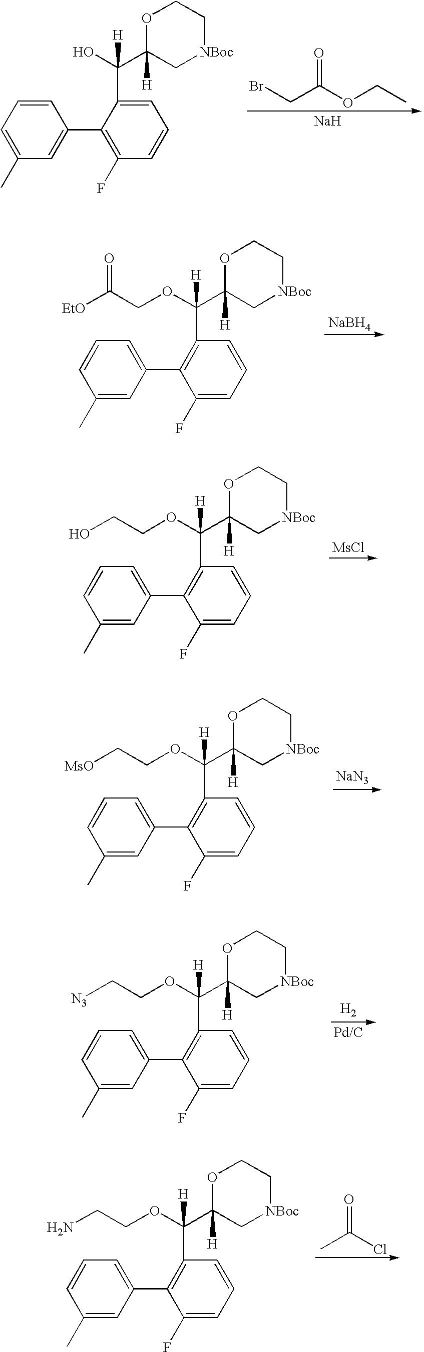

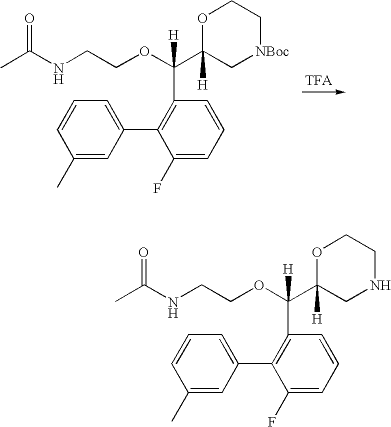

Step 1. (R)-tent-butyl 2-(S)-(2-ethoxy-2-oxoethoxy)(6-fluoro-3′-methylbiphenyl-2-yl)methyl)morpholine-4-carboxylate: To a slurry of 60% NaH in oil (0.75 g, 18.7 mmol) in THF (30 mL) was added a solution of (R)-tent-butyl 2-((S)-(6-fluoro-3′-methylbiphenyl-2-yl)(hydroxy)methyl)morpholine-4-carboxylate (2.5 g, 6.23 mmol) in THF (20 mL) dropwise at and then the reaction mixture was stirred for about 1 h at rt. A solution of ethyl 3-bromopropionate (1.55 g, 9.35 mmol) in THF (20 mL) was added dropwise while the temperature was maintained at −15 to −5° C. The mixture was allowed to warm slowly to rt and stirred for ±2 h until the reaction was complete by tlc analysis. The reaction was cooled in an ice bath, quenched with satd aq NH₄Cl (120 mL) and extracted with EtOAc. The combined organic extracts were washed with brine, dried over NaSO₄, concentrated and purified by flash chromatography to afford (R)-tent-butyl 2-(S)-(2-ethoxy-2-oxoethoxy)(6-fluoro-3′-methylbiphenyl-2-yl)methyl)morpholine-4-carboxylate (570 mg, 19%). MS (E/Z): 488 (M+H⁺)

Step 2. (R)-tent-butyl 2-(S)-(6-fluoro-3′-methylbiphenyl-2-yl)(2-hydroxyethoxy)methyl)morpholine-4-carboxylate: To a solution of (R)-tent-butyl 2-((S)-(2-ethoxy-2-oxoethoxy)(6-fluoro-3′-methylbiphenyl-2-yl)methyl)morpholine-4-carboxylate (570 mg, 1.17 mmol) in CH₃OH (20 mL) at rt, NaBH₄ (355 mg, 9.36 mmol) was added in portions. The mixture was stirred for ±0.5 h at rt and then evaporated. The residue was partitioned between water and EtOAc. The combined organic layers were washed with brine, dried over anhydrous NaSO₄ and evaporated to give semi-crude (R)-tent-butyl 2-(S)-(6-fluoro-3′-methylbiphenyl-2-yl)(2-hydroxyethoxy)methyl)morpholine-4-carboxylate (498 mg, 96%), which was used in the next step reaction without further purification. MS (E/Z): 446 (M+H⁺)

Step 3. (R)-tent-butyl 2-(S)-(6-fluoro-3′-methylbiphenyl-2-yl)(2-(methylsulfonyloxy)ethoxy)methyl)morpholine-4-carboxylate: To a solution of (R)-tert-butyl 2-(S)-(6-fluoro-3′-methylbiphenyl-2-yl)(2-hydroxyethoxy)methyl) morpholine-4-carboxylate (498 mg, 1.12 mmol) in dry CH₂Cl₂(15 mL) was added Et₃N (472 mg, 4.68 mmol) at −0 to −5° C. A solution of MsCl (267 mg, 2.34 mmol) in dry CH₂Cl₂ (10 mL) was added dropwise at the same temperature. The mixture was allowed to warm to room temperature gradually. Water (10 mL) was added and the aqueous layer was extracted with CH₂Cl₂ (3×20 mL). The combined organic layers were washed with 10% aq citric acid, satd aq NaHCO₃ and brine, dried over Na₂SO₄, filtered and concentrated to afford crude (R)-tent-butyl 2-(S)-(6-fluoro-3′-methylbiphenyl-2-yl)(2-(methylsulfonyloxy)ethoxy)methyl)morpholine-4-carboxylate (554 mg, 95%). which was used in the next step without further purification. MS (E/Z): 524 (M+H⁺)

Step 4. (R)-tent-butyl 2-((S)-(2-azidoethoxy)(6-fluoro-3′-methylbiphenyl-2-yl)methyl)morpholine-4-carboxylate: To a solution of (R)-tent-butyl 2-45)-(6-fluoro-3′-methylbiphenyl-2-yl)(2-(methylsulfonyloxy)ethoxy)methyl)morpholine-4-carboxylate (554 mg, 1.0 mmol) in anhydrous DMF (18 mL), solid NaN₃ (230 mg, 3.51 mmol) was added and the reaction mixture was heated to 70° C. for overnight. The reaction mixture was cooled to rt and diluted with EtOAc (110 mL), and water (30 ml). The organic phase was washed with water (3×30 mL), dried over Na₂SO₄ and evaporated to give (R)-tent-butyl 2-(S)-(2-azidoethoxy)(6-fluoro-3′-methylbiphenyl-2-yl)methyl)morpholine-4-carboxylate (423 mg, 90%). MS (E/Z): 471 (M+H⁺)

Step 5. (R)-tent-butyl 2-((S)-(2-aminoethoxy)(6-fluoro-3′-methylbiphenyl-2-yl)methyl)morpholine-4-carboxylate: To a solution of (R)-tent-butyl 2-((S)-(2-azidoethoxy)(6-fluoro-3′-methylbiphenyl-2-yl)methyl)morpholine-4-carboxylate (423 mg, 0.9 mmol) in EtOAc (20 mL) was added wetted Pd/C (42 mg) and the mixture was hydrogenated overnight using a balloon of hydrogen. The mixture was filtered through a pad of Celite and the solvent was removed to give (R)-tent-butyl 2-((S)-(2-aminoethoxy)(6-fluoro-3′-methylbiphenyl-2-yl)methyl)morpholine-4-carboxylate (430 mg, 100%). MS (E/Z): 445 (M+H⁺)

Step 6. (R)-tent-butyl 2-((S)-(2-acetamidoethoxy)(6-fluoro-3′-methylbiphenyl-2-yl)methyl)morpholine-4-carboxylate: To a round-bottom flask were added (R)-tert-butyl 2-(S)-(2-aminoethoxy)(6-fluoro-3′-methylbiphenyl-2-yl)methyl)morpholine-4-carboxylate (280 mg, 0.63 mmol), triethylamine (0.19 mL, 1.89 mmol) and anhydrous CH₂Cl₂ (15 mL). The mixture was cooled in an ice bath and a solution of acetyl chloride (49.2 mg, 0.045 mL, 0.63 mmol) was added. The reaction mixture was allowed to warm slowly to rt and stirred until the reaction was complete (ca 1-2 h). The solvent was removed by evaporation, and the residue was purified by preparative tlc to give (R)-tent-butyl 2-45)-(2-acetamidoethoxy)(6-fluoro-3′-methylbiphenyl-2-yl)methyl)morpholine-4-carboxylate (202 mg, 66%). ¹H NMR (300 MHz, CDCl₃): δ=1.45 (s, 9H), 1.93 (s, 3H), 2.38 (s, 3H), 2.87˜3.2 (m, 6H), 3.32-3.92 (m, 5H), 4.28 (d, 1H), 7.01-7.25 (m, 3H), 7.28-7.37 (m, 4H), 9.41-9.54 (s, 1H). MS (E/Z): 487 (M+H⁺)

Step 7. N-(2-(S)-(6-fluoro-3′-methylbiphenyl-2-yl)((R)-morpholin-2-yl)methoxy)ethyl)acetamide: (R)-tert-butyl 2-(S)-(2-acetamidoethoxy)(6-fluoro-3′-methylbiphenyl-2-yl)methyl)morpholine-4-carboxylate (202 mg, 0.42 mmol) was dissolved in 20% TFA in CH₂Cl₂ (8 mL) and stirred for about 1 h at rt. The mixture was neutralized with satd aq NaHCO₃ and the product was extracted with CH₂Cl₂. The combined organic layers were washed with brine, dried over Na₂SO₄ and concentrated to give N-(2-(S)-(6-fluoro-3′-methylbiphenyl-2-yl)((R)-morpholin-2-yl)methoxy)ethyl)acetamide (130 mg, 82%). ¹H NMR (300 MHz, CDCl₃): δ=1.98 (s, 3H), 2.39 (s, 3H), 2.90˜3.3 (m, 6H), 3.31˜3.41 (m, 2H), 3.6˜4.0 (m, 3H), 4.33 (d, 1H), 6.56-6.57 (s, 1H), 6.97˜7.14 (m, 3H), 7.27˜7.40 (m, 4H), 9.40˜9.55 (s, 1H). MS (E/Z): 387 (M+H⁺).

The following compounds were prepared using procedures analogous to those described above:

### methyl 2-45)-(6-fluoro-3′-methylbiphenyl-2-yl)((R)-morpholin-2-yl)methoxy)ethylcarbamate using methyl chloroformate in place of acetyl chloride in Step 6

### Preparation 13

### 1-bromo-3-chloro-2-[(3-methylphenyl)methyl]benzene

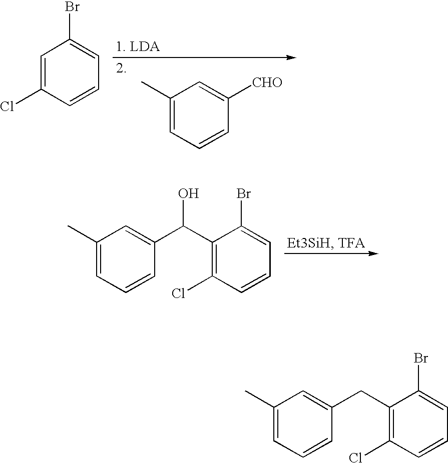

Step 1. (2-bromo-6-chlorophenyl)(m-tolyl)methanol: To a −78° C. solution of diisopropylamine (9.9 mL, 70 mmol) in anhydrous THF (80 mL) was added dropwise a n-BuLi solution (31.5 mL, 50 mmol, 1.6M hexanes). The reaction was stirred for 20 min at −78° C. and 1-chloro-3-bromobenzene (5.9 mL, 50 mmol) was added. After stirring for 30 min at −78° C., m-tolualdehyde (5.9 mL, 50 mmol) was added. The reaction was gradually allowed to warm to rt and then stirred overnight. The reaction was quenched with the addition of water and then extracted with EtOAc. The organic extracts were dried over MgSO₄, filtered and concentrated. The crude residue was purified by flash chromatography on silica gel (ISCO Combiflash, 120 gm column, Hexane/EtOAc 0→10%) and isolated 10.7 g of (2-bromo-6-chlorophenyl)(m-tolyl)methanol.

Step 2. 1-bromo-3-chloro-2-[(3-methylphenyl)methyl]benzene: (2-bromo-6-chlorophenyl)(m-tolyl)methanol (10.7 g, 34.4 mmol) was dissolved in CH₂Cl₂ (50 mL) and then Et₃SiH (22 mL, 138 mmol) and trifluoroacetic acid (10.6 mL, 138 mmol) were added. After stirring at rt overnight, the reaction was concentrated to remove solvent. The crude residue was purified by flash chromatography on silica gel (ISCO Combiflash, 120 gm column, Hexane/EtOAc 0→10%) and isolated 8.7 g of 1-bromo-3-chloro-2-[(3-methylphenyl)methyl]benzene as a white solid.

1-bromo-3-chloro-2-[(2-methylphenyl)methyl]benzene was prepared using procedures analogous to those described above using o-tolualdehyde in Step 1.

### Preparation 14

### 5-(2-bromo-6-chlorophenyl)-3-methyl-1,2,4-oxadiazole

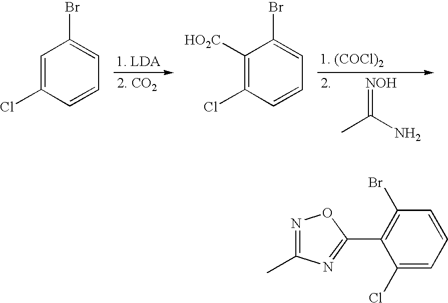

Step 1. 2-bromo-6-chlorobenzoic acid: To a −78° C. solution of n-BuLi (10 mL, 25 mmol, 2.5M Hexanes) in anhydrous THF (70 mL) was added diisopropylamine (3.5 mL, 25 mmol). After stirring for 15 min, 1-chloro-3-bromobenzene (4.32 g, 25 mmol) was added and stirred for 2 h at −78° C. Dry ice (CO₂) was added and after 15 min a 2N aq HCl solution (100 mL) was added. The reaction mixture was extracted with EtOAc. The product was re-crystallized from hexanes and isolated 5 g (85%) of 2-bromo-6-chlorobenzoic acid.

Step 2. 5-(2-bromo-6-chlorophenyl)-3-methyl-1,2,4-oxadiazole: To a solution of 2-bromo-6-chlorobenzoic acid (1 g, 4.25 mmol) in anhydrous CH₂Cl₂ were added dropwise oxalyl chloride (0.45 mL, 5.1 mmol) and 2-3 drops of DMF. The solution was stirred at rt for 2 h and then the solvent was evaporated. The crude residue was added dropwise to a stirred suspension of the acetamide oxime (315 mg, 4.25 mmol) in pyridine (6 mL). After the addition the mixture was refluxed overnight. The solvent was evaporated and the crude residue purified by flash chromatography to afford 376 mg (32%) of 5-(2-bromo-6-chlorophenyl)-3-methyl-1,2,4-oxadiazole.

### Preparation 15

### 3,5-dimethoxyphenylboronic acid

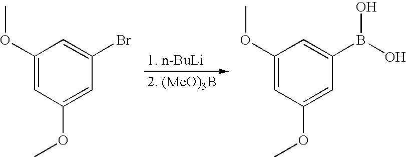

To a solution of 1-bromo-3,5-dimethoxybenzene (5 g, 23 mmol) in THF (100 mL) at −78° C. was added n-Bu-Li (2.5M in hexane, 10 mL, 25 mmol). The mixture was stirred at −78° C. for 30 min and transferred to a solution of B(OCH₃)₃ (3.1 ml) in THF at −78° C. The resulting mixture was warmed up to rt and allowed to stir overnight. The reaction was quenched with 2N aq HCl and extracted with EtOAc. The combined organic extracts were dried over Na₂SO₄ and concentrated. The residue was washed with hexane to give 2.2 g (53% yield) of 3,5-dimethoxyphenylboronic acid as a solid. MS m/z=182.2 (M+H)⁺.

### Preparation 16

### 3-methoxy-5-methylphenylboronic acid

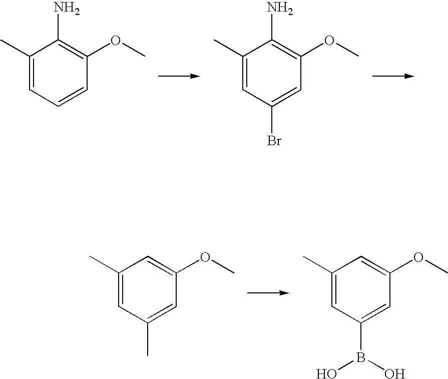

Step 1. 4-bromo-2-methoxy-6-methylaniline: 2-methoxy-6-methylaniline (24.2 g, 182 mmol) was dissolved in MeOH (81 mL) and acetic acid (27 mL) and a solution of bromine (28 g, 182 mmol) in acetic acid (81 mL) was added dropwise. The reaction was allowed to stand at rt for 2 h and concentrated to remove solvents. The crude product was recrystallized from hexanes to give 36 g of 4-bromo-2-methoxy-6-methylaniline as a brown solid.

Step 2. 1-bromo-3-methoxy-5-methylbenzene: To a cold (0° C.) solution of 4-bromo-2-methoxy-6-methylaniline (36 g, 167 mmol) in a mixture of acetic acid (280 mL), water (120 mL) and concentrated HCl (32 mL) was added dropwise a solution of NaNO₂ (13.8 g, 200 mmol) in water (40 mL). The reaction mixture was stirred for 30 min at 0° C. and 50% aq H₃PO₂ (320 mL) was added. After stirring for 8 h at 0° C., the reaction mixture was allowed to stand at rt for 48 h. The reaction mixture was extracted with EtOAc/Et₂O. The crude residue was purified by flash chromatography on silica gel (ISCO Combiflash, 330 g column, 100% hexane) to afford 27.5 g of 1-bromo-3-methoxy-5-methylbenzene as a colorless oil.

Step 3. 3-methoxy-5-methylphenylboronic acid: To a −78° C. solution of 1-bromo-3-methoxy-5-methylbenzene (10 g, 49.8 mmol) in anhydrous THF (200 mL) was added dropwise a n-BuLi solution (37.3 mL, 59.7 mmol, 1.6 M Hexane). After stirring for 30 min at −78° C., trimethyl borate (13.9 mL, 124.3 mmol) was added. The resulting mixture was stirred at −78° C. for 30 min and then warmed to rt and stirred for an additional 60 min. The reaction mixture was poured into an ice/H₂O mixture and acidified with 2N HCl to pH=3. The aqueous solution was extracted with Et₂O. The combined organic extracts were dried over Na₂SO₄, filtered and concentrated in vacuo. The crude residue (13 g) was washed with hexanes. The precipitate was collected and recrystallized from hexanes to give 6.5 g (79%) of 3-methoxy-5-methylphenylboronic acid as a white solid.

### Preparation 17

### 4-((tert-butoxycarbonyl(methyl)amino)methyl)benzoic acid

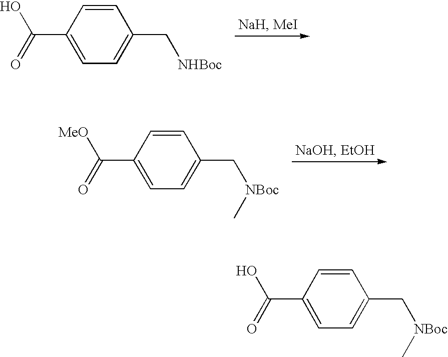

Step 1. Methyl 4-((tert-butoxycarbonyl(methyl)amino)methyl)benzoate: A solution of 4-((tert-butoxycarbonylamino)methyl)benzoic acid (1.01 g, 4.0 mmol) in 10 mL of DMF at 0° C. was treated with NaH (60% in oil, 400 mg, 10 mmol) and warmed to 25° C. After 10 min, methyl iodide (3 mL) was added and the mixture was stirred at 25° C. for 16 h before being concentrated under reduced pressure. The residue was treated with water (20 mL) and extracted with EtOAc (3×20 mL). The combined organic extracts were washed (brine), dried (Na₂SO₄), concentrated, and subjected to flash chromatography to provide methyl 4-((tert-butoxycarbonyl(methyl)amino)methyl)benzoate as a clear oil (849 mg, 76%). MS (m/z) 280.3 (M+H⁺).

Step 2. 4-((tert-butoxycarbonyl(methyl)amino)methyl)benzoic acid: A solution of methyl 4-((tert-butoxycarbonyl(methyl)amino)methyl)benzoate (300 mg, 1.08 mmol) in EtOH (10 ml) at 25° C. was treated with aqueous 1N NaOH (2.16 mL, 2.16 mmol) and the mixture was stirred for 16 h before being extracted with EtOAc (2×5 mL). The aqueous layer was acidified by the addition of aqueous 1N HCl and then extracted with EtOAc (3×10 ml). The combined organic extracts were washed (brine), dried (Na₂SO₄), and concentrated to provide 4-((tert-butoxycarbonyl(methyl)amino)methyl)benzoic acid as a white solid (215 mg, 75%). MS (m/z) 266.1 (M+H⁺).

The following benzoic acids were prepared following procedures analogous to those described above by using the indicated starting material and alkylating agent in Step 1:

| | | | Alkylating |
|---|---|---|---|
| Structure | Name | Starting Material | Agent |
| 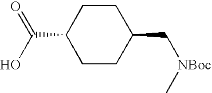 | trans-4-{[{[(1,1- dimethylethyl)oxy]carbonyl} (methyl)amino]methyl} cyclohexanecarboxylic acid | trans-4-[({[(1,1- dimethylethyl)oxy] carbonyl}amino)methyl] cyclohexanecarboxylic acid | Methyl iodide |
| 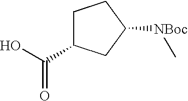 | (1R,3S)-3-[{[(1,1- dimethylethyl)oxy] carbonyl}(methyl)amino] cyclopentanecarboxylic acid | (1R,3S)-3-({[(1,1- dimethylethyl)oxy] carbonyl}amino) cyclopentanecarboxylic acid | Methyl iodide |
| 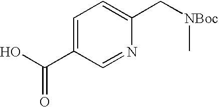 | 6-{[{[(1,1- dimethylethyl)oxy]carbonyl} (methyl)amino]methyl}-3- pyridinecarboxylic acid | 6-[({[(1,1- dimethylethyl)oxy]carbonyl} amino)methyl]-3- pyridinecarboxylic acid | Methyl iodide |
| 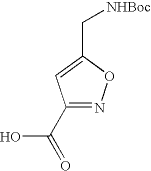 | 5-[({[(1,1- dimethylethyl)oxy]carbonyl} amino)methyl]-3- isoxazolecarboxylic acid | ethyl 5-[({[(1,1- dimethylethyl)oxy]carbonyl} amino)methyl]-3- isoxazolecarboxylate | Omit Step 1 |
| 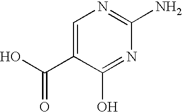 | 2-amino-4-oxo-1,4-dihydro- 5-pyrimidinecarboxylic acid | ethyl 2-amino-4-oxo- 1,4-dihydro-5- pyrimidinecarboxylate | Omit Step 1 |

### Preparation 18

### (R)-1-(6-chloro-3′-ethylbiphenyl-2-yl)-1-((R)-morpholin-2-yl)pent-4-en-1-ol

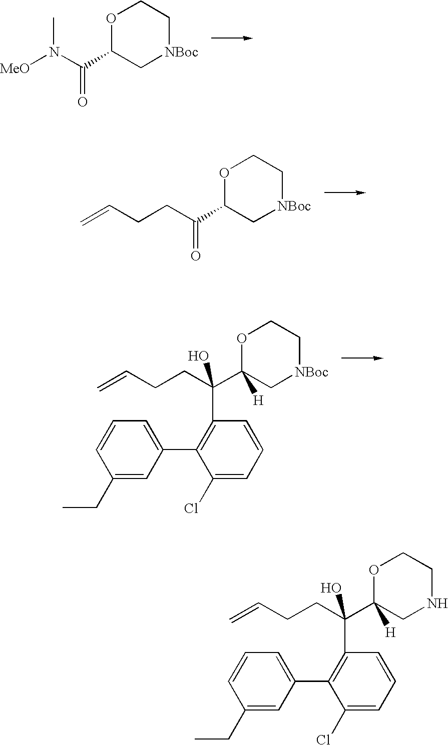

Step 1. (R)-tent-butyl 2-pent-4-enoylmorpholine-4-carboxylate: To a solution of (R)-tent-butyl 2-(methoxy(methyl)carbamoyl)morpholine-4-carboxylate (1.2 g, 4.38 mmol) in 50 mL of THF at −78° C. under a nitrogen atmosphere was slowly added 26 mL (13.3 mmol, 0.5M) of (4-penten-1-yl)magnesium bromide in THF using a syringe. The solution was stirred overnight, allowing it to slowly warm to rt. A saturated solution of NH₄Cl in water (50 mL) was added to the reaction flask. The solution was extracted using EtOAc (3×25 mL). The combined organic extracts were dried over Na₂SO₄ and filtered, followed by concentration under reduced pressure to give 810 mg of (R)-tent-butyl 2-pent-4-enoylmorpholine-4-carboxylate.

Step 2. (R)-tent-butyl 2-((R)-1-(6-chloro-3′-ethylbiphenyl-2-yl)-1-hydroxypent-4-enyl)morpholine-4-carboxylate: To a solution of 2-bromo-6-chloro-3′-ethylbiphenyl, 2.2 g (7.44 mmol) in 20 mL of THF at −78° C. under a nitrogen atmosphere was slowly added a hexane solution of n-BuLi (3.7 ml, 2.5M) using a syringe. The resulting solution was stirred for 0.5 h. 1,1-dimethylethyl (2R)-2-(4-pentenoyl)-4-morpholinecarboxylate (0.8 g, 2.97 mmol) in 20 mL of THF was slowly added to the above solution using a syringe. The reaction was then allowed to stir and warm to rt overnight. A saturated solution of NH₄Cl in water (50 mL) was added to the reaction flask. The solution was extracted using EtOAc (3×25 mL). The combined organic extracts were dried over Na₂SO₄ and filtered, followed by concentration under reduced pressure. This afforded 550 mg of (R)-tent-butyl 2-((R)-1-(6-chloro-3′-ethylbiphenyl-2-yl)-1-hydroxypent-4-enyl)morpholine-4-carboxylate which was used without purification. LC-MS t_{R}=3.74 min, (m/z) 508.2 (M+H⁺).

Step 3. (R)-1-(6-chloro-3′-ethylbiphenyl-2-yl)-1-((R)-morpholin-2-yl)pent-4-en-1-ol; To a solution of 1,1-dimethylethyl (2R)-2-[(1R)-1-(6-chloro-3′-ethyl-2-biphenylyl)-1-hydroxy-4-penten-1-yl]-4-morpholinecarboxylate (73 mg, 0.15 mmol) in 5 ml of acetonitrile was added 5 ml of 2N aqueous HCl. The reaction was stirred overnight. It was basified with 10N aqueous NaOH to pH=14 and extracted with DCM (3×10 ml). The combined organic extracts were dried over Na₂SO₄ and filtered, followed by concentration under reduced pressure. This afforded (R)-1-(6-chloro-3′-ethylbiphenyl-2-yl)-1-((R)-morpholin-2-yl)pent-4-en-1-ol which was used without purification.

### Preparation 19

### (R)-1-(6-chloro-3′-ethylbiphenyl-2-yl)-1-((R)-morpholin-2-yl)pent-4-en-1-ol

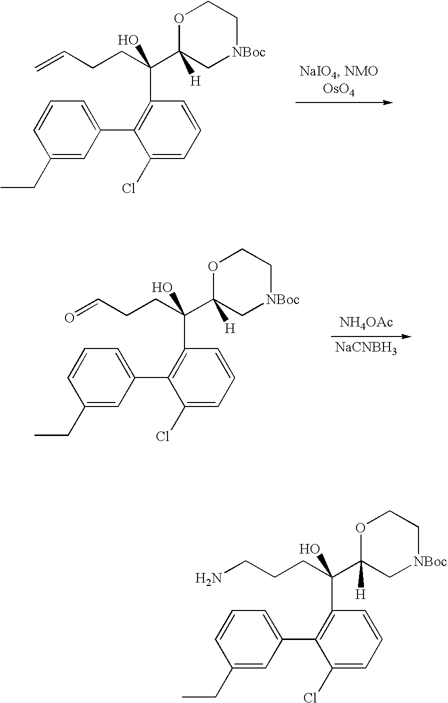

Step 1. (R)-tent-butyl 2-((R)-1-(6-chloro-3′-ethylbiphenyl-2-yl)-1-hydroxy-4-oxobutyl)morpholine-4-carboxylate: To a solution of (R)-tent-butyl 2-((R)-1-(6-chloro-3′-ethylbiphenyl-2-yl)-1-hydroxypent-4-enyl)morpholine-4-carboxylate (350 mg, 0.72 mmol) in 10 mL of THF and 5 mL of water was added NMO (255 mg, 2.18 mmol), followed by NaIO₄ (310 mg, 1.44 mmol) and a few small crystals of OsO₄. The reaction was stirred overnight. The solution was diluted with 10 mL of water and extracted with CH₂Cl₂ (3×10 ml). The combined organic extracts were dried over Na₂SO₄ and filtered, followed by concentration under reduced pressure. This afforded (R)-tent-butyl 2-((R)-1-(6-chloro-3′-ethylbiphenyl-2-yl)-1-hydroxy-4-oxobutyl)morpholine-4-carboxylate which was used without purification. LC-MS t_{R}=3.36 min, (m/z) 510.2 (M+Na⁺).

Step 2. (R)-tent-butyl 2-((R)-4-amino-1-(6-chloro-3′-ethylbiphenyl-2-yl)-1-hydroxybutyl)morpholine-4-carboxylate: To a refluxing solution of (R)-tent-butyl 2-((R)-1-(6-chloro-3′-ethylbiphenyl-2-yl)-1-hydroxy-4-oxobutyl)morpholine-4-carboxylate (350 mg, 0.7 mmol) in 20 mL of MeOH was added NH₃.AcOH (550 mg, 7.2 mmol), followed by NaCNBH₃ (135 mg, 2.2 mmol). After a few h at reflux the reaction was cooled to rt and diluted with 20 mL of water. The solution was extracted using EtOAc (3×10 ml). The combined organic extracts were dried over Na₂SO₄ and filtered, followed by concentration under reduced pressure. This afforded (R)-tert-butyl 2-((R)-4-amino-1-(6-chloro-3′-ethylbiphenyl-2-yl)-1-hydroxybutyl)morpholine-4-carboxylate which was used without purification. LC-MS t_{R}=2.56 min, (m/z) 489.2 (M+H⁺).

### Preparation 20

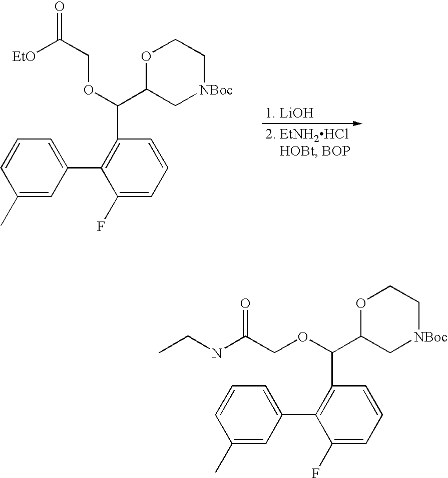

Step 1. 2-44-(tert-butoxycarbonyl)morpholin-2-yl)(6-fluoro-3′-methylbiphenyl-2-yl)methoxy)acetic acid: To a solution of tert-butyl 2-((2-ethoxy-2-oxoethoxy)(6-fluoro-3′-methylbiphenyl-2-yl)methyl)morpholine-4-carboxylate (450 mg, 0.924 mmol) in THF (4 mL) were added water (1 mL) and LiOH (78 mg, 1.86 mmol). The reaction mixture was stirred at rt for 3 h. LC-MS indicated complete hydrolysis of the ester. The reaction mixture was concentrated and redissolved in water. The resulting solution was neutralized with 1N aq HCl. The precipitate was collected and dried to give 350 mg of 2-(4-(tert-butoxycarbonyl)morpholin-2-yl)(6-fluoro-3′-methylbiphenyl-2-yl)methoxy)acetic acid as a white solid.

Step 2. tert-butyl 2-42-(ethylamino)-2-oxoethoxy)(6-fluoro-3′-methylbiphenyl-2-yl)methyl)morpholine-4-carboxylate: To a solution of 2-((4-(tert-butoxycarbonyl)morpholin-2-yl)(6-fluoro-3′-methylbiphenyl-2-yl)methoxy)acetic acid (250 mg, 0.545 mmol), HOBT (147 mg, 1.09 mmol) and BOP (481 mg, 1.09 mmol) in DMF (3 mL) were added i-Pr₂NEt (0.76 mL, 4.36 mmol) and ethylamine hydrochloride (266 mg, 3.27 mmol). The reaction mixture was stirred overnight at rt. LC-MS indicated complete conversion. EtOAc was added to the reaction and then washed with water and brine. The organic phase was dried over MgSO₄, filtered and concentrated to give 0.6 g of an oil. The crude residue was purified by flash chromatography on silica gel [ISCO Combiflash, 40 g column, Hexanes/EtOAc 0%→50%] and isolated 300 mg of tent-butyl 2-((2-(ethylamino)-2-oxoethoxy)(6-fluoro-3′-methylbiphenyl-2-yl)methyl)morpholine-4-carboxylate as a white foam.

### Preparation 21

### 4-((tert-butoxycarbonylamino)methyl)-2-fluorobenzoic acid

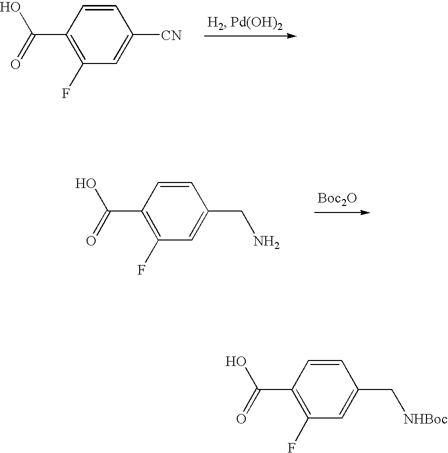

### Step 1. 4-(aminomethyl)-2-fluorobenzoic acid

A solution of 4-cyano-2-fluorobenzoic acid (1.0 g, 6.06 mmol) in 20 mL of MeOH at 25° C. was treated with of 20% Pd(OH)₂/C (300 mg, wet) and stirred overnight under an atmosphere of hydrogen. The reaction mixture was filtered and concentrated under reduced pressure to provide 4-(aminomethyl)-2-fluorobenzoic acid (1.0 g, quantitative).

### Step 2. 4-((tert-butoxycarbonylamino)methyl)-2-fluorobenzoic acid

A solution of 4-(aminomethyl)-2-fluorobenzoic acid (1.0 g, 6.0 mmol) in 50 mL of THF at 25° C. was treated with 50 mL of 1N aq NaOH and Boc₂O (1.5 g, 6.9 mmol) and the mixture was stirred overnight before being diluted with the addition of 25 mL of water and 10 mL of brine, acidified slowly to pH 3 using 1N aq HCl, and extracted with EtOAc (3×20 ml). The combined organic extracts were dried (Na₂SO₄) and concentrated under reduced pressure to provide 4-((tert-butoxycarbonylamino)methyl)-2-fluorobenzoic acid.

The following benzoic acids were prepared following procedures analogous to those described above by using the indicated starting material and catalyst in Step 1:

| Structure | Name | Starting Material | Catalyst |
|---|---|---|---|
| 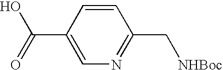 | 6-[({[(1,1-dimethylethyl)oxy] carbonyl}amino)methyl]-3- pyridinecarboxylic acid | 6-cyano-3- pyridinecarboxylic acid | Pd/C |
| 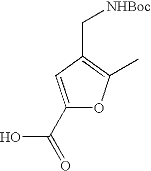 | 4-[({[(1,1-dimethylethyl)oxy] carbonyl}amino)methyl]-5- methyl-2-furancarboxylic acid | 4-(aminomethyl)-5- methyl-2-furancarboxylic acid | Omit Step 1 |

### Preparation 22

### methyl 1,2,3-oxathiazolidine-3-carboxylate 2,2-dioxide

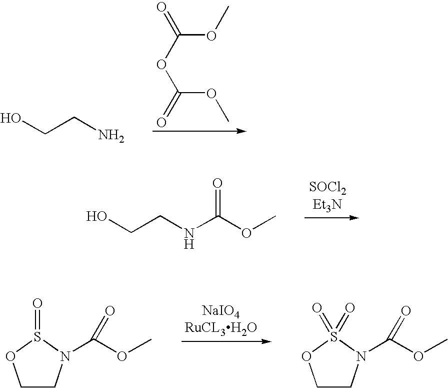

### Step 1. Methyl (2-hydroxyethyl)carbamate

To a stirred solution of 2-aminoethanol (6.11 g, 100 mmol) in dry dichloromethane (120 mL) at room temperature was added dropwise a solution of dimethyl dicarbonate (14.1 g, 105 mmol) in 20 mL. The resulting mixture was stirred for 5 hours before the solvent was removed in vacuo to afford methyl (2-hydroxyethyl)carbamate (12.1 g) as a colorless oil. ¹H NMR (400 MHz, CDCl₃): 2.21 (broad, 1H), 3.37 (q, 2H), 3.70-3.73 [s (3H)+q (2H)], 5.20 (broad, 1H).

### Step 2. Methyl 1,2,3-oxathiazolidine-3-carboxylate 2-oxide

To a stirred suspension of methyl (2-hydroxyethyl)carbamate (100 mmol, 12.1 g) in dry dichloromethane (700 mL) at −78° C. was added triethylamine (30.4 g, 42 ml, 300 mmol) followed by thionyl chloride (17.9 g, 11 mL, 150 mmol). The resulting yellow suspension was stirred at −78° C. for 3 hours before it was quenched with methanol (3.2 g, 4 ml, 100 mmol) and warmed to room temperature. The reaction mixture was filtered and the Filtrate concentrated to remove all the dichloromethane before being re-dissolved in 900 ml Et₂O, filtered and concentrated again. The resulting crude was purified by passing through a 15 cm silica plug eluted with 30% ethyl acetate in hexane to afford methyl 1,2,3-oxathiazolidine-3-carboxylate 2-oxide (7.905 g, 48%) as a yellowish oil. ¹H NMR (400 MHz, CDCl₃): 3.64 (m, 1H), 3.88 (s, 3H), 3.97 (m, 1H), 4.77 (m, 1H), 5.03 (m, 1H).

### Step 3. Methyl 1,2,3-oxathiazolidine-3-carboxylate 2,2-dioxide

To a stirred solution of methyl 1,2,3-oxathiazolidine-3-carboxylate 2-oxide (7.905 g, 47.9 mmol) in acetonitrile (45 mL) at 0° C. was added RuCl₃H₂O (54 mg, 0.24 mmol) followed by NaIO₄ (15.4 g, 71.8 mmol) and water (45 mL). The resulting mixture was allowed to warm to room temperature and stir for two hours before it was filtered. The filtrate was concentrated in vacuo and then redistributed in 800 mL MTBE and filtered again. The resulting solution was washed with water (50 mL), brine (2×100 mL), dried over Na₂SO₄, filtered and concentrated in vacuo to provide methyl 1,2,3-oxathiazolidine-3-carboxylate 2,2-dioxide (6.5 g, 75%) as an off-white solid. ¹H NMR (400 MHz, CDCl₃): 3.95 (s, 3H), 4.14 (t, 2H), 4.69 (t, 2H).

### Preparation 23

### methyl [2-({(S)-(6-chloro-3′-ethyl-2-biphenylyl)[(2R)-2-morpholinyl]methyl}oxy)ethyl]carbamate

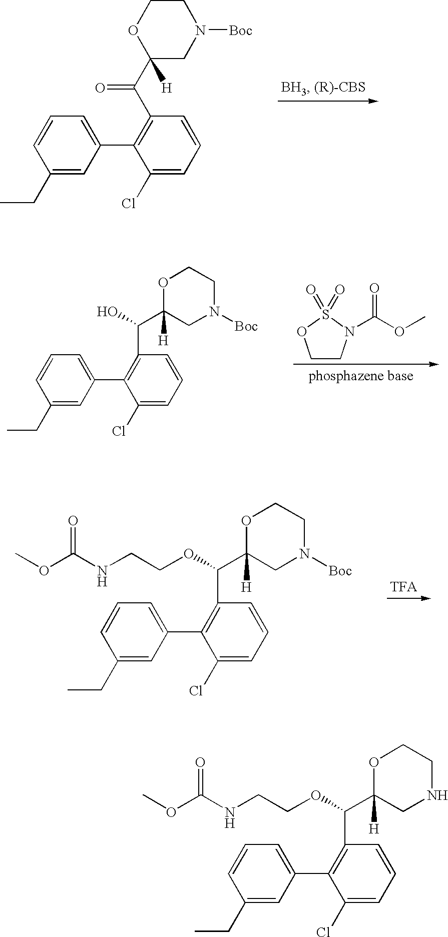

### Step 1. (1,1-dimethylethyl (2R)-2-[(S)-(6-chloro-3′-ethyl-2-biphenylyl)(hydroxy)methyl]-4-morpholinecarboxylate

To a stirred solution of 1,1-dimethylethyl (2R)-2-[(6-chloro-3′-ethyl-2-biphenylyl)carbonyl]-4-morpholinecarboxylate (2.87 g, 6.7 mmol) in TBME (75 mL) under argon at room temperature was added drop-wise borane-methyl sulfide complex (2M in toluene, 4.5 mL, 9 mmol) and (R)-2-methyl-CBS-oxazaborolidine (1 M in toluene, 0.7 mL, 0.7 mmol). The resulting solution was heated at 40° C. for 4 hours at which time TLC analysis showed complete consumption of the ketone. The reaction was quenched with 2 mL of water added slowly and then partitioned between 700 mL Et₂O and 150 mL brine. The organic layer was washed with brine (1×50 mL), dried over Na₂SO₄, filtered, and concentrated under reduced pressure. The crude was purified by flash chromatography (ISCO, 120 g column, EtOAc Hexane, 0-25%) to provide (1,1-dimethylethyl (2R)-2-[(S)-(6-chloro-3′-ethyl-2-biphenylyl)(hydroxy)methyl]-4-morpholinecarboxylate (1.392 g, 48%, the less polar diastereomer).

### Step 2. 1,1-dimethylethyl (2R)-2-{(S)-(6-chloro-3′-ethyl-2-biphenylyl)[(2-{[(methyloxy) carbonyl]amino}ethyl)oxy]methyl}-4-morpholinecarboxylate

To a stirred solution of (1,1-dimethylethyl (2R)-2-[(S)-(6-chloro-3′-ethyl-2-biphenylyl)(hydroxy)methyl]-4-morpholinecarboxylate (0.432 g, 1 mmol) in 6 mL of dry DMF at room temperature was added phosphazene base P₄-t-Bu (1.0M in n-hexane, 2 ml, 2 mmol). The resulting mixture was stirred for 10 minutes under argon before a solution of methyl 1,2,3-oxathiazolidine-3-carboxylate 2,2-dioxide (0.362 g, 2 mmol) in 2 mL dry DMF was added. The resulting solution was stirred at room temperature overnight (18 hours). The crude reaction mixture was purified via HPLC (Gilson, C₁₈ column, 5 um, 50×100 mm), CH₃CN/water (w/0.1% TFA) 65-95%) to provide 1,1-dimethylethyl (2R)-2-{(S)-(6-chloro-3′-ethyl-2-biphenylyl)[(2-{[(methyloxy) carbonyl]amino}ethyl)oxy]methyl}-4-morpholinecarboxylate as colorless oil. MS (E/Z): 533.4 (M+H⁺).

### Step 3. Methyl [2-({(S)-(6-chloro-3′-ethyl-2-biphenylyl)[(2R)-2-morpholinyl]methyl}oxy)ethyl]carbamate

To a stirred solution of 1,1-dimethylethyl (2R)-2-{(S)-(6-chloro-3′-ethyl-2-biphenylyl)[(2-{[(methyloxy) carbonyl]amino}ethyl)oxy]methyl}-4-morpholinecarboxylate in DCM (4 mL) at room temperature was added TFA (4 mL). The resulting mixture was stirred at room temperature for 1.5 h. The crude was concentrated under reduced pressure and then partitioned between 450 mL DCM and 50 mL saturated Na₂CO₃ solution. The organic layer was washed with brine, dried over Na₂SO₄, filtered and concentrated to provide methyl [2-{(S)-(6-chloro-3′-ethyl-2-biphenylyl)[(2R)-2-morpholinyl]methyl}oxy)ethyl]carbamate (407 mg, 68% for steps 2 and 3). MS (E/Z): 433.0 (M+H⁺).

### Preparation 24

### Methyl [2-({(R)-(6-chloro-3′-ethyl-2-biphenylyl)[(3R)-3-piperidinyl]methyl}oxy)ethyl]carbamate

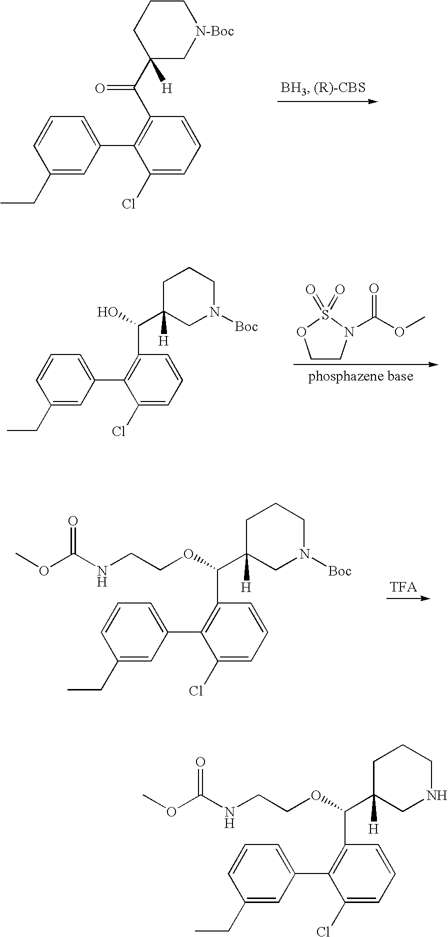

### Step 1. 1,1-dimethylethyl (3R)-3-[(R)-(6-chloro-3′-ethyl-2-biphenylyl)(hydroxy)methyl]-1-piperidinecarboxylate

To a stirred solution of 1,1-dimethylethyl (3R)-3-[(6-chloro-3′-ethyl-2-biphenylyl) carbonyl]-1-piperidinecarboxylate (1.6 g, 3.74 mmol) in TBME (60 mL) under argon at room temperature was added drop-wise simultaneously borane-methyl sulfide complex (2M in toluene, 2.5 ml, 5 mmol) and (R)-2-methyl-CBS-oxazaborolidine (1 M in toluene, 0.4 ml, 0.4 mmol). The resulting solution was heated at 40° C. for 3 h at which time TLC showed that the reaction was complete. The reaction was quenched with 1 mL water added slowly and then partitioned between 600 mL Et₂O and 50 mL water. The organic layer was washed with brine (1×50 mL), dried over Na₂SO₄, filtered, and concentrated under reduced pressure. The crude was purified by flash chromatography (ISCO, 120 g column, 0-30% ethyl acetate/hexane) to provide 1,1-dimethylethyl (3R)-3-[(R)-(6-chloro-3′-ethyl-2-biphenylyl)(hydroxy)methyl]-1-piperidinecarboxylate (1.2 g, 74.6%, less polar diastereomer). MS (E/Z): 430.4 (M+H⁺)

### Step 2. 1,1-dimethylethyl (3R)-3-{(R)-(6-chloro-3′-ethyl-2-biphenylyl)[(2-{[(methyloxy) carbonyl]amino}ethyl)oxy]methyl}-1-piperidinecarboxylate

To a stirred solution of 1,1-dimethylethyl (3R)-3-[(R)-(6-chloro-3′-ethyl-2-biphenylyl)(hydroxy)methyl]-1-piperidinecarboxylate (0.354 g, 0.82 mmol) in 8 mL of dry DMF at room temperature was added phosphazene base P₄-t-Bu (1.0M in n-hexane, 1.64 mL, 1.64 mmol). The resulting mixture was stirred for 10 minutes under argon before a solution of methyl 1,2,3-oxathiazolidine-3-carboxylate 2,2-dioxide (0.298 g, 1.64 mmol) in 2 ml dry DMF was added. The resulting solution was stirred at room temperature overnight (18 hours) before it was quenched with 10 mL saturated NH₄Cl solution. The product was extracted with 300 mL EtOAc and the organic layer was washed with saturated NH₄Cl solution (3×50 mL), HCl (2M, 3×50 mL), brine, and dried over Na₂SO₄, filtered and concentrated in vacuo to provide 1,1-dimethylethyl (3R)-3-{(R)-(6-chloro-3′-ethyl-2-biphenylyl)[(2-{[(methyloxy) carbonyl]amino}ethyl)oxy]methyl}-1-piperidinecarboxylate (452 mg), which was directly used in the next step without further purification. MS (E/Z): 531.4 (M+H⁺)

### Step 3. Methyl [2-({(R)-(6-chloro-3′-ethyl-2-biphenylyl)[(3R)-3-piperidinyl]methyl}oxy)ethyl]carbamate

To a solution of 1,1-dimethylethyl (3R)-3-{(R)-(6-chloro-3′-ethyl-2-biphenylyl)[(2-{[(methyloxy) carbonyl]amino}ethyl)oxy]methyl}-1-piperidinecarboxylate (0.452 g, 0.82 mmol) in DCM (14 mL) at room temperature was added TFA (4 mL). The resulting solution was stirred for 1.5 h. At this time the solvent was removed under reduced pressure and the crude material partitioned between 200 mL DCM and 50 mL 5% Na₂CO₃ solution. The organic layer was washed with water (50 mL), brine, dried over Na₂SO₄, filtered, and concentrated under reduced pressure to provide methyl [2-({(R)-(6-chloro-3′-ethyl-2-biphenylyl)[(3R)-3-piperidinyl]methyl}oxy)ethyl]carbamate (0.35 g, 99%). MS (E/Z): 431.5 (M+H⁺).

### Preparation 25

5-{[{[(1,1-dimethylethyl)oxy]carbonyl}(methyl)amino]methyl}-2-thiophenecarboxylic acid

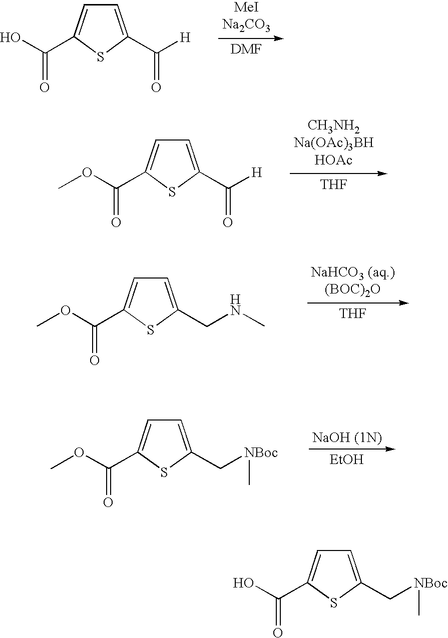

### Step 1. Methyl 5-formyl-2-thiophenecarboxylate

A mixture of 5-formyl-2-thiophene carboxylic acid (2.34 g, 15 mmol) and sodium carbonate (5.57 g, 52.5 mmol) in DMF (25 ml) at 25° C. was treated with iodomethane (1.15 ml, 18 mmol) and the mixture was stirred for 20 h before being quenched with the addition of water and saturated aqueous NH₄Cl. The resulting solid precipitate was collected by filtration to give methyl 5-formyl-2-thiophenecarboxylate as a solid. The filtrate was extracted with EtOAc (3×30 ml) and the combined organic extracts were washed with water and brine, dried (Na₂SO₄), concentrated under reduced pressure, and subjected to flash chromatography to give solid material that was combined with the solid collected by precipitation to give methyl 5-formyl-2-thiophenecarboxylate as a white solid (1.60 g, 62%): ESI-MS (m/z): 171.2 (M+H⁺).

### Step 2. Methyl 5-[(methylamino)methyl]-2-thiophenecarboxylate

A solution of methyl 5-formyl-2-thiophenecarboxylate (1.58 g, 9.3 mmol), methyl amine (5.6 ml, 2N, 11.2 mmol) and glacial acetic acid (3.0 ml) in THF (30 ml) at 25° C. was treated with sodium triacetoxyborohydride (11.83 g, 55.8 mmol) and the mixture was stirred for 18 h. Aqueous 2N NaOH was added until the solution reached pH 5. The resulting mixture was extracted with Et₂O (3×50 ml), and the combined organic extracts were washed with brine, dried (Na₂SO₄), concentrated under reduced pressure, and subjected to flash chromatography to give methyl 5-[(methylamino)methyl]-2-thiophenecarboxylate as a white solid (650 mg, 41%): ESI-MS (m/z): 186.0 (M+H⁺).

### Step 3. Methyl 5-{[{[(1,1-dimethylethyl)oxy]carbonyl}(methyl)amino]methyl}-2-thiophenecarboxylate

A solution of methyl 5-[(methylamino)methyl]-2-thiophenecarboxylate (640 mg, 3.5 mmol) in THF (10 ml) at 25° C. was treated with saturated aqueous NaHCO₃ (15 ml) and (Boc)₂O (802 mg, 3.7 mmol) and the mixture was stirred vigorously for 16 h before being diluted with EtOAc. The organic phase was separated, washed with water and brine, dried (Na₂SO₄), and concentrated under reduced pressure to give methyl 5-{[{[(1,1-dimethylethyl)oxy]carbonyl}(methyl)amino]methyl}-2-thiophenecarboxylate as a clear oil (957 mg, 95%): ESI-MS (m/z): 286.3 (M+H⁺).

### Step 4. 5-{[{[(1,1-dimethylethyl)oxy]carbonyl} (methyl)amino]methyl}-2-thiophenecarboxylic acid

A solution of methyl 5-{[{[(1,1-dimethylethyl)oxy]carbonyl}(methyl)amino]methyl}-2-thiophenecarboxylate (939 mg, 3.3 mmol) in EtOH (10 ml) at 25° C. was treated with aqueous NaOH (6.6 ml of 1N, 6.6 mmol) and the mixture was stirred for 18 h before being concentrated under reduced pressure. The residue was dissolved in water (10 ml), washed with EtOAc (2×5 ml), and the aqueous layer was acidified by addition of 1N aqueous HCl and then extracted with EtOAc (3×10 ml). The combined organic extracts were washed with brine, dried (MgSO₄), and concentrated under reduced pressure to give 5-{[{[(1,1-dimethylethyl)oxy]carbonyl} (methyl)amino]methyl}-2-thiophenecarboxylic acid as an amber oil (626 mg, 70%). ESI-MS (m/z): 272.4 (M+H⁺).

### Preparation 26

### 1,1-dimethylethyl [(4-phenyl-4-piperidinyl)methyl]carbamate

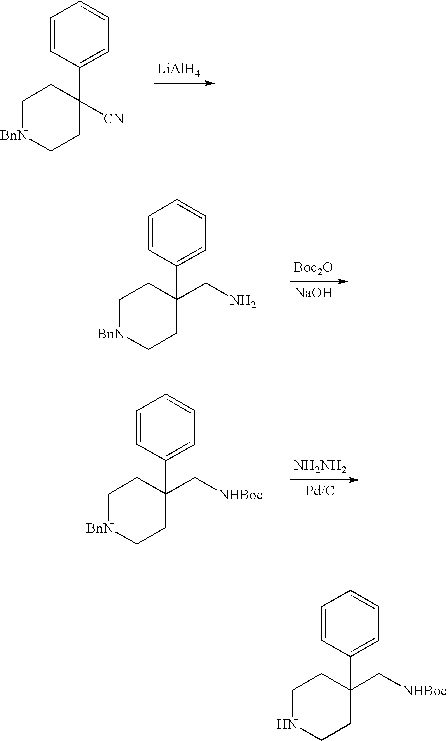

Step 1. 1[4-phenyl-1-(phenylmethyl)-4-piperidinyl]methanamine: A solution of 4-phenyl-1-(phenylmethyl)-4-piperidinecarbonitrile (4 g, 12.8 mmol) in dry Et₂O (60 ml) at 0° C. was treated with LiAlH₄ (0.585 g, 15.3 mmol) and the suspension was stirred at 25° C. overnight before being filtered through celite and concentrated under reduced pressure. The residue was treated with a cold saturated NaHCO₃ solution (gas and heat evolved) and then extracted with CH₂Cl₂ (2×40 mL). The combined organic extracts were dried over MgSO₄ and concentrated under reduced pressure to give 1-[4-phenyl-1-(phenylmethyl)-4-piperidinyl]methanamine as a colorless oil (3.4 g, 92%): ESI-MS (m/z): 281.2 (M+H⁺).

Step 2. 1,1-dimethylethyl {[4-phenyl-1-(phenylmethyl)-4-piperidinyl]methyl}carbamate: A solution of 1-[4-phenyl-1-(phenylmethyl)-4-piperidinyl]methanamine (3.4 g, 12.13 mmol) in 28 mL of 2N NaOH (aq) and 12 mL of THF was treated with BOC₂O, and the mixture was stirred at 25° C. overnight before being partitioned between 40 mL of water and 60 mL of dichloromethane. The organic layer was dried over MgSO₄ and concentrated under reduced pressure to give 1,1-dimethylethyl {[4-phenyl-1-(phenylmethyl)-4-piperidinyl]methyl}carbamate as an oil (4.32 g, 91%): LCMS (m/z): 381.2 (M+W).

Step 3. 1,1-dimethylethyl [(4-phenyl-4-piperidinyl)methyl]carbamate: A solution of 1,1-dimethylethyl {[4-phenyl-1-(phenylmethyl)-4-piperidinyl]methyl}carbamate (4.3 g, 11.3 mmol) in EtOH (60 mL) was treated with 10% Pd/C (1.0 g) and hydrazine hydrate (0.660 ml, 22.6 mmol), and the mixture was heated at 78° C. for 5 h before being cooled, filtered through celite, and concentrated under reduced pressure to give 1,1-dimethylethyl [(4-phenyl-4-piperidinyl)methyl]carbamate as a colorless oil (3.6 g, 99%): ESI-MS (m/z): 291.2 (M+H⁺).

The following procedures describe preparation of compounds of this invention.

### Example 1

### 1-((3R)-1-{[(1R,3S)-3-aminocyclopentyl]carbonyl}-3-piperidinyl)-5-(methyloxy)-1-[2-(phenyloxy)phenyl]-1-pentanol

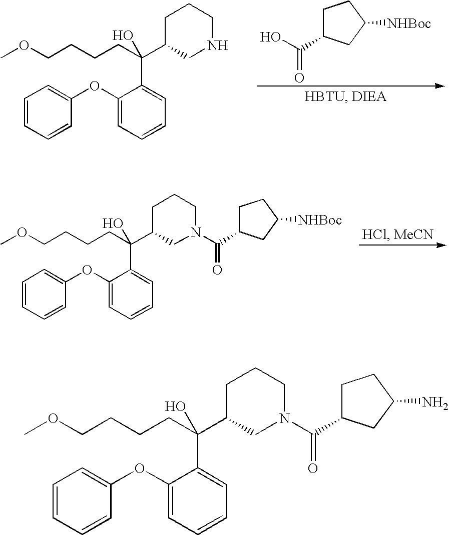

Step 1. ((1R,3S)-3-(tert-butoxycarbonylamino)cyclopentyl)((3R)-3-(1-hydroxy-5-methoxy-1-(2-phenoxyphenyl)pentyl)piperidin-1-yl)methanone: To a solution of 5-methoxy-1-(2-phenoxyphenyl)-1-((R)-piperidin-3-yl)pentan-1-ol (18.5 mg, 0.05 mmol) and (1R,3S)-3-(t-butoxycarbonylamino)cyclopentanecarboxylic acid (12.1 mg, 0.05 mmol) in DMF (0.5 mL) were added DIEA (26 μL. 0.15 mmol), HBTU (19.0 mg, 0.05 mmol), and HOBt (6.8 mg, 0.05 mmol). The resulting solution was stirred at rt for 20 min. Preparative HPLC gave ((1R,3S)-3-(tert-butoxycarbonylamino)cyclopentyl)((3R)-3-(1-hydroxy-5-methoxy-1-(2-phenoxyphenyl)pentyl)piperidin-1-yl)methanone (19.5 mg, 67%) as a oil. LC-MS (3 min) m/z 581 (M+H⁺).

Step 2. ((1R,3S)-3-Aminocyclopentyl)((3R)-3-(1-hydroxy-5-methoxy-1-(2-phenoxy phenyl)pentyl)piperidin-1-yl)methanone: To a stirred solution of ((1R,3S)-3-(tert-butoxycarbonylamino)cyclopentyl)((3R)-3-(1-hydroxy-5-methoxy-1-(2-phenoxyphenyl)pentyl)piperidin-1-yl)methanone (19.5 mg) in MeCN (2 mL) was added 5% aq HCl (2 mL). The resulting solution was stirred at rt until no starting material remained (˜16 h), basified to pH=10 with 10 N aq NaOH, and evaporated under reduced pressure to remove MeCN. The aq layer was extracted with CH₂Cl₂ (4×10 mL). The combined organic layers were washed with brine and dried over Na₂SO₄. The crude product was purified by preparative HPLC to give ((1R,3S)-3-Aminocyclopentyl)((3R)-3-(1-hydroxy-5-methoxy-1-(2-phenoxy phenyl)pentyl)piperidin-1-yl)methanone (I-4A, 17.4 mg) as its TFA salt. ¹H NMR (400 MHz, CD₃OD): 7.64 (m, 1H), 7.38 (m, 2H), 7.08-7.24 (m, 3H), 6.92 (m, 2H), 6.80 (two d, 1H), 4.44, 4.86 (m, 1H), 3.96, 4.26 (m, 1H), 3.68 (m, 1H), 3.36, 3.44 (m, 1H), 3.28 (t, 2H), 3.24 (s, 3H), 2.94, 3.14 (m, 1H), 2.63 (m, 1H), 2.40 (m, 1H), 1.8-2.2 (m, 6H), 1.0-1.8 (m, 8H), 0.92 (m, 1H); LC-MS (3 min) m/z 481 (M+H).

### Example 2

The compounds below were prepared by coupling the appropriate piperidines and Boc protected amino acids followed by deprotection according to the procedures described in Example 1.
#1 methyl 4-((R)-1-((1R,3S)-3-aminocyclopentanecarbonyl)piperidin-3-yl)-4-hydroxy-4-(2-(pyridin-4-yl)phenyl)butylcarbamate
#2 2-((R)-((R)-4-((1R,3S)-3-aminocyclopentanecarbonyl)morpholin-2-yl)(6-fluoro-3′-methylbiphenyl-2-yl)methoxy)-N-ethylacetamide
#3 N-(4-((R)-1-((1R,3S)-3-aminocyclopentanecarbonyl)piperidin-3-yl)-4-hydroxy-4-(2-(o-tolyloxy)phenyl)butyl)acetamide
#4 ((1R,3S)-3-aminocyclopentyl)((3R)-3-(1-(2-(2,6-dimethylphenoxy)phenyl)-1-hydroxy-5-methoxypentyl)piperidin-1-yl)methanone
#7 methyl 2-((4-((1R,3S)-3-aminocyclopentanecarbonyl)morpholin-2-yl)(6-fluoro-3′-methylbiphenyl-2-yl)methoxy)ethylcarbamate
#9 ((1R,3S)-3-aminocyclopentyl)((R)-2-((R)-1-(4′,6-difluoro-3′-methylbiphenyl-2-yl)-1-hydroxy-5-methoxypentyl)morpholino)methanone
#12 methyl 4-((R)-1-((1R,3S)-3-aminocyclopentanecarbonyl)piperidin-3-yl)-4-hydroxy-4-(2-(o-tolyloxy)phenyl)butylcarbamate
#15 ((1R,3S)-3-aminocyclopentyl)((3R)-3-(1-(6-fluoro-3′-methoxy-5′-methylbiphenyl-2-yl)-1-hydroxy-5-methoxypentyl)piperidin-1-yl)methanone
#16 ((1R,3S)-3-aminocyclopentyl)((3R)-3-(1-(6-chloro-3′-ethylbiphenyl-2-yl)-1-hydroxy-5-methoxypentyl)piperidin-1-yl)methanone
#17 ((1R,3S)-3-aminocyclopentyl)((3R)-3-(1-(3-chloro-2-(2-methylbenzyl)phenyl)-1-hydroxy-5-methoxypentyl)piperidin-1-yl)methanone
#18 ((1R,3S)-3-aminocyclopentyl)((3R)-3-(1-(3-chloro-2-(3-methylbenzyl)phenyl)-1-hydroxy-5-methoxypentyl)piperidin-1-yl)methanone
#19 ((1R,3S)-3-aminocyclopentyl)(2-(1-(6-fluoro-3′-methoxy-5′-methylbiphenyl-2-yl)-1-hydroxy-5-methoxypentyl)morpholino)methanone
#20 ((1R,3S)-3-aminocyclopentyl)((2R)-2-(1-(6-chloro-3′-ethylbiphenyl-2-yl)-1-hydroxy-5-methoxypentyl)morpholino)methanone
#21 ((1R,3S)-3-aminocyclopentyl)((3R)-3-(1-(3-chloro-2-(o-tolyloxy)phenyl)-1-hydroxy-5-methoxypentyl)piperidin-1-yl)methanone
#22 ((1R,3S)-3-aminocyclopentyl)((3R)-3-(1-(2-(2-chloro-6-methylphenoxy)phenyl)-1-hydroxy-5-methoxypentyl)piperidin-1-yl)methanone
#26 methyl 4-((R)-1-((1R,3S)-3-aminocyclopentanecarbonyl)piperidin-3-yl)-4-(3′-ethyl-6-fluorobiphenyl-2-yl)-4-hydroxybutylcarbamate
#31 ((1R,3S)-3-aminocyclopentyl)((3R)-3-(1-(3-chloro-2-(2-ethylphenoxy)phenyl)-1-hydroxy-5-methoxypentyl)piperidin-1-yl)methanone
#32 ((1R,3S)-3-aminocyclopentyl)((3R)-3-(1-(3-chloro-2-(3-ethylphenoxy)phenyl)-1-hydroxy-5-methoxypentyl)piperidin-1-yl)methanone
#39 ((1R,3S)-3-aminocyclopentyl)((R)-3-((S)-1-(3-chloro-2-(quinolin-3-yl)phenyl)-1-hydroxy-5-methoxypentyl)piperidin-1-yl)methanone
#40 ((1R,3S)-3-aminocyclopentyl)((R)-2-((R)-1-(3-chloro-2-(naphthalen-2-yl)phenyl)-1-hydroxy-5-methoxypentyl)morpholino)methanone
#41 ((1R,3S)-3-aminocyclopentyl)(2-(1-(3-chloro-2-(quinolin-3-yl)phenyl)-1-hydroxy-5-methoxypentyl)morpholino)methanone
#45 methyl 4-((R)-1-((1R,3S)-3-aminocyclopentanecarbonyl)piperidin-3-yl)-4-(6-chloro-3′-ethylbiphenyl-2-yl)-4-hydroxybutylcarbamate
#46 methyl 4-((R)-4-((1R,3S)-3-aminocyclopentanecarbonyl)morpholin-2-yl)-4-(6-chloro-3′-ethylbiphenyl-2-yl)-4-hydroxybutylcarbamate
#56 methyl 4-((R)-1-((1R,3S)-3-aminocyclopentanecarbonyl)piperidin-3-yl)-4-(6-chloro-3′-isopropylbiphenyl-2-yl)-4-hydroxybutylcarbamate
#62 ((1R,3S)-3-aminocyclopentyl)((3R)-3-(1-(3′-ethoxy-6-fluoro-5′-(trifluoromethyl)biphenyl-2-yl)-1-hydroxy-5-methoxypentyl)piperidin-1-yl)methanone
#64 ((1R,3S)-3-aminocyclopentyl)((R)-2-((R)-1-(3′-ethoxy-6-fluoro-5′-(trifluoromethyl)biphenyl-2-yl)-1-hydroxy-5-methoxypentyl)morpholino)methanone
#67 ((1R,3S)-3-aminocyclopentyl)((R)-2-((R)-1-(6-fluoro-3′,5′-dimethoxybiphenyl-2-yl)-1-hydroxy-5-methoxypentyl)morpholino)methanone
#68 ((1R,3S)-3-aminocyclopentyl)((2R)-2-((1R)-1-(3-chloro-2-(isoquinolin-4-yl)phenyl)-1-hydroxy-5-methoxypentyl)morpholino)methanone
#69 methyl (4-((3R)-1-{[(1R,3S)-3-aminocyclopentyl]carbonyl}-3-piperidinyl)-4-{3-chloro-2-[(2-methylphenyl)oxy]phenyl}-4-hydroxybutyl)carbamate
#70 methyl (4-((3R)-1-{[(1R,3S)-3-aminocyclopentyl]carbonyl}-3-piperidinyl)-4-{3-chloro-2-[(2-ethylphenyl)oxy]phenyl}-4-hydroxybutyl)carbamate
#100 (1R)-1-((2R)-4-{[(1R,3S)-3-aminocyclopentyl]carbonyl}-2-morpholinyl)-1-[2-chloro-3-(3-ethylphenyl)-4-pyridinyl]-5-(methyloxy)-1-pentanol
#102 (1R)-1-((2R)-4-{[(1R,3S)-3-aminocyclopentyl]carbonyl}-2-morpholinyl)-1-[2-(1-benzothien-3-yl)-3-chlorophenyl]-5-(methyloxy)-1-pentanol
#108 1-((3R)-1-{[(1R,3S)-3-aminocyclopentyl]carbonyl}-3-piperidinyl)-1-{3-chloro-2-[(3-methylphenyl)oxy]phenyl}-5-(methyloxy)-1-pentanol
#110 methyl (4-((3R)-1-{[(1R,3S)-3-aminocyclopentyl]carbonyl}-3-piperidinyl)-4-{3-chloro-2-[(3-methylphenyl)oxy]phenyl}-4-hydroxybutyl)carbamate
#112 methyl (4-((3R)-1-{[(1R,3S)-3-aminocyclopentyl]carbonyl}-3-piperidinyl)-4-{2-[(2,6-dimethylphenyl)oxy]phenyl}-4-hydroxybutyl)carbamate
#134 1-((3R)-1-{[(1R,3S)-3-aminocyclopentyl]carbonyl}-3-piperidinyl)-1-{3-fluoro-2-[(3-methylphenyl)oxy]phenyl}-5-(methyloxy)-1-pentanol
#136 methyl (4-((3R)-1-{[(1R,3S)-3-aminocyclopentyl]carbonyl}-3-piperidinyl)-4-{3-chloro-2-[(3-ethylphenyl)oxy]phenyl}-4-hydroxybutyl)carbamate
#138 methyl (4-((3R)-1-{[(1R,3S)-3-aminocyclopentyl]carbonyl}-3-piperidinyl)-4-{3-fluoro-2-[(3-methylphenyl)oxy]phenyl}-4-hydroxybutyl)carbamate
#141 methyl (4-((3R)-1-{[(1R,3S)-3-aminocyclopentyl]carbonyl}-3-piperidinyl)-4-{3-chloro-2-[(3-methylphenyl)methyl]phenyl}-4-hydroxybutyl)carbamate

### Example 3

### N—((S)-4-(6-chloro-3′-ethylbiphenyl-2-yl)-4-hydroxy-4-((R)-1-(4-((methylamino)methyl)benzoyl)piperidin-3-yl)butyl)acetamide

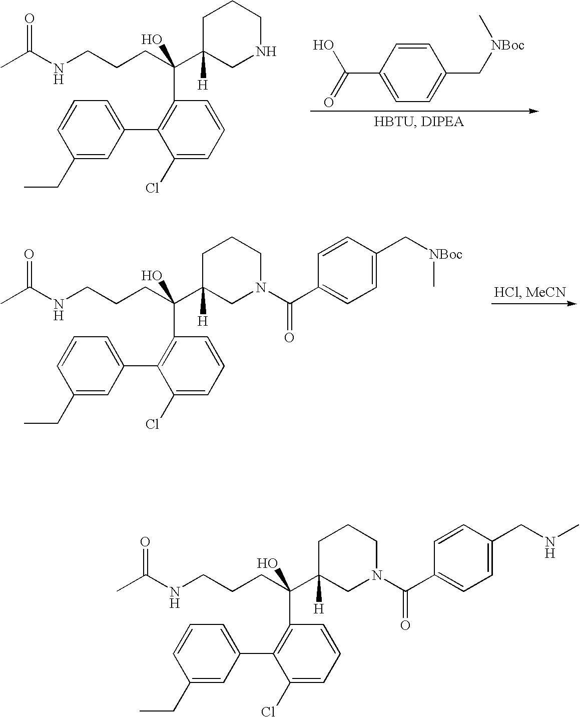

Step 1. tert-butyl 4-((R)-3-((S)-4-acetamido-1-(6-chloro-3′-ethylbiphenyl-2-yl)-1-hydroxybutyl)piperidine-1-carbonyl)benzyl(methyl)carbamate: A solution of N—((S)-4-(6-chloro-3′-ethylbiphenyl-2-yl)-4-hydroxy-4-((R)-piperidin-3-yl)butyl)acetamide (48 mg, 0.10 mmol) in 1 mL of DMF at 25° C. was treated with 4-((tert-butoxycarbonyl(methyl)amino)methyl)benzoic acid (33 mg, 0.12 mmol), i-Pr₂NEt (0.089 mL, 0.5 mmol), and HBTU (47 mg, 0.12 mmol). After 24 h, H₂O was added and the mixture was extracted with EtOAc. The organic extracts were washed (1N aq HCl, 1N aq NaOH, H₂O, brine), dried (Na₂SO₄), concentrated under reduced pressure, and subjected to flash chromatography to provide tent-butyl 4-((R)-3-((S)-4-acetamido-1-(6-chloro-3′-ethylbiphenyl-2-yl)-1-hydroxybutyl)piperidine-1-carbonyl)benzyl(methyl)carbamate as a colorless oil (50 mg, 71%). MS (m/z) 676.3 (M+H⁺).

Step 2. N—((S)-4-(6-chloro-3′-ethylbiphenyl-2-yl)-4-hydroxy-4-4R)-1-(4-((methylamino)methyl)benzoyl)piperidin-3-yl)butyl)acetamide: A solution of tert-butyl 4-((R)-3-((S)-4-acetamido-1-(6-chloro-3′-ethylbiphenyl-2-yl)-1-hydroxybutyl)piperidine-1-carbonyl)benzyl(methyl)carbamate (50 mg, 0.074 mmol) in 3 mL of CH₃CN at 25° C. was treated with 3 mL of aqueous 2N HCl. After 24 h, the mixture was concentrated under reduced pressure to provide N-[(4S)-4-(6-chloro-3′-ethyl-2-biphenylyl)-4-hydroxy-4-[(3R)-1-([4-[(methylamino)methyl]phenyl]carbonyl)-3-piperidinyl]butyl]acetamide as a white solid (39 mg, quantitative). MS (m/z) 576.2 (M+H⁺).

### Example 4

The following compounds were prepared following procedures analogous to those described in Example 3.
#28 (6-(aminomethyl)pyridine-3-yl)((3R)-3-(1-(6-chloro-3′-ethylbiphenyl-2-yl)-1-hydroxy-5-methoxypentyl)piperidin-1-yl)methanone
#42 methyl (4-hydroxy-4-((3R)-1-{[(1R,3S)-3-(methylamino)cyclopentyl]carbonyl}-3-piperidinyl)-4-{2-[(2-methylphenyl)oxy]phenyl}butyl)carbamate
#43 methyl 4-(6-fluoro-3′-methoxybiphenyl-2-yl)-4-hydroxy-4-(R)-1-((1R,3S)-3-(methylamino)cyclopentanecarbonyl)piperidin-3-yl)butylcarbamate
#58 methyl 4-(6-chloro-3′-methoxybiphenyl-2-yl)-4-hydroxy-4-(R)-1-41R,3S)-3-(methylamino)cyclopentanecarbonyl)piperidin-3-yl)butylcarbamate
#76 (6-(aminomethyl)pyridine-3-yl)((R)-2-(R)-1-(6-chloro-3′-ethylbiphenyl-2-yl)-1-hydroxy-5-methoxypentyl)morpholino)methanone
#77 methyl 4-((R)-1-(6-(aminomethyl)nicotinoyl)piperidin-3-yl)-4-(6-chloro-3′-ethylbiphenyl-2-yl)-4-hydroxybutylcarbamate

### Example 5

### Methyl (S)-4-(6-chloro-3′-ethylbiphenyl-2-yl)-4-hydroxy-4-((R)-1-(4-((methylamino)methyl)benzoyl)piperidin-3-yl)butylcarbamate

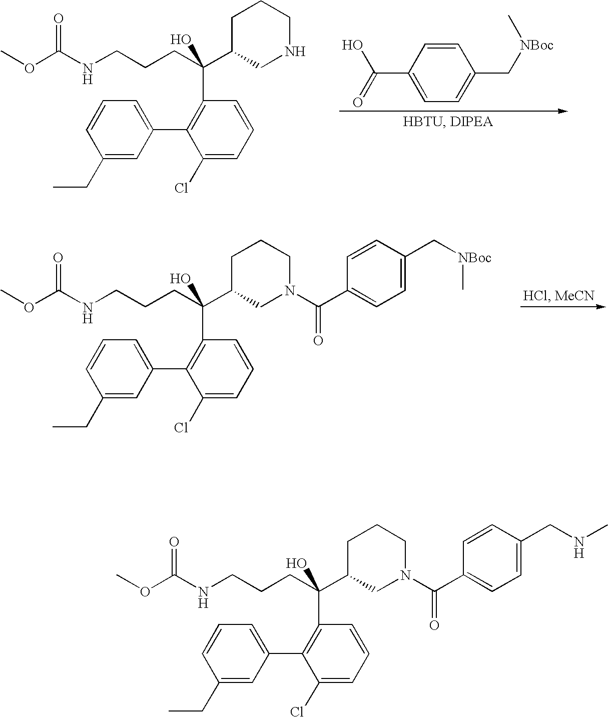

Step 1. methyl (S)-4-(6-chloro-3′-ethylbiphenyl-2-yl)-4-hydroxy-4-((R)-1-(4-(((N-t-butoxycarbonyl-N-methyl)amino)methyl)benzoyl)piperidin-3-yl)butylcarbamate: A solution of methyl (S)-4-(6-chloro-3′-ethylbiphenyl-2-yl)-4-hydroxy-4-((R)-piperidin-3-yl)butylcarbamate (30 mg, 0.07 mmol) in 1 mL of DMF at 25° C. was treated with 4-((tert-butoxycarbonyl(methyl)amino)methyl)benzoic acid (21 mg, 0.08 mmol), i-Pr₂NEt (0.063 mL, 0.37 mmol), and HBTU (30 mg, 0.08 mmol). After 1 h, H₂O was added and the mixture was extracted with EtOAc. The organic extracts were washed (1N HCl, 1N NaOH, H₂O, brine), dried (Na₂SO₄), concentrated under reduced pressure, and subjected to flash chromatography to provide methyl (S)-4-(6-chloro-3′-ethylbiphenyl-2-yl)-4-hydroxy-4-((R)-1-(4-(((N-t-butoxycarbonyl-N-methyl)amino)methyl)benzoyl)piperidin-3-yl)butylcarbamate as a colorless oil (24 mg, 51%). MS (m/z) 692.3 (M+H⁺).

Step 2. methyl {(4S)-4-(6-chloro-3′-ethyl-2-biphenylyl)-4-hydroxy-4-[(3R)-1-({4-[(methylamino)methyl]phenyl}carbonyl)-3-piperidinyl]butyl}carbamate: A solution of methyl (S)-4-(6-chloro-3′-ethylbiphenyl-2-yl)-4-hydroxy-4-((R)-1-(4-(((N-t-butoxycarbonyl-N-methyl)amino)methyl)benzoyl)piperidin-3-yl)butylcarbamate (24 mg, 0.034 mmol) in 3 mL of CH₃CN at 25° C. was treated with 3 mL of aqueous 2N HCl. After 24 h, the mixture was concentrated under reduced pressure to provide methyl {(4S)-4-(6-chloro-3′-ethyl-2-biphenylyl)-4-hydroxy-4-[(3R)-1-({4-[(methylamino)methyl]phenyl}carbonyl)-3-piperidinyl]butyl}carbamate as a white solid (17 mg, 81%). MS (m/z) 592.2 (M+H⁺).

### Example 6

The following piperidines were prepared following procedures analogous to those described in Example 5 using the appropriate amine intermediate and the indicated acid in place of 4-((tert-butoxycarbonyl(methyl)amino)methyl)benzoic acid in Step 1.

| Cpd. No. | Product | Acid used in Step 1 |
|---|---|---|
| #25 | methyl 4-((R)-1-((1R,2S)-2- | (1R,2S)-2-({[(1,1- |
| | aminocyclopentanecarbonyl)piperidin-3-yl)- | dimethylethyl)oxy]carbonyl}amino)cyclopentanecarboxylic |
| | 4-(3′-ethyl-6-fluorobiphenyl-2-yl)-4- | acid |
| | hydroxybutylcarbamate | |
| #27 | (trans-4-aminocyclohexyl)((3R)-3-(1- | trans-4-({[(1,1- |
| | (6-chloro-3′-ethylbiphenyl-2-yl)-1-hydroxy- | dimethylethyl)oxy]carbonyl}amino)cyclohexanecarboxylic |
| | 5-methoxypentyl)piperidin-1-yl)methanone | acid |
| #50 | (4-(aminomethyl)cyclohexyl)((2R)-2- | 4-[({[(1,1-dimethylethyl)oxy]carbonyl}amino)methyl]cyclohexanecarboxylic |
| | ((1R)-1-(6-chloro-2′-fluoro-5′- | acid |
| | methylbiphenyl-2-yl)-1-hydroxy-5- | |
| | methoxypentyl)morpholino)methanone | |
| #55 | methyl 4-(6-chloro-3′-ethylbiphenyl-2- | (1R,3S)-3-[{[(1,1-dimethylethyl)oxy]carbonyl}(methyl)amino]cyclopentanecarboxylic |
| | yl)-4-hydroxy-4-((R)-1-((1R,3S)-3- | acid |
| | (methylamino)cyclopentanecarbonyl)piperidin- | |
| | 3-yl)butylcarbamate | |
| #60 | methyl 4-((R)-1-(trans-4- | trans-4-[({[(1,1-dimethylethyl)oxy]carbonyl}amino)methyl]cyclohexanecarboxylic |
| | (aminomethyl)cyclohexanecarbonyl)piperidin- | acid |
| | 3-yl)-4-(6-chloro-3′-ethylbiphenyl-2-yl)- | |
| | 4-hydroxybutylcarbamate | |
| #74 | methyl {4-(6-chloro-3′-ethyl-2- | trans-4-{[{[(1,1-dimethylethyl)oxy]carbonyl}(methyl)amino]methyl}cyclohexanecarboxylic |
| | biphenylyl)-4-hydroxy-4-[(3R)-1- | acid |
| | ({trans-4- | |
| | [(methylamino)methyl]cyclohexyl}carbonyl)- | |
| | 3-piperidinyl]butyl}carbamate | |
| #75 | methyl [4-((3R)-1-{[(1R,2S)-2- | (1R,2S)-2-({[(1,1-dimethylethyl)oxy]carbonyl}amino)cyclopentanecarboxylic |
| | aminocyclopentyl]carbonyl}-3- | acid |
| | piperidinyl)-4-(3′-ethyl-6-fluoro-2- | |
| | biphenylyl)-4-hydroxybutyl]carbamate | |
| #78 | 1-{(3R)-1-[(cis-4- | [cis-4-({[(1,1- |
| | aminocyclohexyl)acetyl]-3-piperidinyl}- | dimethylethyl)oxy]carbonyl}amino)cyclohexyl]acetic |
| | 1-(6-chloro-3′-ethyl-2-biphenylyl)-5- | acid |
| | (methyloxy)-1-pentanol | |
| #79 | 1-{(3R)-1-[(trans-4- | [trans-4-({[(1,1- |
| | aminocyclohexyl)acetyl]-3-piperidinyl}- | dimethylethyl)oxy]carbonyl}amino)cyclohexyl]acetic |
| | 1-(6-chloro-3′-ethyl-2-biphenylyl)-5- | acid |
| | (methyloxy)-1-pentanol | |
| #80 | 1-((3R)-1-{[(1R,2S)-2- | (1R,2S)-2-(tert- |
| | aminocyclopentyl]carbonyl}-3- | butoxycarbonylamino)cyclopentanecarboxylic |
| | piperidinyl)-1-(6-chloro-3′-ethyl-2- | acid |
| | biphenylyl)-5-(methyloxy)-1-pentanol | |
| #81 | (1R)-1-(6-chloro-3′-ethyl-2- | (1R,3S)-3-(tert- |
| | biphenylyl)-1-((2R)-4-{[(1R,3S)-3- | butoxycarbonyl(methyl)amino)cyclopentanecarboxylic |
| | (methylamino)cyclopentyl]carbonyl}-2- | acid |
| | morpholinyl)-5-(methyloxy)-1-pentanol | |
| #82 | 1-(6-chloro-3′-ethyl-2-biphenylyl)- | (1R,3S)-3-(tert- |
| | 1-((3R)-1-{[(1R,3S)-3- | butoxycarbonyl(methyl)amino)cyclopentanecarboxylic |
| | (methylamino)cyclopentyl]carbonyl}-3- | acid |
| | piperidinyl)-5-(methyloxy)-1-pentanol | |
| #85 | 1-(6-chloro-3′-ethyl-2-biphenylyl)- | (1R,4R)-4-((tert- |
| | 1-[(3R)-1-({trans-4- | butoxycarbonyl(methyl)amino)methyl)cyclohexanecarboxylic |
| | [(methylamino)methyl]cyclohexyl}carbonyl)- | acid |
| | 3-piperidinyl]-5-(methyloxy)-1- | |
| | pentanol | |
| #87 | methyl [4-((3R)-1-{[(1R,3S)-3- | (1R,3S)-3-(tert- |
| | aminocyclohexyl]carbonyl}-3- | butoxycarbonylamino)cyclohexanecarboxylic |
| | piperidinyl)-4-(6-chloro-3′-ethyl-2- | acid |
| | biphenylyl)-4-hydroxybutyl]carbamate | |
| #88 | N-{4-(6-chloro-3′-methyl-2- | (1R,4R)-4-((tert- |
| | biphenylyl)-4-hydroxy-4-[(3R)-1- | butoxycarbonyl(methyl)amino)methyl)cyclohexanecarboxylic |
| | ({trans-4- | acid |
| | [(methylamino)methyl]cyclohexyl}carbonyl)- | |
| | 3-piperidinyl]butyl}acetamide | |
| #89 | N-[4-(6-chloro-3′-methyl-2- | (1R,3S)-3-(tert- |
| | biphenylyl)-4-hydroxy-4-((3R)-1- | butoxycarbonyl(methyl)amino)cyclopentanecarboxylic |
| | {[(1R,3S)-3- | acid |
| | (methylamino)cyclopentyl]carbonyl}-3- | |
| | piperidinyl)butyl]acetamide | |
| #91 | N-[4-((3R)-1-{[trans-4- | (1R,4R)-4-((tert- |
| | (aminomethyl)cyclohexyl]carbonyl}-3- | butoxycarbonylamino)methyl)cyclohexanecarboxylic |
| | piperidinyl)-4-(6-chloro-3′-methyl-2- | acid |
| | biphenylyl)-4-hydroxybutyl]acetamide | |
| #92 | methyl {4-(4,6-difluoro-3′-methyl- | (1R,4R)-4-((tert- |
| | 2-biphenylyl)-4-hydroxy-4-[(3R)-1- | butoxycarbonyl(methyl)amino)methyl)cyclohexanecarboxylic |
| | ({trans-4- | acid |
| | [(methylamino)methyl]cyclohexyl}carbonyl)- | |
| | 3-piperidinyl]butyl}carbamate | |
| #93 | methyl [4-hydroxy-4-((3R)-1- | (1R,3S)-3-(tert- |
| | {[(1R,3S)-3- | butoxycarbonyl(methyl)amino)cyclopentanecarboxylic |
| | (methylamino)cyclopentyl]carbonyl}-3- | acid |
| | piperidinyl)-4-(2′,4,6-trifluoro-5′- | |
| | methyl-2-biphenylyl)butyl]carbamate | |
| #94 | methyl [4-hydroxy-4-[(3R)-1- | (1R,4R)-4-((tert- |
| | ({trans-4- | butoxycarbonyl(methyl)amino)methyl)cyclohexanecarboxylic |
| | [(methylamino)methyl]cyclohexyl}carbonyl)- | acid |
| | 3-piperidinyl]-4-(2′,4,6-trifluoro- | |
| | 5′-methyl-2-biphenylyl)butyl]carbamate | |
| #95 | methyl [4-(6-chloro-3′-methyl-2- | (1R,3S)-3-(tert- |
| | biphenylyl)-4-hydroxy-4-((3R)-1- | butoxycarbonyl(methyl)amino)cyclopentanecarboxylic |
| | {[(1R,3S)-3- | acid |
| | (methylamino)cyclopentyl]carbonyl}-3- | |
| | piperidinyl)butyl]carbamate | |
| #96 | (1R)-1-(6-chloro-3′-ethyl-2- | (1R,4R)-4-((tert- |
| | biphenylyl)-1-[(2R)-4-({trans-4- | butoxycarbonyl(methyl)amino)methyl)cyclohexanecarboxylic |
| | [(methylamino)methyl]cyclohexyl}carbonyl)- | acid |
| | 2-morpholinyl]-5-(methyloxy)-1- | |
| | pentanol | |
| #98 | methyl {(4R)-4-(3′-ethyl-6-fluoro- | (1R,4R)-4-((tert- |
| | 2-biphenylyl)-4-hydroxy-4-[(2R)-4- | butoxycarbonyl(methyl)amino)methyl)cyclohexanecarboxylic |
| | ({trans-4- | acid |
| | [(methylamino)methyl]cyclohexyl}carbonyl)- | |
| | 2-morpholinyl]butyl}carbamate | |
| #103 | (1R)-1-[4-chloro-3-(3-ethylphenyl)- | (1R,3S)-3-(tert- |
| | 2-pyridinyl]-1-((2R)-4-{[(1R,3S)-3- | butoxycarbonyl(methyl)amino)cyclopentanecarboxylic |
| | (methylamino)cyclopentyl]carbonyl}-2- | acid |
| | morpholinyl)-5-(methyloxy)-1-pentanol | |
| #104 | methyl {4-[2-chloro-3-(3- | (1R,4R)-4-((tert- |
| | ethylphenyl)-4-pyridinyl]-4-hydroxy-4- | butoxycarbonyl(methyl)amino)methyl)cyclohexanecarboxylic |
| | [(3R)-1-({trans-4- | acid |
| | [(methylamino)methyl]cyclohexyl}carbonyl)- | |
| | 3-piperidinyl]butyl}carbamate | |
| #105 | methyl [4-[2-chloro-3-(3- | (1R,3S)-3-(tert- |
| | ethylphenyl)-4-pyridinyl]-4-hydroxy-4- | butoxycarbonyl(methyl)amino)cyclopentanecarboxylic |
| | ((3R)-1-{[(1R,3S)-3- | acid |
| | (methylamino)cyclopentyl]carbonyl}-3- | |
| | piperidinyl)butyl]carbamate | |
| #106 | methyl {4-{3-chloro-2-[4-(1- | (1R,4R)-4-((tert- |
| | methylethyl)-2-quinazolinyl]phenyl}-4- | butoxycarbonyl(methyl)amino)methyl)cyclohexanecarboxylic |
| | hydroxy-4-[(3R)-1-({trans-4- | acid |
| | [(methylamino)methyl]cyclohexyl}carbonyl)- | |
| | 3-piperidinyl]butyl}carbamate | |
| #107 | methyl [4-{3-chloro-2-[4-(1- | (1R,3S)-3-(tert- |
| | methylethyl)-2-quinazolinyl]phenyl}-4- | butoxycarbonyl(methyl)amino)cyclopentanecarboxylic |
| | hydroxy-4-((3R)-1-{[(1R,3S)-3- | acid |
| | (methylamino)cyclopentyl]carbonyl}-3- | |
| | piperidinyl)butyl]carbamate | |
| #114 | methyl {4-{3-chloro-2-[8-(1- | (1R,4R)-4-((tert- |
| | methylethyl)-2-quinolinyl]phenyl}-4- | butoxycarbonyl(methyl)amino)methyl)cyclohexanecarboxylic |
| | hydroxy-4-[(3R)-1-({trans-4- | acid |
| | [(methylamino)methyl]cyclohexyl}carbonyl)- | |
| | 3-piperidinyl]butyl}carbamate | |
| #115 | methyl {4-[3-chloro-2-(8-methyl-2- | (1R,4R)-4-((tert- |
| | quinolinyl)phenyl]-4-hydroxy-4-[(3R)- | butoxycarbonyl(methyl)amino)methyl)cyclohexanecarboxylic |
| | 1-({trans-4- | acid |
| | [(methylamino)methyl]cyclohexyl}carbonyl)- | |
| | 3-piperidinyl]butyl}carbamate | |
| #116 | methyl [4-[3-fluoro-2-(3- | (1R,3S)-3-(tert- |
| | quinolinyl)phenyl]-4-hydroxy-4-((3R)- | butoxycarbonyl(methyl)amino)cyclopentanecarboxylic |
| | 1-{[(1R,3S)-3- | acid |
| | (methylamino)cyclopentyl]carbonyl}-3- | |
| | piperidinyl)butyl]carbamate | |
| #117 | methyl [4-{2-chloro-3-[3-(1- | (1R,3S)-3-(tert- |
| | methylethyl)phenyl]-4-pyridinyl}-4- | butoxycarbonyl(methyl)amino)cyclopentanecarboxylic |
| | hydroxy-4-((3R)-1-{[(1R,3S)-3- | acid |
| | (methylamino)cyclopentyl]carbonyl}-3- | |
| | piperidinyl)butyl]carbamate | |
| #118 | methyl [4-[3-chloro-2-(5-methyl-2- | (1R,3S)-3-(tert- |
| | furanyl)phenyl]-4-hydroxy-4-((3R)-1- | butoxycarbonyl(methyl)amino)cyclopentanecarboxylic |
| | {[(1R,3S)-3- | acid |
| | (methylamino)cyclopentyl]carbonyl}-3- | |
| | piperidinyl)butyl]carbamate | |
| #120 | (1R)-1-{2-chloro-3-[3-(1- | (1R,4R)-4-((tert- |
| | methylethyl)phenyl]-4-pyridinyl}-1- | butoxycarbonyl(methyl)amino)methyl)cyclohexanecarboxylic |
| | [(2R)-4-({trans-4- | acid |
| | [(methylamino)methyl]cyclohexyl}carbonyl)- | |
| | 2-morpholinyl]-5-(methyloxy)-1- | |
| | pentanol | |
| #121 | (1R)-1-{2-chloro-3-[3-(1- | (1R,3S)-3-(tert- |
| | methylethyl)phenyl]-4-pyridinyl}-1- | butoxycarbonyl(methyl)amino)cyclopentanecarboxylic |
| | ((2R)-4-{[(1R,3S)-3- | acid |
| | (methylamino)cyclopentyl]carbonyl}-2- | |
| | morpholinyl)-5-(methyloxy)-1-pentanol | |
| #123 | methyl {4-[5-chloro-4-(3- | (1R,4R)-4-((tert- |
| | ethylphenyl)-3-pyridinyl]-4-hydroxy-4- | butoxycarbonyl(methyl)amino)methyl)cyclohexanecarboxylic |
| | [(3R)-1-({trans-4- | acid |
| | [(methylamino)methyl]cyclohexyl}carbonyl)- | |
| | 3-piperidinyl]butyl}carbamate | |
| #124 | methyl [4-[5-chloro-4-(3- | (1R,3S)-3-(tert- |
| | ethylphenyl)-3-pyridinyl]-4-hydroxy-4- | butoxycarbonyl(methyl)amino)cyclopentanecarboxylic |
| | ((3R)-1-{[(1R,3S)-3- | acid |
| | (methylamino)cyclopentyl]carbonyl}-3- | |
| | piperidinyl)butyl]carbamate | |
| #125 | methyl {4-{5-chloro-4-[3-(1- | (1R,4R)-4-((tert- |
| | methylethyl)phenyl]-3-pyridinyl}-4- | butoxycarbonyl(methyl)amino)methyl)cyclohexanecarboxylic |
| | hydroxy-4-[(3R)-1-({trans-4- | acid |
| | [(methylamino)methyl]cyclohexyl}carbonyl)- | |
| | 3-piperidinyl]butyl}carbamate | |
| #126 | methyl [4-{5-chloro-4-[3-(1- | (1R,3S)-3-(tert- |
| | methylethyl)phenyl]-3-pyridinyl}-4- | butoxycarbonyl(methyl)amino)cyclopentanecarboxylic |
| | hydroxy-4-((3R)-1-{[(1R,3S)-3- | acid |
| | (methylamino)cyclopentyl]carbonyl}-3- | |
| | piperidinyl)butyl]carbamate | |
| #127 | methyl {4-[6-fluoro-3′-(1- | (1R,4R)-4-((tert- |
| | methylethyl)-2-biphenylyl]-4-hydroxy- | butoxycarbonyl(methyl)amino)methyl)cyclohexanecarboxylic |
| | 4-[(3R)-1-({trans-4- | acid |
| | [(methylamino)methyl]cyclohexyl}carbonyl)- | |
| | 3-piperidinyl]butyl}carbamate | |
| #128 | methyl [4-(6-chloro-3′-fluoro-5′- | (1R,3S)-3-(tert- |
| | methyl-2-biphenylyl)-4-hydroxy-4- | butoxycarbonyl(methyl)amino)cyclopentanecarboxylic |
| | ((3R)-1-{[(1R,3S)-3- | acid |
| | (methylamino)cyclopentyl]carbonyl}-3- | |
| | piperidinyl)butyl]carbamate | |
| #129 | methyl [4-(3′,6-difluoro-5′-methyl- | (1R,3S)-3-(tert- |
| | 2-biphenylyl)-4-hydroxy-4-((3R)-1- | butoxycarbonyl(methyl)amino)cyclopentanecarboxylic |
| | {[(1R,3S)-3- | acid |
| | (methylamino)cyclopentyl]carbonyl}-3- | |
| | piperidinyl)butyl]carbamate | |
| #131 | methyl {4-(3′-ethyl-6-fluoro-2- | (1R,4R)-4-((tert- |
| | biphenylyl)-4-hydroxy-4-[(3R)-1- | butoxycarbonyl(methyl)amino)methyl)cyclohexanecarboxylic |
| | ({trans-4- | acid |
| | [(methylamino)methyl]cyclohexyl}carbonyl)- | |
| | 3-piperidinyl]butyl}carbamate | |
| #144 | methyl [4-((3R)-1-{[4- | 4-((tert- |
| | (aminomethyl)-5-methyl-2- | butoxycarbonylamino)methyl)-5- |
| | furanyl]carbonyl}-3-piperidinyl)-4-(6- | methylfuran-2-carboxylic acid |
| | chloro-3′-ethyl-2-biphenylyl)-4- | |
| | hydroxybutyl]carbamate | |
| #145 | methyl {4-(6-chloro-3′-ethyl-2- | 6-((tert- |
| | biphenylyl)-4-hydroxy-4-[(3R)-1-({6- | butoxycarbonyl(methyl)amino)methyl)nicotinic |
| | [(methylamino)methyl]-3- | acid |
| | pyridinyl}carbonyl)-3- | |
| | piperidinyl]butyl}carbamate | |
| #146 | methyl [4-{(3R)-1-[(2-amino-4- | 2-amino-4-hydroxypyrimidine-5- |
| | oxo-1,4-dihydro-5- | carboxylic acid |
| | pyrimidinyl)carbonyl]-3-piperidinyl}-4- | |
| | (6-chloro-3′-methyl-2-biphenylyl)-4- | |
| | hydroxybutyl]carbamate | |
| #147 | methyl [4-(6-chloro-3′-methyl-2- | 2-(methylamino)pyrimidine-5- |
| | biphenylyl)-4-hydroxy-4-((3R)-1-{[2- | carboxylic acid |
| | (methylamino)-5- | |
| | pyrimidinyl]carbonyl}-3- | |
| | piperidinyl)butyl]carbamate | |
| #148 | methyl [4-((3R)-1-{[5- | 5-((tert- |
| | (aminomethyl)-3-isoxazolyl]carbonyl}- | butoxycarbonylamino)methyl)isoxazole- |
| | 3-piperidinyl)-4-(6-chloro-3′-methyl-2- | 3-carboxylic acid |
| | biphenylyl)-4-hydroxybutyl]carbamate | |
| #149 | methyl {4-(6-chloro-3′-ethyl-2- | 5-((tert- |
| | biphenylyl)-4-hydroxy-4-[(3R)-1-({5- | butoxycarbonyl(methyl)amino)methyl)thiophene- |
| | [(methylamino)methyl]-2- | 2-carboxylic acid |
| | thienyl}carbonyl)-3- | |
| | piperidinyl]butyl}carbamate | |
| #150 | methyl [4-{(3R)-1-[(6-amino-3- | 6-aminonicotinic acid |
| | pyridinyl)carbonyl]-3-piperidinyl}-4- | |
| | (3′,6-difluoro-5′-methyl-2-biphenylyl)- | |
| | 4-hydroxybutyl]carbamate | |
| #151 | methyl [2-({(R)-(6-chloro-3′-ethyl- | 4-{[{[(1,1- |
| | 2-biphenylyl)[(3R)-1-({trans-4- | dimethylethyl)oxy]carbonyl}-trans- |
| | [(methylamino)methyl]cyclohexyl}carbonyl)- | (methyl)amino]methyl}cyclohexanecarboxylic |
| | 3-piperidinyl]methyl}oxy)ethyl]carbamate | acid |
| #152 | methyl [2-({(S)-(6-chloro-3′-ethyl- | 4-{[{[(1,1- |
| | 2-biphenylyl)[(2R)-4-({trans-4- | dimethylethyl)oxy]carbonyl}-trans- |
| | [(methylamino)methyl]cyclohexyl}carbonyl)- | (methyl)amino]methyl}cyclohexanecarboxylic |
| | 2-morpholinyl]methyl}oxy)ethyl]carbamate | acid |
| #153 | N-[4-((3R)-1-{[6-(aminomethyl)-3- | 6-((tert- |
| | pyridinyl]carbonyl}-3-piperidinyl)-4-(6- | butoxycarbonylamino)methyl)nicotinic |
| | chloro-3′-methyl-2-biphenylyl)-4- | acid |
| | hydroxybutyl]acetamide | |

### Example 7

### ((1S,3R,4S)-3-amino-4-hydroxycyclopentyl)((R)-2-((R)-1-(6-chloro-3′-ethylbiphenyl-2-yl)-1-hydroxypent-4-enyl)morpholino)methanone (#6)

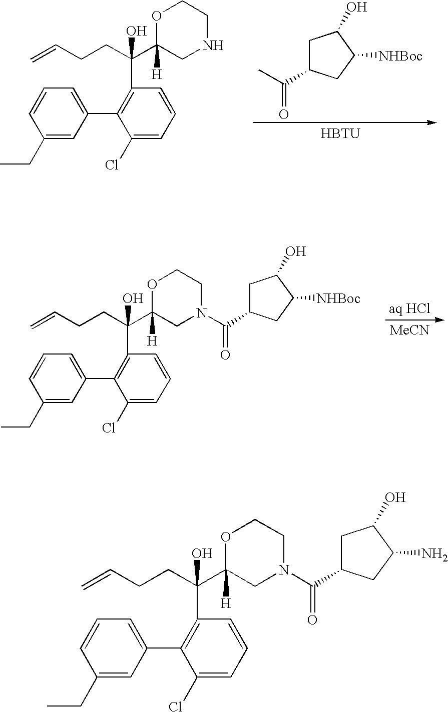

Step 1. tert-butyl (1R,2S,4S)-4-((R)-2-((R)-1-(6-chloro-3′-ethylbiphenyl-2-yl)-1-hydroxypent-4-enyl)morpholine-4-carbonyl)-2-hydroxycyclopentylcarbamate: To a solution of (R)-1-(6-chloro-3′-ethylbiphenyl-2-yl)-1-((R)-morpholin-2-yl)pent-4-en-1-ol (55 mg, 0.14 mmol), (1S,3R,4S)-3-(tert-butoxycarbonylamino)-4-hydroxycyclopentanecarboxylic acid (35 mg, 0.14 mmol), and i-Pr₂NEt (54 mg, 0.42 mmol) in 2 mL of DMF was added HBTU (64 mg, 0.17 mmol). The reaction was stirred for 2 h and diluted with 10 mL water. It was extracted with EtOAc (3×10 mL). The combined organic extracts were dried over Na₂SO₄ and filtered, followed by concentration under reduced pressure. This afforded tert-butyl (1R,2S,4S)-4-((R)-2-((R)-1-(6-chloro-3′-ethylbiphenyl-2-yl)-1-hydroxypent-4-enyl)morpholine-4-carbonyl)-2-hydroxycyclopentylcarbamate which was used without purification.

Step 2. ((1S,3R,4S)-3-amino-4-hydroxycyclopentyl)((R)-2-((R)-1-(6-chloro-3′-ethylbiphenyl-2-yl)-1-hydroxypent-4-enyl)morpholino)methanone: To a solution of tent-butyl (1R,2S,4S)-4-((R)-2-((R)-1-(6-chloro-3′-ethylbiphenyl-2-yl)-1-hydroxypent-4-enyl)morpholine-4-carbonyl)-2-hydroxycyclopentylcarbamate (85 mg, 0.14 mmol) in 10 mL of MeCN was added 10 mL of 2N aq HCl. The reaction was stirred overnight. It was basified with 10N aq NaOH to pH=14 and extracted with CH₂Cl₂ (3×10 ml). The combined organic extracts were dried over Na₂SO₄ and filtered, followed by concentration under reduced pressure. This afforded ((1S,3R,4S)-3-amino-4-hydroxycyclopentyl)((R)-2-((R)-1-(6-chloro-3′-ethylbiphenyl-2-yl)-1-hydroxypent-4-enyl)morpholino)methanone which was purified by reverse phase HPLC. LC-MS t_{R}=2.52 min, (m/z) 513.2 (M+H⁺).

### Example 8

The following compounds were prepared following procedures analogous to those described in Example 7.
#5 methyl 4-(1-((1S,3R,4S)-3-amino-4-hydroxycyclopentanecarbonyl)piperidin-3-yl)-4-hydroxy-4-(2-(pyridin-3-yl)phenyl)butylcarbamate
#8 2-((S)-((R)-4-((1S,3R,4S)-3-amino-4-hydroxycyclopentanecarbonyl)morpholin-2-yl)(6-fluoro-3′-methylbiphenyl-2-yl)methoxy)-N-ethylacetamide
#10 ((1S,3R,4S)-3-amino-4-hydroxycyclopentyl)((R)-2-((R)-1-(3-chloro-2-(pyridin-3-yl)phenyl)-1-hydroxy-5-methoxypentyl)morpholino)methanone
#11 ((1S,3R,4S)-3-amino-4-hydroxycyclopentyl)(2-(1-(3-chloro-2-(3-methyl-1,2,4-oxadiazol-5-yl)phenyl)-1-hydroxy-5-methoxypentyl)morpholino)methanone
#13 N-(4-((R)-1-((1S,3R,4S)-3-amino-4-hydroxycyclopentanecarbonyl)piperidin-3-yl)-4-hydroxy-4-(2-(o-tolyloxy)phenyl)butyl)acetamide
#14 ((1S,3R,4S)-3-amino-4-hydroxycyclopentyl)((3R)-3-(1-(2-(2,6-dimethylphenoxy)phenyl)-1-hydroxy-5-methoxypentyl)piperidin-1-yl)methanone
#23 ((1S,3R,4S)-3-amino-4-hydroxycyclopentyl)((R)-2-((R)-1-(4′,6-difluoro-3′-methylbiphenyl-2-yl)-1-hydroxy-5-methoxypentyl)morpholino)methanone
#24 methyl 4-((R)-1-((1S,3R,4S)-3-amino-4-hydroxycyclopentanecarbonyl)piperidin-3-yl)-4-hydroxy-4-(2-(o-tolyloxy)phenyl)butylcarbamate
#29 ((1S,3R,4S)-3-amino-4-hydroxycyclopentyl)((3R)-3-(1-(6-fluoro-3′-methoxy-5′-methylbiphenyl-2-yl)-1-hydroxy-5-methoxypentyl)piperidin-1-yl)methanone
#30 ((1S,3R,4S)-3-amino-4-hydroxycyclopentyl)((3R)-3-(1-(6-chloro-3′-ethylbiphenyl-2-yl)-1-hydroxy-5-methoxypentyl)piperidin-1-yl)methanone
#33 ((1S,3R,4S)-3-amino-4-hydroxycyclopentyl)((3R)-3-(1-(3-chloro-2-(2-methylbenzyl)phenyl)-1-hydroxy-5-methoxypentyl)piperidin-1-yl)methanone
#34 ((1S,3R,4S)-3-amino-4-hydroxycyclopentyl)((3R)-3-(1-(3-chloro-2-(3-methylbenzyl)phenyl)-1-hydroxy-5-methoxypentyl)piperidin-1-yl)methanone
#35 ((1S,3R,4S)-3-amino-4-hydroxycyclopentyl)(2-(1-(6-fluoro-3′-methoxy-5′-methylbiphenyl-2-yl)-1-hydroxy-5-methoxypentyl)morpholino)methanone
#36 ((1S,3R,4S)-3-amino-4-hydroxycyclopentyl)((3R)-3-(1-(3-chloro-2-(o-tolyloxy)phenyl)-1-hydroxy-5-methoxypentyl)piperidin-1-yl)methanone
#37 ((1S,3R,4S)-3-amino-4-hydroxycyclopentyl)(2-(1-(6-chloro-3′-ethylbiphenyl-2-yl)-1-hydroxy-5-methoxypentyl)morpholino)methanone
#38 ((1S,3R,4S)-3-amino-4-hydroxycyclopentyl)((2R)-2-((1R)-1-(6-chloro-2′-fluoro-5′-methylbiphenyl-2-yl)-1-hydroxy-5-methoxypentyl)morpholino)methanone
#44 N-(4-((R)-1-((1S,3R,4S)-3-amino-4-hydroxycyclopentanecarbonyl)piperidin-3-yl)-4-(6-chloro-3′-ethylbiphenyl-2-yl)-4-hydroxybutyl)acetamide
#47 ((1S,3R,4S)-3-amino-4-hydroxycyclopentyl)((3R)-3-(1-(3-chloro-2-(3-ethylphenoxy)phenyl)-1-hydroxy-5-methoxypentyl)piperidin-1-yl)methanone
#48 ((1S,3R,4S)-3-amino-4-hydroxycyclopentyl)((R)-2-((R)-1-(6-fluoro-3′,5′-dimethoxybiphenyl-2-yl)-1-hydroxy-5-methoxypentyl)morpholino)methanone
#49 ((1S,3R,4S)-3-amino-4-hydroxycyclopentyl)((R)-2-((R)-1-(6-chloro-3′-(methoxymethyl)biphenyl-2-yl)-1-hydroxy-5-methoxypentyl)morpholino)methanone
#51 ((1S,3R,4S)-3-amino-4-hydroxycyclopentyl)((R)-3-((R)-1-(3-chloro-2-(quinolin-3-yl)phenyl)-1-hydroxy-5-methoxypentyl)piperidin-1-yl)methanone
#52 ((1S,3R,4S)-3-amino-4-hydroxycyclopentyl)((R)-2-((R)-1-(3-chloro-2-(naphthalen-2-yl)phenyl)-1-hydroxy-5-methoxypentyl)morpholino)methanone
#53 ((1S,3R,4S)-3-amino-4-hydroxycyclopentyl)(2-(1-(3-chloro-2-(quinolin-3-yl)phenyl)-1-hydroxy-5-methoxypentyl)morpholino)methanone
#54 ((1S,3R,4S)-3-amino-4-hydroxycyclopentyl)((2R)-2-((1R)-1-(3-chloro-2-(isoquinolin-4-yl)phenyl)-1-hydroxy-5-methoxypentyl)morpholino)methanone
#57 methyl 4-((R)-1-((1S,3R,4S)-3-amino-4-hydroxycyclopentanecarbonyl)piperidin-3-yl)-4-(6-chloro-3′-ethylbiphenyl-2-yl)-4-hydroxybutylcarbamate
#59 ((1S,3R,4S)-3-amino-4-hydroxycyclopentyl)((R)-2-((R)-1-(6-chloro-3′,5′-dimethoxybiphenyl-2-yl)-1-hydroxy-5-methoxypentyl)morpholino)methanone
#61 methyl 4-((R)-1-((1S,3R,4S)-3-amino-4-hydroxycyclopentanecarbonyl)piperidin-3-yl)-4-(6-chloro-3′-isopropylbiphenyl-2-yl)-4-hydroxybutylcarbamate
#63 methyl 4-((R)-1-((1S,3R,4S)-3-amino-4-hydroxycyclopentanecarbonyl)piperidin-3-yl)-4-(3-chloro-2-(quinolin-3-yl)phenyl)-4-hydroxybutylcarbamate
#65 ((1S,3R,4S)-3-amino-4-hydroxycyclopentyl)((R)-3-45)-1-(3′-ethoxy-6-fluoro-5′-(trifluoromethyl)biphenyl-2-yl)-1-hydroxy-5-methoxypentyl)piperidin-1-yl)methanone
#66 ((1S,3R,4S)-3-amino-4-hydroxycyclopentyl)(2-(1-(3′-ethoxy-6-fluoro-5′-(trifluoromethyl)biphenyl-2-yl)-1-hydroxy-5-methoxypentyl)morpholino)methanone
#71 methyl (4-((3R)-1-{[(1S,3R,4S)-3-amino-4-hydroxycyclopentyl]carbonyl}-3-piperidinyl)-4-{3-chloro-2-[(2-methylphenyl)oxy]phenyl}-4-hydroxybutyl)carbamate
#72 methyl (4-((3R)-1-{[(1S,3R,4S)-3-amino-4-hydroxycyclopentyl]carbonyl}-3-piperidinyl)-4-{3-chloro-2-[(2-ethylphenyl)oxy]phenyl}-4-hydroxybutyl)carbamate
#73 methyl [4-((3R)-1-{[(1S,3R,4S)-3-amino-4-hydroxycyclopentyl]carbonyl}-3-piperidinyl)-4-(3′-ethyl-6-fluoro-2-biphenylyl)-4-hydroxybutyl] carbamate
#97 methyl [(4R)-4-((2R)-4-{[(1S,3R,4S)-3-amino-4-hydroxycyclopentyl]carbonyl}-2-morpholinyl)-4-(3′-ethyl-6-fluoro-2-biphenylyl)-4-hydroxybutyl]carbamate
#99 (1S,2R,4S)-2-amino-4-({(2R)-2-[(1R)-1-[2-chloro-3-(3-ethylphenyl)-4-pyridinyl]-1-hydroxy-5-(methyloxy)pentyl]-4-morpholinyl}carbonyl)cyclopentanol
#101 (1S,2R,4S)-2-amino-4-({(2R)-2-[(1R)-1-[2-(1-benzothien-3-yl)-3-chlorophenyl]-1-hydroxy-5-(methyloxy)pentyl]-4-morpholinyl}carbonyl)cyclopentanol
#109 (1S,2R,4S)-2-amino-4-({(3R)-3-[1-{3-chloro-2-[(3-methylphenyl)oxy]phenyl}-1-hydroxy-5-(methyloxy)pentyl]-1-piperidinyl}carbonyl)cyclopentanol
#111 methyl (4-((3R)-1-{[(1S,3R,4S)-3-amino-4-hydroxycyclopentyl]carbonyl}-3-piperidinyl)-4-{3-chloro-2-[(3-methylphenyl)oxy]phenyl}-4-hydroxybutyl)carbamate
#113 methyl (4-((3R)-1-{[(1S,3R,4S)-3-amino-4-hydroxycyclopentyl]carbonyl}-3-piperidinyl)-4-{2-[(2,6-dimethylphenyl)oxy]phenyl}-4-hydroxybutyl)carbamate
#119 methyl {4-((3R)-1-{[(1S,3R,4S)-3-amino-4-hydroxycyclopentyl]carbonyl}-3-piperidinyl)-4-[3-chloro-2-(5-methyl-2-furanyl)phenyl]-4-hydroxybutyl}carbamate
#122 (1S,2R,4S)-2-amino-4-({(2R)-2-[(1R)-1-{2-chloro-3-[3-(1-methylethyl)phenyl]-4-pyridinyl}-1-hydroxy-5-(methyloxy)pentyl]-4-morpholinyl}carbonyl)cyclopentanol
#130 (1S,2R,4S)-2-amino-4-({(3R)-3-[1-{3-chloro-2-[(2-ethylphenyl)oxy]phenyl}-1-hydroxy-5-(methyloxy)pentyl]-1-piperidinyl}carbonyl)cyclopentanol
#132 (1S,2R,4S)-2-amino-4-({(3R)-3-[1-{2-[(2-chloro-6-methylphenyl)oxy]phenyl}-1-hydroxy-5-(methyloxy)pentyl]-1-piperidinyl}carbonyl)cyclopentanol
#133 (1S,2R,4S)-2-amino-4-({(3R)-3-[1-{3-fluoro-2-[(3-methylphenyl)oxy]phenyl}-1-hydroxy-5-(methyloxy)pentyl]-1-piperidinyl}carbonyl)cyclopentanol
#135 methyl (4-((3R)-1-{[(1S,3R,4S)-3-amino-4-hydroxycyclopentyl]carbonyl}-3-piperidinyl)-4-{3-chloro-2-[(3-ethylphenyl)oxy]phenyl}-4-hydroxybutyl)carbamate
#137 methyl (4-((3R)-1-{[(1S,3R,4S)-3-amino-4-hydroxycyclopentyl]carbonyl}-3-piperidinyl)-4-{3-fluoro-2-[(3-methylphenyl)oxy]phenyl}-4-hydroxybutyl)carbamate
#139 methyl (4-((3R)-1-{[(1S,3R,4S)-3-amino-4-hydroxycyclopentyl]carbonyl}-3-piperidinyl)-4-{3-chloro-2-[(3-methylphenyl)methyl]phenyl}-4-hydroxybutyl)carbamate
#140 methyl [4-{3-chloro-2-[(3-methylphenyl)methyl]phenyl}-4-hydroxy-4-((3R)-1-{[(1R,3S)-3-(methylamino)cyclopentyl]carbonyl}-3-piperidinyl)butyl]carbamate
#142 methyl (2-{[(S)-((2R)-4-{[(1S,3R,4S)-3-amino-4-hydroxycyclopentyl]carbonyl}-2-morpholinyl)(6-chloro-3′-ethyl-2-biphenylyl)methyl]oxy}ethyl)carbamate
#143 methyl (2-{[(R) — ((3R)-1-{[(1S,3R,4S)-3-amino-4-hydroxycyclopentyl]carbonyl}-3-piperidinyl)(6-chloro-3′-ethyl-2-biphenylyl)methyl]oxy}ethyl)carbamate

### Example 9

### (1S,2R,4S)-4-({(3R)-3-[1-(6-chloro-3′-ethyl-2-biphenylyl)-1-hydroxy-5-methyloxy)pentyl]-1-piperidinyl}carbonyl)-2-[(2-hydroxyethyl)amino]cyclopentanol (#84)

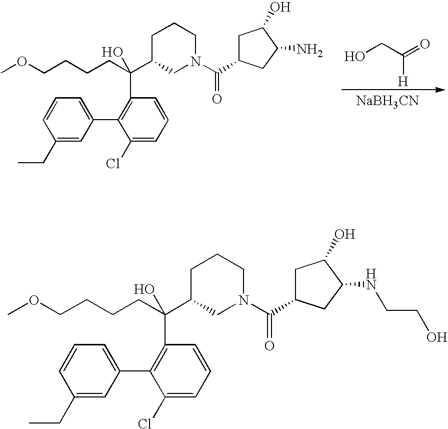

To a solution of (1S,2R,4S)-2-amino-4-({(3R)-3-[1-(6-chloro-3′-ethyl-2-biphenylyl)-1-hydroxy-5-(methyloxy)pentyl]-1-piperidinyl}carbonyl)cyclopentanol (44 mg, 0.064 mmol) in 1,2-dichloroethane (2 ml) was added hydroxyacetaldehyde (16 mg, 0.13 mmol) and sodium cyanoborohydride (20 mg, 0.32 mmol). The reaction mixture was stirred at room temperature overnight. To the mixture was added 2N HCl solution and the organic products were extracted into methylene chloride. The organic materials were dried and concentrated. The product was purified by reverse phase HPLC to give (1S,2R,4S)-4-({(3R)-3-[1-(6-chloro-3′-ethyl-2-biphenylyl)-1-hydroxy-5-(methyloxy)pentyl]-1-piperidinyl}carbonyl)-2-[(2-hydroxyethyl)amino]cyclopentanol (6.2 mg).

### Example 10

The following compounds were prepared following procedures analogous to those described in Example 9 using the indicated aldehyde.

| Cpd. No. | Product | Aldehyde |
|---|---|---|
| #83 | (1S,2R,4S)-4-({(2R)-2-[(1R)-1-(6-chloro-3′- | hydroxyacetaldehyde |
| | ethyl-2-biphenylyl)-1-hydroxy-5- | |
| | (methyloxy)pentyl]-4-morpholinyl}carbonyl)-2- | |
| | [(2-hydroxyethyl)amino]cyclopentanol | |
| #86 | (1S,2R,4S)-4-({(2R)-2-[(1R)-1-(6-chloro-3′- | thiazole-2-carbaldehyde |
| | ethyl-2-biphenylyl)-1-hydroxy-5- | |
| | (methyloxy)pentyl]-4-morpholinyl}carbonyl)-2- | |
| | [(1,3-thiazol-2-ylmethyl)amino]cyclopentanol | |
| #90 | (1R)-1-(6-chloro-3′-ethyl-2-biphenylyl)-1- | 2,5-dimethyloxazole-4- |
| | {(2R)-4-[((1R,3S)-3-{[(2,5-dimethyl-1,3-oxazol-4- | carbaldehyde |
| | yl)methyl]amino}cyclopentyl)carbonyl]-2- | |
| | morpholinyl}-5-(methyloxy)-1-pentanol | |

### Example 11

### methyl [4-((3R)-1-{[4-(aminomethyl)-1-piperidinyl]carbonyl}-3-piperidinyl)-4-(6-chloro-3′-ethyl-2-biphenylyl)-4-hydroxybutyl]carbamate (#156)

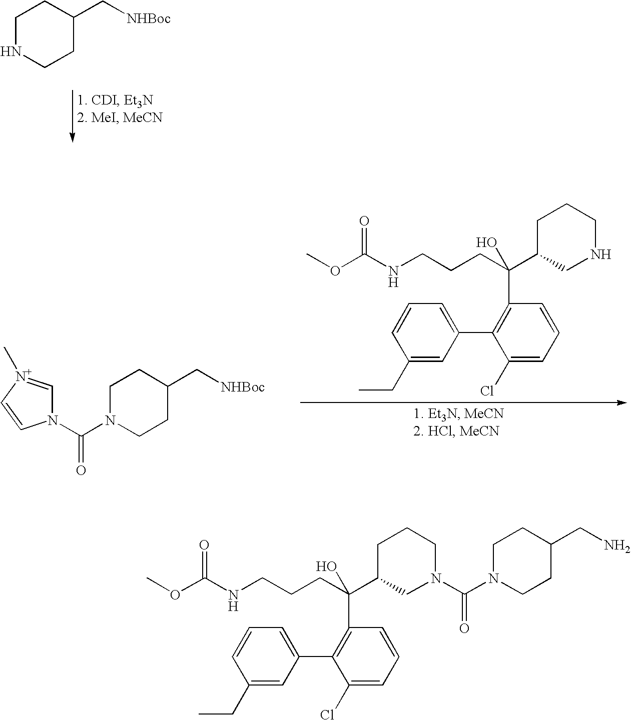

Step 1. 1-({4-[({[(1,1-dimethylethyl)oxy]carbonyl}amino)methyl]-1-piperidinyl}carbonyl)-3-methyl-1H-imidazol-3-ium: A solution of 1,1-dimethylethyl (4-piperidinylmethyl)carbamate (3.0 g, 14.0 mmol), CDI (2.72 g, 16.8 mmol), and Et₃N (5.85 ml, 42.0 mmol) in CH₂Cl₂ (20 ml) was stirred at 25° C. for 60 hours before being concentrated under reduced pressure and subjected to flash chromatography to give a colorless solid. The solid was dissolved in 20 mL of CH₃CN and the mixture was treated with iodomethane (3.49 ml, 56.0 mmol) and stirred overnight. The solvent was removed to give 1-({4-[({[(1,1-dimethylethyl)oxy]carbonyl}amino)methyl]-1-piperidinyl}carbonyl)-3-methyl-1H-imidazol-3-ium as a colorless solid (2.75 g, 60%) which was used without purification. ESI-MS (m/z): 323.2 (M⁺).

Step 2. Methyl [4-((3R)-1-{[4-(aminomethyl)-1-piperidinyl]carbonyl}-3-piperidinyl)-4-(6-chloro-3′-ethyl-2-biphenylyl)-4-hydroxybutyl]carbamate: A solution of methyl {4-(6-chloro-3′-ethyl-2-biphenylyl)-4-hydroxy-4-[(3R)-3-piperidinyl]butyl}carbamate (0.25 g, 0.562 mmol) in CH₃CN (5 ml) was treated with 1-({4-[({[(1,1-dimethylethyl)oxy]carbonyl} amino)methyl]-1-piperidinyl}carbonyl)-3-methyl-1H-imidazol-3-ium (0.363 g, 1.124 mmol) and stirred at 50° C. for 1 h before being subjected to reverse phase HPLC to give a solid. The solid was treated with 5 mL of 10% 4N HCl/dioxane in MeCN and the resulting solution was stirred at 25° C. overnight before being concentrated to give a tan solid, which was dissolved in water and lyophilized to give methyl [4-((3R)-1-{[4-(aminomethyl)-1-piperidinyl]carbonyl}-3-piperidinyl)-4-(6-chloro-3′-ethyl-2-biphenylyl)-4-hydroxybutyl]carbamate as a tan solid (0.31 g, 92%): LCMS (m/z): 585.2 (M+H⁺).

### Example 12

The following compounds were prepared following procedures analogous to those described in Example 11:
#154 methyl 4-((R)-1-(4-(aminomethyl)piperidine-1-carbonyl)piperidin-3-yl)-4-(3′-ethyl-6-fluorobiphenyl-2-yl)-4-hydroxybutylcarbamate using methyl (R)-4-(3′-ethyl-6-fluorobiphenyl-2-yl)-4-hydroxy-4-((R)-piperidin-3-yl)butylcarbamate instead of methyl {4-(6-chloro-3′-ethyl-2-biphenylyl)-4-hydroxy-4-[(3R)-3-piperidinyl]butyl}carbamate in Step 2.
#157 methyl [4-((3R)-1-{[4-(aminomethyl)-4-phenyl-1-piperidinyl]carbonyl}-3-piperidinyl)-4-(6-chloro-3′-ethyl-2-biphenylyl)-4-hydroxybutyl]carbamate using 1,1-dimethylethyl [(4-phenyl-4-piperidinyl)methyl]carbamate instead of 1,1-dimethylethyl (4-piperidinylmethyl)carbamate in Step 1.
#158 N-(4-((R)-1-(4-(aminomethyl)piperidine-1-carbonyl)piperidin-3-yl)-4-(2′,6-difluoro-5′-methylbiphenyl-2-yl)-4-hydroxybutyl)-2-hydroxyacetamide using N-44R)-4-(2′,6-difluoro-5′-methylbiphenyl-2-yl)-4-hydroxy-4-((R)-piperidin-3-yl)butyl)-2-hydroxyacetamide instead of methyl {4-(6-chloro-3′-ethyl-2-biphenylyl)-4-hydroxy-4-[(3R)-3-piperidinyl]butyl}carbamate in Step 2.

### Example 13

### methyl {4-(6-chloro-3′-ethyl-2-biphenylyl)-4-hydroxy-4-[(3R)-1-({4-[(methylamino)methyl]-1-piperidinyl}carbonyl)-3-piperidinyl]butyl}carbamate (#159)

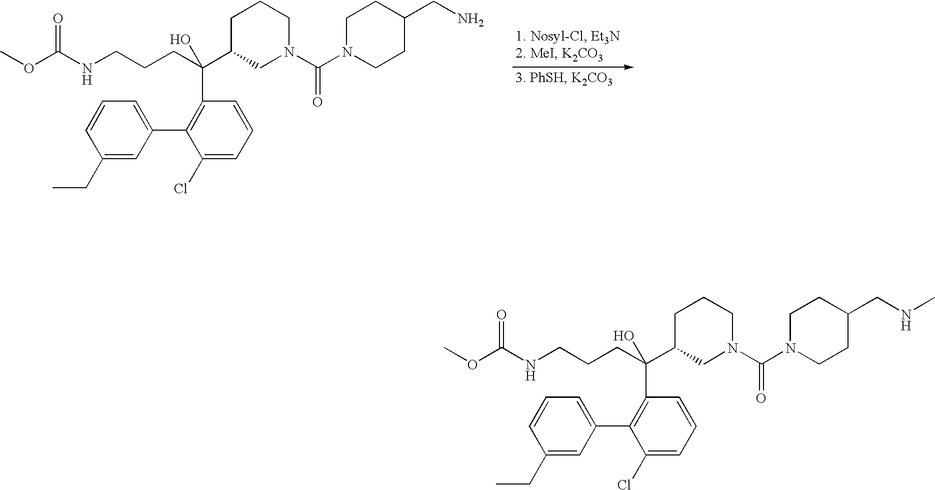

Step 1. methyl [4-(6-chloro-3′-ethyl-2-biphenylyl)-4-hydroxy-4-43R)-1-{[4-({[(2-nitrophenyl)sulfonyl]amino}methyl)-1-piperidinyl]carbonyl}-3-piperidinyl)butyl]carbamate: A solution of methyl [4-((3R)-1-{[4-(aminomethyl)-1-piperidinyl]carbonyl}-3-piperidinyl)-4-(6-chloro-3′-ethyl-2-biphenylyl)-4-hydroxybutyl]carbamate (0.25 g, 0.42 mmol) in CH₂Cl₂ (10 ml) at 0° C. was treated with 2-nitrobenzenesulfonyl chloride (0.114 g, 0.51 mmol) and Et₃N (0.179 ml, 1.28 mmol), and the mixture was stirred at for 45 min before being concentrated under reduced pressure to give methyl [4-(6-chloro-3′-ethyl-2-biphenylyl)-4-hydroxy-4-((3R)-1-{[4-({[(2-nitrophenyl)sulfonyl]amino}methyl)-1-piperidinyl]carbonyl}-3-piperidinyl)butyl]carbamate as a tan solid. ESI-MS (m/z): 770.3 (M+H⁺).

Step 2. methyl {4-(6-chloro-3′-ethyl-2-biphenylyl)-4-hydroxy-4-[(3R)-1-({4-[(methylamino)methyl]-1-piperidinyl}carbonyl)-3-piperidinyl]butyl}carbamate: A solution of methyl [4-(6-chloro-3′-ethyl-2-biphenylyl)-4-hydroxy-4-43R)-1-{[4-({[(2-nitrophenyl)sulfonyl]amino}methyl)-1-piperidinyl]carbonyl}-3-piperidinyl)butyl]carbamate (0.10 g, 0.13 mmol) in DMF (2 ml) at 25° C. was treated with iodomethane (0.032 ml, 0.52 mmol) and K₂CO₃ (0.036 g, 0.260 mmol) and stirred overnight before being filtered through celite and subjected to reverse phase HPLC to give methyl [4-(6-chloro-3′-ethyl-2-biphenylyl)-4-hydroxy-4-43R)-1-{[4-({methyl [(2-nitrophenyl)sulfonyl]amino}methyl)-1-piperidinyl]carbonyl}-3-piperidinyl)butyl]carbamate a solid. This material was dissolved in DMF (2 ml) and treated with thiophenol (0.053 ml, 0.52 mmol) and K₂CO₃ (0.036 g, 0.260 mmol) and the mixture was stirred at 25° C. overnight before being filtered and subjected to reverse phase HPLC to give methyl {4-(6-chloro-3′-ethyl-2-biphenylyl)-4-hydroxy-4-[(3R)-1-({4-[(methylamino)methyl]-1-piperidinyl}carbonyl)-3-piperidinyl]butyl}carbamate as a solid (0.056 g, 57%). ESI-MS (m/z): 599.3 (M+H⁺).

### Example 14

The following compounds were prepared following procedures analogous to those described in Example 13:
#155 methyl 4-(3′-ethyl-6-fluorobiphenyl-2-yl)-4-hydroxy-4-((R)-1-(4-((methylamino)methyl)piperidine-1-carbonyl)piperidin-3-yl)butylcarbamate using methyl (R)-4-((R)-1-(4-(aminomethyl)piperidine-1-carbonyl)piperidin-3-yl)-4-(3′-ethyl-6-fluorobiphenyl-2-yl)-4-hydroxybutylcarbamate instead of methyl [4-((3R)-1-{[4-(aminomethyl)-1-piperidinyl]carbonyl}-3-piperidinyl)-4-(6-chloro-3′-ethyl-2-biphenylyl)-4-hydroxybutyl]carbamate in Step 1.
#160 methyl {4-(6-chloro-3′-ethyl-2-biphenylyl)-4-hydroxy-4-[(3R)-1-({4-[(2-propen-1-ylamino)methyl]-1-piperidinyl}carbonyl)-3-piperidinyl]butyl}carbamate by replacing methyl iodide with allyl bromide in Step 2.

The following is a compound of the invention:

| | | Synthetic | LC_MS | | |
|---|---|---|---|---|---|
| | | Method | Method 1 | Mass | |
| Cpd. #. | Name | Example No. | t_{R}(min) | observed | Selected 1H NMR resonances^{a} |
| 37 | ((1S,3R,4S)-3-amino-4- | 8 | | 567 | 0.8-1.0 (m, 1H), 1.20-1.40 (m, 6H), 1.40- |
| | hydroxycyclopentyl)(2-(1-(6-chloro-3′- | | | | 1.55 (m, 3H), 1.60-1.80 (m, 1H), 1.90-2.15 |
| | ethylbiphenyl-2-yl)-1-hydroxy-5- | | | | (m, 2H), 2.20-2.35 (m, 1H), 2.60-2.80 (m, |
| | methoxypentyl)morpholino)methanone | | | | 3H), 3.10-3.25 (m, 2H), 3.25-3.35 (m, 5H), |
| | | | | | 3.50 (m, 1H), 3.65-3.90 (m, 2H), 4.20-4.37 |
| | | | | | (m, 2H), 6.90-7.00 (m, 2H), 7.20-7.42 (m, |
| | | | | | 4H), 7.80 (m, 1H). |

^{a1}H NMR spectra were acquired in CD₃OD unless otherwise indicated.

The following are compounds of the invention:

| | | Synthetic | | | |
|---|---|---|---|---|---|
| | | Method | LC_MS | | Mass |
| Cpd. #. | Name | Example No. | Method | t_{R}(min) | observed |
| 1 | methyl 4-((R)-1-((1R,3S)-3- | 2 | 1 | 1.76 | 495.3 |
| | aminocyclopentanecarbonyl)piperidin-3-yl)-4- | | | | |
| | hydroxy-4-(2-(pyridin-4- | | | | |
| | yl)phenyl)butylcarbamate | | | | |
| 2 | 2-((R)-((R)-4-((1R,3S)-3- | 2 | 1 | 2.22 | 498.2 |
| | aminocyclopentanecarbonyl)morpholin-2- | | | | |
| | yl)(6-fluoro-3′-methylbiphenyl-2-yl)methoxy)- | | | | |
| | N-ethylacetamide | | | | |
| 3 | N-(4-((R)-1-((1R,3S)-3- | 2 | 1 | 2.11 | 508.3 |
| | aminocyclopentanecarbonyl)piperidin-3-yl)-4- | | | | |
| | hydroxy-4-(2-(o- | | | | |
| | tolyloxy)phenyl)butyl)acetamide | | | | |
| 4 | ((1R,3S)-3-aminocyclopentyl)((3R)-3-(1- | 2 | | | 509.2 |
| | (2-(2,6-dimethylphenoxy)phenyl)-1-hydroxy-5- | | | | |
| | methoxypentyl)piperidin-1-yl)methanone | | | | |
| 5 | methyl 4-(1-((1S,3R,4S)-3-amino-4- | 8 | 1 | 1.72 | 511.2 |
| | hydroxycyclopentanecarbonyl)piperidin-3-yl)- | | | | |
| | 4-hydroxy-4-(2-(pyridin-3- | | | | |
| | yl)phenyl)butylcarbamate | | | | |
| 6 | ((1S,3R,4S)-3-amino-4- | 7 | 1 | 2.52 | 513.2 |
| | hydroxycyclopentyl)((R)-2-((R)-1-(6-chloro-3′- | | | | |
| | ethylbiphenyl-2-yl)-1-hydroxypent-4- | | | | |
| | enyl)morpholino)methanone | | | | |
| 7 | methyl 2-((4-((1R,3S)-3- | 2 | 1 | 2.31 | 514.2 |
| | aminocyclopentanecarbonyl)morpholin-2- | | | | |
| | yl)(6-fluoro-3′-methylbiphenyl-2- | | | | |
| | yl)methoxy)ethylcarbamate | | | | |
| 8 | 2-((S)-((R)-4-((1S,3R,4S)-3-amino-4- | 8 | 1 | 2.18 | 514.2 |
| | hydroxycyclopentanecarbonyl)morpholin-2- | | | | |
| | yl)(6-fluoro-3′-methylbiphenyl-2-yl)methoxy)- | | | | |
| | N-ethylacetamide | | | | |
| 9 | ((1R,3S)-3-aminocyclopentyl)((R)-2-((R)- | 2 | 1 | 2.26 | 517.2 |
| | 1-(4′,6-difluoro-3′-methylbiphenyl-2-yl)-1- | | | | |
| | hydroxy-5- | | | | |
| | methoxypentyl)morpholino)methanone | | | | |
| 10 | ((1S,3R,4S)-3-amino-4- | 8 | 1 | 1.87 | 518.2 |
| | hydroxycyclopentyl)((R)-2-((R)-1-(3-chloro-2- | | | | |
| | (pyridin-3-yl)phenyl)-1-hydroxy-5- | | | | |
| | methoxypentyl)morpholino)methanone | | | | |
| 11 | ((1S,3R,4S)-3-amino-4- | 8 | 1 | 2.12 | 523.2 |
| | hydroxycyclopentyl)(2-(1-(3-chloro-2-(3- | | | | |
| | methyl-1,2,4-oxadiazol-5-yl)phenyl)-1- | | | | |
| | hydroxy-5- | | | | |
| | methoxypentyl)morpholino)methanone | | | | |
| 12 | methyl 4-((R)-1-((1R,3S)-3- | 2 | 1 | 2.35 | 524.3 |
| | aminocyclopentanecarbonyl)piperidin-3-yl)-4- | | | | |
| | hydroxy-4-(2-(o- | | | | |
| | tolyloxy)phenyl)butylcarbamate | | | | |
| 13 | N-(4-((R)-1-((1S,3R,4S)-3-amino-4- | 8 | 1 | 2.07 | 524.3 |
| | hydroxycyclopentanecarbonyl)piperidin-3-yl)- | | | | |
| | 4-hydroxy-4-(2-(o- | | | | |
| | tolyloxy)phenyl)butyl)acetamide | | | | |
| 14 | ((1S,3R,4S)-3-amino-4- | 8 | | | 525.4 |
| | hydroxycyclopentyl)((3R)-3-(1-(2-(2,6- | | | | |
| | dimethylphenoxy)phenyl)-1-hydroxy-5- | | | | |
| | methoxypentyl)piperidin-1-yl)methanone | | | | |
| 15 | ((1R,3S)-3-aminocyclopentyl)((3R)-3-(1- | 2 | 1 | 2.41 | 527.3 |
| | (6-fluoro-3′-methoxy-5′-methylbiphenyl-2-yl)- | | | | |
| | 1-hydroxy-5-methoxypentyl)piperidin-1- | | | | |
| | yl)methanone | | | | |
| 16 | ((1R,3S)-3-aminocyclopentyl)((3R)-3-(1- | 2 | 1 | 2.56 | 527.3 |
| | (6-chloro-3′-ethylbiphenyl-2-yl)-1-hydroxy-5- | | | | |
| | methoxypentyl)piperidin-1-yl)methanone | | | | |
| 17 | ((1R,3S)-3-aminocyclopentyl)((3R)-3-(1- | 2 | 1 | 2.56 | 527.3 |
| | (3-chloro-2-(2-methylbenzyl)phenyl)-1- | | | | |
| | hydroxy-5-methoxypentyl)piperidin-1- | | | | |
| | yl)methanone | | | | |
| 18 | ((1R,3S)-3-aminocyclopentyl)((3R)-3-(1- | 2 | 1 | 2.47 | 527.3 |
| | (3-chloro-2-(3-methylbenzyl)phenyl)-1- | | | | |
| | hydroxy-5-methoxypentyl)piperidin-1- | | | | |
| | yl)methanone | | | | |
| 19 | ((1R,3S)-3-aminocyclopentyl)(2-(1-(6- | 2 | 1 | 2.31 | 529.3 |
| | fluoro-3′-methoxy-5′-methylbiphenyl-2-yl)-1- | | | | |
| | hydroxy-5- | | | | |
| | methoxypentyl)morpholino)methanone | | | | |
| 20 | ((1R,3S)-3-aminocyclopentyl)((2R)-2-(1- | 2 | 1 | 2.45 | 529.2 |
| | (6-chloro-3′-ethylbiphenyl-2-yl)-1-hydroxy-5- | | | | |
| | methoxypentyl)morpholino)methanone | | | | |
| 21 | ((1R,3S)-3-aminocyclopentyl)((3R)-3-(1- | 2 | 3 | 2.94; 3.03 | 529.2 |
| | (3-chloro-2-(o-tolyloxy)phenyl)-1-hydroxy-5- | | | | |
| | methoxypentyl)piperidin-1-yl)methanone | | | | |
| 22 | ((1R,3S)-3-aminocyclopentyl)((3R)-3-(1- | 2 | 3 | 1.97 | 529.3 |
| | (2-(2-chloro-6-methylphenoxy)phenyl)-1- | | | | |
| | hydroxy-5-methoxypentyl)piperidin-1- | | | | |
| | yl)methanone | | | | |
| 23 | ((1S,3R,4S)-3-amino-4- | 8 | 1 | 2.26 | 533.3 |
| | hydroxycyclopentyl)((R)-2-((R)-1-(4′,6- | | | | |
| | difluoro-3′-methylbiphenyl-2-yl)-1-hydroxy-5- | | | | |
| | methoxypentyl)morpholino)methanone | | | | |
| 24 | methyl 4-((R)-1-((1S,3R,4S)-3-amino-4- | 8 | 1 | 2.31 | 540.2 |
| | hydroxycyclopentanecarbonyl)piperidin-3-yl)- | | | | |
| | 4-hydroxy-4-(2-(o | | | | |
| | tolyloxy)phenyl)butylcarbamate | | | | |
| 25 | methyl 4-((R)-1-((1R,2S)-2- | 6 | 1 | 2.40 | 540.3 |
| | aminocyclopentanecarbonyl)piperidin-3-yl)-4- | | | | |
| | (3′-ethyl-6-fluorobiphenyl-2-yl)-4- | | | | |
| | hydroxybutylcarbamate | | | | |
| 26 | methyl 4-((R)-1-((1R,3S)-3- | 2 | 1 | 2.32 | 540.3 |
| | aminocyclopentanecarbonyl)piperidin-3-yl)-4- | | | | |
| | (3′-ethyl-6-fluorobiphenyl-2-yl)-4- | | | | |
| | hydroxybutylcarbamate | | | | |
| 27 | (trans-4-aminocyclohexyl)((3R)-3-(1-(6- | 6 | 1 | 2.60 | 555.3 |
| | chloro-3′-ethylbiphenyl-2-yl)-1-hydroxy-5- | | | | |
| | methoxypentyl)piperidin-1-yl)methanone | | | | |
| 28 | 1-((3R)-1-{[6-(aminomethyl)-3- | 4 | 1 | 2.51 | 550.2 |
| | pyridinyl]carbonyl}-3-piperidinyl)-1-(6-chloro- | | | | |
| | 3′-ethyl-2-biphenylyl)-5-(methyloxy)-1- | | | | |
| | pentanol | | | | |
| 29 | ((1S,3R,4S)-3-amino-4- | 8 | 1 | 2.35 | 543.3 |
| | hydroxycyclopentyl)((3R)-3-(1-(6-fluoro-3′- | | | | |
| | methoxy-5′-methylbiphenyl-2-yl)-1-hydroxy-5- | | | | |
| | methoxypentyl)piperidin-1-yl)methanone | | | | |
| 30 | ((1S,3R,4S)-3-amino-4- | 8 | 1 | 2.52 | 543.2 |
| | hydroxycyclopentyl)((3R)-3-(1-(6-chloro-3′- | | | | |
| | ethylbiphenyl-2-yl)-1-hydroxy-5- | | | | |
| | methoxypentyl)piperidin-1-yl)methanone | | | | |
| 31 | ((1R,3S)-3-aminocyclopentyl)((3R)-3-(1- | 2 | 3 | 3.14; 3.38 | 543.3 |
| | (3-chloro-2-(2-ethylphenoxy)phenyl)-1- | | | | |
| | hydroxy-5-methoxypentyl)piperidin-1- | | | | |
| | yl)methanone | | | | |
| 32 | ((1R,3S)-3-aminocyclopentyl)((3R)-3-(1- | 2 | 3 | 2.05 | 543.1 |
| | (3-chloro-2-(3-ethylphenoxy)phenyl)-1- | | | | |
| | hydroxy-5-methoxypentyl)piperidin-1- | | | | |
| | yl)methanone | | | | |
| 33 | ((1S,3R,4S)-3-amino-4- | 8 | 1 | 2.48 | 543.2 |
| | hydroxycyclopentyl)((3R)-3-(1-(3-chloro-2-(2- | | | | |
| | methylbenzyl)phenyl)-1-hydroxy-5- | | | | |
| | methoxypentyl)piperidin-1-yl)methanone | | | | |
| 34 | ((1S,3R,4S)-3-amino-4- | 8 | 1 | 2.44 | 543.2 |
| | hydroxycyclopentyl)((3R)-3-(1-(3-chloro-2-(3- | | | | |
| | methylbenzyl)phenyl)-1-hydroxy-5- | | | | |
| | methoxypentyl)piperidin-1-yl)methanone | | | | |
| 35 | ((1S,3R,4S)-3-amino-4- | 8 | 1 | 2.27 | 545.2 |
| | hydroxycyclopentyl)(2-(1-(6-fluoro-3′- | | | | |
| | methoxy-5′-methylbiphenyl-2-yl)-1-hydroxy-5- | | | | |
| | methoxypentyl)morpholino)methanone | | | | |
| 36 | ((1S,3R,4S)-3-amino-4- | 8 | 3 | 2.89; 2.92 | 545.3 |
| | hydroxycyclopentyl)((3R)-3-(1-(3-chloro-2-(o- | | | | |
| | tolyloxy)phenyl)-1-hydroxy-5- | | | | |
| | methoxypentyl)piperidin-1-yl)methanone | | | | |
| 38 | ((1S,3R,4S)-3-amino-4- | 8 | 1 | 2.32 | 549.3 |
| | hydroxycyclopentyl)((2R)-2-((1R)-1-(6-chloro- | | | | |
| | 2′-fluoro-5′-methylbiphenyl-2-yl)-1-hydroxy-5- | | | | |
| | methoxypentyl)morpholino)methanone | | | | |
| 39 | ((1R,3S)-3-aminocyclopentyl)((R)-3-((S)- | 2 | 1 | 2.20 | 550.3 |
| | 1-(3-chloro-2-(quinolin-3-yl)phenyl)-1-hydroxy- | | | | |
| | 5-methoxypentyl)piperidin-1-yl)methanone | | | | |
| 40 | ((1R,3S)-3-aminocyclopentyl)((R)-2-((R)- | 2 | 1 | 2.44 | 551.2 |
| | 1-(3-chloro-2-(naphthalen-2-yl)phenyl)-1- | | | | |
| | hydroxy-5- | | | | |
| | methoxypentyl)morpholino)methanone | | | | |
| 41 | ((1R,3S)-3-aminocyclopentyl)(2-(1-(3- | 2 | 1 | 2.10 | 552.2 |
| | chloro-2-(quinolin-3-yl)phenyl)-1-hydroxy-5- | | | | |
| | methoxypentyl)morpholino)methanone | | | | |
| 42 | methyl (4-hydroxy-4-((3R)-1-{[(1R,3S)-3- | 4 | 1 | 2.36 | 538.2 |
| | (methylamino)cyclopentyl]carbonyl}-3- | | | | |
| | piperidinyl)-4-{2-[(2- | | | | |
| | methylphenyl)oxy]phenyl}butyl)carbamate | | | | |
| 43 | methyl 4-(6-fluoro-3′-methoxybiphenyl-2- | 4 | 1 | 2.41 | 556.3 |
| | yl)-4-hydroxy-4-((R)-1-((1R,3S)-3- | | | | |
| | (methylamino)cyclopentanecarbonyl)piperidin- | | | | |
| | 3-yl)butylcarbamate | | | | |
| 44 | N-(4-((R)-1-((1S,3R,4S)-3-amino-4- | 8 | 1 | 2.27 | 556.3 |
| | hydroxycyclopentanecarbonyl)piperidin-3-yl)- | | | | |
| | 4-(6-chloro-3′-ethylbiphenyl-2-yl)-4- | | | | |
| | hydroxybutyl)acetamide | | | | |
| 45 | methyl 4-((R)-1-((1R,3S)-3- | 2 | 1 | 2.42 | 556.2 |
| | aminocyclopentanecarbonyl)piperidin-3-yl)-4- | | | | |
| | (6-chloro-3′-ethylbiphenyl-2-yl)-4- | | | | |
| | hydroxybutylcarbamate | | | | |
| 46 | methyl 4-((R)-4-((1R,3S)-3- | 2 | 1 | 2.31 | 558.2 |
| | aminocyclopentanecarbonyl)morpholin-2-yl)- | | | | |
| | 4-(6-chloro-3′-ethylbiphenyl-2-yl)-4- | | | | |
| | hydroxybutylcarbamate | | | | |
| 47 | ((1S,3R,4S)-3-amino-4- | 8 | 3 | 1.82 | 559.0 |
| | hydroxycyclopentyl)((3R)-3-(1-(3-chloro-2-(3- | | | | |
| | ethylphenoxy)phenyl)-1-hydroxy-5- | | | | |
| | methoxypentyl)piperidin-1-yl)methanone | | | | |
| 48 | ((1S,3R,4S)-3-amino-4- | 8 | 1 | 2.17 | 561.2 |
| | hydroxycyclopentyl)((R)-2-((R)-1-(6-fluoro- | | | | |
| | 3′,5′-dimethoxybiphenyl-2-yl)-1-hydroxy-5- | | | | |
| | methoxypentyl)morpholino)methanone | | | | |
| 49 | ((1S,3R,4S)-3-amino-4- | 8 | 2 | 1.64 | 561.3 |
| | hydroxycyclopentyl)((R)-2-((R)-1-(6-chloro-3′- | | | | |
| | (methoxymethyl)biphenyl-2-yl)-1-hydroxy-5- | | | | |
| | methoxypentyl)morpholino)methanone | | | | |
| 50 | (4-(aminomethyl)cyclohexyl)((2R)-2- | 6 | 1 | 3.03 | 561.3 |
| | ((1R)-1-(6-chloro-2′-fluoro-5′-methylbiphenyl- | | | | |
| | 2-yl)-1-hydroxy-5- | | | | |
| | methoxypentyl)morpholino)methanone | | | | |
| 51 | ((1S,3R,4S)-3-amino-4- | 8 | 1 | 2.12 | 566.2 |
| | hydroxycyclopentyl)((R)-3-((R)-1-(3-chloro-2- | | | | |
| | (quinolin-3-yl)phenyl)-1-hydroxy-5- | | | | |
| | methoxypentyl)piperidin-1-yl)methanone | | | | |
| 52 | ((1S,3R,4S)-3-amino-4- | 8 | 1 | 2.41 | 567.2 |
| | hydroxycyclopentyl)((R)-2-((R)-1-(3-chloro-2- | | | | |
| | (naphthalen-2-yl)phenyl)-1-hydroxy-5- | | | | |
| | methoxypentyl)morpholino)methanone | | | | |
| 53 | ((1S,3R,4S)-3-amino-4- | 8 | 1 | 2.07 | 568.2 |
| | hydroxycyclopentyl)(2-(1-(3-chloro-2- | | | | |
| | (quinolin-3-yl)phenyl)-1-hydroxy-5- | | | | |
| | methoxypentyl)morpholino)methanone | | | | |
| 54 | ((1S,3R,4S)-3-amino-4- | 8 | 1 | 2.09 | 568.2 |
| | hydroxycyclopentyl)((2R)-2-((1R)-1-(3-chloro- | | | | |
| | 2-(isoquinolin-4-yl)phenyl)-1-hydroxy-5- | | | | |
| | methoxypentyl)morpholino)methanone | | | | |
| 55 | methyl 4-(6-chloro-3′-ethylbiphenyl-2-yl)- | 6 | 2 | 1.80 | 570.5 |
| | 4-hydroxy-4-((R)-1-((1R,3S)-3- | | | | |
| | (methylamino)cyclopentanecarbonyl)piperidin- | | | | |
| | 3-yl)butylcarbamate | | | | |
| 56 | methyl 4-((R)-1-((1R,3S)-3- | 2 | 1 | 2.40 | 570.2 |
| | aminocyclopentanecarbonyl)piperidin-3-yl)-4- | | | | |
| | (6-chloro-3′-isopropylbiphenyl-2-yl)-4- | | | | |
| | hydroxybutylcarbamate | | | | |
| 57 | methyl 4-((R)-1-((1S,3R,4S)-3-amino-4- | 8 | 1 | 2.35 | 572.3 |
| | hydroxycyclopentanecarbonyl)piperidin-3-yl)- | | | | |
| | 4-(6-chloro-3′-ethylbiphenyl-2-yl)-4- | | | | |
| | hydroxybutylcarbamate | | | | |
| 58 | methyl 4-(6-chloro-3′-methoxybiphenyl-2- | 4 | 1 | 2.40 | 572.2 |
| | yl)-4-hydroxy-4-((R)-1-((1R,3S)-3- | | | | |
| | (methylamino)cyclopentanecarbonyl)piperidin- | | | | |
| | 3-yl)butylcarbamate | | | | |
| 59 | ((1S,3R,4S)-3-amino-4- | 8 | 1 | 2.31 | 577.3 |
| | hydroxycyclopentyl)((R)-2-((R)-1-(6-chloro- | | | | |
| | 3′,5′-dimethoxybiphenyl-2-yl)-1-hydroxy-5- | | | | |
| | methoxypentyl)morpholino)methanone | | | | |
| 60 | methyl 4-((R)-1-(trans-4- | 6 | 1 | 2.43 | 584.3 |
| | (aminomethyl)cyclohexanecarbonyl)piperidin- | | | | |
| | 3-yl)-4-(6-chloro-3′-ethylbiphenyl-2-yl)-4- | | | | |
| | hydroxybutylcarbamate | | | | |
| 61 | methyl 4-((R)-1-((1S,3R,4S)-3-amino-4- | 8 | 1 | 2.37 | 586.2 |
| | hydroxycyclopentanecarbonyl)piperidin-3-yl)- | | | | |
| | 4-(6-chloro-3′-isopropylbiphenyl-2-yl)-4- | | | | |
| | hydroxybutylcarbamate | | | | |
| 62 | ((1R,3S)-3-aminocyclopentyl)((R)-3-((S)- | 2 | 1 | 2.61 | 595.3 |
| | 1-(3′-ethoxy-6-fluoro-5′- | | | | |
| | (trifluoromethyl)biphenyl-2-yl)-1-hydroxy-5- | | | | |
| | methoxypentyl)piperidin-1-yl)methanone | | | | |
| 63 | methyl 4-((R)-1-((1S,3R,4S)-3-amino-4- | 8 | 1 | 2.34 | 595.2 |
| | hydroxycyclopentanecarbonyl)piperidin-3-yl)- | | | | |
| | 4-(3-chloro-2-(quinolin-3-yl)phenyl)-4- | | | | |
| | hydroxybutylcarbamate | | | | |
| 64 | ((1R,3S)-3-aminocyclopentyl)((R)-2-((R)- | 2 | 1 | 2.54 | 597.2 |
| | 1-(3′-ethoxy-6-fluoro-5′- | | | | |
| | (trifluoromethyl)biphenyl-2-yl)-1-hydroxy-5- | | | | |
| | methoxypentyl)morpholino)methanone | | | | |
| 65 | ((1S,3R,4S)-3-amino-4- | 8 | 1 | 2.54 | 611.3 |
| | hydroxycyclopentyl)((R)-3-((S)-1-(3′-ethoxy-6- | | | | |
| | fluoro-5′-(trifluoromethyl)biphenyl-2-yl)-1- | | | | |
| | hydroxy-5-methoxypentyl)piperidin-1- | | | | |
| | yl)methanone | | | | |
| 66 | ((1S,3R,4S)-3-amino-4- | 8 | 1 | 2.50 | 613.2 |
| | hydroxycyclopentyl)(2-(1-(3′-ethoxy-6-fluoro- | | | | |
| | 5′-(trifluoromethyl)biphenyl-2-yl)-1-hydroxy-5- | | | | |
| | methoxypentyl)morpholino)methanone | | | | |
| 67 | ((1R,3S)-3-aminocyclopentyl)((R)-2-((R)- | 2 | 1 | 2.21 | 545.2 |
| | 1-(6-fluoro-3′,5′-dimethoxybiphenyl-2-yl)-1- | | | | |
| | hydroxy-5- | | | | |
| | methoxypentyl)morpholino)methanone | | | | |
| 68 | ((1R,3S)-3-aminocyclopentyl)((2R)-2- | 2 | 1 | 2.07 | 552.2 |
| | ((1R)-1-(3-chloro-2-(isoquinolin-4-yl)phenyl)- | | | | |
| | 1-hydroxy-5- | | | | |
| | methoxypentyl)morpholino)methanone | | | | |
| 69 | methyl (4-((3R)-1-{[(1R,3S)-3- | 2 | 3 | 2.65; 2.78 | 558.2 |
| | aminocyclopentyl]carbonyl}-3-piperidinyl)-4- | | | | |
| | {3-chloro-2-[(2-methylphenyl)oxy]phenyl}-4- | | | | |
| | hydroxybutyl)carbamate | | | | |
| 70 | methyl (4-((3R)-1-{[(1R,3S)-3- | 2 | 3 | 2.78; 2.92 | 572.3 |
| | aminocyclopentyl]carbonyl}-3-piperidinyl)-4- | | | | |
| | {3-chloro-2-[(2-ethylphenyl)oxy]phenyl}-4- | | | | |
| | hydroxybutyl)carbamate | | | | |
| 71 | methyl (4-((3R)-1-{[(1S,3R,4S)-3-amino- | 8 | 3 | 2.61; 2.70 | 574.2 |
| | 4-hydroxycyclopentyl]carbonyl}-3-piperidinyl)- | | | | |
| | 4-{3-chloro-2-[(2-methylphenyl)oxy]phenyl}-4- | | | | |
| | hydroxybutyl)carbamate | | | | |
| 72 | methyl (4-((3R)-1-{[(1S,3R,4S)-3-amino- | 8 | 3 | 2.75; 2.86 | 588.2 |
| | 4-hydroxycyclopentyl]carbonyl}-3-piperidinyl)- | | | | |
| | 4-{3-chloro-2-[(2-ethylphenyl)oxy]phenyl}-4- | | | | |
| | hydroxybutyl)carbamate | | | | |
| 73 | methyl [4-((3R)-1-{[(1S,3R,4S)-3-amino- | 8 | 1 | 2.36 | 556.3 |
| | 4-hydroxycyclopentyl]carbonyl}-3-piperidinyl)- | | | | |
| | 4-(3′-ethyl-6-fluoro-2-biphenylyl)-4- | | | | |
| | hydroxybutyl]carbamate | | | | |
| 74 | methyl {4-(6-chloro-3′-ethyl-2-biphenylyl)- | 6 | 1 | 2.46 | 598.3 |
| | 4-hydroxy-4-[(3R)-1-({trans-4- | | | | |
| | [(methylamino)methyl]cyclohexyl}carbonyl)-3- | | | | |
| | piperidinyl]butyl}carbamate | | | | |
| 75 | methyl [4-((3R)-1-{[(1R,2S)-2- | 6 | 1 | 2.39 | 540.3 |
| | aminocyclopentyl]carbonyl}-3-piperidinyl)-4- | | | | |
| | (3′-ethyl-6-fluoro-2-biphenylyl)-4- | | | | |
| | hydroxybutyl]carbamate | | | | |
| 76 | (1R)-1-((2R)-4-{[6-(aminomethyl)-3- | 4 | 1 | 2.42 | 552.2 |
| | pyridinyl]carbonyl}-2-morpholinyl)-1-(6-chloro- | | | | |
| | 3′-ethyl-2-biphenylyl)-5-(methyloxy)-1- | | | | |
| | pentanol | | | | |
| 77 | methyl [4-((3R)-1-{[6-(aminomethyl)-3- | 4 | 1 | 2.40 | 579.2 |
| | pyridinyl]carbonyl}-3-piperidinyl)-4-(6-chloro- | | | | |
| | 3′-ethyl-2-biphenylyl)-4- | | | | |
| | hydroxybutyl]carbamate | | | | |
| 78 | 1-{(3R)-1-[(cis-4- | 6 | 1 | 2.55 | 555.3 |
| | aminocyclohexyl)acetyl]-3-piperidinyl}-1-(6- | | | | |
| | chloro-3′-ethyl-2-biphenylyl)-5-(methyloxy)-1- | | | | |
| | pentanol | | | | |
| 79 | (1S)-1-{(3R)-1-[(trans-4- | 6 | 1 | 2.55 | 555.3 |
| | aminocyclohexyl)acetyl]-3-piperidinyl}-1-(6- | | | | |
| | chloro-3′-ethyl-2-biphenylyl)-5-(methyloxy)-1- | | | | |
| | pentanol | | | | |
| 80 | 1-((3R)-1-{[(1R,2S)-2- | 6 | 1 | 2.63 | 527.3 |
| | aminocyclopentyl]carbonyl}-3-piperidinyl)-1- | | | | |
| | (6-chloro-3′-ethyl-2-biphenylyl)-5-(methyloxy)- | | | | |
| | 1-pentanol | | | | |
| 81 | (1R)-1-(6-chloro-3′-ethyl-2-biphenylyl)-1- | 6 | 1 | 2.48 | 543.2 |
| | ((2R)-4-{[(1R,3S)-3- | | | | |
| | (methylamino)cyclopentyl]carbonyl}-2- | | | | |
| | morpholinyl)-5-(methyloxy)-1-pentanol | | | | |
| 82 | 1-(6-chloro-3′-ethyl-2-biphenylyl)-1-((3R)- | 6 | 1 | 2.52 | 541.3 |
| | 1-{[(1R,3S)-3- | | | | |
| | (methylamino)cyclopentyl]carbonyl}-3- | | | | |
| | piperidinyl)-5-(methyloxy)-1-pentanol | | | | |
| 83 | (1S,2R,4S)-4-({(2R)-2-[(1R)-1-(6-chloro- | 10 | 1 | 2.39 | 589.2 |
| | 3′-ethyl-2-biphenylyl)-1-hydroxy-5- | | | | |
| | (methyloxy)pentyl]-4-morpholinyl}carbonyl)-2- | | | | |
| | [(2-hydroxyethyl)aminolcyclopentanol | | | | |
| 84 | methyl {4-(6-chloro-3′-ethyl-2-biphenylyl)- | 9 | 1 | 2.49 | 616.2 |
| | 4-hydroxy-4-[(3R)-1-({(1S,3S,4R)-3-hydroxy- | | | | |
| | 4-[(2- | | | | |
| | hydroxyethyl)amino]cyclopentyl}carbonyl)-3- | | | | |
| | piperidinyl]butyl}carbamate | | | | |
| 85 | 1-(6-chloro-3′-ethyl-2-biphenylyl)-1-[(3R)- | 6 | 1 | 2.45 | 569.3 |
| | 1-({trans-4- | | | | |
| | [(methylamino)methyl]cyclohexyl}carbonyl)-3- | | | | |
| | piperidinyl]-5-(methyloxy)-1-pentanol | | | | |
| 86 | (1S,2R,4S)-4-({(2R)-2-[(1R)-1-(6-chloro- | 10 | 1 | 2.53 | 642.2 |
| | 3′-ethyl-2-biphenylyl)-1-hydroxy-5- | | | | |
| | (methyloxy)pentyl]-4-morpholinyl}carbonyl)-2- | | | | |
| | [(1,3-thiazol-2-ylmethyl)amino]cyclopentanol | | | | |
| 87 | methyl [4-((3R)-1-{[(1R,3S)-3- | 6 | 1 | 2.47 | 570.2 |
| | aminocyclohexyl]carbonyl}-3-piperidinyl)-4-(6- | | | | |
| | chloro-3′-ethyl-2-biphenylyl)-4- | | | | |
| | hydroxybutyl]carbamate | | | | |
| 88 | N-{4-(6-chloro-3′-methyl-2-biphenylyl)-4- | 6 | 1 | 2.46 | 568.3 |
| | hydroxy-4-[(3R)-1-({trans-4- | | | | |
| | [(methylamino)methyl]cyclohexyl}carbonyl)-3- | | | | |
| | piperidinyl]butyl}acetamide | | | | |
| 89 | N-[4-(6-chloro-3′-methyl-2-biphenylyl)-4- | 6 | 1 | 2.44 | 540.3 |
| | hydroxy-4-((3R)-1-{[(1R,3S)-3- | | | | |
| | (methylamino)cyclopentyl]carbonyl}-3- | | | | |
| | piperidinyl)butyl]acetamide | | | | |
| 90 | (1R)-1-(6-chloro-3′-ethyl-2-biphenylyl)-1- | 10 | 1 | 2.53 | 638.3 |
| | {(2R)-4-[((1R,3S)-3-{[(2,5-dimethyl-1,3-oxazol- | | | | |
| | 4-yl)methyl]amino}cyclopentyl)carbonyl]-2- | | | | |
| | morpholinyl}-5-(methyloxy)-1-pentanol | | | | |
| 91 | N-[4-((3R)-1-{[trans-4- | 6 | 1 | 2.42 | 554.3 |
| | (aminomethyl)cyclohexyl]carbonyl}-3- | | | | |
| | piperidinyl)-4-(6-chloro-3′-methyl-2- | | | | |
| | biphenylyl)-4-hydroxybutyl]acetamide | | | | |
| 92 | methyl {4-(4,6-difluoro-3′-methyl-2- | 6 | 1 | 2.43 | 586.3 |
| | biphenylyl)-4-hydroxy-4-[(3R)-1-({trans-4- | | | | |
| | [(methylamino)methyl]cyclohexyl}carbonyl)-3- | | | | |
| | piperidinyl]butyl}carbamate | | | | |
| 93 | methyl [4-hydroxy-4-((3R)-1-{[(1R,3S)-3- | 6 | 1 | 2.51 | 576.3 |
| | (methylamino)cyclopentyl]carbonyl}-3- | | | | |
| | piperidinyl)-4-(2′,4,6-trifluoro-5′-methyl-2- | | | | |
| | biphenylyl)butyl]carbamate | | | | |
| 94 | methyl [4-hydroxy-4-[(3R)-1-({trans-4- | 6 | 1 | 2.38 | 604.3 |
| | [(methylamino)methyl]cyclohexyl}carbonyl)-3- | | | | |
| | piperidinyl]-4-(2′,4,6-trifluoro-5′-methyl-2- | | | | |
| | biphenylyl)butyl]carbamate | | | | |
| 95 | methyl [4-(6-chloro-3′-methyl-2- | 6 | 1 | 2.39 | 556.3 |
| | biphenylyl)-4-hydroxy-4-((3R)-1-{[(1R,3 S)-3- | | | | |
| | (methylamino)cyclopentyl]carbonyl}-3- | | | | |
| | piperidinyl)butyl]carbamate | | | | |
| 96 | (1R)-1-(6-chloro-3′-ethyl-2-biphenylyl)-1- | 6 | 1 | 2.53 | 571.3 |
| | [(2R)-4-({trans-4- | | | | |
| | [(methylamino)methyl]cyclohexyl}carbonyl)-2- | | | | |
| | morpholinyl]-5-(methyloxy)-1-pentanol | | | | |
| 97 | methyl [(4R)-4-((2R)-4-{[(1S,3R,4S)-3- | 8 | 1 | 2.44 | 558.3 |
| | amino-4-hydroxycyclopentyl]carbonyl}-2- | | | | |
| | morpholinyl)-4-(3′-ethyl-6-fluoro-2-biphenylyl)- | | | | |
| | 4-hydroxybutyl]carbamate | | | | |
| 98 | methyl {(4R)-4-(3′-ethyl-6-fluoro-2- | 6 | 1 | 2.18 | 584.3 |
| | biphenylyl)-4-hydroxy-4-[(2R)-4-({trans-4- | | | | |
| | [(methylamino)methyl]cyclohexyl}carbonyl)-2- | | | | |
| | morpholinyl]butyl}carbamate | | | | |
| 99 | (1S,2R,4S)-2-amino-4-({(2R)-2-[(1R)-1- | 8 | 1 | 2.3 | 546.2 |
| | [2-chloro-3-(3-ethylphenyl)-4-pyridinyl]-1- | | | | |
| | hydroxy-5-(methyloxy)pentyl]-4- | | | | |
| | morpholinyl}carbonyl)cyclopentanol | | | | |
| 100 | (1R)-1-((2R)-4-{[(1R,3S)-3- | 2 | 1 | 2.37 | 530.2 |
| | aminocyclopentyl]carbonyl}-2-morpholinyl)-1- | | | | |
| | [2-chloro-3-(3-ethylphenyl)-4-pyridinyl]-5- | | | | |
| | (methyloxy)-1-pentanol | | | | |
| 101 | (1S,2R,4S)-2-amino-4-({(2R)-2-[(1R)-1- | 8 | 1 | 2.43 | 573.2 |
| | [2-(1-benzothien-3-yl)-3-chlorophenyl]-1- | | | | |
| | hydroxy-5-(methyloxy)pentyl]-4- | | | | |
| | morpholinyl}carbonyl)cyclopentanol | | | | |
| 102 | (1R)-1-((2R)-4-{[(1R,3S)-3- | 2 | 1 | 2.42 | 557.2 |
| | aminocyclopentyl]carbonyl}-2-morpholinyl)-1- | | | | |
| | [2-(1-benzothien-3-yl)-3-chlorophenyl]-5- | | | | |
| | (methyloxy)-1-pentanol | | | | |
| 103 | (1R)-1-[4-chloro-3-(3-ethylphenyl)-2- | 6 | 1 | 2.14 | 544.3 |
| | pyridinyl]-1-((2R)-4-{[(1R,3S)-3- | | | | |
| | (methylamino)cyclopentyl]carbonyl}-2- | | | | |
| | morpholinyl)-5-(methyloxy)-1-pentanol | | | | |
| 104 | methyl {4-[2-chloro-3-(3-ethylphenyl)-4- | 6 | 1 | 2.33 | 599.3 |
| | pyridinyl]-4-hydroxy-4-[(3R)-1-({trans-4- | | | | |
| | [(methylamino)methyl]cyclohexyl}carbonyl)-3- | | | | |
| | piperidinyl]butyl}carbamate | | | | |
| 105 | methyl [4-[2-chloro-3-(3-ethylphenyl)-4- | 6 | 1 | 2.49 | 571.2 |
| | pyridinyl]-4-hydroxy-4-((3R)-1-{[(1R,3S)-3- | | | | |
| | (methylamino)cyclopentyl]carbonyl}-3- | | | | |
| | piperidinyl)butyl]carbamate | | | | |
| 106 | methyl {4-{3-chloro-2-[4-(1-methylethyl)- | 6 | 1 | 2.42 | 664.3 |
| | 2-quinazolinyl]phenyl}-4-hydroxy-4-[(3R)-1- | | | | |
| | ({trans-4- | | | | |
| | [(methylamino)methyl]cyclohexyl}carbonyl)-3- | | | | |
| | piperidinyl]butyl}carbamate | | | | |
| 107 | methyl [4-{3-chloro-2-[4-(1-methylethyl)- | 6 | 1 | 2.45 | 636.3 |
| | 2-quinazolinyl]phenyl}-4-hydroxy-4-((3R)-1- | | | | |
| | {[(1R,3S)-3- | | | | |
| | (methylamino)cyclopentyl]carbonyl}-3- | | | | |
| | piperidinyl)butyl]carbamate | | | | |
| 108 | 1-((3R)-1-{[(1R,3S)-3- | 2 | 3 | 3.01 | 529.2 |
| | aminocyclopentyl]carbonyl}-3-piperidinyl)-1- | | | | |
| | {3-chloro-2-[(3-methylphenyl)oxy]phenyl}-5- | | | | |
| | (methyloxy)-1-pentanol | | | | |
| 109 | (1S,2R,4S)-2-amino-4-({(3R)-3-[1-{3- | 8 | 3 | 2.87 | 545.2 |
| | chloro-2-[(3-methylphenyl)oxy]phenyl}-1- | | | | |
| | hydroxy-5-(methyloxy)pentyl]-1- | | | | |
| | piperidinyl}carbonyl)cyclopentanol | | | | |
| 110 | methyl (4-((3R)-1-{[(1R,3S)-3- | 2 | 3 | 3.4 | 558.1 |
| | aminocyclopentyl]carbonyl}-3-piperidinyl)-4- | | | | |
| | {3-chloro-2-[(3-methylphenyl)oxy]phenyl}-4- | | | | |
| | hydroxybutyl)carbamate | | | | |
| 111 | methyl (4-((3R)-1-{[(1S,3R,4S)-3-amino- | 8 | 3 | 3.31 | 574.1 |
| | 4-hydroxycyclopentyl]carbonyl}-3-piperidinyl)- | | | | |
| | 4-{3-chloro-2-[(3-methylphenyl)oxy]phenyl}-4- | | | | |
| | hydroxybutyl)carbamate | | | | |
| 112 | methyl (4-((3R)-1-{[(1R,3S)-3- | 2 | 3 | 3.52 | 538.3 |
| | aminocyclopentyl]carbonyl}-3-piperidinyl)-4- | | | | |
| | {2-[(2,6-dimethylphenyl)oxy]phenyl}-4- | | | | |
| | hydroxybutyl)carbamate | | | | |
| 113 | methyl (4-((3R)-1-{[(1S,3R,4S)-3-amino- | 8 | 3 | 3.41 | 554.2 |
| | 4-hydroxycyclopentyl]carbonyl}-3-piperidinyl)- | | | | |
| | 4-{2-[(2,6-dimethylphenyl)oxy]phenyl}-4- | | | | |
| | hydroxybutyl)carbamate | | | | |
| 114 | methyl {4-{3-chloro-2-[8-(1-methylethyl)- | 6 | 3 | 1.88 | 663.2 |
| | 2-quinolinyl]phenyl}-4-hydroxy-4-[(3R)-1- | | | | |
| | ({trans-4- | | | | |
| | [(methylamino)methyl]cyclohexyl}carbonyl)-3- | | | | |
| | piperidinyl]butyl}carbamate | | | | |
| 115 | methyl {4-[3-chloro-2-(8-methyl-2- | 6 | 3 | 1.67 | 635.2 |
| | quinolinyl)phenyl]-4-hydroxy-4-[(3R)-1-({trans- | | | | |
| | 4-[(methylamino)methyl]cyclohexyl}carbonyl)- | | | | |
| | 3-piperidinyl]butyl}carbamate | | | | |
| 116 | methyl [4-[3-fluoro-2-(3- | 6 | 1 | 2.28 | 577.3 |
| | quinolinyl)phenyl]-4-hydroxy-4-((3R)-1- | | | | |
| | {[(1R,3S)-3- | | | | |
| | (methylamino)cyclopentyl]carbonyl}-3- | | | | |
| | piperidinyl)butyl]carbamate | | | | |
| 117 | methyl [4-{2-chloro-3-[3-(1- | 6 | 1 | 2.37 | 585.3 |
| | methylethyl)phenyl]-4-pyridinyl}-4-hydroxy-4- | | | | |
| | ((3R)-1-{[(1R,3S)-3- | | | | |
| | (methylamino)cyclopentyl]carbonyl}-3- | | | | |
| | piperidinyl)butyl]carbamate | | | | |
| 118 | methyl [4-[3-chloro-2-(5-methyl-2- | 6 | 1 | 2.52 | 546.2 |
| | furanyl)phenyl]-4-hydroxy-4-((3R)-1-{[(1R,3S)- | | | | |
| | 3-(methylamino)cyclopentyl]carbonyl}-3- | | | | |
| | piperidinyl)butyl]carbamate | | | | |
| 119 | methyl {4-((3R)-1-{[(1S,3R,4S)-3-amino- | 8 | 1 | 2.36 | 548.2 |
| | 4-hydroxycyclopentyl]carbonyl}-3-piperidinyl)- | | | | |
| | 4-[3-chloro-2-(5-methyl-2-furanyl)phenyl]-4- | | | | |
| | hydroxybutyl}carbamate | | | | |
| 120 | (1R)-1-{2-chloro-3-[3-(1- | 6 | 1 | 2.27 | 586.2 |
| | methylethyl)phenyl]-4-pyridinyl}-1-[(2R)-4- | | | | |
| | ({trans-4- | | | | |
| | [(methylamino)methyl]cyclohexyl}carbonyl)-2- | | | | |
| | morpholinyl]-5-(methyloxy)-1-pentanol | | | | |
| 121 | (1R)-1-{2-chloro-3-[3-(1- | 6 | 1 | 2.27 | 558.2 |
| | methylethyl)phenyl]-4-pyridinyl}-1-((2R)-4- | | | | |
| | {[(1R,3S)-3- | | | | |
| | (methylamino)cyclopentyl]carbonyl}-2- | | | | |
| | morpholinyl)-5-(methyloxy)-1-pentanol | | | | |
| 122 | (1S,2R,4S)-2-amino-4-({(2R)-2-[(1R)-1- | 8 | 1 | 2.2 | 560.2 |
| | {2-chloro-3-[3-(1-methylethyl)phenyl]-4- | | | | |
| | pyridinyl}-1-hydroxy-5-(methyloxy)pentyl]-4- | | | | |
| | morpholinyl}carbonyl)cyclopentanol | | | | |
| 123 | methyl {4-[5-chloro-4-(3-ethylphenyl)-3- | 6 | 1 | 2.14 | 599.3 |
| | pyridinyl]-4-hydroxy-4-[(3R)-1-({trans-4- | | | | |
| | [(methylamino)methyl]cyclohexyl}carbonyl)-3- | | | | |
| | piperidinyl]butyl}carbamate | | | | |
| 124 | methyl [4-[5-chloro-4-(3-ethylphenyl)-3- | 6 | 1 | 2.14 | 571.2 |
| | pyridinyl]-4-hydroxy-4-((3R)-1-{[(1R,3S)-3- | | | | |
| | (methylamino)cyclopentyl]carbonyl}-3- | | | | |
| | piperidinyl)butyl]carbamate | | | | |
| 125 | methyl {4-{5-chloro-4-[3-(1- | 6 | 1 | 2.2 | 613.2 |
| | methylethyl)phenyl]-3-pyridinyl}-4-hydroxy-4- | | | | |
| | [(3R)-1-({trans-4- | | | | |
| | [(methylamino)methyl]cyclohexyl}carbonyl)-3- | | | | |
| | piperidinyl]butyl}carbamate | | | | |
| 126 | methyl [4-{5-chloro-4-[3-(1- | 6 | 1 | 2.21 | 585.3 |
| | methylethyl)phenyl]-3-pyridinyl}-4-hydroxy-4- | | | | |
| | ((3R)-1-{[(1R,3S)-3- | | | | |
| | (methylamino)cyclopentyl]carbonyl}-3- | | | | |
| | piperidinyl)butyl]carbamate | | | | |
| 127 | methyl {4-[6-fluoro-3′-(1-methylethyl)-2- | 6 | 1 | 2.44 | 596.3 |
| | biphenylyl]-4-hydroxy-4-[(3R)-1-({trans-4- | | | | |
| | [(methylamino)methyl]cyclohexyl}carbonyl)-3- | | | | |
| | piperidinyl]butyl}carbamate | | | | |
| 128 | methyl [4-(6-chloro-3′-fluoro-5′-methyl-2- | 6 | 1 | 2.26 | 574.3 |
| | biphenylyl)-4-hydroxy-4-((3R)-1-{[(1R,3S)-3- | | | | |
| | (methylamino)cyclopentyl]carbonyl}-3- | | | | |
| | piperidinyl)butyl]carbamate | | | | |
| 129 | methyl [4-(3′,6-difluoro-5′-methyl-2- | 6 | 1 | 2.36 | 558.3 |
| | biphenylyl)-4-hydroxy-4-((3R)-1-{[(1R,3S)-3- | | | | |
| | (methylamino)cyclopentyl]carbonyl}-3- | | | | |
| | piperidinyl)butyl]carbamate | | | | |
| 130 | (1S,2R,4S)-2-amino-4-({(3R)-3-[1-{3- | 8 | 3 | 3.23 | 559.2 |
| | chloro-2-[(2-ethylphenyl)oxy]phenyl}-1- | | | | |
| | hydroxy-5-(methyloxy)pentyl]-1- | | | | |
| | piperidinyl}carbonyl)cyclopentanol | | | | |
| 131 | methyl {4-(3′-ethyl-6-fluoro-2-biphenylyl)- | 6 | 1 | 2.43 | 582.3 |
| | 4-hydroxy-4-[(3R)-1-({trans-4- | | | | |
| | [(methylamino)methyl]cyclohexyl}carbonyl)-3- | | | | |
| | piperidinyl]butyl}carbamate | | | | |
| 132 | (1S,2R,4S)-2-amino-4-({(3R)-3-[1-{2-[(2- | 8 | 3 | 3.21 | 545.1 |
| | chloro-6-methylphenyl)oxy]phenyl}-1-hydroxy- | | | | |
| | 5-(methyloxy)pentyl]-1- | | | | |
| | piperidinyl}carbonyl)cyclopentanol | | | | |
| 133 | (1S,2R,4S)-2-amino-4-({(3R)-3-[1-{3- | 8 | 3 | 1.66 | 529.2 |
| | fluoro-2-[(3-methylphenyl)oxy]phenyl}-1- | | | | |
| | hydroxy-5-(methyloxy)pentyl]-1- | | | | |
| | piperidinyl}carbonyl)cyclopentanol | | | | |
| 134 | 1-((3R)-1-{[(1R,3S)-3- | 2 | 3 | 1.71 | 513.3 |
| | aminocyclopentyl]carbonyl}-3-piperidinyl)-1- | | | | |
| | {3-fluoro-2-[(3-methylphenyl)oxy]phenyl}-5- | | | | |
| | (methyloxy)-1-pentanol | | | | |
| 135 | methyl (4-((3R)-1-{[(1S,3R,4S)-3-amino- | 8 | 3 | 1.71 | 588.2 |
| | 4-hydroxycyclopentyl]carbonyl}-3-piperidinyl)- | | | | |
| | 4-{3-chloro-2-[(3-ethylphenyl)oxy]phenyl}-4- | | | | |
| | hydroxybutyl)carbamate | | | | |
| 136 | methyl (4-((3R)-1-{[(1R,3S)-3- | 2 | 3 | 1.74 | 572.2 |
| | aminocyclopentyl]carbonyl}-3-piperidinyl)-4- | | | | |
| | {3-chloro-2-[(3-ethylphenyl)oxy]phenyl}-4- | | | | |
| | hydroxybutyl)carbamate | | | | |
| 137 | methyl (4-((3R)-1-{[(1S,3R,4S)-3-amino- | 8 | 3 | 1.57 | 558.2 |
| | 4-hydroxycyclopentyl]carbonyl}-3-piperidinyl)- | | | | |
| | 4-{3-fluoro-2-[(3-methylphenyl)oxy]phenyl}-4- | | | | |
| | hydroxybutyl)carbamate | | | | |
| 138 | methyl (4-((3R)-1-{[(1R,3S)-3- | 2 | 3 | 1.62 | 542.3 |
| | aminocyclopentyl]carbonyl}-3-piperidinyl)-4- | | | | |
| | {3-fluoro-2-[(3-methylphenyl)oxy]phenyl}-4- | | | | |
| | hydroxybutyl)carbamate | | | | |
| 139 | methyl (4-((3R)-1-{[(1S,3R,4S)-3-amino- | 8 | 1 | 2.33 | 572.2 |
| | 4-hydroxycyclopentyl]carbonyl}-3-piperidinyl)- | | | | |
| | 4-{3-chloro-2-[(3- | | | | |
| | methylphenyl)methyl]phenyl}-4- | | | | |
| | hydroxybutyl)carbamate | | | | |
| 140 | methyl [4-{3-chloro-2-[(3- | 8 | 1 | 2.36 | 570.2 |
| | methylphenyl)methyl]phenyl}-4-hydroxy-4- | | | | |
| | ((3R)-1-{[(1R,3S)-3- | | | | |
| | (methylamino)cyclopentyl]carbonyl}-3- | | | | |
| | piperidinyl)butyl]carbamate | | | | |
| 141 | methyl (4-((3R)-1-{[(1R,3S)-3- | 2 | 1 | 2.4 | 556.2 |
| | aminocyclopentyl]carbonyl}-3-piperidinyl)-4- | | | | |
| | {3-chloro-2-[(3-methylphenyl)methyl]phenyl}- | | | | |
| | 4-hydroxybutyl)carbamate | | | | |
| 142 | methyl (2-{[(S)-((2R)-4-{[(1S,3R,4S)-3- | 8 | 2 | 1.77 | 560.4 |
| | amino-4-hydroxycyclopentyl]carbonyl}-2- | | | | |
| | morpholinyl)(6-chloro-3′-ethyl-2- | | | | |
| | biphenylyl)methyl]oxy}ethyl)carbamate | | | | |
| 143 | methyl (2-{[(R)-((3R)-1-{[(1S,3R,4S)-3- | 8 | 2 | 1.84 | 558.8 |
| | amino-4-hydroxycyclopentyl]carbonyl}-3- | | | | |
| | piperidinyl)(6-chloro-3′-ethyl-2- | | | | |
| | biphenylyl)methyl]oxy}ethyl)carbamate | | | | |
| 144 | methyl [4-((3R)-1-{[4-(aminomethyl)-5- | 6 | 1 | 2.5 | 582.2 |
| | methyl-2-furanyl]carbonyl}-3-piperidinyl)-4-(6- | | | | |
| | chloro-3′-ethyl-2-biphenylyl)-4- | | | | |
| | hydroxybutyl]carbamate | | | | |
| 145 | methyl {4-(6-chloro-3′-ethyl-2-biphenylyl)- | 6 | 1 | 2.36 | 593.3 |
| | 4-hydroxy-4-[(3R)-1-({6- | | | | |
| | [(methylamino)methyl]-3-pyridinyl}carbonyl)-3- | | | | |
| | piperidinyl]butyl}carbamate | | | | |
| 146 | methyl [4-{(3R)-1-[(2-amino-4-oxo-1,4- | 6 | 1 | 2.29 | 569.4 |
| | dihydro-5-pyrimidinyl)carbonyl]-3-piperidinyl}- | | | | |
| | 4-(6-chloro-3′-methyl-2-biphenylyl)-4- | | | | |
| | hydroxybutyl]carbamate | | | | |
| 147 | methyl [4-(6-chloro-3′-methyl-2- | 6 | 1 | 2.67 | 566.2 |
| | biphenylyl)-4-hydroxy-4-((3R)-1-{[2- | | | | |
| | (methylamino)-5-pyrimidinyl]carbonyl}-3- | | | | |
| | piperidinyl)butyl]carbamate | | | | |
| 148 | methyl [4-((3R)-1-{[5-(aminomethyl)-3- | 6 | 1 | 2.47 | 555.2 |
| | isoxazolyl]carbonyl}-3-piperidinyl)-4-(6-chloro- | | | | |
| | 3′-methyl-2-biphenylyl)-4- | | | | |
| | hydroxybutyl]carbamate | | | | |
| 149 | methyl {4-(6-chloro-3′-ethyl-2-biphenylyl)- | 6 | 2 | 1.32 | 598.6 |
| | 4-hydroxy-4-[(3R)-1-({5- | | | | |
| | [(methylamino)methyl]-2-thienyl}carbonyl)-3- | | | | |
| | piperidinyl]butyl}carbamate | | | | |
| 150 | methyl [4-{(3R)-1-[(6-amino-3- | 6 | 1 | 2.46 | 553.2 |
| | pyridinyl)carbonyl]-3-piperidinyl}-4-(3′,6- | | | | |
| | difluoro-5′-methyl-2-biphenylyl)-4- | | | | |
| | hydroxybutyl]carbamate | | | | |
| 151 | methyl [2-({(R)-(6-chloro-3′-ethyl-2- | 6 | 2 | 1.46 | 584.6 |
| | biphenylyl)[(3R)-1-({trans-4- | | | | |
| | [(methylamino)methyl]cyclohexyl}carbonyl)-3- | | | | |
| | piperidinyl]methyl}oxy)ethyl]carbamate | | | | |
| 152 | methyl [2-({(S)-(6-chloro-3′-ethyl-2- | 6 | 2 | 1.69 | 586.5 |
| | biphenylyl)[(2R)-4-({trans-4- | | | | |
| | [(methylamino)methyl]cyclohexyl}carbonyl)-2- | | | | |
| | morpholinyl]methyl}oxy)ethyl]carbamate | | | | |
| 153 | N-[4-((3R)-1-{[6-(aminomethyl)-3- | 6 | 1 | 2.42 | 549.2 |
| | pyridinyl]carbonyl}-3-piperidinyl)-4-(6-chloro- | | | | |
| | 3′-methyl-2-biphenylyl)-4- | | | | |
| | hydroxybutyl]acetamide | | | | |
| 154 | methyl 4-((R)-1-(4- | 12 | 1 | 2.47 | 569.3 |
| | (aminomethyl)piperidine-1-carbonyl)piperidin- | | | | |
| | 3-yl)-4-(3′-ethyl-6-fluorobiphenyl-2-yl)-4- | | | | |
| | hydroxybutylcarbamate | | | | |
| 155 | methyl 4-(3′-ethyl-6-fluorobiphenyl-2-yl)- | 14 | 1 | 2.47 | 583.3 |
| | 4-hydroxy-4-((R)-1-(4- | | | | |
| | ((methylamino)methyl)piperidine-1- | | | | |
| | carbonyl)piperidin-3-yl)butylcarbamate | | | | |
| 156 | methyl [4-((3R)-1-{[4-(aminomethyl)-1- | 11 | 1 | 2.5 | 585.3 |
| | piperidinyl]carbonyl}-3-piperidinyl)-4-(6-chloro- | | | | |
| | 3′-ethyl-2-biphenylyl)-4- | | | | |
| | hydroxybutyl]carbamate | | | | |
| 157 | methyl [4-((3R)-1-{[4-(aminomethyl)-4- | 12 | 1 | 2.5 | 661.3 |
| | phenyl-1-piperidinyl]carbonyl}-3-piperidinyl)-4- | | | | |
| | (6-chloro-3′-ethyl-2-biphenylyl)-4- | | | | |
| | hydroxybutyl]carbamate | | | | |
| 158 | N-(4-((R)-1-(4-(aminomethyl)piperidine- | 12 | 1 | 2.42 | 573.3 |
| | 1-carbonyl)piperidin-3-yl)-4-(2′,6-difluoro-5′- | | | | |
| | methylbiphenyl-2-yl)-4-hydroxybutyl)-2- | | | | |
| | hydroxyacetamide | | | | |
| 159 | methyl {4-(6-chloro-3′-ethyl-2-biphenylyl)- | 13 | 1 | 2.4 | 599.3 |
| | 4-hydroxy-4-[(3R)-1-({4- | | | | |
| | [(methylamino)methyl]-1-piperidinyl}carbonyl)- | | | | |
| | 3-piperidinyl]butyl}carbamate | | | | |
| 160 | methyl {4-(6-chloro-3′-ethyl-2-biphenylyl)- | 14 | 1 | 2.51 | 625.3 |
| | 4-hydroxy-4-[(3R)-1-({4-[(2-propen-1- | | | | |
| | ylamino)methyl]-1-piperidinyl}carbonyl)-3- | | | | |
| | piperidinyl]butyl}carbamate | | | | |

^{a}Minor isomer separated by chromatography

### Example 15

### In Vitro Activity Studies

The compounds of the invention have enzyme-inhibiting properties. In particular, they inhibit the action of the natural enzyme renin. The latter passes from the kidneys into the blood where it affects the cleavage of angiotensinogen, releasing the decapeptide angiotensin I which is then cleaved in the blood, lungs, the kidneys and other organs by angiotensin converting enzyme to form the octapeptide angiotensin II. The octapeptide increases blood pressure both directly by binding to its receptor, causing arterial vasoconstriction, and indirectly by liberating from the adrenal glands the sodium-ion-retaining hormone aldosterone, accompanied by an increase in extracellular fluid volume. That increase can be attributed to the action of angiotensin II. Inhibitors of the enzymatic activity of renin bring about a reduction in the formation of angiotensin I. As a result a smaller amount of angiotensin II is produced. The reduced concentration of that active peptide hormone is the direct cause of the hypotensive effect of renin inhibitors.

The action of renin inhibitors in vitro is demonstrated experimentally by means of a test which measures the increase in fluorescence of an internally quenched peptide substrate. The sequence of this peptide corresponds to the sequence of human angiotensinogen. The following test protocol is used: All reactions are carried out in a flat bottom white opaque microtiter plate. A 4 μL aliquot of 400 μM renin substrate (DABCYL-γ-Abu-Ile-His-Pro-Phe-His-Leu-Val-Ile-His-Thr-EDANS) in 192 μL assay buffer (50 mM BES, 150 mM NaCl, 0.25 mg/mL bovine serum albumin, pH7.0) is added to 4 μL of test compound in DMSO at various concentrations ranging from 10 μM to 1 nM final concentrations. Next, 100 μL of trypsin-activated recombinant human renin (final enzyme concentration of 0.2-2 nM) in assay buffer is added, and the solution is mixed by pipetting. The increase in fluorescence at 495 nm (excitation at 340 nm) is measured for 60-360 min at rt using a Perkin-Elmer Fusion microplate reader. The slope of a linear portion of the plot of fluorescence increase as a function of time is then determined, and the rate is used for calculating percent inhibition in relation to uninhibited control. The percent inhibition values are plotted as a function of inhibitor concentration, and the IC₅₀ is determined from a fit of this data to a four parameter equation. The IC₅₀ is defined as the concentration of a particular inhibitor that reduces the formation of product by 50% relative to a control sample containing no inhibitor. (Wang G. T. et al. Anal. Biochem. 1993, 210, 351; Nakamura, N. et al. J. Biochem. (Tokyo) 1991, 109, 741; Murakami, K. et al. Anal Biochem. 1981, 110, 232).

In this in vitro systems the compounds of the invention (Compound #1-160) exhibit 50% inhibition at concentrations of from approximately 5000 nM to approximately 0.01 nM. Preferred compounds of the invention exhibit 50% inhibition at concentrations of from approximately 50 n M to approximately 0.01 nM. More preferred compounds of the invention exhibit 50% inhibition at concentrations of from approximately 5 nM to approximately 0.01 nM. Highly preferred compounds of the invention exhibit 50% inhibition at concentrations of from approximately 5 nM to approximately 0.01 nM and exhibit 50% inhibition at concentrations of from approximately 10 nM to approximately 0.01 nM in the in vitro assay in the presence of human plasma described below.

### Example 16

### In Vitro Activity Studies

All reactions are carried out in a low volume, black, 384 well microtiter plate (greiner bio-one). Compounds were diluted in 100% DMSO, and a 100mL aliquot of each compound concentration was stamped into the plate using a Hummingbird (Genomic Solutions). 5 μL of 600 pM renin (trypsin-activated recombinant human renin) was then added to the plate, followed by 5 μL of 2 μM substrate (Arg-Glu-Lys(5-FAM)-Ile-His-Pro-Phe-His-Leu-Val-Ile-His-Thr-Lys(5,6-TAMRA)-Arg-CONH₂). Both renin and substrate were made up in buffer containing 50 mM HEPES, 125 mM NaCl, 0.1% CHAPS, with the pH adjusted to 7.4. After 2 hours of reaction at room temperature, the plates were read on a Viewlux (PerkinElmer) with an excitation/emission of 485/530 nm, and using a 505 nm cutoff filter. The percent inhibition values are plotted as a function of inhibitor concentration, and the IC₅₀ is determined from a fit of this data to a four parameter equation. The IC₅₀ is defined as the concentration of a particular inhibitor that reduces the formation of product by 50% relative to a control sample containing no inhibitor. In the in vitro systems the compounds of the invention exhibit inhibiting activities at minimum concentrations of from approximately 5×10⁻⁵ M to approximately 10⁻¹² M. Preferred compounds of the invention exhibit inhibiting activities at minimum concentrations of from approximately 10⁻⁷ M to approximately 10⁻¹² M.

### Example 17

### In Vitro Activity Studies

The potency of renin inhibitors was measured using an in vitro renin assay. In this assay, renin-catalyzed proteolysis of a fluorescently labeled peptide converts the peptide from a weakly fluorescent to a strongly fluorescent molecule. The following test protocol was used. Substrate solution (5 μl; 2 μM Arg-Glu-Lys(5-Fam)-Ile-His-Pro-Phe-His-Leu-Val-Ile-His-Thr-Lys(5,6 Tamra)-Arg-CONH₂ in 50 mM Hepes, 125 mM NaCl, 0.1% CHAPS, pH 7.4) then trypsin-activated recombinant human renin (Scott, Martin J. et. al. Protein Expression and Purification 2007, 52(1), 104-116; 5 μL; 600 μM renin in 50 mM Hepes, 125 mM NaCl, 0.1% CHAPS, pH 7.4) were added sequentially to a black Greiner low volume 384-well plate (cat.#784076) pre-stamped with a 100 nl DMSO solution of compound at the desired concentration. The assay plates were incubated at room temperature for 2 hours with a cover plate then quenched by the addition of a stop solution (2 μL; 5 μM of Bachem C-3195 in 50 mM Hepes, 125 mM NaCl, 0.1% CHAPS, pH 7.4, 10% DMSO). The assay plates were read on an LJL Acquest using a 485 nm excitation filter, a 530 nm emission filter, and a 505 nm dichroic filter. Compounds were initially prepared in neat DMSO at a concentration of 10 mM. For inhibition curves, compounds were diluted using a three fold serial dilution and tested at 11 concentrations (e.g. 50 μM-0.8 nM or 25 μM-0.42 nM or 2.5 μM to 42 μM). Curves were analyzed using ActivityBase and XLfit, and results were expressed as pIC₅₀ values.

### Example 18

### In Vitro Activity of the Disclosed Compounds in Human Plasma

The action of renin inhibitors in vitro in human plasma is demonstrated experimentally by the decrease in plasma renin activity (PRA) levels observed in the presence of the compounds. Incubations mixtures contain in the final volume of 250 μL 95.5 mM N,N-bis(2-hydroxyethyl)-2-aminoethanesulfonic acid, pH 7.0, 8 mM EDTA, 0.1 mM neomycin sulfate, 1 mg/ml sodium azide, 1 mM phenylmethanesulfonyl fluoride, 2% DMSO and 87.3% of pooled mixed-gender human plasma stabilized with EDTA. For plasma batches with low PRA (less than 1 ng/ml/hr) ˜2 μM of recombinant human renin IS added to achieve PRA of 3-4 ng/ml/hr. The cleavage of endogenous angiotensinogen in plasma is carried out at 37° C. for 90 min and the product angiotensin I is measured by competitive radioimmunoassay using DiaSorin PRA kit. Uninhibited incubations containing 2% DMSO and fully inhibited controls with 2 μM of isovaleryl-Phe-Nle-Sta-Ala-Sta-OH are used for deriving percent of inhibition for each concentration of inhibitors and fitting dose-response data into a four parametric model from which IC₅₀ values, defined as concentrations of inhibitors at which 50% inhibition occurs, is determined.

### Example 19

### Efficacy of the Disclosed Inhibitors in a Transgenic Rat Model

The efficacy of the renin inhibitors is also evaluated in vivo in double transgenic rats engineered to express human renin and human angiotensinogen (Bohlender J, Fukamizu A, Lippoldt A, Nomura T, Dietz R, Menard J, Murakami K, Luft F C, Ganten D. High human renin hypertension in transgenic rats. Hypertension 1997, 29, 428-434).

Experiments are conducted in 5-10 week-old double transgenic rats (dTGRs). The model has been described in detail earlier. Briefly, the human renin construct are used to generate transgenic animals (hRen) made up the entire genomic human renin gene (10 exons and 9 introns), with 3.0 kB of the 5′-promoter region and 1.2 kB of 3′ additional sequences. The human angiotensinogen construct made up the entire human angiotensinogen gene (5 exons and 4 introns), with 1.3 kB of 5′-flanking and 2.4 kB of 3′-flanking sequences are used to generate rats producing human angiotensinogen (hAogen). The hRen and hAogen rats are rederived using embryo transfer from breeding pairs obtained under license from Ascencion Gmbh (Germany). The hAogen and hRen are then crossed to produce the double transgenic dTGR) off-spring. The dTGr rats are maintained on irradiated rodent chow (5VO2, Purina Mills Inc) and normal water. Radio telemetry transmitters (TA11PAC40, Data Sciences International) are surgically implanted at 5-6 weeks of age. The telemetry system provided 24-h recordings of systolic, mean, diastolic arterial pressure (SAP, MAP, DAP, respectively) and heart rate (HR). Prior to dosing, baseline hemodynamic measures are obtained for 24 hours. Rats are then dosed orally with vehicle or drug and monitored up to 48 hours post-dose.

While this invention has been particularly shown and described with references to preferred embodiments thereof, it will be understood by those skilled in the art that various changes in form and details may be made therein without departing from the scope of the invention encompassed by the appended claims.

1. A compound selected from the group:
methyl (S)-4-((R)-1-((1R,3S)-3-aminocyclopentanecarbonyl)piperidin-3-yl)-4-hydroxy-4-(2-(pyridin-4-yl)phenyl)butylcarbamate,
2-((S)-((R)-4-((1R,3S)-3-aminocyclopentanecarbonyl)morpholin-2-yl)(6-fluoro-3′-methylbiphenyl-2-yl)methoxy)-N-ethylacetamide,
N—((S)-4-((R)-1-((1R,3S)-3-aminocyclopentanecarbonyl)piperidin-3-yl)-4-hydroxy-4-(2-(o-tolyloxy)phenyl)butyl)acetamide,
((1R,3S)-3-aminocyclopentyl)((R)-3-((S)-1-(2-(2,6-dimethylphenoxy)phenyl)-1-hydroxy-5-methoxypentyl)piperidin-1-yl)methanone,
methyl (S)-4-((R)-1-((1S,3R,4S)-3-amino-4-hydroxycyclopentanecarbonyl)piperidin-3-yl)-4-hydroxy-4-(2-(pyridin-3-yl)phenyl)butylcarbamate,
((1S,3R,4S)-3-amino-4-hydroxycyclopentyl)((R)-2-((R)-1-(6-chloro-3′-ethylbiphenyl-2-yl)-1-hydroxypent-4-enyl)morpholino)methanone,
methyl 2-((S)-((R)-4-((1R,3S)-3-aminocyclopentanecarbonyl)morpholin-2-yl)(6-fluoro-3′-methylbiphenyl-2-yl)methoxy)ethylcarbamate,
2-((S)-((R)-4-((1S,3R,4S)-3-amino-4-hydroxycyclopentanecarbonyl)morpholin-2-yl)(6-fluoro-3′-methylbiphenyl-2-yl)methoxy)-N-ethylacetamide,
((1R,3S)-3-aminocyclopentyl)((R)-2-((R)-1-(4′,6-difluoro-3′-methylbiphenyl-2-yl)-1-hydroxy-5-methoxypentyl)morpholino) methanone,
((1S,3R,4S)-3-amino-4-hydroxycyclopentyl)((R)-2-((R)-1-(3-chloro-2-(pyridin-3-yl)phenyl)-1-hydroxy-5-methoxypentyl)morpholino)methanone,
((1S,3R,4S)-3-amino-4-hydroxycyclopentyl)((R)-2-0)-1-(3-chloro-2-(3-methyl-1,2,4-oxadiazol-5-yl)phenyl)-1-hydroxy-5-methoxypentyl)morpholino)methanone,
methyl (S)-4-((R)-1-((1R,3S)-3-aminocyclopentanecarbonyl)piperidin-3-yl)-4-hydroxy-4-(2-(o-tolyloxy)phenyl)butylcarbamate,
N—((S)-4-((R)-1-((1S,3R,4S)-3-amino-4-hydroxycyclopentanecarbonyl)piperidin-3-yl)-4-hydroxy-4-(2-(o-tolyloxy)phenyl)butyl)acetamide,
((1S,3R,4S)-3-amino-4-hydroxycyclopentyl)((R)-3-((S)-1-(2-(2,6-dimethylphenoxy)phenyl)-1-hydroxy-5-methoxypentyl)piperidin-1-yl)methanone,
((1R,3S)-3-aminocyclopentyl)((R)-3-((S)-1-(6-fluoro-3′-methoxy-5′-methylbiphenyl-2-yl)-1-hydroxy-5-methoxypentyl)piperidin-1-yl)methanone,
((1R,3S)-3-aminocyclopentyl)((R)-3-((S)-1-(6-chloro-3′-ethylbiphenyl-2-yl)-1-hydroxy-5-methoxypentyl)piperidin-1-yl)methanone,
((1R,3S)-3-aminocyclopentyl)((R)-3-((S)-1-(3-chloro-2-(2-methylbenzyl)phenyl)-1-hydroxy-5-methoxypentyl)piperidin-1-yl)methanone,
((1R,3S)-3-aminocyclopentyl)((R)-3-((S)-1-(3-chloro-2-(3-methylbenzyl)phenyl)-1-hydroxy-5-methoxypentyl)piperidin-1-yl)methanone,
((1R,3S)-3-aminocyclopentyl)((R)-2-((R)-1-(6-fluoro-3′-methoxy-5′-methylbiphenyl-2-yl)-1-hydroxy-5-methoxypentyl)morpholino)methanone,
((1R,3S)-3-aminocyclopentyl)((R)-2-((R)-1-(6-chloro-3′-ethylbiphenyl-2-yl)-1-hydroxy-5-methoxypentyl)morpholino) methanone,
((1R,3S)-3-aminocyclopentyl)((R)-3-((S)-1-(3-chloro-2-(o-tolyloxy)phenyl)-1-hydroxy-5-methoxypentyl)piperidin-1-yl)methanone,
((1R,3S)-3-aminocyclopentyl)((R)-3-((S)-1-(2-(2-chloro-6-methylphenoxy)phenyl)-1-hydroxy-5-methoxypentyl)piperidin-1-yl)methanone,
((1S,3R,4S)-3-amino-4-hydroxycyclopentyl)((R)-2-((R)-1-(4′,6-difluoro-3′-methylbiphenyl-2-yl)-1-hydroxy-5-methoxypentyl)morpholino) methanone,
methyl (S)-4-((R)-1-((1R,2S)-2-aminocyclopentanecarbonyl)piperidin-3-yl)-4-(3′-ethyl-6-fluorobiphenyl-2-yl)-4-hydroxybutylcarbamate,
(trans-4-aminocyclohexyl)((R)-3-((S)-1-(6-chloro-3′-ethylbiphenyl-2-yl)-1-hydroxy-5-methoxypentyl)piperidin-1-yl)methanone,
(1S)-1-((3R)-1-{[6-(aminomethyl)-3-pyridinyl]carbonyl}-3-piperidinyl)-1-(6-chloro-3′-ethyl-2-biphenylyl)-5-(methyloxy)-1-pentanol,
((1S,3R,4S)-3-amino-4-hydroxycyclopentyl)((R)-3-((S)-1-(6-fluoro-3′-methoxy-5′-methylbiphenyl-2-yl)-1-hydroxy-5-methoxypentyl)piperidin-1-yl)methanone,
((1S,3R,4S)-3-amino-4-hydroxycyclopentyl)((R)-3-((S)-1-(6-chloro-3′-ethylbiphenyl-2-yl)-1-hydroxy-5-methoxypentyl)piperidin-1-yl)methanone,
((1R,3S)-3-aminocyclopentyl)((R)-3-((S)-1-(3-chloro-2-(2-ethylphenoxy)phenyl)-1-hydroxy-5-methoxypentyl)piperidin-1-yl)methanone,
((1R,3S)-3-aminocyclopentyl)((R)-3-((S)-1-(3-chloro-2-(3-ethylphenoxy)phenyl)-1-hydroxy-5-methoxypentyl)piperidin-1-yl)methanone,
((1S,3R,4S)-3-amino-4-hydroxycyclopentyl)((R)-3-((S)-1-(3-chloro-2-(2-methylbenzyl)phenyl)-1-hydroxy-5-methoxypentyl)piperidin-1-yl)methanone,
((1S,3R,4S)-3-amino-4-hydroxycyclopentyl)((R)-3-((S)-1-(3-chloro-2-(3-methylbenzyl)phenyl)-1-hydroxy-5-methoxypentyl)piperidin-1-yl)methanone,
((1S,3R,4S)-3-amino-4-hydroxycyclopentyl)((R)-2-((R)-1-(6-fluoro-3′-methoxy-5′-methylbiphenyl-2-yl)-1-hydroxy-5-methoxypentyl)morpholino)methanone,
((1S,3R,4S)-3-amino-4-hydroxycyclopentyl)((R)-3-((S)-1-(3-chloro-2-(o-tolyloxy)phenyl)-1-hydroxy-5-methoxypentyl)piperidin-1-yl)methanone,
((1S,3R,4S)-3-amino-4-hydroxycyclopentyl)((2R)-2-((1R)-1-(6-chloro-2′-fluoro-5′-methylbiphenyl-2-yl)-1-hydroxy-5-methoxypentyl)morpholino) methanone,
((1R,3S)-3-aminocyclopentyl)((R)-3-((S)-1-(3-chloro-2-(quinolin-3-yl)phenyl)-1-hydroxy-5-methoxypentyl)piperidin-1-yl)methanone,
((1R,3S)-3-aminocyclopentyl)((R)-2-((R)-1-(3-chloro-2-(naphthalen-2-yl)phenyl)-1-hydroxy-5-methoxypentyl)morpholino) methanone,
((1R,3S)-3-aminocyclopentyl)((R)-2-((R)-1-(3-chloro-2-(quinolin-3-yl)phenyl)-1-hydroxy-5-methoxypentyl)morpholino) methanone,
methyl ((4S)-4-hydroxy-4-((3R)-1-{[(1R,3S)-3-(methylamino)cyclopentyl]carbonyl}-3-piperidinyl)-4-{2-[(2-methylphenyl)oxy]phenyl}butyl)carbamate,
methyl (S)-4-(6-fluoro-3′-methoxybiphenyl-2-yl)-4-hydroxy-4-((R)-1-((1R,3S)-3-(methylamino)cyclopentanecarbonyl)piperidin-3-yl)butylcarbamate,
((1S,3R,4S)-3-amino-4-hydroxycyclopentyl)((R)-3-((S)-1-(3-chloro-2-(3-ethylphenoxy)phenyl)-1-hydroxy-5-methoxypentyl)piperidin-1-yl)methanone,
((1S,3R,4S)-3-amino-4-hydroxycyclopentyl)((R)-2-((R)-1-(6-fluoro-3′,5′-dimethoxybiphenyl-2-yl)-1-hydroxy-5-methoxypentyl)morpholino) methanone,
((1S,3R,4S)-3-amino-4-hydroxycyclopentyl)((R)-2-((R)-1-(6-chloro-3′-(methoxymethyl)biphenyl-2-yl)-1-hydroxy-5-methoxypentyl)morpholino) methanone,
(4-(aminomethyl)cyclohexyl)((2R)-2-((1R)-1-(6-chloro-2′-fluoro-5′-methylbiphenyl-2-yl)-1-hydroxy-5-methoxypentyl)morpholino)methanone,
((1S,3R,4S)-3-amino-4-hydroxycyclopentyl)((R)-3-((S)-1-(3-chloro-2-(quinolin-3-yl)phenyl)-1-hydroxy-5-methoxypentyl)piperidin-1-yl)methanone,
((1S,3R,4S)-3-amino-4-hydroxycyclopentyl)((R)-2-((R)-1-(3-chloro-2-(naphthalen-2-yl)phenyl)-1-hydroxy-5-methoxypentyl)morpholino) methanone,
((1S,3R,4S)-3-amino-4-hydroxycyclopentyl)((R)-2-((R)-1-(3-chloro-2-(quinolin-3-yl)phenyl)-1-hydroxy-5-methoxypentyl)morpholino) methanone,
((1S,3R,4S)-3-amino-4-hydroxycyclopentyl)((2R)-2-((1R)-1-(3-chloro-2-(isoquinolin-4-yl)phenyl)-1-hydroxy-5-methoxypentyl)morpholino) methanone,
methyl (S)-4-((R)-1-((1R,3S)-3-aminocyclopentanecarbonyl)piperidin-3-yl)-4-(6-chloro-3′-isopropylbiphenyl-2-yl)-4-hydroxybutylcarbamate,
methyl (S)-4-(6-chloro-3′-methoxybiphenyl-2-yl)-4-hydroxy-4-((R)-1-((1R,3S)-3-(methylamino)cyclopentanecarbonyl)piperidin-3-yl)butylcarbamate,
((1S,3R,4S)-3-amino-4-hydroxycyclopentyl)((R)-2-((R)-1-(6-chloro-3′,5′-dimethoxybiphenyl-2-yl)-1-hydroxy-5-methoxypentyl)morpholino) methanone,
((1R,3S)-3-aminocyclopentyl)((R)-3-((S)-1-(3′-ethoxy-6-fluoro-5′-(trifluoromethyl)biphenyl-2-yl)-1-hydroxy-5-methoxypentyl)piperidin-1-yl)methanone,
methyl (S)-4-((R)-1-((1S,3R,4S)-3-amino-4-hydroxycyclopentanecarbonyl)piperidin-3-yl)-4-(3-chloro-2-(quinolin-3-yl)phenyl)-4-hydroxybutylcarbamate,
((1R,3S)-3-aminocyclopentyl)((R)-2-((R)-1-(3′-ethoxy-6-fluoro-5′-(trifluoromethyl)biphenyl-2-yl)-1-hydroxy-5-methoxypentyl)morpholino) methanone,
((1S,3R,4S)-3-amino-4-hydroxycyclopentyl)((R)-3-((S)-1-(3′-ethoxy-6-fluoro-5′-(trifluoromethyl)biphenyl-2-yl)-1-hydroxy-5-methoxypentyl)piperidin-1-yl)methanone,
((1S,3R,4S)-3-amino-4-hydroxycyclopentyl)((R)-2-((R)-1-(3′-ethoxy-6-fluoro-5′-(trifluoromethyl)biphenyl-2-yl)-1-hydroxy-5-methoxypentyl)morpholino) methanone,
((1R,3S)-3-aminocyclopentyl)((R)-2-((R)-1-(6-fluoro-3′,5′-dimethoxybiphenyl-2-yl)-1-hydroxy-5-methoxypentyl)morpholino) methanone,
((1R,3S)-3-aminocyclopentyl)((2R)-2-((1R)-1-(3-chloro-2-(isoquinolin-4-yl)phenyl)-1-hydroxy-5-methoxypentyl)morpholino)methanone,
methyl (4-((3R)-1-{[(1S,3R,4S)-3-amino-4-hydroxycyclopentyl]carbonyl}-3-piperidinyl)-4-{3-chloro-2-[(2-methylphenyl)oxy]phenyl}-4-hydroxybutyl)carbamate,
methyl [(4S)-4-((3R)-1-{[(1R,2S)-2-aminocyclopentyl]carbonyl}-3-piperidinyl)-4-(3′-ethyl-6-fluoro-2-biphenylyl)-4-hydroxybutyl]carbamate,
(1R)-1-((2R)-4-{[6-(aminomethyl)-3-pyridinyl]carbonyl}-2-morpholinyl)-1-(6-chloro-3′-ethyl-2-biphenylyl)-5-(methyloxy)-1-pentanol,
methyl [(4S)-4-((3R)-1-{[6-(aminomethyl)-3-pyridinyl]carbonyl}-3-piperidinyl)-4-(6-chloro-3′-ethyl-2-biphenylyl)-4-hydroxybutyl]carbamate,
(1S)-1-{(3R)-1-[(cis-4-aminocyclohexyl)acetyl]-3-piperidinyl}-1-(6-chloro-3′-ethyl-2-biphenylyl)-5-(methyloxy)-1-pentanol,
(1S)-1-{(3R)-1-[(trans-4-aminocyclohexyl)acetyl]-3-piperidinyl}-1-(6-chloro-3′-ethyl-2-biphenylyl)-5-(methyloxy)-1-pentanol,
(1S)-1-((3R)-1-{[(1R,2S)-2-aminocyclopentyl]carbonyl}-3-piperidinyl)-1-(6-chloro-3′-ethyl-2-biphenylyl)-5-(methyloxy)-1-pentanol,
(1R)-1-(6-chloro-3′-ethyl-2-biphenylyl)-1-((2R)-4-{[(1R,3S)-3-(methylamino)cyclopentyl]carbonyl}-2-morpholinyl)-5-(methyloxy)-1-pentanol,
(1S)-1-(6-chloro-3′-ethyl-2-biphenylyl)-1-((3R)-1-{[(1R,3S)-3-(methylamino)cyclopentyl]carbonyl}-3-piperidinyl)-5-(methyloxy)-1-pentanol,
(1S,2R,4S)-4-({(2R)-2-[(1R)-1-(6-chloro-3′-ethyl-2-biphenylyl)-1-hydroxy-5-(methyloxy)pentyl]-4-morpholinyl}carbonyl)-2-[(2-hydroxyethyl)amino]cyclopentanol,
methyl {(4S)-4-(6-chloro-3′-ethyl-2-biphenylyl)-4-hydroxy-4-[(3R)-1-({(1S,3S,4R)-3-hydroxy-4-[(2-hydroxyethyl)amino]cyclopentyl}carbonyl)-3-piperidinyl]butyl}carbamate,
(1S)-1-(6-chloro-3′-ethyl-2-biphenylyl)-1-[(3R)-1-({trans-4-[(methylamino)methyl]cyclohexyl}carbonyl)-3-piperidinyl]-5-(methyloxy)-1-pentanol,
(1S,2R,4S)-4-({(2R)-2-[(1R)-1-(6-chloro-3′-ethyl-2-biphenylyl)-1-hydroxy-5-(methyloxy)pentyl]-4-morpholinyl}carbonyl)-2-[(1,3-thiazol-2-ylmethyl)amino]cyclopentanol,
methyl [(4S)-4-((3R)-1-{[(1R,3S)-3-aminocyclohexyl]carbonyl}-3-piperidinyl)-4-(6-chloro-3′-ethyl-2-biphenylyl)-4-hydroxybutyl]carbamate,
N-{(4S)-4-(6-chloro-3′-methyl-2-biphenylyl)-4-hydroxy-4-[(3R)-1-({trans-4-[(methylamino)methyl]cyclohexyl}carbonyl)-3-piperidinyl]butyl}acetamide,
N-[(4S)-4-(6-chloro-3′-methyl-2-biphenylyl)-4-hydroxy-4-((3R)-1-{[(1R,3S)-3-(methylamino)cyclopentyl]carbonyl}-3-piperidinyl)butyl]acetamide,
(1R)-1-(6-chloro-3′-ethyl-2-biphenylyl)-1-{(2R)-4-[((1R,3S)-3-{[(2,5-dimethyl-1,3-oxazol-4-yl)methyl]amino}cyclopentyl)carbonyl]-2-morpholinyl}-5-(methyloxy)-1-pentanol,
N-[(4S)-4-((3R)-1-{[trans-4-(aminomethyl)cyclohexyl]carbonyl}-3-piperidinyl)-4-(6-chloro-3′-methyl-2-biphenylyl)-4-hydroxybutyl]acetamide,
methyl {(4S)-4-(4,6-difluoro-3′-methyl-2-biphenylyl)-4-hydroxy-4-[(3R)-1-({trans-4-[(methylamino)methyl]cyclohexyl}carbonyl)-3-piperidinyl]butyl}carbamate,
methyl [(4S)-4-hydroxy-4-((3R)-1-{[(1R,3S)-3-(methylamino)cyclopentyl]carbonyl}-3-piperidinyl)-4-(2′,4,6-trifluoro-5′-methyl-2-biphenylyl)butyl]carbamate,
methyl [(4S)-4-(6-chloro-3′-methyl-2-biphenylyl)-4-hydroxy-4-((3R)-1-{[(1R,3S)-3-(methylamino)cyclopentyl]carbonyl}-3-piperidinyl)butyl]carbamate,
(1R)-1-(6-chloro-3′-ethyl-2-biphenylyl)-1-[(2R)-4-({trans-4-[(methylamino)methyl]cyclohexyl}carbonyl)-2-morpholinyl]-5-(methyloxy)-1-pentanol,
(1S,2R,4S)-2-amino-4-({(2R)-2-[(1R)-1-[2-chloro-3-(3-ethylphenyl)-4-pyridinyl]-1-hydroxy-5-(methyloxy)pentyl]-4-morpholinyl}carbonyl)cyclopentanol,
(1R)-1-((2R)-4-{[(1R,3S)-3-aminocyclopentyl]carbonyl}-2-morpholinyl)-1-[2-chloro-3-(3-ethylphenyl)-4-pyridinyl]-5-(methyloxy)-1-pentanol,
(1S,2R,4S)-2-amino-4-({(2R)-2-[(1R)-1-[2-(1-benzothien-3-yl)-3-chlorophenyl]-1-hydroxy-5-(methyloxy)pentyl]-4-morpholinyl}carbonyl)cyclopentanol,
(1R)-1-((2R)-4-{[(1R,3S)-3-aminocyclopentyl]carbonyl}-2-morpholinyl)-1-[2-(1-benzothien-3-yl)-3-chlorophenyl]-5-(methyloxy)-1-pentanol,
(1R)-1-[4-chloro-3-(3-ethylphenyl)-2-pyridinyl]-1-((2R)-4-{[(1R,3S)-3-(methylamino)cyclopentyl]carbonyl}-2-morpholinyl)-5-(methyloxy)-1-pentanol,
methyl {(4S)-4-[2-chloro-3-(3-ethylphenyl)-4-pyridinyl]-4-hydroxy-4-[(3R)-1-({trans-4-[(methylamino)methyl]cyclohexyl}carbonyl)-3-piperidinyl]butyl}carbamate,
methyl [(4S)-4-[2-chloro-3-(3-ethylphenyl)-4-pyridinyl]-4-hydroxy-4-((3R)-1-{[(1R,3S)-3-(methylamino)cyclopentyl]carbonyl}-3-piperidinyl)butyl]carbamate,
methyl {(4S)-4-{3-chloro-2-[4-(1-methylethyl)-2-quinazolinyl]phenyl}-4-hydroxy-4-[(3R)-1-({trans-4-[(methylamino)methyl]cyclohexyl}carbonyl)-3-piperidinyl]butyl)carbamate,
methyl [(4S)-4-{3-chloro-2-[4-(1-methylethyl)-2-quinazolinyl]phenyl}-4-hydroxy-4-((3R)-1-{[(1R,3S)-3-(methylamino)cyclopentyl]carbonyl}-3-piperidinyl)butyl}carbamate,
(1S)-1-((3R)-1-{[(1R,3S)-3-aminocyclopentyl]carbonyl}-3-piperidinyl)-1-{3-chloro-2-[(3-methylphenyl)oxy]phenyl}-5-(methyloxy)-1-pentanol,
(1S,2R,4S)-2-amino-4-({(3R)-3-[(1S)-1-{3-chloro-2-[(3-methylphenyl)oxy]phenyl}-1-hydroxy-5-(methyloxy)pentyl]-1-piperidinyl}carbonyl)cyclopentanol,
methyl ((4S)-4-((3R)-1-{[(1R,3S)-3-aminocyclopentyl]carbonyl}-3-piperidinyl)-4-{3-chloro-2-[(3-methylphenyl)oxy]phenyl}-4-hydroxybutyl)carbamate,
methyl ((4S)-4-((3R)-1-{[(1S,3R,4S)-3-amino-4-hydroxycyclopentyl]carbonyl}-3-piperidinyl)-4-{3-chloro-2-[(3-methylphenyl)oxy]phenyl}-4-hydroxybutyl)carbamate,
methyl (4S)-4-((3R)-1-{[(1R,3S)-3-aminocyclopentyl]carbonyl}-3-piperidinyl)-4-{2-[(2,6-dimethylphenyl)oxy]phenyl}-4-hydroxybutyl)carbamate,
methyl ((4S)-4-((3R)-1-{[(1S,3R,4S)-3-amino-4-hydroxycyclopentyl]carbonyl}-3-piperidinyl)-4-{2-[(2,6-dimethylphenyl)oxy]phenyl}-4-hydroxybutyl)carbamate,
methyl {(4S)-4-{3-chloro-2-[8-(1-methylethyl)-2-quinolinyl]phenyl}-4-hydroxy-4-[(3R)-1-({trans-4-[(methylamino)methyl]cyclohexyl}carbonyl)-3-piperidinyl]butyl}carbamate,
methyl [(4S)-4-[3-fluoro-2-(3-quinolinyl)phenyl]-4-hydroxy-4-((3R)-1-{[(1R,3S)-3-(methylamino)cyclopentyl]carbonyl}-3-piperidinyl)butyl]carbamate,
methyl [(4S)-4-{2-chloro-3-[3-(1-methylethyl)phenyl]-4-pyridinyl}-4-hydroxy-4-((3R)-1-{[(1R,3S)-3-(methylamino)cyclopentyl]carbonyl}-3-piperidinyl)butyl]carbamate,
methyl [(4S)-4-[3-chloro-2-(5-methyl-2-furanyl)phenyl]-4-hydroxy-4-((3R)-1-{[(1R,3S)-3-(methylamino)cyclopentyl]carbonyl}-3-piperidinyl)butyl]carbamate,
methyl {(4S)-4-((3R)-1-{[(1S,3R,4S)-3-amino-4-hydroxycyclopentyl]carbonyl}-3-piperidinyl)-4-[3-chloro-2-(5-methyl-2-furanyl)phenyl]-4-hydroxybutyl}carbamate,
(1R)-1-{2-chloro-3-[3-(1-methylethyl)phenyl]-4-pyridinyl}-1-[(2R)-4-({trans-4-[(methylamino)methyl]cyclohexyl}carbonyl)-2-morpholinyl]-5-(methyloxy)-1-pentanol,
(1R)-1-{2-chloro-3-[3-(1-methylethyl)phenyl]-4-pyridinyl}-1-((2R)-4-{[(1R,3S)-3-(methylamino)cyclopentyl]carbonyl}-2-morpholinyl)-5-(methyloxy)-1-pentanol,
(1S,2R,4S)-2-amino-4-({(2R)-2-[(1R)-1-{2-chloro-3-[3-(1-methylethyl)phenyl]-4-pyridinyl}-1-hydroxy-5-(methyloxy)pentyl]-4-morpholinyl}carbonyl)cyclopentanol,
methyl {(4S)-4-[5-chloro-4-(3-ethylphenyl)-3-pyridinyl]-4-hydroxy-4-[(3R)-1-({trans-4-[(methylamino)methyl]cyclohexyl}carbonyl)-3-piperidinyl]butyl}carbamate,
methyl [(4S)-4-[5-chloro-4-(3-ethylphenyl)-3-pyridinyl]-4-hydroxy-4-((3R)-1-{[(1R,3S)-3-(methylamino)cyclopentyl]carbonyl}-3-piperidinyl)butyl]carbamate,
methyl {(4S)-4-{5-chloro-4-[3-(1-methylethyl)phenyl]-3-pyridinyl}-4-hydroxy-4-[(3R)-1-({trans-4-[(methylamino)methyl]cyclohexyl}carbonyl)-3-piperidinyl]butyl}carbamate,
methyl [(4S)-4-{5-chloro-4-[3-(1-methylethyl)phenyl]-3-pyridinyl}-4-hydroxy-4-((3R)-1-{[(1R,3S)-3-(methylamino)cyclopentyl]carbonyl}-3-piperidinyl)butyl]carbamate,
methyl [(4S)-4-(6-chloro-3′-fluoro-5′-methyl-2-biphenylyl)-4-hydroxy-4-((3R)-1-{[(1R,3S)-3-(methylamino)cyclopentyl]carbonyl}-3-piperidinyl)butyl]carbamate,
methyl [(4S)-4-(3′,6-difluoro-5′-methyl-2-biphenylyl)-4-hydroxy-4-((3R)-1-{[(1R,3S)-3-(methylamino)cyclopentyl]carbonyl}-3-piperidinyl)butyl]carbamate,
(1S,2R,4S)-2-amino-4-({(3R)-3-[(1S)-1-{3-chloro-2-[(2-ethylphenyl)oxy]phenyl}-1-hydroxy-5-(methyloxy)pentyl]-1-piperidinyl}carbonyl)cyclopentanol,
(1S,2R,4S)-2-amino-4-({(3R)-3-[(1S)-1-{2-[(2-chloro-6-methylphenyl)oxy]phenyl}-1-hydroxy-5-(methyloxy)pentyl]-1-piperidinyl}carbonyl)cyclopentanol,
(1S,2R,4S)-2-amino-4-({(3R)-3-[(1S)-1-{3-fluoro-2-[(3-methylphenyl)oxy]phenyl}-1-hydroxy-5-(methyloxy)pentyl]-1-piperidinyl}carbonyl)cyclopentanol,
(1S)-1-((3R)-1-{[(1R,3S)-3-aminocyclopentyl]carbonyl}-3-piperidinyl)-1-{3-fluoro-2-[(3-methylphenyl)oxy]phenyl}-5-(methyloxy)-1-pentanol,
methyl ((4S)-4-((3R)-1-{[(1S,3R,4S)-3-amino-4-hydroxycyclopentyl]carbonyl}-3-piperidinyl)-4-{3-chloro-2-[(3-ethylphenyl)oxy]phenyl}-4-hydroxybutyl)carbamate,
methyl ((4S)-4-((3R)-1-{[(1R,3S)-3-aminocyclopentyl]carbonyl}-3-piperidinyl)-4-{3-chloro-2-[(3-ethylphenyl)oxy]phenyl}-4-hydroxybutyl)carbamate,
methyl ((4S)-4-((3R)-1-{[(1S,3R,4S)-3-amino-4-hydroxycyclopentyl]carbonyl}-3-piperidinyl)-4-{3-fluoro-2-[(3-methylphenyl)oxy]phenyl}-4-hydroxybutyl)carbamate,
methyl ((4S)-4-((3R)-1-{[(1R,3S)-3-aminocyclopentyl]carbonyl}-3-piperidinyl)-4-{3-fluoro-2-[(3-methylphenyl)oxy]phenyl}-4-hydroxybutyl)carbamate,
methyl ((4S)-4-((3R)-1-{[(1S,3R,4S)-3-amino-4-hydroxycyclopentyl]carbonyl}-3-piperidinyl)-4-{3-chloro-2-[(3-methylphenyl)methyl]phenyl}-4-hydroxybutyl)carbamate,
methyl [(4S)-4-{3-chloro-2-[(3-methylphenyl)methyl]phenyl}-4-hydroxy-443R)-1-{[(1R,3S)-3-(methylamino)cyclopentyl]carbonyl}-3-piperidinyl)butyl]carbamate,
methyl ((4S)-4-((3R)-1-{[(1R,3S)-3-aminocyclopentyl]carbonyl}-3-piperidinyl)-4-{3-chloro-2-[(3-methylphenyl)methyl]phenyl}-4-hydroxybutyl)carbamate,
methyl (2-{[(S)-((2R)-4-{[(1S,3R,4S)-3-amino-4-hydroxycyclopentyl]carbonyl}-2-morpholinyl)(6-chloro-3′-ethyl-2-biphenylyl)methyl]oxy}ethyl)carbamate,
methyl (2-{[(R)-((3R)-1-{[(1S,3R,4S)-3-amino-4-hydroxycyclopentyl]carbonyl}-3-piperidinyl)(6-chloro-3′-ethyl-2-biphenylyl)methyl]oxy}ethyl)carbamate,
methyl [(4S)-4-((3R)-1-{[4-(aminomethyl)-5-methyl-2-furanyl]carbonyl}-3-piperidinyl)-4-(6-chloro-3′-ethyl-2-biphenylyl)-4-hydroxybutyl]carbamate,
methyl {(4S)-4-(6-chloro-3′-ethyl-2-biphenylyl)-4-hydroxy-4-[(3R)-1-({6-[(methylamino)methyl]-3-pyridinyl}carbonyl)-3-piperidinyl]butyl}carbamate,
methyl [(4S)-4-{(3R)-1-[(2-amino-4-oxo-1,4-dihydro-5-pyrimidinyl)carbonyl]-3-piperidinyl}-4-(6-chloro-3′-methyl-2-biphenylyl)-4-hydroxybutyl]carbamate,
methyl [(4S)-4-(6-chloro-3′-methyl-2-biphenylyl)-4-hydroxy-4-((3R)-1-{[2-(methylamino)-5-pyrimidinyl]carbonyl}-3-piperidinyl)butyl]carbamate,
methyl [(4S)-4-((3R)-1-{[5-(aminomethyl)-3-isoxazolyl]carbonyl}-3-piperidinyl)-4-(6-chloro-3′-methyl-2-biphenylyl)-4-hydroxybutyl]carbamate,
methyl {(4S)-4-(6-chloro-3′-ethyl-2-biphenylyl)-4-hydroxy-4-[(3R)-1-({5-[(methylamino)methyl]-2-thienyl}carbonyl)-3-piperidinyl]butyl}carbamate,
methyl [(4S)-4-{(3R)-1-[(6-amino-3-pyridinyl)carbonyl]-3-piperidinyl}-4-(3′,6-difluoro-5′-methyl-2-biphenylyl)-4-hydroxybutyl]carbamate,
methyl [2-({(R)-(6-chloro-3′-ethyl-2-biphenylyl)[(3R)-1-({trans-4-[(methylamino)methyl]cyclohexyl}carbonyl)-3-piperidinyl]methyl}oxy)ethyl]carbamate,
methyl [2-({(S)-(6-chloro-3′-ethyl-2-biphenylyl)[(2R)-4-({trans-4-[(methylamino)methyl]cyclohexyl}carbonyl)-2-morpholinyl]methyl}oxy)ethyl]carbamate,
N-[(4S)-4-((3R)-1-{[6-(aminomethyl)-3-pyridinyl]carbonyl}-3-piperidinyl)-4-(6-chloro-3′-methyl-2-biphenylyl)-4-hydroxybutyl]acetamide,
methyl [(4S)-4-((3R)-1-{[4-(aminomethyl)-4-phenyl-1-piperidinyl]carbonyl}-3-piperidinyl)-4-(6-chloro-3′-ethyl-2-biphenylyl)-4-hydroxybutyl]carbamate, and
methyl {(4S)-4-(6-chloro-3′-ethyl-2-biphenylyl)-4-hydroxy-4-[(3R)-1-({4-[(2-propen-1-ylamino)methyl]-1-piperidinyl}carbonyl)-3-piperidinyl]butyl}carbamate,
and a salt thereof.

2. A compound selected from the group:
methyl (S)-4-((R)-1-((1S,3R,4S)-3-amino-4-hydroxycyclopentanecarbonyl)piperidin-3-yl)-4-hydroxy-4-(2-(o-tolyloxy)phenyl)butylcarbamate,
methyl (S)-4-((R)-1-((1R,3S)-3-aminocyclopentanecarbonyl)piperidin-3-yl)-4-(3′-ethyl-6-fluorobiphenyl-2-yl)-4-hydroxybutylcarbamate,
((1S,3R,4S)-3-amino-4-hydroxycyclopentyl)((R)-2-((R)-1-(6-chloro-3′-ethyl biphenyl-2-yl)-1-hydroxy-5-methoxypentyl)morpholino)methanone,
N—((S)-4-((R)-1-((1S,3R,4S)-3-amino-4-hydroxycyclopentanecarbonyl)piperidin-3-yl)-4-(6-chloro-3′-ethylbiphenyl-2-yl)-4-hydroxybutyl)acetamide,
methyl (S)-4-((R)-1-((1R,3S)-3-aminocyclopentanecarbonyl)piperidin-3-yl)-4-(6-chloro-3′-ethylbiphenyl-2-yl)-4-hydroxybutylcarbamate,
methyl (R)-4-((R)-4-((1R,3S)-3-aminocyclopentanecarbonyl)morpholin-2-yl)-4-(6-chloro-3′-ethylbiphenyl-2-yl)-4-hydroxybutylcarbamate,
methyl (S)-4-(6-chloro-3′-ethylbiphenyl-2-yl)-4-hydroxy-4-(((R)-1-((1R,3S)-3-(methylamino)cyclopentanecarbonyl)piperidin-3-yl)butylcarbamate,
methyl (S)-4-((R)-1-((1S,3R,4S)-3-amino-4-hydroxycyclopentanecarbonyl)piperidin-3-yl)-4-(6-chloro-3′-ethylbiphenyl-2-yl)-4-hydroxybutylcarbamate,
methyl (S)-4-((R)-1-(trans-4-(aminomethyl)cyclohexanecarbonyl)piperidin-3-yl)-4-(6-chloro-3′-ethylbiphenyl-2-yl)-4-hydroxybutylcarbamate,
methyl (S)-4-(((R)-1-((1S,3R,4S)-3-amino-4-hydroxycyclopentanecarbonyl)piperidin-3-yl)-4-(6-chloro-3′-isopropylbiphenyl-2-yl)-4-hydroxybutylcarbamate,
methyl (4-((3R)-1-{[(1R,3S)-3-aminocyclopentyl]carbonyl}-3-piperidinyl)-4-{3-chloro-2-[(2-methylphenyl)oxy]phenyl}-4-hydroxybutyl)carbamate,
methyl (4-((3R)-1-{[(1R,3S)-3-aminocyclopentyl]carbonyl}-3-piperidinyl)-4-{3-chloro-2-[(2-ethylphenyl)oxy]phenyl}-4-hydroxybutyl)carbamate,
methyl (4-((3R)-1-{[(1S,3R,4S)-3-amino-4-hydroxycyclopentyl]carbonyl}-3-piperidinyl)-4-{3-chloro-2-[(2-ethylphenyl)oxy]phenyl}-4-hydroxybutyl)carbamate,
methyl [4-((3R)-1-{[(1S,3R,4S)-3-amino-4-hydroxycyclopentyl]carbonyl}-3-piperidinyl)-4-(3′-ethyl-6-fluoro-2-biphenylyl)-4-hydroxybutyl]carbamate,
methyl {4-(6-chloro-3′-ethyl-2-biphenylyl)-4-hydroxy-4-[(3R)-1-({trans-4-[(methylamino)methyl]cyclohexyl}carbonyl)-3-piperidinyl]butyl}carbamate,
methyl [(4S)-4-hydroxy-4-[(3R)-1-({trans-4-[(methylamino)methyl]cyclohexyl}carbonyl)-3-piperidinyl]-4-(2′,4,6-trifluoro-5′-methyl-2-biphenylyl)butyl]carbamate,
methyl [(4R)-4-((2R)-4-{[(1S,3R,4S)-3-amino-4-hydroxycyclopentyl]carbonyl}-2-morpholinyl)-4-(3′-ethyl-6-fluoro-2-biphenylyl)-4-hydroxybutyl]carbamate,
methyl {(4R)-4-(3′-ethyl-6-fluoro-2-biphenylyl)-4-hydroxy-4-[(2R)-4-({trans-4-[(methylamino)methyl]cyclohexyl}carbonyl)-2-morpholinyl]butyl}carbamate,
methyl {(4S)-4-[3-chloro-2-(8-methyl-2-quinolinyl)phenyl]-4-hydroxy-4-[(3R)-1-({trans-4-[(methylamino)methyl]cyclohexyl}carbonyl)-3-piperidinyl]butyl}carbamate,
methyl {(4S)-4-[6-fluoro-3′-(1-methylethyl)-2-biphenyl)-1]-4-hydroxy-4-[(3R)-1-({trans-4-[(methylamino)methyl]cyclohexyl}carbonyl)-3-piperidinyl]butyl}carbamate,
methyl {(4S)-4-(3′-ethyl-6-fluoro-2-biphenylyl)-4-hydroxy-4-[(3R)-1-({trans-4-[(methylamino)methyl]cyclohexyl}carbonyl)-3-piperidinyl]butyl}carbamate,
methyl [(4S)-4-((3R)-1-{[4-(aminomethyl)-1-piperidinyl]carbonyl}-3-piperidinyl)-4-(3′-ethyl-6-fluoro-2-biphenylyl)-4-hydroxybutyl]carbamate,
methyl {(4S)-4-(3′-ethyl-6-fluoro-2-biphenylyl)-4-hydroxy-4-[(3R)-1-({4-[(methylamino)methyl]-1-piperidinyl}carbonyl)-3-piperidinyl]butyl}carbamate,
methyl [(4S)-4-((3R)-1-{[4-(aminomethyl)-1-piperidinyl]carbonyl}-3-piperidinyl)-4-(6-chloro-3′-ethyl-2-biphenylyl)-4-hydroxybutyl]carbamate,
N-[(4S)-4-((3R)-1-{[4-(aminomethyl)-1-piperidinyl]carbonyl}-3-piperidinyl)-4-(2′,6-difluoro-5′-methyl-2-biphenylyl)-4-hydroxybutyl]-2-hydroxyacetamide, and
methyl {(4S)-4-(6-chloro-3′-ethyl-2-biphenylyl)-4-hydroxy-4-[(3R)-1-({4-[(methylamino)methyl]-1-piperidinyl}carbonyl)-3-piperidinyl]butyl}carbamate,
and a salt thereof.

3. A pharmaceutical composition comprising the compound according to any one of claims 1-2 or a salt thereof and a pharmaceutically acceptable carrier therefore.

4. The pharmaceutical composition according to claim 3, further comprising an additional agent selected from the group consisting of an α-blocker, a β-blocker, a calcium channel blocker, a diuretic, an angiotensin converting enzyme inhibitor, a dual angiotensin converting enzyme-neutral endopeptidase inhibitor, an angiotensin-receptor blocker, an aldosterone synthase inhibitor, an aldosterone-receptor antagonist, and an endothelin receptor antagonist.

5. A method of inhibiting an aspartic protease in a subject in need thereof comprising administering to the subject a therapeutically effective amount of the compound according to any one of claims 1-2 or a salt thereof.

6. The method according to claim 5, wherein the aspartic protease is renin.

7. A method for treating or ameliorating an aspartic protease mediated disorder in a subject in need thereof comprising administering to said subject a therapeutically effective amount of the compound according to any one of claims 1-2, or a salt thereof.

8. The method according to claim 7, wherein the aspartic protease is β-secretase.

9. The method according to claim 7, wherein the aspartic protease is plasmepsin.

10. The method according to claim 7, wherein the aspartic protease is HIV protease.

11. A method for treating or ameliorating a renin mediated disorder in a subject in need thereof comprising administering to the subject an effective amount of the compound according to any one of claims 1-2, or a salt thereof.

12. The method according to claim 11, wherein the renin mediated disorder is hypertension, congestive heart failure, cardiac hypertrophy, cardiac fibrosis, cardiomyopathy post-infarction, complications resulting from diabetes, such as nephropathy, vasculopathy and neuropathy, diseases of the coronary vessels, post-surgical hypertension, restenosis following angioplasty, raised intra-ocular pressure, glaucoma, abnormal vascular growth, hyperaldosteronism, anxiety states, or a cognitive disorder.

13. A method for the treatment of hypertension in a subject in need thereof comprising administering to the subject the compound according to any one of claims 1-2 in combination therapy with one or more additional agents, wherein each of said additional agents is independently selected from the group consisting of an α-blocker, a β-blocker, a calcium channel blocker, a diuretic, an angiotensin converting enzyme inhibitor, a dual angiotensin converting enzyme-neutral endopeptidase inhibitor, an angiotensin-receptor blocker, an aldosterone synthase inhibitor, an aldosterone-receptor antagonist, and an endothelin receptor antagonist.

14. The method according to claim 13, wherein: the α-blocker is selected from the group consisting of doxazosin, prazosin, tamsulosin, and terazosin; the β-blocker is selected from the group consisting of atenolol, bisoprol, metoprolol, acetutolol, esmolol, celiprolol, taliprolol, acebutolol, oxprenolol, pindolol, propanolol, bupranolol, penbutolol, mepindolol, carteolol, nadolol, and carvedilol, or pharmaceutically acceptable salts thereof; the calcium channel blocker is selected from the group consisting of dihydropyridines (DHPs) and non-DHPs, wherein the DHPs are selected from the group consisting of amlodipine, felodipine, ryosidine, isradipine, lacidipine, nicardipine, nifedipine, nigulpidine, nimodiphine, nisoldipine, nitrendipine, and nivaldipine and their pharmaceutically acceptable salts and the non-DHPs are selected from the group consisting of flunarizine, prenylamine, diltiazem, fendiline, gallopamil, mibefradil, anipamil, tiapamil, and verampimil, or pharmaceutically acceptable salts thereof; the diuretic is a thiazide derivative selected from the group consisting of an amiloride, chlorothiazide, hydrochlorothiazide, methylchlorothiazide, and chlorothalidon; the ACE inhibitor is selected from the group consisting of alacepril, benazepril, benazaprilat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril; the dual angiotensin converting enzyme-neutral endopeptidase inhibitor is selected from the group consisting of include omapatrilat, fasidotril, and fasidotrilat; the angiotensin-receptor blocker is selected from the group consisting of candesartan, eprosartan, irbesartan, losartan, olmesartan, tasosartan, telmisartan, and valsartan; the aldosterone synthase inhibitor is selected from the group consisting of anastrozole, fadrozole, and exemestane; the aldosterone-receptor antagonist is selected from the group consisting of spironolactone and eplerenone; and the endothelin antagonist is selected from the group consisting of bosentan, enrasentan, atrasentan, darusentan, sitaxentan, and tezosentan, or pharmaceutically acceptable salts thereof.

15. The method according to claim 14, wherein the compound and the additional agents are administered by sequential administration or simultaneous administration.

16-19. (canceled)
